SEQUENCE LISTING (369 sequences)

SEQ ID NO: 1 - protein, 13 aa, synthetic construct
  CROSSLNK 5..9: note = glutamic acid lysine amide
SRLEEELRKRLTE

SEQ ID NO: 2 - protein, 13 aa, synthetic construct
  CROSSLNK 5..9: note = aspartatic acid lysine amide
SRLEDELRKRLTE

SEQ ID NO: 3 - protein, 13 aa, synthetic construct
  CROSSLNK 5..8: note = glutamic acid lysine amide
SRLEEELKRRLTE

SEQ ID NO: 4 - protein, 13 aa, synthetic construct
  CROSSLNK 9..13: note = lysine glutamic acid amide
SRLEEELRKRLTE

SEQ ID NO: 5 - protein, 13 aa, synthetic construct
  DISULFID 5..9: note = cysteine cysteine disulfide
SRLECELRCRLTE

SEQ ID NO: 6 - protein, 13 aa, synthetic construct
  CROSSLNK 5..8: note = aspartic acid lysine amide
SRLEDELKRRLTE

SEQ ID NO: 7 - protein, 13 aa, synthetic construct
  CROSSLNK 9..13: note = lysine aspartic acid amide
SRLEEELRKRLTD

SEQ ID NO: 8 - protein, 13 aa, synthetic construct
  CROSSLNK 5..9: note = glutamic acid D-lysine amide
  SITE 9: note = D-lysine
SRLEEELRKRLTE

SEQ ID NO: 9 - protein, 14 aa, synthetic construct
  SITE 6..10: note = glutamic acid lysine amide
PSRLEEELRKRLTE

SEQ ID NO: 10 - protein, 14 aa, synthetic construct
  SITE 6..10: note = D-glutamic acid lysine amide
  SITE 6: note = D-glutamic acid
PSRLEEELRKRLTE

SEQ ID NO: 11 - protein, 14 aa, synthetic construct
  SITE 6..10: note = glutamic acid D-lysine amide
  SITE 10: note = D-lysine
PSRLEEELRKRLTE

SEQ ID NO: 12 - protein, 14 aa, synthetic construct
  SITE 6..10: note = lysine glutamic acid amide
PSRLEKELRERLTE

SEQ ID NO: 13 - protein, 14 aa, synthetic construct
  CROSSLNK 5..9: note = glutamic acid lysine amide
PSRLEEELKRRLTE

SEQ ID NO: 14 - protein, 14 aa, synthetic construct
  SITE 6..9: note = cysteine D-cysteine disulfide
  SITE 9: note = D-cysteine
PSRLECELCRRLTE

SEQ ID NO: 15 - protein, 14 aa, synthetic construct
  SITE 10..14: note = cysteine cysteine disulfide
PSRLEEELRCRLTC

SEQ ID NO: 16 - protein, 14 aa, synthetic construct
  SITE 6..10: note = cysteine cysteine disulfide
PSRLECELRCRLTE

SEQ ID NO: 17 - protein, 14 aa, synthetic construct
  SITE 11..14: note = cysteine cysteine disulfide
PSRLEEELRRCLTC

SEQ ID NO: 18 - protein, 14 aa, synthetic construct
  SITE 7..11: note = cysteine cysteine disulfide
PSRLEECLRRCLTE

SEQ ID NO: 19 - protein, 13 aa, synthetic construct
  SITE 9..13: note = cysteine cysteine disulfide
SRLEEELRCRLTC

SEQ ID NO: 20 - protein, 13 aa, synthetic construct
  DISULFID 5..9: note = cysteine cysteine disulfide
SRLECELRCRLTE

SEQ ID NO: 21 - protein, 13 aa, synthetic construct
  SITE 5..8: note = cysteine cysteine disulfide
SRLECELCRRLTE

SEQ ID NO: 22 - protein, 13 aa, synthetic construct
  SITE 10..13: note = cysteine cysteine disulfide
SRLEEELRRCLTC

SEQ ID NO: 23 - protein, 13 aa, synthetic construct
  SITE 6..10: note = cysteine cysteine disulfide
SRLEECLRRCLTE

SEQ ID NO: 24 - protein, 13 aa, synthetic construct
  CROSSLNK 5..9: note = D-glutamic acid D-lysine amide
  SITE 5: note = D-glutamic acid
  SITE 9: note = D-lysine
SRLEEELRKRLTE

SEQ ID NO: 25 - protein, 13 aa, synthetic construct
  CROSSLNK 5..9: note = D-lysine glutamic acid amide
  SITE 5: note = D-lysine
SRLEKELRERLTE

SEQ ID NO: 26 - protein, 13 aa, synthetic construct
  CROSSLNK 5..9: note = lysine D-glutamic acid amide
  SITE 9: note = D-glutamic acid
SRLEKELRERLTE

SEQ ID NO: 27 - protein, 13 aa, synthetic construct
  CROSSLNK 5..9: note = D-lysine D-glutamic acid amide
  SITE 5: note = D-lysine
  SITE 9: note = D-glutamic acid
SRLEKELRERLTE

SEQ ID NO: 28 - protein, 14 aa, synthetic construct
  SITE 6..10: note = D-glutamic acid D-lysine amide
  SITE 6: note = D-glutamic acid
  SITE 10: note = D-lysine
PSRLEEELRKRLTE

SEQ ID NO: 29 - protein, 14 aa, synthetic construct
  SITE 6..10: note = D-lysine glutamic acid amide
  SITE 6: note = D-lysine
PSRLEKELRERLTE

SEQ ID NO: 30 - protein, 14 aa, synthetic construct
  SITE 6..10: note = lysine D-glutamic amide
  SITE 10: note = D-glutamic acid
PSRLEKELRERLTE

SEQ ID NO: 31 - protein, 14 aa, synthetic construct
  SITE 6..10: note = D-lysine D-glutamic acid amide
  SITE 6: note = D-lysine
  SITE 10: note = D-glutamic acid
PSRLEKELRERLTE

SEQ ID NO: 32 - protein, 13 aa, synthetic construct
  CROSSLNK 5..9: note = D-lysine aspartic acid amide
  SITE 5: note = D-lysine
SRLEKELRDRLTE

SEQ ID NO: 33 - protein, 13 aa, synthetic construct
  CROSSLNK 5..9: note = D-aspartic acid D-lysine amide
  SITE 5: note = D-aspartic acid
  SITE 9: note = D-lysine
SRLEDELRKRLTE

SEQ ID NO: 34 - protein, 13 aa, synthetic construct
  CROSSLNK 5..9: note = D-aspartic acid lysine amide
  SITE 5: note = D-aspartic acid
SRLEDELRKRLTE

SEQ ID NO: 35 - protein, 13 aa, synthetic construct
  CROSSLNK 5..9: note = aspartic acid D-lysine amide
  SITE 9: note = D-lysine
SRLEDELRKRLTE

SEQ ID NO: 36 - protein, 13 aa, synthetic construct
  CROSSLNK 5..9: note = lysine D-aspartic acid amide
  SITE 9: note = D-aspartic acid
SRLEKELRDRLTE

SEQ ID NO: 37 - protein, 13 aa, synthetic construct
  CROSSLNK 5..9: note = D-lysine D-aspartic acid amide
  SITE 5: note = D-lysine
  SITE 9: note = D-aspartic acid
SRLEKELRDRLTE

SEQ ID NO: 38 - protein, 14 aa, synthetic construct
  SITE 6..10: note = D-lysine aspartic acid amide
  SITE 6: note = D-lysine
PSRLEKELRDRLTE

SEQ ID NO: 39 - protein, 14 aa, synthetic construct
  SITE 6..10: note = D-aspartic acid D-lysine amide
  SITE 6: note = D-aspartic acid
  SITE 10: note = D-lysine
PSRLEDELRKRLTE

SEQ ID NO: 40 - protein, 14 aa, synthetic construct
  SITE 6..10: note = D-aspartic acid lysine amide
  SITE 6: note = D-aspartic acid
PSRLEDELRKRLTE

SEQ ID NO: 41 - protein, 14 aa, synthetic construct
  SITE 6..10: note = aspartic acid D-lysine amide
  SITE 10: note = D-lysine
PSRLEDELRKRLTE

SEQ ID NO: 42 - protein, 14 aa, synthetic construct
  SITE 6..10: note = lysine D-aspartic acid amide
  SITE 10: note = D-aspartic acid
PSRLEKELRDRLTE

SEQ ID NO: 43 - protein, 14 aa, synthetic construct
  SITE 6..10: note = D-lysine D-aspartic acid amide
  SITE 6: note = D-lysine
  SITE 10: note = D-spartic acid
PSRLEKELRDRLTE

SEQ ID NO: 44 - protein, 13 aa, synthetic construct
  SITE 5..8: note = D-glutamic acid lysine amide
  SITE 5: note = D-glutamic acid
SRLEEELKRRLTE

SEQ ID NO: 45 - protein, 13 aa, synthetic construct
  SITE 5..8: note = glutamic acid D-lysine amide
  SITE 8: note = D-lysine
SRLEEELKRRLTE

SEQ ID NO: 46 - protein, 13 aa, synthetic construct
  SITE 5..8: note = D-glutamic acid D-lysine amide
  SITE 5: note = D-glutamic acid
  SITE 8: note = D-lysine
SRLEEELKRRLTE

SEQ ID NO: 47 - protein, 13 aa, synthetic construct
  SITE 5..8: note = lysine glutamic acid amide
SRLEKELERRLTE

SEQ ID NO: 48 - protein, 13 aa, synthetic construct
  SITE 5..8: note = lysine D-glutamic acid amide
  SITE 8: note = D-glutamic acid
SRLEKELERRLTE

SEQ ID NO: 49 - protein, 13 aa, synthetic construct
  SITE 5..8: note = D-lysine D-glutamic acid amide
  SITE 5: note = D-lysine
  SITE 8: note = D-glutamic acid
SRLEKELERRLTE

SEQ ID NO: 50 - protein, 13 aa, synthetic construct
  SITE 5..8: note = aspartic acid lysine amide
SRLEDELKRRLTE

SEQ ID NO: 51 - protein, 13 aa, synthetic construct
  SITE 5..8: note = aspartic acid D-lysine amide
  SITE 8: note = D-lysine
SRLEDELKRRLTE

SEQ ID NO: 52 - protein, 13 aa, synthetic construct
  SITE 5..8: note = lysine aspartic acid amide
SRLEKELDRRLTE

SEQ ID NO: 53 - protein, 13 aa, synthetic construct
  SITE 5..8: note = lysine D-aspartic acid amide
  SITE 8: note = D-aspartic acid
SRLEKELDRRLTE

SEQ ID NO: 54 - protein, 13 aa, synthetic construct
  SITE 5..8: note = D-aspartic acid lysine amide
  SITE 5: note = D-aspartic acid
SRLEDELKRRLTE

SEQ ID NO: 55 - protein, 13 aa, synthetic construct
  SITE 5..8: note = D-aspartic acid D-lysine amide
  SITE 5: note = D-aspartic acid
  SITE 8: note = D-lysine
SRLEDELKRRLTE

SEQ ID NO: 56 - protein, 13 aa, synthetic construct
  SITE 5..8: note = D-lysine D-aspartic acid amide
  SITE 5: note = D-lysine
  SITE 8: note = D-aspartic acid
SRLEKELDRRLTE

SEQ ID NO: 57 - protein, 13 aa, synthetic construct
  SITE 5..8: note = D-lysine aspartic acid amide
  SITE 5: note = D-lysine
SRLEKELDRRLTE

SEQ ID NO: 58 - protein, 14 aa, synthetic construct
  SITE 6..9: note = glutamic acid D-lysine amide
  SITE 9: note = D-lysine
PSRLEEELKRRLTE

SEQ ID NO: 59 - protein, 14 aa, synthetic construct
  SITE 6..9: note = D-glutamic acid D-lysine amide
  SITE 6: note = D-glutamic acid
  SITE 9: note = D-lysine
PSRLEEELKRRLTE

SEQ ID NO: 60 - protein, 14 aa, synthetic construct
  SITE 6..9: note = lysine glutamic acid amide
PSRLEKELERRLTE

SEQ ID NO: 61 - protein, 14 aa, synthetic construct
  SITE 6..9: note = lysine D-glutamic acid amide
  SITE 9: note = D-glutamic acid
PSRLEKELERRLTE

SEQ ID NO: 62 - protein, 14 aa, synthetic construct
  SITE 6..9: note = D-lysine D-glutamic acid amide
  SITE 6: note = D-lysine
  SITE 9: note = D-glutamic acid
PSRLEKELERRLTE

SEQ ID NO: 63 - protein, 14 aa, synthetic construct
  SITE 6..9: note = aspartic acid lysine amide
PSRLEDELKRRLTE

SEQ ID NO: 64 - protein, 14 aa, synthetic construct
  SITE 6..9: note = aspartic acid D-lysine amide
  SITE 9: note = D-lysine
PSRLEDELKRRLTE

SEQ ID NO: 65 - protein, 14 aa, synthetic construct
  SITE 6..9: note = lysine aspartic acid amide
PSRLEKELDRRLTE

SEQ ID NO: 66 - protein, 14 aa, synthetic construct
  SITE 6..9: note = lysine D-aspartic acid amide
  SITE 9: note = D-aspartic acid
PSRLEKELDRRLTE

SEQ ID NO: 67 - protein, 14 aa, synthetic construct
  SITE 6..9: note = D-aspartic acid lysine amide
  SITE 6: note = D-aspartic acid
PSRLEDELKRRLTE

SEQ ID NO: 68 - protein, 14 aa, synthetic construct
  SITE 6..9: note = D-aspartic acid D-lysine amide
  SITE 6: note = D-aspartic acid
  SITE 9: note = D-lysine
PSRLEDELKRRLTE

SEQ ID NO: 69 - protein, 14 aa, synthetic construct
  SITE 6..9: note = D-lysine D-aspartic acid amide
  SITE 6: note = D-lysine
  SITE 9: note = D-aspartic acid
PSRLEKELDRRLTE

SEQ ID NO: 70 - protein, 14 aa, synthetic construct
  SITE 6..9: note = D-lysine aspartic acid amide
  SITE 6: note = D-lysine
PSRLEKELDRRLTE

SEQ ID NO: 71 - protein, 13 aa, synthetic construct
  SITE 6..9: note = aspartic acid lysine amide
SRLEEDLRKRLTE

SEQ ID NO: 72 - protein, 13 aa, synthetic construct
  SITE 6..9: note = lysine aspartic acid amide
SRLEEKLRDRLTE

SEQ ID NO: 73 - protein, 13 aa, synthetic construct
  SITE 6..9: note = D-aspartic acid lysine amide
  SITE 6: note = D-aspartic acid
SRLEEDLRKRLTE

SEQ ID NO: 74 - protein, 13 aa, synthetic construct
  SITE 6..9: note = lysine D-aspartic acid amide
  SITE 9: note = D-aspartic acid
SRLEEKLRDRLTE

SEQ ID NO: 75 - protein, 13 aa, synthetic construct
  SITE 6..9: note = aspartic acid D-lysine amide
  SITE 9: note = D-lysine
SRLEEDLRKRLTE

SEQ ID NO: 76 - protein, 13 aa, synthetic construct
  SITE 6..9: note = D-lysine aspartic acid amide
  SITE 6: note = D-lysine
SRLEEKLRDRLTE

SEQ ID NO: 77 - protein, 13 aa, synthetic construct
  SITE 6..9: note = D-lysine D-aspartic acid amide
  SITE 6: note = D-lysine
  SITE 9: note = D-aspartic acid
SRLEEKLRDRLTE

SEQ ID NO: 78 - protein, 13 aa, synthetic construct
  SITE 6..9: note = D-aspartic acid D-lysine amide
  SITE 6: note = D-aspartic acid
  SITE 9: note = D-lysine
SRLEEDLRKRLTE

SEQ ID NO: 79 - protein, 14 aa, synthetic construct
  SITE 7..10: note = aspartic acid lysine amide
PSRLEEDLRKRLTE

SEQ ID NO: 80 - protein, 14 aa, synthetic construct
  SITE 7..10: note = lysine aspartic acid amide
PSRLEEKLRDRLTE

SEQ ID NO: 81 - protein, 14 aa, synthetic construct
  SITE 7..10: note = D-aspartic acid lysine amide
  SITE 7: note = D-aspartic acid
PSRLEEDLRKRLTE

SEQ ID NO: 82 - protein, 14 aa, synthetic construct
  SITE 7..10: note = lysine D-aspartic acid amide
  SITE 10: note = D-aspartic acid
PSRLEEKLRDRLTE

SEQ ID NO: 83 - protein, 14 aa, synthetic construct
  SITE 7..10: note = aspartic acid D-lysine amide
  SITE 10: note = D-lysine
PSRLEEDLRKRLTE

SEQ ID NO: 84 - protein, 14 aa, synthetic construct
  SITE 7..10: note = D-lysine aspartic acid amide
  SITE 7: note = D-lysine
PSRLEEKLRDRLTE

SEQ ID NO: 85 - protein, 14 aa, synthetic construct
  SITE 7..10: note = D-lysine D-aspartic acid amide
  SITE 7: note = D-lysine
  SITE 10: note = D-aspartic acid
PSRLEEKLRDRLTE

SEQ ID NO: 86 - protein, 14 aa, synthetic construct
  SITE 7..10: note = D-aspartic acid D-lysine amide
  SITE 7: note = D-aspartic acid
  SITE 10: note = D-lysine
PSRLEEDLRKRLTE

SEQ ID NO: 87 - protein, 13 aa, synthetic construct
  SITE 5..8: note = cysteine cysteine disulfide
SRLECELCRRLTE

SEQ ID NO: 88 - protein, 13 aa, synthetic construct
  SITE 10..13: note = cysteine cysteine disulfide
SRLEEELRRCLTC

SEQ ID NO: 89 - protein, 13 aa, synthetic construct
  SITE 9..12: note = cysteine cysteine disulfide
SRLEEELRCRLCE

SEQ ID NO: 90 - protein, 13 aa, synthetic construct
  SITE 6..9: note = cysteine cysteine disulfide
SRLEECLRCRLTE

SEQ ID NO: 91 - protein, 14 aa, synthetic construct
  SITE 6..9: note = cysteine cysteine disulfide
PSRLECELCRRLTE

SEQ ID NO: 92 - protein, 14 aa, synthetic construct
  SITE 11..14: note = cysteine cysteine disulfide
PSRLEEELRRCLTC

SEQ ID NO: 93 - protein, 14 aa, synthetic construct
  SITE 10..13: note = cysteine cysteine disulfide
PSRLEEELRCRLCE

SEQ ID NO: 94 - protein, 14 aa, synthetic construct
  SITE 7..10: note = cysteine cysteine disulfide
PSRLEECLRCRLTE

SEQ ID NO: 95 - protein, 13 aa, synthetic construct
  SITE 5..8: note = D-cysteine D-cysteine disulfide
  SITE 5: note = D-cysteine
  SITE 8: note = D-cysteine
SRLECELCRRLTE

SEQ ID NO: 96 - protein, 13 aa, synthetic construct
  SITE 5..8: note = D-cysteine cysteine disulfide
  SITE 5: note = D-cysteine
SRLECELCRRLTE

SEQ ID NO: 97 - protein, 13 aa, synthetic construct
  SITE 5..8: note = homocysteine cysteine disulfide
  SITE 5: note = homocysteine
SRLEXELCRRLTE

SEQ ID NO: 98 - protein, 13 aa, synthetic construct
  SITE 5..8: note = homocysteine homocysteine disulfide
  SITE 5: note = homocysteine
  SITE 8: note = homocysteine
SRLEXELXRRLTE

SEQ ID NO: 99 - protein, 13 aa, synthetic construct
  SITE 5..8: note = cysteine homocysteine disulfide
  SITE 8: note = homocysteine
SRLECELXRRLTE

SEQ ID NO: 100 - protein, 13 aa, synthetic construct
  SITE 5..8: note = homocysteine D-cysteine disulfide
  SITE 5: note = homocysteine
  SITE 8: note = D-cysteine
SRLEXELCRRLTE

SEQ ID NO: 101 - protein, 13 aa, synthetic construct
  SITE 5..8: note = D-cysteine homocysteine disulfide
  SITE 5: note = D-cysteine
  SITE 8: note = homocysteine
SRLECELXRRLTE

SEQ ID NO: 102 - protein, 13 aa, synthetic construct
  SITE 5..8: note = homocysteine D-homocysteine disulfide
  SITE 5: note = homocysteine
  SITE 8: note = D-homocysteine
SRLEXELXRRLTE

SEQ ID NO: 103 - protein, 13 aa, synthetic construct
  SITE 5..8: note = D-homocysteine D-homocysteine disulfide
  SITE 5: note = D-homocysteine
  SITE 8: note = D-homocysteine
SRLEXELXRRLTE

SEQ ID NO: 104 - protein, 13 aa, synthetic construct
  SITE 5..8: note = cysteine D-homocysteine disulfide
  SITE 8: note = D-homocysteine
SRLECELXRRLTE

SEQ ID NO: 105 - protein, 13 aa, synthetic construct
  SITE 5..8: note = D-cysteine D-homocysteine disulfide
  SITE 5: note = D-cysteine
  SITE 8: note = D-homocysteine
SRLECELXRRLTE

SEQ ID NO: 106 - protein, 13 aa, synthetic construct
  SITE 5..8: note = D-homocysteine cysteine disulfide
  SITE 5: note = D-homocysteine
SRLEXELCRRLTE

SEQ ID NO: 107 - protein, 13 aa, synthetic construct
  SITE 5..8: note = D-homocysteine D-cysteine disulfide
  SITE 5: note = D-homocysteine
  SITE 8: note = D-cysteine
SRLEXELCRRLTE

SEQ ID NO: 108 - protein, 14 aa, synthetic construct
  SITE 6..9: note = D-cysteine D-cysteine disulfide
  SITE 6: note = D-cysteine
  SITE 9: note = D-cysteine
PSRLECELCRRLTE

SEQ ID NO: 109 - protein, 14 aa, synthetic construct
  SITE 6..9: note = D-cysteine cysteine disulfide
  SITE 6: note = D-cysteine
PSRLECELCRRLTE

SEQ ID NO: 110 - protein, 14 aa, synthetic construct
  SITE 6..9: note = homocysteine cysteine disulfide
  SITE 6: note = homocysteine
PSRLEXELCRRLTE

SEQ ID NO: 111 - protein, 14 aa, synthetic construct
  SITE 6..9: note = homocysteine homocysteine disulfide
  SITE 6: note = homocysteine
  SITE 9: note = homocysteine
PSRLEXELXRRLTE

SEQ ID NO: 112 - protein, 14 aa, synthetic construct
  SITE 6..9: note = cysteine homocysteine disulfide
  SITE 9: note = homocysteine
PSRLECELXRRLTE

SEQ ID NO: 113 - protein, 14 aa, synthetic construct
  SITE 6..9: note = homocysteine D-cysteine disulfide
  SITE 6: note = homocysteine
  SITE 9: note = D-cysteine
PSRLEXELCRRLTE

SEQ ID NO: 114 - protein, 14 aa, synthetic construct
  SITE 6..9: note = D-cysteine homocysteine disulfide
  SITE 6: note = D-cysteine
  SITE 9: note = homocysteine
PSRLECELXRRLTE

SEQ ID NO: 115 - protein, 14 aa, synthetic construct
  SITE 6..9: note = homocysteine D-homocysteine disulfide
  SITE 6: note = homocysteine
  SITE 9: note = D-homocysteine
PSRLEXELXRRLTE

SEQ ID NO: 116 - protein, 14 aa, synthetic construct
  SITE 6..9: note = D-homocysteine D-homocysteine disulfide
  SITE 6: note = D-homocysteine
  SITE 9: note = D-homocysteine
PSRLEXELXRRLTE

SEQ ID NO: 117 - protein, 14 aa, synthetic construct
  SITE 6..9: note = cysteine D-homocysteine disulfide
  SITE 9: note = D-homocysteine
PSRLECELXRRLTE

SEQ ID NO: 118 - protein, 14 aa, synthetic construct
  SITE 6..9: note = D-cysteine D-homocysteine disulfide
  SITE 6: note = D-cysteine
  SITE 9: note = D-homocysteine
PSRLECELXRRLTE

SEQ ID NO: 119 - protein, 14 aa, synthetic construct
  SITE 6..9: note = D-homocysteine cysteine disulfide
  SITE 6: note = D-homocysteine
PSRLEXELCRRLTE

SEQ ID NO: 120 - protein, 14 aa, synthetic construct
  SITE 6..9: note = D-homocysteine D-cysteine disulfide
  SITE 6: note = D-homocysteine
  SITE 9: note = D-cysteine
PSRLEXELCRRLTE

SEQ ID NO: 121 - protein, 13 aa, synthetic construct
  SITE 10..13: note = cysteine D-cysteine disulfide
  SITE 13: note = D-cysteine
SRLEEELRRCLTC

SEQ ID NO: 122 - protein, 13 aa, synthetic construct
  SITE 10..13: note = D-cysteine cysteine disulfide
  SITE 10: note = D-cysteine
SRLEEELRRCLTC

SEQ ID NO: 123 - protein, 13 aa, synthetic construct
  SITE 10..13: note = D-cysteine D-cysteine disulfide
  SITE 10: note = D-cysteine
  SITE 13: note = D-cysteine
SRLEEELRRCLTC

SEQ ID NO: 124 - protein, 13 aa, synthetic construct
  SITE 10..13: note = homocysteine cysteine disulfide
  SITE 10: note = homocysteine
SRLEEELRRXLTC

SEQ ID NO: 125 - protein, 13 aa, synthetic construct
  SITE 10..13: note = homocysteine D-cysteine disulfide
  SITE 10: note = homocysteine
  SITE 13: note = D-cysteine
SRLEEELRRXLTC

SEQ ID NO: 126 - protein, 13 aa, synthetic construct
  SITE 10..13: note = homocysteine homocysteine disulfide
  SITE 10: note = homocysteine
  SITE 13: note = homocysteine
SRLEEELRRXLTX

SEQ ID NO: 127 - protein, 13 aa, synthetic construct
  SITE 10..13: note = cysteine homocysteine disulfide
  SITE 13: note = homocysteine
SRLEEELRRCLTX

SEQ ID NO: 128 - protein, 13 aa, synthetic construct
  SITE 10..13: note = D-cysteine homocysteine disulfide
  SITE 10: note = D-cysteine
  SITE 13: note = homocysteine
SRLEEELRRCLTX

SEQ ID NO: 129 - protein, 13 aa, synthetic construct
  SITE 10..13: note = cysteine homocysteine disulfide
  SITE 13: note = homocysteine
SRLEEELRRCLTX

SEQ ID NO: 130 - protein, 13 aa, synthetic construct
  SITE 10..13: note = D-homocysteine cysteine disulfide
  SITE 10: note = D-homocysteine
SRLEEELRRXLTC

SEQ ID NO: 131 - protein, 13 aa, synthetic construct
  SITE 10..13: note = D-homocysteine D-cysteine disulfide
  SITE 10: note = D-homocysteine
  SITE 13: note = D-cysteine
SRLEEELRRXLTC

SEQ ID NO: 132 - protein, 13 aa, synthetic construct
  SITE 10..13: note = D-homocysteine homocysteine disulfide
  SITE 10: note = D-homocysteine
  SITE 13: note = homocysteine
SRLEEELRRXLTX

SEQ ID NO: 133 - protein, 13 aa, synthetic construct
  SITE 10..13: note = cysteine D-homocysteine disulfide
  SITE 13: note = D-homocysteine
SRLEEELRRCLTX

SEQ ID NO: 134 - protein, 13 aa, synthetic construct
  SITE 10..13: note = D-cysteine D-homocysteine disulfide
  SITE 10: note = D-cysteine
  SITE 13: note = D-homocysteine
SRLEEELRRCLTX

SEQ ID NO: 135 - protein, 13 aa, synthetic construct
  SITE 10..13: note = cysteine D-homocysteine disulfide
  SITE 13: note = D-homocysteine
SRLEEELRRCLTX

SEQ ID NO: 136 - protein, 13 aa, synthetic construct
  SITE 10..13: note = penicillamine penicillamine disulfide
  SITE 10: note = penicillamine
  SITE 13: note = penicillamine
SRLEEELRRXLTX

SEQ ID NO: 137 - protein, 13 aa, synthetic construct
  SITE 10..13: note = penicillamine D-penicillamine disulfide
  SITE 10: note = penicillamine
  SITE 13: note = D-penicillamine
SRLEEELRRXLTX

SEQ ID NO: 138 - protein, 13 aa, synthetic construct
  SITE 10..13: note = D-penicillamine penicillamine disulfide
  SITE 10: note = D-penicillamine
  SITE 13: note = penicillamine
SRLEEELRRXLTX

SEQ ID NO: 139 - protein, 13 aa, synthetic construct
  SITE 10..13: note = D-penicillamine D-penicillamine disulfide
  SITE 10: note = D-penicillamine
  SITE 13: note = D-penicillamine
SRLEEELRRXLTX

SEQ ID NO: 140 - protein, 13 aa, synthetic construct
  SITE 9..12: note = D-cysteine cysteine disulfide
  SITE 9: note = D-cysteine
SRLEEELRCRLCE

SEQ ID NO: 141 - protein, 13 aa, synthetic construct
  SITE 9..12: note = D-cysteine D-cysteine disulfide
  SITE 9: note = D-cysteine
  SITE 12: note = D-cysteine
SRLEEELRCRLCE

SEQ ID NO: 142 - protein, 13 aa, synthetic construct
  SITE 9..12: note = cysteine D-cysteine disulfide
  SITE 12: note = D-cysteine
SRLEEELRCRLCE

SEQ ID NO: 143 - protein, 13 aa, synthetic construct
  SITE 9..12: note = homocysteine cysteine disulfide
  SITE 9: note = homocysteine
SRLEEELRXRLCE

SEQ ID NO: 144 - protein, 13 aa, synthetic construct
  SITE 9..12: note = homocysteine D-cysteine disulfide
  SITE 9: note = homocysteine
  SITE 12: note = D-cysteine
SRLEEELRXRLCE

SEQ ID NO: 145 - protein, 13 aa, synthetic construct
  SITE 9..12: note = cysteine homocysteine disulfide
  SITE 12: note = homocysteine
SRLEEELRCRLXE

SEQ ID NO: 146 - protein, 13 aa, synthetic construct
  SITE 9..12: note = homocysteine homocysteine disulfide
  SITE 9: note = homocysteine
  SITE 12: note = homocysteine
SRLEEELRXRLXE

SEQ ID NO: 147 - protein, 13 aa, synthetic construct
  SITE 9..12: note = homocysteine D-homocysteine disulfide
  SITE 9: note = homocysteine
  SITE 12: note = D-homocysteine
SRLEEELRXRLXE

SEQ ID NO: 148 - protein, 13 aa, synthetic construct
  SITE 9..12: note = D-homocysteine homocysteine disulfide
  SITE 9: note = D-homocysteine
  SITE 12: note = homocysteine
SRLEEELRXRLXE

SEQ ID NO: 149 - protein, 14 aa, synthetic construct
  SITE 11..14: note = cysteine D-cysteine disulfide
  SITE 14: note = D-cysteine
PSRLEEELRRCLTC

SEQ ID NO: 150 - protein, 14 aa, synthetic construct
  SITE 11..14: note = D-cysteine cysteine disulfide
  SITE 11: note = D-cysteine
PSRLEEELRRCLTC

SEQ ID NO: 151 - protein, 14 aa, synthetic construct
  SITE 11..14: note = D-cysteine D-cysteine disulfide
  SITE 11: note = D-cysteine
  SITE 14: note = D-cysteine
PSRLEEELRRCLTC

SEQ ID NO: 152 - protein, 14 aa, synthetic construct
  SITE 11..14: note = homocysteine cysteine disulfide
  SITE 11: note = homocysteine
PSRLEEELRRXLTC

SEQ ID NO: 153 - protein, 14 aa, synthetic construct
  SITE 11..14: note = homocysteine D-cysteine disulfide
  SITE 11: note = homocysteine
  SITE 14: note = D-cysteine
PSRLEEELRRXLTC

SEQ ID NO: 154 - protein, 14 aa, synthetic construct
  SITE 11..14: note = homocysteine homocysteine disulfide
  SITE 11: note = homocysteine
  SITE 14: note = homocysteine
PSRLEEELRRXLTX

SEQ ID NO: 155 - protein, 14 aa, synthetic construct
  SITE 11..14: note = cysteine homocysteine disulfide
  SITE 14: note = homocysteine
PSRLEEELRRCLTX

SEQ ID NO: 156 - protein, 14 aa, synthetic construct
  SITE 11..14: note = D-cysteine homocysteine disulfide
  SITE 11: note = D-cysteine
  SITE 14: note = homocysteine
PSRLEEELRRCLTX

SEQ ID NO: 157 - protein, 14 aa, synthetic construct
  SITE 11..14: note = cysteine homocysteine disulfide
  SITE 14: note = homocysteine
PSRLEEELRRCLTX

SEQ ID NO: 158 - protein, 14 aa, synthetic construct
  SITE 11..14: note = D-homocysteine cysteine disulfide
  SITE 11: note = D-homocysteine
PSRLEEELRRXLTC

SEQ ID NO: 159 - protein, 14 aa, synthetic construct
  SITE 11..14: note = D-homocysteine D-cysteine disulfide
  SITE 11: note = D-homocysteine
  SITE 14: note = D-cysteine
PSRLEEELRRXLTC

SEQ ID NO: 160 - protein, 14 aa, synthetic construct
  SITE 11..14: note = D-homocysteine homocysteine disulfide
  SITE 11: note = D-homocysteine
  SITE 14: note = homocysteine
PSRLEEELRRXLTX

SEQ ID NO: 161 - protein, 14 aa, synthetic construct
  SITE 11..14: note = cysteine D-homocysteine disulfide
  SITE 14: note = D-homocysteine
PSRLEEELRRCLTX

SEQ ID NO: 162 - protein, 14 aa, synthetic construct
  SITE 11..14: note = D-cysteine D-homocysteine disulfide
  SITE 11: note = D-cysteine
  SITE 14: note = D-homocysteine
PSRLEEELRRCLTX

SEQ ID NO: 163 - protein, 14 aa, synthetic construct
  SITE 11..14: note = cysteine D-homocysteine disulfide
  SITE 14: note = D-homocysteine
PSRLEEELRRCLTX

SEQ ID NO: 164 - protein, 14 aa, synthetic construct
  SITE 11..14: note = penicillamine penicillamine disulfide
  SITE 11: note = penicillamine
  SITE 14: note = penicillamine
PSRLEEELRRXLTX

SEQ ID NO: 165 - protein, 14 aa, synthetic construct
  SITE 11..14: note = penicillamine D-penicillamine disulfide
  SITE 11: note = penicillamine
  SITE 14: note = D-penicillamine
PSRLEEELRRXLTX

SEQ ID NO: 166 - protein, 14 aa, synthetic construct
  SITE 11..14: note = D-penicillamine penicillamine diulfide
  SITE 11: note = D-penicillamine
  SITE 14: note = penicillamine
PSRLEEELRRXLTX

SEQ ID NO: 167 - protein, 14 aa, synthetic construct
  SITE 11..14: note = D-penicillamine D-penicillamine disulfide
  SITE 11: note = D-penicillamine
  SITE 14: note = D-penicillamine
PSRLEEELRRXLTX

SEQ ID NO: 168 - protein, 14 aa, synthetic construct
  SITE 10..13: note = D-cysteine cysteine disulfide
  SITE 10: note = D-cysteine
PSRLEEELRCRLCE

SEQ ID NO: 169 - protein, 14 aa, synthetic construct
  SITE 10..13: note = D-cysteine D-cysteine disulfide
  SITE 10: note = D-cysteine
  SITE 13: note = D-cysteine
PSRLEEELRCRLCE

SEQ ID NO: 170 - protein, 14 aa, synthetic construct
  SITE 10..13: note = cysteine D-cysteine disulfide
  SITE 13: note = D-cysteine
PSRLEEELRCRLCE

SEQ ID NO: 171 - protein, 14 aa, synthetic construct
  SITE 10..13: note = homocysteine cysteine disulfide
  SITE 10: note = homocysteine
PSRLEEELRXRLCE

SEQ ID NO: 172 - protein, 14 aa, synthetic construct
  SITE 10..13: note = homocysteine D-cysteine disulfide
  SITE 10: note = homocysteine
  SITE 13: note = D-cysteine
PSRLEEELRXRLCE

SEQ ID NO: 173 - protein, 14 aa, synthetic construct
  SITE 10..13: note = cysteine homocysteine disulfide
  SITE 13: note = homocysteine
PSRLEEELRCRLXE

SEQ ID NO: 174 - protein, 14 aa, synthetic construct
  SITE 10..13: note = homocysteine homocysteine disulfide
  SITE 10: note = homocysteine
  SITE 13: note = homocysteine
PSRLEEELRXRLXE

SEQ ID NO: 175 - protein, 14 aa, synthetic construct
  SITE 10..13: note = homocysteine D-homocysteine disulfide
  SITE 10: note = homocysteine
  SITE 13: note = D-homocysteine
PSRLEEELRXRLXE

SEQ ID NO: 176 - protein, 14 aa, synthetic construct
  SITE 10..13: note = D-homocysteine homocysteine disulfide
  SITE 10: note = D-homocysteine
  SITE 13: note = homocysteine
PSRLEEELRXRLXE

SEQ ID NO: 177 - protein, 13 aa, synthetic construct
  SITE 9..12: note = lysine aspartic acid amide
SRLEEELRKRLDE

SEQ ID NO: 178 - protein, 13 aa, synthetic construct
  SITE 9..12: note = aspartic acid lysine amide
SRLEEELRDRLKE

SEQ ID NO: 179 - protein, 13 aa, synthetic construct
  SITE 9..12: note = lysine glutamic acid amide
SRLEEELRKRLEE

SEQ ID NO: 180 - protein, 13 aa, synthetic construct
  SITE 9..12: note = glutamic acid lysine amide
SRLEEELRERLKE

SEQ ID NO: 181 - protein, 14 aa, synthetic construct
  SITE 10..13: note = lysine aspartic acid amide
PSRLEEELRKRLDE

SEQ ID NO: 182 - protein, 14 aa, synthetic construct
  SITE 10..13: note = aspartic acid lysine amide
PSRLEEELRDRLKE

SEQ ID NO: 183 - protein, 14 aa, synthetic construct
  SITE 10..13: note = lysine glutamic acid amide
PSRLEEELRKRLEE

SEQ ID NO: 184 - protein, 14 aa, synthetic construct
  SITE 10..13: note = glutamic acid lysine amide
PSRLEEELRERLKE

SEQ ID NO: 185 - protein, 14 aa, synthetic construct
  SITE 10..14: note = cysteine cysteine disulfide
PSRLEEELRCRLTC

SEQ ID NO: 186 - protein, 14 aa, synthetic construct
  SITE 6..10: note = cysteine cysteine disulfide
PSRLECELRCRLTE

SEQ ID NO: 187 - protein, 14 aa, synthetic construct
  SITE 11..14: note = cysteine cysteine disulfide
PSRLEEELRRCLTC

SEQ ID NO: 188 - protein, 14 aa, synthetic construct
  SITE 7..11: note = cysteine cysteine disulfide
PSRLEECLRRCLTE

SEQ ID NO: 189 - protein, 14 aa, synthetic construct
  CROSSLNK 5..9: note = D-glutamic acid D-lysine amide
  SITE 5: note = D-glutamic acid
  SITE 9: note = D-lysine
SRLEEELRKRLTEP

SEQ ID NO: 190 - protein, 14 aa, synthetic construct
  CROSSLNK 5..9: note = D-lysine glutamic acid amide
  SITE 5: note = D-lysine
SRLEKELRERLTEP

SEQ ID NO: 191 - protein, 14 aa, synthetic construct
  CROSSLNK 5..9: note = lysine D-glutamic acid amide
  SITE 9: note = D-glutamic acid
SRLEKELRERLTEP

SEQ ID NO: 192 - protein, 14 aa, synthetic construct
  CROSSLNK 5..9: note = D-lysine D-glutamic acid amide
  SITE 5: note = D-lysine
  SITE 9: note = D-glutamic acid
SRLEKELRERLTEP

SEQ ID NO: 193 - protein, 15 aa, synthetic construct
  SITE 6..10: note = D-glutamic acid D-lysine amide
  SITE 6: note = D-glutamic acid
  SITE 10: note = D-lysine
PSRLEEELRKRLTEP

SEQ ID NO: 194 - protein, 15 aa, synthetic construct
  SITE 6..10: note = D-lysine glutamic acid amide
  SITE 6: note = D-lysine
PSRLEKELRERLTEP

SEQ ID NO: 195 - protein, 15 aa, synthetic construct
  SITE 6..10: note = lysine D-glutamic acid amide
  SITE 10: note = D-glutamic acid
PSRLEKELRERLTEP

SEQ ID NO: 196 - protein, 15 aa, synthetic construct
  SITE 6..10: note = D-lysine D-glutamic acid amide
  SITE 6: note = D-lysine
  SITE 10: note = D-glutamic acid
PSRLEKELRERLTEP

SEQ ID NO: 197 - protein, 14 aa, synthetic construct
  CROSSLNK 5..9: note = D-lysine aspartic acid amide
  SITE 5: note = D-lysine
SRLEKELRDRLTEP

SEQ ID NO: 198 - protein, 14 aa, synthetic construct
  CROSSLNK 5..9: note = D-aspartic acid D-lysine amide
  SITE 5: note = D-aspartic acid
  SITE 9: note = D-lysine
SRLEDELRKRLTEP

SEQ ID NO: 199 - protein, 14 aa, synthetic construct
  CROSSLNK 5..9: note = D-aspartic acid lysine amide
  SITE 5: note = D-aspartic acid
SRLEDELRKRLTEP

SEQ ID NO: 200 - protein, 14 aa, synthetic construct
  CROSSLNK 5..9: note = aspartic acid D-lysine amide
  SITE 9: note = D-lysine
SRLEDELRKRLTEP

SEQ ID NO: 201 - protein, 14 aa, synthetic construct
  CROSSLNK 5..9: note = lysine D-aspartic acid amide
  SITE 9: note = D-aspartic acid
SRLEKELRDRLTEP

SEQ ID NO: 202 - protein, 14 aa, synthetic construct
  CROSSLNK 5..9: note = D-lysine D-aspartic acid amide
  SITE 5: note = D-lysine
  SITE 9: note = D-aspartic acid
SRLEKELRDRLTEP

SEQ ID NO: 203 - protein, 15 aa, synthetic construct
  SITE 6..10: note = D-lysine aspartic acid amide
  SITE 6: note = D-lysine
PSRLEKELRDRLTEP

SEQ ID NO: 204 - protein, 15 aa, synthetic construct
  SITE 6..10: note = D-aspartic acid D-lysine amide
  SITE 6: note = D-aspartic acid
  SITE 10: note = D-lysine
PSRLEDELRKRLTEP

SEQ ID NO: 205 - protein, 15 aa, synthetic construct
  SITE 6..10: note = D-aspartic acid lysine amide
  SITE 6: note = D-aspartic acid
PSRLEDELRKRLTEP

SEQ ID NO: 206 - protein, 15 aa, synthetic construct
  SITE 6..10: note = aspartic acid D-lysine amide
  SITE 10: note = D-lysine
PSRLEDELRKRLTEP

SEQ ID NO: 207 - protein, 15 aa, synthetic construct
  SITE 6..10: note = lysine D-aspartic acid amide
  SITE 10: note = D-aspartic acid
PSRLEKELRDRLTEP

SEQ ID NO: 208 - protein, 15 aa, synthetic construct
  SITE 6..10: note = D-lysine D-aspartic acid amide
  SITE 6: note = D-lysine
  SITE 10: note = D-aspartic acid
PSRLEKELRDRLTEP

SEQ ID NO: 209 - protein, 14 aa, synthetic construct
  SITE 5..8: note = D-glutamic acid lysine amide
  SITE 5: note = D-glutamic acid
SRLEEELKRRLTEP

SEQ ID NO: 210 - protein, 14 aa, synthetic construct
  SITE 5..8: note = glutamic acid D-lysine amide
  SITE 8: note = D-lysine
SRLEEELKRRLTEP

SEQ ID NO: 211 - protein, 14 aa, synthetic construct
  SITE 5..8: note = D-glutamic acid D-lysine amide
  SITE 5: note = D-glutamic acid
  SITE 8: note = D-lysine
SRLEEELKRRLTEP

SEQ ID NO: 212 - protein, 14 aa, synthetic construct
  SITE 5..8: note = lysine glutamic acid amide
SRLEKELERRLTEP

SEQ ID NO: 213 - protein, 14 aa, synthetic construct
  SITE 5..8: note = lysine D-glutamic acid amide
  SITE 8: note = D-glutamic acid
SRLEKELERRLTEP

SEQ ID NO: 214 - protein, 14 aa, synthetic construct
  SITE 5..8: note = D-lysine D-glutamic acid amide
  SITE 5: note = D-lysine
  SITE 8: note = D-glutamic acid
SRLEKELERRLTEP

SEQ ID NO: 215 - protein, 14 aa, synthetic construct
  SITE 5..8: note = aspartic acid lysine amide
SRLEDELKRRLTEP

SEQ ID NO: 216 - protein, 14 aa, synthetic construct
  SITE 5..8: note = aspartic acid D-lysine amide
  SITE 8: note = D-lysine
SRLEDELKRRLTEP

SEQ ID NO: 217 - protein, 14 aa, synthetic construct
  SITE 5..8: note = lysine aspartic acid amide
SRLEKELDRRLTEP

SEQ ID NO: 218 - protein, 14 aa, synthetic construct
  SITE 5..8: note = lysine D-aspartic acid amide
  SITE 8: note = D-aspartic acid
SRLEKELDRRLTEP

SEQ ID NO: 219 - protein, 14 aa, synthetic construct
  SITE 5..8: note = D-aspartic acid lysine amide
  SITE 5: note = D-aspartic acid
SRLEDELKRRLTEP

SEQ ID NO: 220 - protein, 14 aa, synthetic construct
  SITE 5..8: note = D-aspartic acid D-lysine amide
  SITE 5: note = D-aspartic acid
  SITE 8: note = D-lysine
SRLEDELKRRLTEP

SEQ ID NO: 221 - protein, 14 aa, synthetic construct
  SITE 5..8: note = D-lysine D-aspartic acid amide
  SITE 5: note = D-lysine
  SITE 8: note = D-aspartic acid
SRLEKELDRRLTEP

SEQ ID NO: 222 - protein, 14 aa, synthetic construct
  SITE 5..8: note = D-lysine aspartic acid amide
  SITE 5: note = D-lysine
SRLEKELDRRLTEP

SEQ ID NO: 223 - protein, 15 aa, synthetic construct
  SITE 6..9: note = glutamic acid D-lysine amide
  SITE 9: note = D-lysine
PSRLEEELKRRLTEP

SEQ ID NO: 224 - protein, 15 aa, synthetic construct
  SITE 6..9: note = D-glutamic acid D-lysine amide
  SITE 6: note = D-glutamic acid
  SITE 9: note = D-lysine
PSRLEEELKRRLTEP

SEQ ID NO: 225 - protein, 15 aa, synthetic construct
  SITE 6..9: note = lysine glutamic acid amide
PSRLEKELERRLTEP

SEQ ID NO: 226 - protein, 15 aa, synthetic construct
  SITE 6..9: note = lysine D-glutamic acid amide
  SITE 9: note = D-glutamic acid
PSRLEKELERRLTEP

SEQ ID NO: 227 - protein, 15 aa, synthetic construct
  SITE 6..9: note = D-lysine D-glutamic acid amide
  SITE 6: note = D-lysine
  SITE 9: note = D-glutamic acid
PSRLEKELERRLTEP

SEQ ID NO: 228 - protein, 15 aa, synthetic construct
  SITE 6..9: note = aspartic acid lysine amide
PSRLEDELKRRLTEP

SEQ ID NO: 229 - protein, 15 aa, synthetic construct
  SITE 6..9: note = aspartic acid D-lysine amide
  SITE 9: note = D-lysine
PSRLEDELKRRLTEP

SEQ ID NO: 230 - protein, 15 aa, synthetic construct
  SITE 6..9: note = lysine aspartic acid amide
PSRLEKELDRRLTEP

SEQ ID NO: 231 - protein, 15 aa, synthetic construct
  SITE 6..9: note = lysine D-aspartic acid amide
  SITE 9: note = D-aspartic acid
PSRLEKELDRRLTEP

SEQ ID NO: 232 - protein, 15 aa, synthetic construct
  SITE 6..9: note = D-aspartic acid lysine amide
  SITE 6: note = D-aspartic acid
PSRLEDELKRRLTEP

SEQ ID NO: 233 - protein, 15 aa, synthetic construct
  SITE 6..9: note = D-aspartic acid D-lysine amide
  SITE 6: note = D-aspartic acid
  SITE 9: note = D-lysine
PSRLEDELKRRLTEP

SEQ ID NO: 234 - protein, 15 aa, synthetic construct
  SITE 6..9: note = D-lysine D-aspartic acid amide
  SITE 6: note = D-lysine
  SITE 9: note = D-aspartic acid
PSRLEKELDRRLTEP

SEQ ID NO: 235 - protein, 15 aa, synthetic construct
  SITE 6..9: note = D-lysine aspartic acid amide
  SITE 6: note = D-lysine
PSRLEKELDRRLTEP

SEQ ID NO: 236 - protein, 14 aa, synthetic construct
  SITE 6..9: note = aspartic acid lysine amide
SRLEEDLRKRLTEP

SEQ ID NO: 237 - protein, 14 aa, synthetic construct
  SITE 6..9: note = lysine aspartic acid amide
SRLEEKLRDRLTEP

SEQ ID NO: 238 - protein, 14 aa, synthetic construct
  SITE 6..9: note = D-aspartic acid lysine amide
  SITE 6: note = D-aspartic acid
SRLEEDLRKRLTEP

SEQ ID NO: 239 - protein, 14 aa, synthetic construct
  SITE 6..9: note = lysine D-aspartic acid amide
  SITE 9: note = D-aspartic acid
SRLEEKLRDRLTEP

SEQ ID NO: 240 - protein, 14 aa, synthetic construct
  SITE 6..9: note = aspartic acid D-lysine amide
  SITE 9: note = D-lysine
SRLEEDLRKRLTEP

SEQ ID NO: 241 - protein, 14 aa, synthetic construct
  SITE 6..9: note = D-lysine aspartic acid amide
  SITE 6: note = D-lysine
SRLEEKLRDRLTEP

SEQ ID NO: 242 - protein, 14 aa, synthetic construct
  SITE 6..9: note = D-lysine D-aspartic acid amide
  SITE 6: note = D-lysine
  SITE 9: note = D-aspartic acid
SRLEEKLRDRLTEP

SEQ ID NO: 243 - protein, 14 aa, synthetic construct
  SITE 6..9: note = D-aspartic acid D-lysine amide
  SITE 6: note = D-aspartic acid
  SITE 9: note = D-lysine
SRLEEDLRKRLTEP

SEQ ID NO: 244 - protein, 15 aa, synthetic construct
  SITE 7..10: note = aspartic acid lysine amide
PSRLEEDLRKRLTEP

SEQ ID NO: 245 - protein, 15 aa, synthetic construct
  SITE 7..10: note = lysine aspartic acid amide
PSRLEEKLRDRLTEP

SEQ ID NO: 246 - protein, 15 aa, synthetic construct
  SITE 7..10: note = D-aspartic acid lysine amide
  SITE 7: note = D-aspartic acid
PSRLEEDLRKRLTEP

SEQ ID NO: 247 - protein, 15 aa, synthetic construct
  SITE 7..10: note = lysine D-aspartic acid amide
  SITE 10: note = D-aspartic acid
PSRLEEKLRDRLTEP

SEQ ID NO: 248 - protein, 15 aa, synthetic construct
  SITE 7..10: note = aspartic acid D-lysine amide
  SITE 10: note = D-lysine
PSRLEEDLRKRLTEP

SEQ ID NO: 249 - protein, 15 aa, synthetic construct
  SITE 7..10: note = D-lysine aspartic acid amide
  SITE 7: note = D-lysine
PSRLEEKLRDRLTEP

SEQ ID NO: 250 - protein, 15 aa, synthetic construct
  SITE 7..10: note = D-lysine D-aspartic acid amide
  SITE 7: note = D-lysine
  SITE 10: note = D-aspartic acid
PSRLEEKLRDRLTEP

SEQ ID NO: 251 - protein, 15 aa, synthetic construct
  SITE 7..10: note = D-aspartic acid D-lysine amide
  SITE 7: note = D-aspartic acid
  SITE 10: note = D-lysine
PSRLEEDLRKRLTEP

SEQ ID NO: 252 - protein, 14 aa, synthetic construct
  SITE 5..8: note = cysteine cysteine disulfide
SRLECELCRRLTEP

SEQ ID NO: 253 - protein, 14 aa, synthetic construct
  SITE 10..13: note = cysteine cysteine disulfide
SRLEEELRRCLTCP

SEQ ID NO: 254 - protein, 14 aa, synthetic construct
  SITE 9..12: note = cysteine cysteine disulfide
SRLEEELRCRLCEP

SEQ ID NO: 255 - protein, 14 aa, synthetic construct
  SITE 6..9: note = cysteine cysteine disulfide
SRLEECLRCRLTEP

SEQ ID NO: 256 - protein, 15 aa, synthetic construct
  SITE 6..9: note = cysteine cysteine disulfide
PSRLECELCRRLTEP

SEQ ID NO: 257 - protein, 15 aa, synthetic construct
  SITE 11..14: note = cysteine cysteine disulfide
PSRLEEELRRCLTCP

SEQ ID NO: 258 - protein, 15 aa, synthetic construct
  SITE 10..13: note = cysteine cysteine disulfide
PSRLEEELRCRLCEP

SEQ ID NO: 259 - protein, 15 aa, synthetic construct
  SITE 7..10: note = cysteine cysteine disulfide
PSRLEECLRCRLTEP

SEQ ID NO: 260 - protein, 14 aa, synthetic construct
  SITE 5..8: note = D-cysteine D-cysteine disulfide
  SITE 5: note = D-cysteine
  SITE 8: note = D-cysteine
SRLECELCRRLTEP

SEQ ID NO: 261 - protein, 14 aa, synthetic construct
  SITE 5..8: note = D-cysteine cysteine disulfide
  SITE 5: note = D-cysteine
SRLECELCRRLTEP

SEQ ID NO: 262 - protein, 14 aa, synthetic construct
  SITE 5..8: note = homocysteine cysteine disulfide
  SITE 5: note = homocysteine
SRLEXELCRRLTEP

SEQ ID NO: 263 - protein, 14 aa, synthetic construct
  SITE 5..8: note = homocysteine homocysteine disulfide
  SITE 5: note = homocysteine
  SITE 8: note = homocysteine
SRLEXELXRRLTEP

SEQ ID NO: 264 - protein, 14 aa, synthetic construct
  SITE 5..8: note = cysteine homocysteine disulfide
  SITE 8: note = homocysteine
SRLECELXRRLTEP

SEQ ID NO: 265 - protein, 14 aa, synthetic construct
  SITE 5..8: note = homocysteine D-cysteine disulfide
  SITE 5: note = homocysteine
  SITE 8: note = D-cysteine
SRLEXELCRRLTEP

SEQ ID NO: 266 - protein, 14 aa, synthetic construct
  SITE 5..8: note = D-cysteine homocysteine disulfide
  SITE 5: note = D-cysteine
  SITE 8: note = homocysteine
SRLECELXRRLTEP

SEQ ID NO: 267 - protein, 14 aa, synthetic construct
  SITE 5..8: note = homocysteine D-homocysteine disulfide
  SITE 5: note = homocysteine
  SITE 8: note = D-homocysteine
SRLEXELXRRLTEP

SEQ ID NO: 268 - protein, 14 aa, synthetic construct
  SITE 5..8: note = D-homocysteine D-homocysteine disulfide
  SITE 5: note = D-homocysteine
  SITE 8: note = D-homocysteine
SRLEXELXRRLTEP

SEQ ID NO: 269 - protein, 14 aa, synthetic construct
  SITE 5..8: note = cysteine D-homocysteine disulfide
  SITE 8: note = D-homocysteine
SRLECELXRRLTEP

SEQ ID NO: 270 - protein, 14 aa, synthetic construct
  SITE 5..8: note = D-cysteine D-homocysteine disulfide
  SITE 5: note = D-cysteine
  SITE 8: note = D-homocysteine
SRLECELXRRLTEP

SEQ ID NO: 271 - protein, 14 aa, synthetic construct
  SITE 5..8: note = D-homocysteine cysteine disulfide
  SITE 5: note = D-homocysteine
SRLEXELCRRLTEP

SEQ ID NO: 272 - protein, 14 aa, synthetic construct
  SITE 5..8: note = D-homocysteine D-cysteine disulfide
  SITE 5: note = D-homocysteine
  SITE 8: note = D-cysteine
SRLEXELCRRLTEP

SEQ ID NO: 273 - protein, 15 aa, synthetic construct
  SITE 6..9: note = D-cysteine D-cysteine disulfide
  SITE 6: note = D-cysteine
  SITE 9: note = D-cysteine
PSRLECELCRRLTEP

SEQ ID NO: 274 - protein, 15 aa, synthetic construct
  SITE 6..9: note = D-cysteine cysteine disulfide
  SITE 6: note = D-cysteine
PSRLECELCRRLTEP

SEQ ID NO: 275 - protein, 15 aa, synthetic construct
  SITE 6..9: note = homocysteine cysteine disulfide
  SITE 6: note = homocysteine
PSRLEXELCRRLTEP

SEQ ID NO: 276 - protein, 15 aa, synthetic construct
  SITE 6..9: note = homocysteine homocysteine disulfide
  SITE 6: note = homocysteine
  SITE 9: note = homocysteine
PSRLEXELXRRLTEP

SEQ ID NO: 277 - protein, 15 aa, synthetic construct
  SITE 6..9: note = cysteine homocysteine disulfide
  SITE 9: note = homocysteine
PSRLECELXRRLTEP

SEQ ID NO: 278 - protein, 15 aa, synthetic construct
  SITE 6..9: note = homocysteine D-cysteine disulfide
  SITE 6: note = homocysteine
  SITE 9: note = D-cysteine
PSRLEXELCRRLTEP

SEQ ID NO: 279 - protein, 15 aa, synthetic construct
  SITE 6..9: note = D-cysteine homocysteine disulfide
  SITE 6: note = D-cysteine
  SITE 9: note = homocysteine
PSRLECELXRRLTEP

SEQ ID NO: 280 - protein, 15 aa, synthetic construct
  SITE 6..9: note = homocysteine D-homocysteine disulfide
  SITE 6: note = homocysteine
  SITE 9: note = D-homocysteine
PSRLEXELXRRLTEP

SEQ ID NO: 281 - protein, 15 aa, synthetic construct
  SITE 6..9: note = D-homocysteine D-homocysteine disulfide
  SITE 6: note = D-homocysteine
  SITE 9: note = D-homocysteine
PSRLEXELXRRLTEP

SEQ ID NO: 282 - protein, 15 aa, synthetic construct
  SITE 6..9: note = cysteine D-homocysteine disulfide
  SITE 9: note = D-homocysteine
PSRLECELXRRLTEP

SEQ ID NO: 283 - protein, 15 aa, synthetic construct
  SITE 6..9: note = D-cysteine D-homocysteine disulfide
  SITE 6: note = D-cysteine
  SITE 9: note = D-homocysteine
PSRLECELXRRLTEP

SEQ ID NO: 284 - protein, 15 aa, synthetic construct
  SITE 6..9: note = D-homocysteine cysteine disulfide
  SITE 6: note = D-homocysteine
PSRLEXELCRRLTEP

SEQ ID NO: 285 - protein, 15 aa, synthetic construct
  SITE 6..9: note = D-homocysteine D-cysteine disulfide
  SITE 6: note = D-homocysteine
  SITE 9: note = D-cysteine
PSRLEXELCRRLTEP

SEQ ID NO: 286 - protein, 14 aa, synthetic construct
  SITE 10..13: note = cysteine D-cysteine disulfide
  SITE 13: note = D-cysteine
SRLEEELRRCLTCP

SEQ ID NO: 287 - protein, 14 aa, synthetic construct
  SITE 10..13: note = D-cysteine cysteine disulfide
  SITE 10: note = D-cysteine
SRLEEELRRCLTCP

SEQ ID NO: 288 - protein, 14 aa, synthetic construct
  SITE 10..13: note = D-cysteine D-cysteine disulfide
  SITE 10: note = D-cysteine
  SITE 13: note = D-cysteine
SRLEEELRRCLTCP

SEQ ID NO: 289 - protein, 14 aa, synthetic construct
  SITE 10..13: note = homocysteine cysteine disulfide
  SITE 10: note = homocysteine
SRLEEELRRXLTCP

SEQ ID NO: 290 - protein, 14 aa, synthetic construct
  SITE 10..13: note = homocysteine D-cysteine disulfide
  SITE 10: note = homocysteine
  SITE 13: note = D-cysteine
SRLEEELRRXLTCP

SEQ ID NO: 291 - protein, 14 aa, synthetic construct
  SITE 10..13: note = homocysteine homocysteine disulfide
  SITE 10: note = homocysteine
  SITE 13: note = homocysteine
SRLEEELRRXLTXP

SEQ ID NO: 292 - protein, 14 aa, synthetic construct
  SITE 10..13: note = cysteine homocysteine disulfide
  SITE 13: note = homocysteine
SRLEEELRRCLTXP

SEQ ID NO: 293 - protein, 14 aa, synthetic construct
  SITE 10..13: note = D-cysteine homocysteine disulfide
  SITE 10: note = D-cysteine
  SITE 13: note = homocysteine
SRLEEELRRCLTXP

SEQ ID NO: 294 - protein, 14 aa, synthetic construct
  SITE 10..13: note = cysteine homocysteine disulfide
  SITE 13: note = homocysteine
SRLEEELRRCLTXP

SEQ ID NO: 295 - protein, 14 aa, synthetic construct
  SITE 10..13: note = D-homocysteine cysteine disulfide
  SITE 10: note = D-homocysteine
SRLEEELRRXLTCP

SEQ ID NO: 296 - protein, 14 aa, synthetic construct
  SITE 10..13: note = D-homocysteine D-cysteine disulfide
  SITE 13: note = D-cysteine
  SITE 10: note = D-homocysteine
SRLEEELRRXLTCP

SEQ ID NO: 297 - protein, 14 aa, synthetic construct
  SITE 10..13: note = D-homocysteine homocysteine disulfide
  SITE 10: note = D-homocysteine
  SITE 13: note = homocysteine
SRLEEELRRXLTXP

SEQ ID NO: 298 - protein, 14 aa, synthetic construct
  SITE 10..13: note = cysteine D-homocysteine disulfide
  SITE 13: note = D-homocysteine
SRLEEELRRCLTXP

SEQ ID NO: 299 - protein, 14 aa, synthetic construct
  SITE 10..13: note = D-cysteine D-homocysteine disulfide
  SITE 10: note = D-cysteine
  SITE 13: note = D-homocysteine
SRLEEELRRCLTXP

SEQ ID NO: 300 - protein, 14 aa, synthetic construct
  SITE 10..13: note = cysteine D-homocysteine disulfide
  SITE 13: note = D-homocysteine
SRLEEELRRCLTXP

SEQ ID NO: 301 - protein, 14 aa, synthetic construct
  SITE 10..13: note = penicillamine penicillamine disulfide
  SITE 10: note = penicillamine
  SITE 13: note = penicillamine
SRLEEELRRXLTXP

SEQ ID NO: 302 - protein, 14 aa, synthetic construct
  SITE 10..13: note = penicillamine D-penicillamine disulfide
  SITE 10: note = penicillamine
  SITE 13: note = D-penicillamine
SRLEEELRRXLTXP

SEQ ID NO: 303 - protein, 14 aa, synthetic construct
  SITE 10..13: note = D-penicillamine penicillamine disulfide
  SITE 10: note = D-penicillamine
  SITE 13: note = penicillamine
SRLEEELRRXLTXP

SEQ ID NO: 304 - protein, 14 aa, synthetic construct
  SITE 10..13: note = D-penicillamine D-penicillamine disulfide
  SITE 10: note = D-penicillamine
  SITE 13: note = D-penicillamine
SRLEEELRRXLTXP

SEQ ID NO: 305 - protein, 14 aa, synthetic construct
  SITE 9..12: note = D-cysteine cysteine disulfide
  SITE 9: note = D-cysteine
SRLEEELRCRLCEP

SEQ ID NO: 306 - protein, 14 aa, synthetic construct
  SITE 9..12: note = D-cysteine D-cysteine disulfide
  SITE 9: note = D-cysteine
  SITE 12: note = D-cysteine
SRLEEELRCRLCEP

SEQ ID NO: 307 - protein, 14 aa, synthetic construct
  SITE 9..12: note = cysteine D-cysteine disulfide
  SITE 12: note = D-cysteine
SRLEEELRCRLCEP

SEQ ID NO: 308 - protein, 14 aa, synthetic construct
  SITE 9..12: note = homocysteine cysteine disulfide
  SITE 9: note = homocysteine
SRLEEELRXRLCEP

SEQ ID NO: 309 - protein, 14 aa, synthetic construct
  SITE 9..12: note = homocysteine D-cysteine disulfide
  SITE 9: note = homocysteine
  SITE 12: note = D-cysteine
SRLEEELRXRLCEP

SEQ ID NO: 310 - protein, 14 aa, synthetic construct
  SITE 9..12: note = cysteine homocysteine disulfide
  SITE 12: note = homocysteine
SRLEEELRCRLXEP

SEQ ID NO: 311 - protein, 14 aa, synthetic construct
  SITE 9..12: note = homocysteine homocysteine disulfide
  SITE 9: note = homocysteine
  SITE 12: note = homocysteine
SRLEEELRXRLXEP

SEQ ID NO: 312 - protein, 14 aa, synthetic construct
  SITE 9..12: note = homocysteine D-homocysteine disulfide
  SITE 12: note = D-homocysteine
  SITE 9: note = homocysteine
SRLEEELRXRLXEP

SEQ ID NO: 313 - protein, 14 aa, synthetic construct
  SITE 9..12: note = D-homocysteine homocysteine disulfide
  SITE 9: note = D-homocysteine
  SITE 12: note = homocysteine
SRLEEELRXRLXEP

SEQ ID NO: 314 - protein, 15 aa, synthetic construct
  SITE 11..14: note = cysteine D-cysteine disulfide
  SITE 14: note = D-cysteine
PSRLEEELRRCLTCP

SEQ ID NO: 315 - protein, 15 aa, synthetic construct
  SITE 11..14: note = D-cysteine cysteine disulfide
  SITE 11: note = D-cysteine
PSRLEEELRRCLTCP

SEQ ID NO: 316 - protein, 15 aa, synthetic construct
  SITE 11..14: note = D-cysteine D-cysteine disulfide
  SITE 11: note = D-cysteine
  SITE 14: note = D-cysteine
PSRLEEELRRCLTCP

SEQ ID NO: 317 - protein, 15 aa, synthetic construct
  SITE 11..14: note = homocysteine cysteine disulfide
  SITE 11: note = homocysteine
PSRLEEELRRXLTCP

SEQ ID NO: 318 - protein, 15 aa, synthetic construct
  SITE 11..14: note = homocysteine D-cysteine disulfide
  SITE 11: note = homocysteine
  SITE 14: note = D-cysteine
PSRLEEELRRXLTCP

SEQ ID NO: 319 - protein, 15 aa, synthetic construct
  SITE 11..14: note = homocysteine homocysteine disulfide
  SITE 11: note = homocysteine
  SITE 14: note = homocysteine
PSRLEEELRRXLTXP

SEQ ID NO: 320 - protein, 15 aa, synthetic construct
  SITE 11..14: note = cysteine homocysteine disulfide
  SITE 14: note = homocysteine
PSRLEEELRRCLTXP

SEQ ID NO: 321 - protein, 15 aa, synthetic construct
  SITE 11..14: note = D-cysteine homocysteine disulfide
  SITE 11: note = D-cysteine
  SITE 14: note = homocysteine
PSRLEEELRRCLTXP

SEQ ID NO: 322 - protein, 15 aa, synthetic construct
  SITE 11..14: note = cysteine homocysteine disulfide
  SITE 14: note = homocysteine
PSRLEEELRRCLTXP

SEQ ID NO: 323 - protein, 15 aa, synthetic construct
  SITE 11..14: note = D-homocysteine cysteine disulfide
  SITE 11: note = D-homocysteine
PSRLEEELRRXLTCP

SEQ ID NO: 324 - protein, 15 aa, synthetic construct
  SITE 11..14: note = D-homocysteine D-cysteine disulfide
  SITE 11: note = D-homocysteine
  SITE 14: note = D-cysteine
PSRLEEELRRXLTCP

SEQ ID NO: 325 - protein, 15 aa, synthetic construct
  SITE 11..14: note = D-homocysteine homocysteine disulfide
  SITE 11: note = D-homocysteine
  SITE 14: note = homocysteine
PSRLEEELRRXLTXP

SEQ ID NO: 326 - protein, 15 aa, synthetic construct
  SITE 11..14: note = cysteine D-homocysteine disulfide
  SITE 14: note = D-homocysteine
PSRLEEELRRCLTXP

SEQ ID NO: 327 - protein, 15 aa, synthetic construct
  SITE 11..14: note = D-cysteine D-homocysteine disulfide
  SITE 14: note = D-homocysteine
  SITE 11: note = D-cysteine
PSRLEEELRRCLTXP

SEQ ID NO: 328 - protein, 15 aa, synthetic construct
  SITE 11..14: note = cysteine D-homocysteine disulfide
  SITE 14: note = D-homocysteine
PSRLEEELRRCLTXP

SEQ ID NO: 329 - protein, 15 aa, synthetic construct
  SITE 11..14: note = penicillamine penicillamine disulfide
  SITE 11: note = penicillamine
  SITE 14: note = penicillamine
PSRLEEELRRXLTXP

SEQ ID NO: 330 - protein, 15 aa, synthetic construct
  SITE 11..14: note = penicillamine D-penicillamine disulfide
  SITE 11: note = penicillamine
  SITE 14: note = D-penicillamine
PSRLEEELRRXLTXP

SEQ ID NO: 331 - protein, 15 aa, synthetic construct
  SITE 11..14: note = D-penicillamine penicillamine disulfide
  SITE 11: note = D-penicillamine
  SITE 14: note = penicillamine
PSRLEEELRRXLTXP

SEQ ID NO: 332 - protein, 15 aa, synthetic construct
  SITE 11..14: note = D-penicillamine D-penicillamine disulfide
  SITE 11: note = D-penicillamine
  SITE 14: note = D-penicillamine
PSRLEEELRRXLTXP

SEQ ID NO: 333 - protein, 15 aa, synthetic construct
  SITE 10..13: note = D-cysteine cysteine disulfide
  SITE 10: note = D-cysteine
PSRLEEELRCRLCEP

SEQ ID NO: 334 - protein, 15 aa, synthetic construct
  SITE 10..13: note = D-cysteine D-cysteine disulfide
  SITE 10: note = D-cysteine
  SITE 13: note = D-cysteine
PSRLEEELRCRLCEP

SEQ ID NO: 335 - protein, 15 aa, synthetic construct
  SITE 10..13: note = cysteine D-cysteine disulfide
  SITE 13: note = D-cysteine
PSRLEEELRCRLCEP

SEQ ID NO: 336 - protein, 15 aa, synthetic construct
  SITE 10..13: note = homocysteine cysteine disulfide
  SITE 10: note = homocysteine
PSRLEEELRXRLCEP

SEQ ID NO: 337 - protein, 15 aa, synthetic construct
  SITE 10..13: note = homocysteine D-cysteine disulfide
  SITE 10: note = homocysteine
  SITE 13: note = D-cysteine
PSRLEEELRXRLCEP

SEQ ID NO: 338 - protein, 15 aa, synthetic construct
  SITE 10..13: note = cysteine homocysteine disulfide
  SITE 13: note = homocysteine
PSRLEEELRCRLXEP

SEQ ID NO: 339 - protein, 15 aa, synthetic construct
  SITE 10..13: note = homocysteine homocysteine disulfide
  SITE 10: note = homocysteine
  SITE 13: note = homocysteine
PSRLEEELRXRLXEP

SEQ ID NO: 340 - protein, 15 aa, synthetic construct
  SITE 10..13: note = homocysteine D-homocysteine disulfide
  SITE 10: note = homocysteine
  SITE 13: note = D-homocysteine
PSRLEEELRXRLXEP

SEQ ID NO: 341 - protein, 15 aa, synthetic construct
  SITE 10..13: note = D-homocysteine homocysteine disulfide
  SITE 10: note = D-homocysteine
  SITE 13: note = homocysteine
PSRLEEELRXRLXEP

SEQ ID NO: 342 - protein, 14 aa, synthetic construct
  SITE 9..12: note = lysine aspartic acid amide
SRLEEELRKRLDEP

SEQ ID NO: 343 - protein, 14 aa, synthetic construct
  SITE 9..12: note = aspartic acid lysine amide
SRLEEELRDRLKEP

SEQ ID NO: 344 - protein, 14 aa, synthetic construct
  SITE 9..12: note = lysine glutamic acid amide
SRLEEELRKRLEEP

SEQ ID NO: 345 - protein, 14 aa, synthetic construct
  SITE 9..12: note = glutamic acid lysine amide
SRLEEELRERLKEP

SEQ ID NO: 346 - protein, 15 aa, synthetic construct
  SITE 10..13: note = lysine aspartic acid amide
PSRLEEELRKRLDEP

SEQ ID NO: 347 - protein, 15 aa, synthetic construct
  SITE 10..13: note = aspartic acid lysine amide
PSRLEEELRDRLKEP

SEQ ID NO: 348 - protein, 15 aa, synthetic construct
  SITE 10..13: note = lysine glutamic acid amide
PSRLEEELRKRLEEP

SEQ ID NO: 349 - protein, 15 aa, synthetic construct
  SITE 10..13: note = glutamic acid lysine amide
PSRLEEELRERLKEP

SEQ ID NO: 350 - protein, 15 aa, synthetic construct
  SITE 10..14: note = cysteine cysteine disulfide
PSRLEEELRCRLTCP

SEQ ID NO: 351 - protein, 15 aa, synthetic construct
  SITE 6..10: note = cysteine cysteine disulfide
PSRLECELRCRLTEP

SEQ ID NO: 352 - protein, 15 aa, synthetic construct
  SITE 11..14: note = cysteine cysteine disulfide
PSRLEEELRRCLTCP

SEQ ID NO: 353 - protein, 15 aa, synthetic construct
  SITE 7..11: note = cysteine cysteine disulfide
PSRLEECLRRCLTEP

SEQ ID NO: 354 - protein, 13 aa, synthetic construct
  SITE 5: note = residues at positions 5 and 9 form different rings
  SITE 9: note = residues at positions 5 and 9 form different rings
SRLEXELRXRLTE

SEQ ID NO: 355 - protein, 14 aa, synthetic construct
PSRLEEELRRRLTE

SEQ ID NO: 356 - protein, 13 aa, synthetic construct
SRLEEELRRRLTE

SEQ ID NO: 357 - protein, 12 aa, synthetic construct
  VARIANT 3: note = isoleucine or valine
VTXSALNAMAMG

SEQ ID NO: 358 - protein, 9 aa, synthetic construct
  VARIANT 4: note = glutamic acid, histidine, asparagine, aspartic acid or serine
AVSXRGNAM

SEQ ID NO: 359 - protein, 11 aa, synthetic construct
LEDRVDSFHDY

SEQ ID NO: 360 - protein, 13 aa, synthetic construct
  VARIANT 4: note = isoleucine or valine
GVTXSALNAMAMG

SEQ ID NO: 361 - protein, 12 aa, synthetic construct
VTISALNAMAMG

SEQ ID NO: 362 - protein, 9 aa, synthetic construct
AVSERGNAM

SEQ ID NO: 363 - protein, 13 aa, synthetic construct
GVTISALNAMAMG

SEQ ID NO: 364 - protein, 122 aa, synthetic construct
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 365 - protein, 14 aa, synthetic construct
SRLEEELRRRLTEP

SEQ ID NO: 366 - protein, 12 aa, synthetic construct
PDRVRAVSHWSS

SEQ ID NO: 367 - protein, 15 aa, synthetic construct
PSRLEEELRRRLTEP

SEQ ID NO: 368 - protein, 13 aa, synthetic construct
  CROSSLNK 5..9: note = lysine glutamic acid amide
SRLEKELRERLTE

SEQ ID NO: 369 - protein, 13 aa, synthetic construct
  SITE 4..8: note = glutamic acid lysine amide
SRLEEELKRRLTE

(19) United States
(12) Patent Application Publication
(10) Pub. No.: US 2026/0041769 A1
(43) Pub. Date: Feb. 12, 2026

(54) AGENTS AND METHODS FOR TARGETED DELIVERY OF NUCLEIC ACIDS TO CELLS

(71) Applicant: BioNTech SE

(72) Inventors: Meike GANGLUFF, Mainz (DE); Zhengxin DONG, Cambridge, MA (US); Philipp HELLER, Mainz (DE); Heinrich HAAS, Mainz (DE); Karl KLINGER, Mainz (DE); Kaushik THANKI, Mainz (DE); Azzurra APRICENO, Mainz (DE); Arne Marten BILLMEIER, Mainz (DE); Tanya KAPOOR, Mainz (DE); Kathrin KUNA, Mainz (DE); Benjamin RENGSTL, Mainz (DE); Hans-Ulrich SCHMOLDT, Mainz (DE); Joycelyn WÜSTEHUBE-LAUSCH, Mainz (DE); Dirk WILDEMANN, Berlin (DE); Jonathan MCGEE, Cambridge, MA (US); Daniel KALLIN, Cambridge, MA (US); Jan Patrick BOGEN, Mainz (DE); Klaus KOCH, Mainz (DE); Olaf OTHERSEN, Mainz (DE); Christoph KRESS, Mainz (DE)

(21) Appl. No.: 18/835,533

(22) PCT Filed: Feb. 2, 2023
(86) PCT No.: PCT/EP2023/052580

§ 371 Date: Aug. 2, 2024

Related U.S. Application Data
(60) Provisional application No. 63/305,905, filed on Feb. 2, 2022.
(60) Provisional application No. 63/370,040, filed on Aug. 1, 2022.
(60) Provisional application No. 63/370,046, filed on Aug. 1, 2022.
(60) Provisional application No. 63/370,050, filed on Aug. 1, 2022.

(30) Foreign Application Priority Data
Feb. 2, 2022 (WO) PCT/EP2022/052465

(51) Int. Cl. A61K 40/31; A61K 40/11; A61K 40/32; A61K 40/41; A61K 48/00; C07K 14/725; C07K 16/28; C07K 17/08; C12N 15/88

(52) U.S. Cl. CPC A61K 40/31; A61K 40/11; A61K 40/32; A61K 40/41; A61K 48/0033; C07K 14/7051; C07K 16/2809; C07K 16/2815; C07K 17/08; C12N 15/88; C07K 2317/569

(57) ABSTRACT

The invention relates to agents and methods for targeted delivery of nucleic acids to cells. In some embodiments, the nucleic acid payload comprises a nucleic acid encoding an antigen receptor such as a T cell receptor (TCR) or chimeric antigen receptor (CAR). The agents and methods for targeted delivery of a nucleic acid encoding an antigen receptor described herein may be used for generating in vitro/ex vivo or in vivo immune effector cells genetically modified to express an antigen receptor.

The invention relates to agents and methods for targeted delivery of nucleic acids to cells. The nucleic acids include DNA and/or RNA. In some embodiments, the nucleic acid comprises RNA such as mRNA. In some embodiments, the nucleic acid encodes an antigen receptor such as a T cell receptor (TCR) or chimeric antigen receptor (CAR). Delivering a nucleic acid encoding an antigen receptor such as a TCR or CAR to cells may be useful for generating immune effector cells genetically modified to express an antigen receptor. In some embodiments, the invention involves a particle, said particle comprising a hydrophobic moiety having a binding moiety covalently attached thereto (targeting compound) and said particle carrying a nucleic acid payload, e.g., a nucleic acid encoding an antigen receptor. Targeting of a cell may be achieved by the direct or indirect binding of the targeting compound to cell surface antigens on the target cell of interest.

In some embodiments, the binding moiety of the targeting compound binds to a cell surface antigen on a target cell, e.g., an immune effector cell, for targeting the particle carrying a nucleic acid payload to target cells. In these embodiments, the binding moiety of the targeting compound may be constructs that have affinity for cell surface targets, e.g., membrane proteins, and include antibodies or antibody fragments.

In some embodiments, the binding moiety of the targeting compound binds to a compound binding to a cell surface antigen on a target cell, e.g., an immune effector cell, (docking compound) for targeting the particle carrying a nucleic acid payload to target cells. In some embodiments, the docking compound comprises a peptide or polypeptide. In some embodiments, the docking compound comprises a binding moiety binding to target cells (primary targeting moiety) and a further binding moiety binding to the binding moiety of the targeting compound. The binding moiety of the targeting compound may bind to its binding moiety on the docking compound and then the primary targeting moiety may bind to a target antigen on target cells such as an antigen on immune effector cells to thereby precisely deliver a nucleic acid payload to the target cells such as immune effector cells.

In many areas of medical therapy and diagnosis, it is desired to selectively deliver an agent, such as a nucleic acid to a specific cell in the body of a subject such as a patient.

The present invention relates to an approach wherein particles comprising a nucleic acid payload and a targeting compound are used. The targeting compound comprises (i) a hydrophobic moiety for incorporation into the particles and (ii) a binding moiety covalently attached to the hydrophobic moiety for direct or indirect targeting of the particles to target cells and delivering the nucleic acid payload to target cells. Cell targeting may be achieved by the direct or indirect binding of the targeting compound to cell surface antigens on the target cell of interest.

In some embodiments, particles comprising a nucleic acid payload and a targeting compound are administered. In some embodiments, the binding moiety of the targeting compound binds to target cells, e.g., by binding to a cell surface antigen, thus resulting in cellular uptake of the nucleic acid payload.

Common examples for target cell binding moieties on the targeting compound are antibodies. In some embodiments, particles comprising a nucleic acid payload and a targeting compound, and a docking compound that binds to target cells, e.g., by binding to a cell surface antigen, are administered. In these embodiments, the targeting compound may be equipped with a binding moiety targeting a moiety on the docking compound. In some embodiments, a docking compound which is bound via a targeting compound to a particle comprising a nucleic acid payload, e.g., a nucleic acid encoding an antigen receptor, is administered. The docking compound may bind to target cells, e.g., by binding to a cell surface antigen, thus resulting in cellular uptake of the nucleic acid payload. Common examples for pairs of interacting moieties on the targeting compound and on the docking compound are antibody/antigen systems. Common examples for target cell binding moieties on the docking compound are antibodies.

### SUMMARY

The invention relates to agents and methods for targeted delivery of nucleic acid payloads to cells. In some embodiments, the nucleic acid payload comprises a nucleic acid encoding an antigen receptor such as a T cell receptor (TCR) or chimeric antigen receptor (CAR). The agents and methods for targeted delivery of a nucleic acid encoding an antigen receptor described herein may be used for generating in vitro/ex vivo or in vivo immune effector cells genetically modified to express an antigen receptor. Genetic modification is achieved using particles described herein comprising nucleic acid encoding an antigen receptor for genetic modification and a targeting compound for targeting immune effector cells directly or via a docking compound binding to the targeting compound. The particles may deliver the nucleic acid to cells in vitro/ex vivo as well as in vivo. Immune effector cells genetically modified to express an antigen receptor described herein are useful in the treatment of diseases wherein targeting cells such as diseased cells expressing an antigen such as a tumor antigen is beneficial. The target cells may express the antigen on the cell surface for recognition by a CAR or in the context of MHC for recognition by a TCR. The treatments described herein may provide for the selective eradication of such cells expressing an antigen, thereby minimizing adverse effects to normal cells not expressing the antigen. Immune effector cells genetically modified to express an antigen receptor, e.g., a CAR or TCR, targeting cells through binding to the antigen (or a procession product thereof) are provided to a subject such as by administration of genetically modified immune effector cells to the subject or generation of genetically modified immune effector cells in the subject. In some embodiments, the immune effector cells are CD3+ T cells. In some embodiments, the target cell binding moiety (which may be present on the targeting compound or on the docking compound described herein) binds to the CD3 receptor on T cells. In some embodiments, the immune effector cells are CD8+ T cells. In some embodiments, the target cell binding moiety binds to the CD8 receptor on T cells. In some embodiments, the immune effector cells are CD4+ T cells. In some embodiments, the target cell binding moiety binds to the CD4 receptor on T cells. The methods and agents described herein and immune effector cells genetically modified to express an antigen receptor are, in particular, useful for the treatment of diseases characterized by diseased cells expressing an antigen the immune effector cells are directed to. In some embodiments, the immune effector cells by means of a CAR have a binding specificity for disease-associated antigen when present on diseased cells. In some embodiments, the immune effector cells by means of a TCR have a binding specificity for a procession product of disease-associated antigen when presented on diseased cells. In some embodiments, a cell is genetically modified to stably express an antigen receptor on its surface. In some embodiments, a cell is genetically modified to transiently express an antigen receptor on its surface.

In one aspect, the invention relates to a compound of the formula:

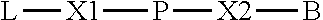

wherein
P comprises a polymer;
L comprises a hydrophobic moiety attached to the polymer;
B comprises a moiety attached to the polymer selected from the group consisting of a moiety binding to
a cell surface antigen, a peptide tag, and a moiety binding to a peptide tag;
X1 is absent or a first linking moiety; and
X2 is absent or a second linking moiety.

This compound is also referred to herein as “targeting compound”.

In some embodiments, the hydrophobic moiety comprises a moiety selected from vitamin E, dialkylamine, diacylglyceride and ceramide.

In some embodiments, the hydrophobic moiety comprises two C8-C24 hydrocarbon chains.

In some embodiments, the hydrophobic moiety comprises a lipid.

In some embodiments, the hydrophobic moiety comprises a phospholipid.

In some embodiments, the hydrophobic moiety comprises a moiety selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof.

In some embodiments, the polymer is a hydrophilic polymer.

In some embodiments, the polymer is selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA), derivatives and combinations thereof.

In some embodiments, X2 comprises the reaction product of a thiol or cysteine reactive group with a thiol or cysteine group of a compound comprising the moiety B.

In some embodiments, the thiol or cysteine reactive group comprises a maleimide group.

In some embodiments, the compound comprises the reaction product of 1,2-distearoyl-sn-glycero-3-phosphoethanolamine-N-[maleimide(polyethylene glycol)] with a compound comprising the formula SH(CH2)nC(O)—B, wherein n ranges from 1 to 5 and preferably n is 2.

In some embodiments, the compound is a compound of the formula

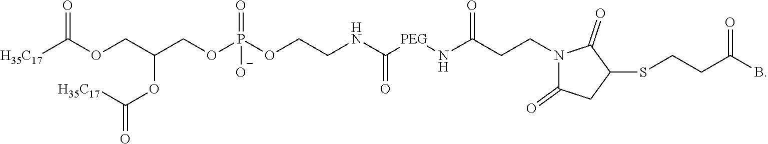

In some embodiments, B comprises a peptide or polypeptide.

In some embodiments, the moiety binding to a cell surface antigen or a peptide tag comprises an antibody or antibody-like molecule.

In some embodiments, the antibody-like molecule comprises an antibody fragment or DARPin.

In some embodiments, the cell surface antigen is characteristic for an immune effector cell.

In some embodiments, the cell surface antigen is selected from the group consisting of CD4, CD8 and CD3.

In some embodiments, the peptide tag comprises an ALFA-tag.

In one aspect, the invention relates to a functionalized particle comprising one or more particle forming components, a nucleic acid payload, and a compound described above (targeting compound), wherein the compound is incorporated into the particle through the hydrophobic moiety.

In some embodiments, B comprises a moiety binding to a cell surface antigen.

In some embodiments, B comprises a peptide tag or a moiety binding to a peptide tag and the functionalized particle further comprises a compound of the formula:

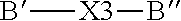

wherein
B′ comprises a moiety binding to B;
X3 is absent or a linking moiety; and
B″ comprises a moiety binding to a cell surface antigen.

The compound of the formula B′-X3-B″ is also referred to herein as “docking compound”.

In some embodiments, the compound of the formula B′-X3-B″ comprises a peptide or polypeptide.

In some embodiments, the moiety binding to a cell surface antigen comprises an antibody or antibody-like molecule.

In some embodiments, the antibody-like molecule comprises an antibody fragment or DARPin.

In some embodiments, the cell surface antigen is characteristic for an immune effector cell.

In some embodiments, the cell surface antigen is selected from the group consisting of CD4, CD8 and CD3.

In some embodiments, B comprises a peptide tag and B′ comprises a moiety binding to the peptide tag.

In some embodiments, B′ comprises a peptide tag and B comprises a moiety binding to the peptide tag.

In some embodiments, the moiety binding to a peptide tag comprises an antibody or antibody-like molecule.

In some embodiments, the antibody-like molecule comprises an antibody fragment or DARPin.

In some embodiments, the peptide tag comprises an ALFA-tag.

In some embodiments, the peptide tag comprises an ALFA-tag and the moiety binding to the peptide tag comprises a VHH domain comprising the CDR1 sequence VTISALNAMAMG, the CDR2 sequence AVSERGNAM, and the CDR3 sequence LEDRVDSFHDY.

In some embodiments, the particle is a lipid particle, a polymer particle, or a mixture thereof.

In some embodiments, the particle is a non-viral particle.

In some embodiments, the particle is a nanoparticle.

In some embodiments, the nucleic acid comprises DNA and/or RNA.

In some embodiments, the nucleic acid comprises a nucleic acid encoding an antigen receptor.

In some embodiments, the antigen receptor comprises a chimeric antigen receptor (CAR) or T cell receptor (TCR).

In one aspect, the invention relates to a method for delivering a nucleic acid to a cell expressing a cell surface antigen, comprising adding to the cell a composition comprising the functionalized particle described above.

In some embodiments, B or B″ comprises a moiety binding to the cell surface antigen.

In some embodiments, the cell is present ex vivo.

In some embodiments, the cell is present in a subject and the method comprises administering the composition to the subject.

In some embodiments, the cell comprises an immune effector cell.

In some embodiments, the immune effector cell comprises a T cell.

In some embodiments, the immune effector cell comprises a CD8+ and/or CD4+ T cell.

In some embodiments, the cell surface antigen is characteristic for an immune effector cell.

In some embodiments, the cell surface antigen is selected from the group consisting of CD4, CD8 and CD3.

In some embodiments, the nucleic acid comprises a nucleic acid encoding an antigen receptor.

In some embodiments, the antigen receptor comprises a chimeric antigen receptor (CAR) or T cell receptor (TCR).

In some embodiments, the method is a method for preparing a genetically modified cell.

In some embodiments, the method is a method for preparing an immune effector cell genetically modified to express an antigen receptor.

In some embodiments, the genetic modification is transient or stable.

In some embodiments, the genetic modification takes place by a virus-based method, transposon-based method, or a gene editing-based method.

In one aspect, the invention relates to a method for preparing an immune effector cell genetically modified to express an antigen receptor, comprising adding to an immune effector cell a composition comprising the functionalized particle described above, wherein the nucleic acid comprises a nucleic acid encoding an antigen receptor.

In some embodiments, B or B″ comprises a moiety binding to a cell surface antigen on the immune effector cell.

In some embodiments, the immune effector cell is present ex vivo.

In some embodiments, the immune effector cell is present in a subject and the method comprises administering the composition to the subject.

In some embodiments, the immune effector cell comprises a T cell.

In some embodiments, the immune effector cell comprises a CD8+ and/or CD4+ T cell.

In some embodiments, the cell surface antigen is characteristic for the immune effector cell.

In some embodiments, the cell surface antigen is selected from the group consisting of CD4, CD8 and CD3.

In some embodiments, the antigen receptor comprises a chimeric antigen receptor (CAR) or T cell receptor (TCR).

In some embodiments, the genetic modification is transient or stable.

In some embodiments, the genetic modification takes place by a virus-based method, transposon-based method, or a gene editing-based method.

In one aspect, the invention relates to a method for treating a subject comprising:
(I) preparing ex vivo an immune effector cell genetically modified to express an antigen receptor using the method described above, and
(II) administering the immune effector cell genetically modified to express an antigen receptor to the subject.

In one aspect, the invention relates to a method for treating a subject comprising preparing in vivo an immune effector cell genetically modified to express an antigen receptor using the method described above.

### BRIEF DESCRIPTION OF THE DRAWINGS

FIG. 1: Formulation and characterization of functionalized lipid nanoparticles (LNPs) containing RNA

LNPs were formulated with varying mol % of Alfa tag lipid (Lipid mix: DODMA/Chol/DOPE/C16-PEG-Ceramide/DSPE-PEG2k-Alfa 40/48/10/2-x/x=0, 0.2, 0.5, 1; N/P ratio: 4; Cargo: Thy1.1/Luc RNA 1:1 w/w; RNA concentration: 0.1 μg/μl). aCD8-DARPin X NbAlfa was added via post-functionalization [w/w*=ligand to cargo ratio 0.55;). Successful RNA incorporation was verified via Agarose gel electrophoresis. Size and PDI were measured via dynamic light scattering at 0.005 g/L RNA concentration for LNPs with or without ligand. **No PDI measurable.

FIG. 2: T-cell targeting using LNPs decorated with aCD3 ligand LNPs were formulated with or without ligand (Cargo: Thy1.1/Luc RNA 1:1 w/w; N/P ratio: 4; Lipid mix: DODMA/Cholesterol/DOPE/C16 PEG2k Ceramide/DSPE-PEG2k-Alfa [40/48/10/1.8/0.2]; aCD3-VHH×NbAlfa post-functionalization [w/w*=ligand to cargo ratio 1.15]; RNA concentration: 0.1 μg/μl). Successful RNA incorporation was verified via Agarose gel electrophoresis. For transfection studies, 15 μl of respective formulations were pre-diluted in 50 μl X-Vivo 15 in an ultra-low adhesion 96 well plate. 1e6 thawed human PBMC were diluted in 50 μl X-Vivo 15 (with 10% PHS) and added to nanoparticle dilution (1500 ng RNA). After 30 min of incubation (37° C., 5% CO2) 200 μl of X-Vivo15+5% PHS was added per well and cells were cultivated for additional 18 h (37° C., 5% CO2). In the following cell-type specific transfection (Thy1.1) and T cell activation (CD69) were analyzed via flowcytometry. (A) LNP characteristics. Diameter and PDI were determined via DLS measurement. (B) Depicted are the cell type specific Thy1.1 (Transfection, y-axes) signals within viable CD14+ Monocytes, CD56+NK cells, CD19+ B cells, CD3+ T cells, CD4+ T cells or CD8+ T cells. (C) Histograms show CD69 activation marker expression on the T cell population within the treated PBMCs.

FIG. 3: T-cell targeting using LPLX decorated with aCD8 DARPin

LPLX were functionalized with two different ligands: aCD8 DARPin X Spycatcher or aCD8 DARPin X NbAlfa. LPLX contain the cationic Viromer L3 polymer for RNA encapsulation and a functionalized lipid (Cargo: Thy1.1/Luc RNA 1:1 w/w; RNA concentration: 0.1 μg/μL; N/P ratio: 7.5; ligand to cargo w/w ratio 0.25 for aCD8 DAPRin X Spycatcher and 0.1 for aCD8 DARPin X NbAlfa; molar ratio ligand to functionalized lipid 1.5:1 for aCD8 DAPRin X Spycatcher/Spy tag PEG2k-DSPE and 3.5:1 for aCD8 DARPin X NbAlfa/Alfa tag PEG2k-DSPE). Diameter and PDI were determined via DLS measurement. Successful RNA incorporation was verified via Agarose gel electrophoresis. For transfection studies, 0.5 or 0.1 μl of respective formulations were pre-diluted in 50 μl X-Vivo 15 in a 96 well plate. 1e6 thawed human T-cells were diluted in 50 μl X-Vivo 15 and added to nanoparticle dilution (50 or 100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Depicted are Thy1.1 (Transfection, y-axes) signals within viable CD4+ T cells or CD8+ T cells.

FIG. 4: T-cell targeting using LPLX decorated with different ligands

LPLX were functionalized with fusion proteins comprised of a CD8 targeting ligand fused to X NbAlfa. The CD8 targeting domain is either a DARPin or a VHH nanobody. One DARPin and four different nanobodies were tested. LPLX contain Viromer L3 for RNA encapsulation and DSPE-PEG2k Alfa tag for ligand conjugation (Cargo: Thy1.1/Luc RNA 1:1 w/w; RNA concentration: 0.1 μg/μL; N/P ratio: 7.5; ligand to cargo w/w ratio 0.05, 0.1 and 0.2 for aCD8 VHH X NbAlfa; ligand to cargo w/w ratio 0.2 for aCD8 DARPin×NbAlfa; molar ratio ligand to functionalized lipid 3.5:1). Diameter and PDI were determined via DLS measurement. Successful RNA incorporation was verified via Agarose gel electrophoresis. For transfection studies, 0.1 μl of respective formulations were pre-diluted in 50 μl X-Vivo 15 in a 96 well plate. 2e5 thawed human T-cells were diluted in 50 μl X-Vivo 15 and added to nanoparticle dilution (100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Middle graph shows particle condition (see lower table) specific CD8+ T cell transfection efficiency calculated via multiplying the percentage of transfected (Thy1.1+) of CD8+ T cells with the MFI of the Thy1.1+CD8+ cell population. Dot blots corresponding to conditions in the graph marker with a box are depicted in the upper part of the figure. These blots show Thy1.1 signals within viable CD8+ T cells (upper blots) or viable CD4+ T cells (lower blots).

FIG. 5: T-cell targeting using LPLX decorated with different ligands

LPLX were functionalized with fusion proteins comprised of a CD8, CD4 or CD3 targeting ligand fused to X NbAlfa. The targeting domain is either a DARPin, a VHH nanobody or a scFv. One DARPin, nine different nanobodies and two different scFvs were tested. LPLX contain Viromer L3 for RNA encapsulation and DSPE-PEG2k Alfa tag for ligand conjugation (Cargo: Thy1.1/Luc RNA 1:1 w/w; RNA concentration: 0.1 μg/μL; N/P ratio: 7.5; ligand to cargo w/w ratio 0.05, 0.1 and 0.2 for aCD4 VHH X NbAlfa and aCD3 scFv×NbAlfa; ligand to cargo w/w ratio 0.2 for aCD8 DARPin X NbAlfa and aCD3 VHH X NbAlfa; molar ratio ligand to functionalized lipid 3.5:1). Diameter and PDI were determined via DLS measurement. Successful RNA incorporation was verified via Agarose gel electrophoresis. For transfection studies, 0.1 μl of respective formulations were pre-diluted in 50 μl X-Vivo 15 in a 96 well plate. 2e5 thawed human T-cells were diluted in 50 μl X-Vivo 15 and added to nanoparticle dilution (100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Middle Graph shows particle condition (see lower table) specific CD8+(left y-axis; dark grey bars) and CD4+(right y-axis; light grey bars) T cell transfection efficiency. Values are calculated via multiplying the percentage of transfected (Thy1.1+) of CD8 or CD4+ T cells with the MFI of the respective Thy1.1+ cell population. Dot blots corresponding to conditions in the graph marker with a box are depicted in the upper part of the figure. These blots show Thy1.1 signals within viable CD8+ T cells (upper blots) or viable CD4+ T cells (lower blots).

FIG. 6: T-cell targeting using LNPs with different lipid compositions

LNPs were formulated with different lipid compositions and equipped with aCD3 VHH X NbAlfa ligand (Cargo: Thy1.1/Luc RNA 1:1 w/w; N/P ratio: 4; Lipid mix: Ionizable lipid/Cholesterol/DOPE/C16 PEG2k Ceramide/DSPE-PEG2k-Alfa [40/48/10/1.8/0.2] with DODMA (C12), HY-501 (H12) or EA-405 (E12) as ionizable lipid; aCD3-VHH X NbAlfa post-functionalization [w/w*=ligand to cargo ratio 1.15; molar ratio ligand to functionalized lipid 1.5:1]; RNA concentration: 0.1 μg/μl). Diameter of all LNPs is below 120 nm with a PDI below 0.2 as determined via DLS measurement. Successful RNA incorporation was verified via Agarose gel electrophoresis. For transfection studies, 5 or 15 μl (500 ng or 1500 ng dose) of respective formulations were pre-diluted in 50 μl X-Vivo 15 in an ultra-low adhesion 96 well plate. 1e6 thawed human PBMC were diluted in 50 μl X-Vivo 15 (with 10% PHS) and added to nanoparticle dilution. After 30 min of incubation (37° C., 5% CO2) 200 μl of X-Vivo15+5% PHS was added per well and cells were cultivated for additional 18 h (37° C., 5% C02). In the following cell-type specific transfection (Thy1.1) was analyzed via flowcytometry. Depicted are the percentages of transfected cell (CD14+ Monocytes, CD56+NK cells, CD19+ B cells, CD3+ T cells, CD4+ T cells or CD8+ T cells) within all transfected PBMCs (Transfection, y-axes) per tested formulation condition (see lower table).

FIG. 7: T-cell targeting using LPLX with different lipids and linkers

LPLX were functionalized with aCD8 DARPin X NbAlfa using three different Alfa tag lipids as well as one control lipid without Alfa tag: DSPE-PEG2k Alfa, DOPE-(G2SG2)2 Alfa, DOPE-(G2SG2)4 Alfa and DOPE-(G2SG2)2. All LPLX contain Viromer L3 for RNA encapsulation (Cargo: Thy1.1/Luc RNA 1:1 w/w; RNA concentration: 0.1 μg/μL; N/P ratio: 15; ligand to cargo w/w ratio 0, 1.5 or 2; molar ratio ligand to functionalized lipid 1.5:1). Diameter and PDI were determined via DLS measurement. Successful RNA incorporation was verified via Agarose gel electrophoresis. For transfection studies, 1e6 CD8+ Jurkat cells were seeded per well (96 deep well plate) in 100 μl RMPI+10% FBS. 3 μl of respective formulations (300 ng RNA) were applied per well followed by resuspension of NP-cell solution and incubation for 10 min at 37° C. Afterwards 900 μl of RPMI+10% FBS were added per well to reach a cell concentration of 1e6 cells/ml. 0.1e6 cells in 100 μl were seeded for luciferase-expression readout on day 1. 0.3e6 cells in 300 μl were seeded for FACs-based detection of Thy1.1 expression on day 1.

FIG. 8: T-cell targeting using LNP with different stealth lipids and Alfa-lipids

LNPs were formulated with different lipid compositions (Cargo: Thy1.1/Luc RNA 1:1 w/w; N/P ratio: 4; Lipid mix: HY501/Cholesterol/DSPC/stealth lipid/Alfa lipid [47.5/38/10/1.8/0.2] with C16 PEG2k Ceramide or C14 Sar20 as stealth lipid and DSPE PEG2k Alfa, DOPE PSar20 Alfa or DOPE PSar10 Alfa. The LNPs were equipped with aCD3 VHH X NbAlfa ligand via post functionalization [w/w*=ligand to cargo ratio 0.48; molar ratio ligand to functionalized lipid 3.5:1]; RNA concentration: 0.1 μg/μl). Diameter of all LNPs is between 90 to 120 nm with a PDI below 0.3 as determined via DLS measurement. Successful RNA incorporation was verified via Agarose gel electrophoresis. For transfection studies, 10 μl (1000 ng dose) of respective formulations were pre-diluted in 50 μl X-Vivo 15 in an ultra-low adhesion 96 well plate. 1e6 thawed human PBMC were diluted in 100% PHS and added to nanoparticle dilution. Plates were incubated over night (37° C., 5% CO2). In the following cell-type specific transfection (Thy1.1) was analyzed via flowcytometry. Depicted are the percentages of transfected cell (CD14+ Monocytes, CD56+NK cells, CD19+ B cells, CD3+ T cells, CD4+ T cells or CD8+ T cells) within all transfected PBMCs (Transfection, y-axes) per tested formulation condition (see lower table).

FIG. 9: T-cell targeting using LNP in transgenic mouse model

LNPs were formulated with different lipid compositions and equipped with aCD3 VHH X NbAlfa ligand (Cargo: Thy1.1/Luc RNA 1:1 w/w; N/P ratio: 4; Lipid mix: DODMA/Cholesterol/DOPE/C16 PEG2k Ceramide/DSPE-PEG2k-Alfa [40/48/10/1.8/0.2]. aCD3-VHH X NbAlfa was added via post functionalization [w/w*=ligand to cargo ratio 1.15; molar ratio ligand to functionalized lipid 1.5:1]; RNA concentration: 0.1 μg/μl). Diameter of all LNPs is between 90 to 120 nm with a PDI below 0.3 as determined via DLS measurement. Successful RNA incorporation was verified via Agarose gel electrophoresis. 20 μg of the respective formulation was injected i.v. in the tail vain of B6-hCD3EDG transgenic mice. Prior to the injection the transgenic human CD3 expression on mouse T cells and the specific binding of the anti-hCD3VHH X NbAlfa ligand to the transgenic receptor was verified in vitro on B6-hCD3EDG splenocytes. 18 h after LNP injection animals were sacrificed and splenocytes were for cell-type specific Thy1.1 expression via flowcytometry. Depicted are Thy1.1 (Transfection, y-axes) signals against CD69 activation marker expression on non T/B cells, mCD19+ B cells and mCD4+ or mCD8+ T cells.

FIG. 10: B-cell targeting using LNP with algM

LNP were formulated with or without ligand (Cargo: Thy1.1/Luc RNA 1:1 w/w; N/P ratio: 4; Lipid mix: DODMA/Chol/DOPE/C16-PEG-Ceramide/DSPE-PEG2k-Alfa [40/48/10/2-x/x=0, 0.2]; algM-VHH×NbAlfa post-functionalization [w/w*=ligand to cargo ratio 0, 0.12, 0.25, 0.4; Final RNA concentration: 0.1 μg/μl). All formulation conditions lead to colloidal stable particles with a diameter between 100 to 150 nm and a PDI below 0.5. Diameter and PDI were determined via DLS measurement. Successful RNA incorporation was verified via Agarose gel electrophoresis. For transfection studies, 10 μl of respective formulations were pre-diluted in 50 μl X-Vivo 15 in a 96 deep-well plate. 1e6 thawed human PBMC were diluted in IMDM media without FCS and added to nanoparticle dilution (1000 ng RNA). After 30 min of incubation (37° C., 5% CO2) 900 μl IMDM+ HI FCS +IL4 and IL21 was added per well and cells were cultivated for additional 18 h (37° C., 5% CO2). In the following cell-type specific transfection (Thy1.1) was analyzed via flowcytometry. Depicted are the cell type specific Thy1.1 (Transfection in % multiplied with MFI, y-axes divided by 100) signals within viable CD14+ Monocytes, CD19+ B cells, CD3+ T cells.

FIG. 11: Freeze thaw studies

Particle size analysis of functionalized lipoplexes subjected to two freeze thaw cycles (A) from −20° C. to room temperature and (B) from −80° C. to room temperature.

FIG. 12: T-cell targeting using LNPs with different stealth lipids and Alfa lipids

LNPs were formulated with different lipid compositions (Cargo: Thy1.1 RNA/Luc RNA/Np proxy Venus 1:1:2 w/w; N/P ratio: 6; Lipid mix: HY501/Cholesterol/DSPC/stealth lipid/Alfa lipid. The lipid ratio has been selected as [47.5/40.5/10/1.8/0.2] for the following combinations of stealth lipid and Alfa lipid: C16 PEG2k Ceramide/DSPE PEG2k Alfa, DSPE PEG2k/DSPE PEG2k Alfa, DSPE pAEEA14/DSPE pAEEA14 Alfa or VitE pAEEA8/DSPE pAEEA14 Alfa. The lipid ratio has been selected as [47.5/38.5/10/3.8/0.2] for the following combinations of stealth lipid and Alfa lipid: VitE PEG1k/DSPE PEG2k Alfa or VitE pAEEA8/DSPE pAEEA14 Alfa. The LNPs were equipped with aCD3 VHH X NbAlfa ligand via post functionalization [w/w*=ligand to cargo ratio 0.48]; RNA concentration: 0.1 μg/μl). Diameter of all LNPs is between 100 to 170 nm with a PDI below 0.4 as determined via DLS measurement.

For transfection studies, 10 μl (1000 ng dose) of respective formulations were pre-diluted in 50 μl X-Vivo 15 in an ultra-low adhesion 96 well plate. 1e6 thawed human PBMC were diluted in 50 μl of 100% clotted PHS and added to nanoparticle dilution. After 30 min of incubation (37° C., 5% CO2) 30 μl of each transfection reaction was transferred to second ultra-low adhesion 96 well plate and 170 μl of X-Vivo 15 medium+100 U/ml IL2 was added per well. Cell dilutions were cultivated for additional 18 h (37° C., 5% CO2). In the following cell-type specific transfection (Thy1.1) was analyzed via flowcytometry. Depicted are the percentages of transfected cell (CD2 negative cells, CD19+ B cells, CD4+ T cells and CD8+ T cells) within all transfected PBMCs (Transfection, y-axes) per tested formulation condition (see lower table).

FIG. 13: Ligand-mediated transfection of T cells in vivo

Intramuscular injection of naked or LNP-formulated luciferase- and Thy1.1-encoding RNA mixtures (1:1 weight-to-weight mix) in B6-hCD3EDG transgenic mice (1 μg RNA dose per injection side; 2 μg total RNA dose per mouse). Analysis performed 18 h after injection. (A) Drainage analyzed via ex vivo bioluminescence imaging of popliteal, inguinal, axially and brachial lymph nodes, and spleens. (B) Cell-type specific transfection analyzed by flow-cytometry via detection of delivered Thy1.1 RNA expression in immune cell subtypes within the popliteal and inguinal lymph nodes, and spleens. (C) T-cell activation status analyzed via mean fluorescent intensity of CD69 surface expression within depicted organ (following staining with anti-CD69-APC antibody). LN, lymph nodes; LNP, lipid nanoparticle; NK, natural killer; PMN, polymorphonuclear cells.

### DETAILED DESCRIPTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art. In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present disclosure will employ, unless otherwise indicated, conventional chemistry, biochemistry, pharmaceutical, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of features, elements, members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

The term “optional” or “optionally” as used herein means that the subsequently described event, circumstance or condition may or may not occur, and that the description includes instances where said event, circumstance, or condition occurs and instances in which it does not occur.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, ±5%, 4%, 3%, 2%, 1%, +0.9%, 0.8%, 0.7%, 0.6%, 0.5%, 0.4%, 0.3%, 0.2%, 0.1%, 0.05%, and for example ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the following, definitions and embodiments will be provided which apply to all aspects of the present disclosure. Terms which are defined in the following have the meanings as defined unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e., a reduction to zero or essentially to zero.

Terms such as “enhance” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

The term “ionic strength” refers to the mathematical relationship between the number of different kinds of ionic species in a particular solution and their respective charges. Thus, ionic strength I is represented mathematically by the formula:

$$I = \frac{1}{2} \cdot {\sum }_{i} {z}_{i}^{2} \cdot {c}_{i}$$

in which c is the molar concentration of a particular ionic species and z the absolute value of its charge.

The sum Σ is taken over all the different kinds of ions (i) in solution.

According to the disclosure, the term “ionic strength” in some embodiments relates to the presence of monovalent ions. Regarding the presence of divalent ions, in particular divalent cations, their concentration or effective concentration (presence of free ions) due to the presence of chelating agents is, in some embodiments, sufficiently low so as to prevent degradation of a nucleic acid. In some embodiments, the concentration or effective concentration of divalent ions is below the catalytic level for hydrolysis of the phosphodiester bonds between nucleotides such as RNA nucleotides. In some embodiments, the concentration of free divalent ions is 20 μM or less. In some embodiments, there are no or essentially no free divalent ions.

“Osmolality” refers to the concentration of a particular solute expressed as the number of osmoles of solute per kilogram of solvent.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

The term “EDTA” refers to ethylenediaminetetraacetic acid disodium salt. All concentrations are given with respect to the EDTA disodium salt.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

According to the present disclosure, the term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. “Peptides” and “polypeptides” are both protein molecules. Thus, the terms “peptide”, “protein” and “polypeptide” are used herein usually as synonyms.

Peptides and polypeptides disclosed herein may comprise a linear or a cyclized peptide sequence.

In some embodiments, the peptides disclosed herein comprises at least one cyclic portion, i.e., a polypeptide chain that contains a circular sequence of bonds that is referred to herein as a “cyclic peptide.” The circular sequence can occur through a connection between the amino and carboxyl ends of the peptide; a connection between the amino end and a side chain; a connection between the carboxyl end and a side chain; or a connection between two side chains including sulfur groups of two cysteine amino acids by forming a disulfide bond, or more complicated arrangements.

In some embodiments, the peptides and polypeptides disclosed herein are composed of naturally occurring amino acids, non-naturally occurring amino acids, amino acid derivatives and non-amino acid components, or a mixture thereof. In some embodiments, the peptides and polypeptides disclosed herein comprise amino acid mimetics and amino acid analogs. In some embodiments, the peptides and polypeptides disclosed herein comprise non-naturally occurring amino acid sequences that are resistant to enzymatic cleavage.

In some embodiments, one or more positions of a peptide or polypeptide disclosed herein are substituted with a non-naturally occurring amino acid. In some embodiments, the substituted amino acid is chemically related to the original residue (e.g., aliphatic, charged, basic, acidic, aromatic, hydrophilic) or an isostere of the original residue.

In its broadest sense, as used herein, the term “amino acid” refers to a compound and/or substance that can be, is, or has been incorporated into a peptide, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid. In some embodiments, an amino acid is a D-amino acid. In some embodiments, an amino acid is an L-amino acid. “Standard amino acid” refers to any of the twenty standard L-amino acids commonly found in naturally occurring peptides and polypeptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a peptide or polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a peptide or polypeptide containing the modified amino acid as compared with one containing an otherwise identical unmodified amino acid. In some embodiments, such modification does not significantly alter a relevant activity of a peptide or polypeptide containing the modified amino acid, as compared with one containing an otherwise identical unmodified amino acid. As will be clear from context, in some embodiments, the term “amino acid” may be used to refer to a free amino acid. In some embodiments it may be used to refer to an amino acid residue of a peptide or polypeptide.

The following table lists the 20 natural amino acids and their abbreviations:

| | |
|---|---|
| Name | Abbr |

| | | |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Generally, amino acids are L-amino acids while D-amino acids are denoted by the prefix “D”. The prefix “homo” or “h” designates an α-amino acid that is otherwise similar to one of the common ones, but that contains one more methylene group in the carbon chain.

As used herein, “Orn” means ornithine or 2,5-diaminopentanoic acid, “Dab” means 2,4-diaminobutanoic acid, “Dap” means 2,3-diaminopropanoic acid, “hLys” means 2,7-diaminoheptanoic acid, “hCys” means 2-amino-4-mercaptobutanoic acid, and “Pen” means penicillamine or 2-amino-3-methyl-3-sulfanylbutanoic acid.

It may also be possible to include non-peptide linkages and other chemical modification. For example, part or all of the peptide or polypeptide may be synthesized as a peptidomimetic, e.g., a peptoid (see, e.g., Simon et al. (1992) Proc. Natl. Acad. Sci. USA 89:9367-71 and Horwell (1995) Trends Biotechnol. 13:132-4). A peptide or polypeptide may include one or more (e.g., all) non-hydrolyzable bonds. Many non-hydrolyzable peptide bonds are known in the art, along with procedures for synthesis of peptides containing such bonds. Exemplary non-hydrolyzable bonds include —[CH₂NH]— reduced amide peptide bonds, —[COCH₂]— ketomethylene peptide bonds, —[CH(CN)NH]— (cyanomethylene)amino peptide bonds, —[CH₂CH(OH)]— hydroxyethylene peptide bonds, —[CH₂O]— oxymethylene peptide bonds, and —[CH₂S]— thiomethylene peptide bonds (see e.g., U.S. Pat. No. 6,172,043).

The term “amide” as used herein, represents a group of formula “—NHC(O)—”.

The term “thioamide” represents a group of formula “—NHC(S)—”.

As used herein the term “disulfide bond”, “disulfide bridge” or “disulfide” includes the covalent bond formed between two sulfur atoms. The amino acid cysteine comprises a thiol group that can form a disulfide bond or bridge with a second thiol group.

The term “ether” refers to a group or compound having an oxygen between two carbon atoms.

The term “thioether” refers to a group or compound having a sulfur between two carbon atoms.

The term “ester” refers a compound derived from an carboxylic acid and an alcohol by linking with formal loss of water the hydroxyl group of the —C(═O)OH group in the former and a hydroxy group of the latter.

Thus, the term refers to the group —C(O)O—.

The term “thioester” refers to the group —C(O)S—.

The term “triazole” refers to chemical compounds that incorporate in their structure any heterocyclic structure having a five-membered ring of two carbon atoms and three nitrogen atoms (e.g., 1,2,3-triazole).

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may be any portion from 0.1% to 99.9% (such as 0.1%, 0.5%, 1%, 5%, 10%, 50%, 90%, or 99%) of said composition.

“Fragment”, with reference to an amino acid sequence (peptide or polypeptide), relates to a part of an amino acid sequence, i.e., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence comprises, e.g., at least 6, in particular at least 8, at least 10, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30 or up to 55, consecutive amino acids of the amino acid sequence.

“Variant,” as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

By “wild type” or “WT” or “native” as used herein and with reference to an amino acid sequence (peptide or polypeptide) is meant an amino acid sequence that is found in nature, including allelic variations. A wild type amino acid sequence, peptide or polypeptide has an amino acid sequence that has not been intentionally modified.

For the purposes of the present disclosure, “variants” of an amino acid sequence (peptide or polypeptide) may comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous peptides or polypeptides and/or to replacing amino acids with other ones having similar properties. In some embodiments, amino acid changes in peptide and polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

In some embodiments, the degree of similarity, such as identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given, e.g., for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, such as sequence identity can be done with art known tools, such as using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of algorithms, e.g., the local homology algorithm by Smith and Waterman, 1981, Ads App. Math. 2, 482, the local homology algorithm by Neddleman and Wunsch, 1970, J. Mol. Biol. 48, 443, the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2se q). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and, e.g., at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or polypeptides having substitutions, additions, insertions or deletions, is described in detail in Molecular Cloning: A Laboratory Manual, 4th Edition, M. R. Green and J. Sambrook et al. (1989), eds., Cold Spring Harbor Laboratory Press, Cold Spring Harbor 2012, for example. Furthermore, the peptides, polypeptides and amino acid variants described herein may be readily prepared with the aid of known peptide synthesis techniques such as, for example, by solid phase synthesis and similar methods.

In some embodiments, a fragment or variant of an amino acid sequence (peptide or polypeptide) is a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to sequences of binding agents such as antibodies, one particular function is one or more binding activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., function of the functional fragment or functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide or polypeptide) “derived from” a designated amino acid sequence (peptide or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the sequences suitable for use herein may be altered such that they vary in sequence from the naturally occurring or native sequences from which they were derived, while retaining the desirable activity of the native sequences.

In some embodiments, “isolated” means removed (e.g., purified) from the natural state or from an artificial composition, such as a composition from a production process. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

The term “bind” or “binding” relates to the non-covalent interaction with a target. In some embodiments, the term “bind” or “binding” relates to a specific binding. By the term “specific binding” or “specifically binds”, as used herein, is meant a molecule such as an antibody or antigen receptor which recognizes a specific target molecule, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binds”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “binding” or “capable of binding” typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI), or, for instance, when determined using surface plasmon resonance (SPR) technology in a BIAcore 3000 instrument. In some embodiments, a binding moiety or agent binds to a predetermined target with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific target (e.g., BSA, casein).

The term “k_{d}” (sec⁻¹), as used herein, refers to the dissociation rate constant of a particular interaction, e.g., antibody-antigen interaction. Said value is also referred to as the k_{off} value.

The term “K_{D}” (M), as used herein, refers to the dissociation equilibrium constant of a particular interaction, e.g., antibody-antigen interaction.

Generally, the terms “bind” or “binding” and “target” or “targeting” are used interchangeably herein.

The term “genetic modification” or simply “modification” includes the transfection of cells with nucleic acid. The term “transfection” relates to the introduction of nucleic acids, e.g., DNA and/or RNA, into a cell. For purposes of the present disclosure, the term “transfection” also includes the introduction of a nucleic acid into a cell or the uptake of a nucleic acid by such cell, wherein the cell may be present in a subject, e.g., a patient, or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. Generally, cells that are genetically modified to express an antigen receptor are stably transfected with nucleic acid encoding the antigen receptor. RNA can be transfected into cells to transiently express its coded protein.

As used herein, the terms “linked”, “fused”, or “fusion” are used interchangeably. These terms refer to the joining together of two or more elements or components or domains.

The term “fusion protein” as used herein refers to a polypeptide or protein comprising two or more subunits. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a linker.

As used herein “endogenous” refers to any material from or produced inside an organism, cell, tissue or system.

As used herein, the term “exogenous” refers to any material introduced from or produced outside an organism, cell, tissue or system.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

According to various embodiments of the present disclosure, a nucleic acid encoding a peptide or polypeptide is taken up by or introduced, i.e. transfected or transduced, into a cell which cell may be present in vitro or in a subject, resulting in expression of said peptide or polypeptide. The cell may, e.g., express the encoded peptide or polypeptide intracellularly (e.g. in the cytoplasm and/or in the nucleus), may secrete the encoded peptide or polypeptide, and/or may express it on the surface. In some embodiments, if the encoded peptide or polypeptide is an antigen receptor, the cell expresses the antigen receptor on the cell surface.

According to the present disclosure, terms such as “nucleic acid expressing” and “nucleic acid encoding” or similar terms are used interchangeably herein and with respect to a particular peptide or polypeptide mean that the nucleic acid, if present in the appropriate environment, e.g. within a cell, can be expressed to produce said peptide or polypeptide.

The term “expression” as used herein includes the transcription and/or translation of a particular nucleotide sequence.

In the context of the present disclosure, the term “transcription” relates to a process, wherein the genetic code in a DNA sequence is transcribed into RNA (especially mRNA). Subsequently, the RNA may be translated into peptide or polypeptide.

With respect to RNA, the term “expression” or “translation” relates to the process in the ribosomes of a cell by which a strand of mRNA directs the assembly of a sequence of amino acids to make a peptide or polypeptide.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions/formulations of the present disclosure or be shipped together with a container which contains the compositions/formulations. Alternatively, the instructional material may be shipped separately from the container with the intention that the instructional material and the compositions be used cooperatively by the recipient.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses mᵢ (i=1, 2, 3, . . . , n), located at fixed distances sᵢ from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors \vec{q} the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; T is the temperature; η is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles contained in a sample or control composition as disclosed herein or the hydrodynamic radius of a particle peak obtained from subjecting such a sample or control composition to field-flow fractionation) is to measure the DLS signal of said particle or population of particles (such as DLS signal of particles contained in a sample or control composition as disclosed herein or the DLS signal of a particle peak obtained from subjecting such a sample or control composition to field-flow fractionation).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In certain embodiments, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{{K}^{*} c} = {M}_{w} P ( θ ) - 2 {A}_{2} c {M}_{w}^{2} {P}^{2} ( θ )$$

wherein c is the mass concentration of the particles in the solvent (g/mL); A₂ is the second virial coefficient (mol·mL/g²); P(ϑ) is a form factor relating to the dependence of scattered light intensity on angle; R_{ϑ} is the excess Rayleigh ratio (cm⁻¹); and K* is an optical constant that is equal to 4π²ηₒ (dn/dc)²λ₀⁻⁴N_{A}⁻¹, where no is the refractive index of the solvent at the incident radiation (vacuum) wavelength, λ₀ is the incident radiation (vacuum) wavelength (nm), N_{A} is Avogadro's number (mol⁻¹), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated using the following term or the reciprocal thereof:

$$\sqrt{\frac{{R}_{θ}}{{K}^{*} c}}$$

wherein c, R_{ϑ} and K* are as defined above. Preferably, the Debye plot is calculated using the following term or the reciprocal thereof:

$$\frac{{K}^{*} c}{{R}_{θ}}$$

wherein c, R_{ϑ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

### Targeting Compound

The particles described herein comprising a nucleic acid payload to be delivered comprise a hydrophobic moiety (e.g., lipid) having a binding moiety covalently attached thereto. This hydrophobic moiety having a binding moiety covalently attached thereto is also referred to herein as “targeting compound”. The hydrophobic moiety of the targeting compound relates to the part of the targeting compound that integrates into the particle comprising a nucleic acid payload. The binding moiety of the targeting compound relates to the part of the targeting compound that binds to target cells or forms the binding partner for a docking compound which binds to target cells. Generally, the targeting compound is non-covalently incorporated into the particle comprising a payload, i.e., it forms an integral part of the particle, and the binding moiety of the targeting compound is covalently attached to a hydrophobic moiety in a manner such that it is available for binding to target cells or a docking compound.

In some embodiments, the binding moiety of the targeting compound comprises a peptide or protein (e.g., an antibody or antibody fragment or a peptide tag).

In some embodiments, the binding moiety of the targeting compound comprises a peptide or protein (e.g., an antibody or antibody fragment or a peptide tag) and is chemically linked, e.g., through a linker, to the hydrophobic moiety (e.g., lipid).

The targeting compound described herein comprises a hydrophobic component (e.g., lipid component) which allows it to be anchored in the particle. In some embodiments, the hydrophobic component comprises a moiety selected from vitamin E, dialkylamine, e.g., dimyristylamine (DMA), diacylglyceride, e.g., 1,2-dimyristoyl-sn-glycerol (DMG) and ceramide. In some embodiments, the hydrophobic moiety comprises two C8-C24 hydrocarbon chains. In some embodiments, the hydrophobic moiety comprises two C10-C18 hydrocarbon chains.

In some embodiments, the targeting compound described herein has as a hydrophobic group (e.g., lipid) a phospholipid, e.g., a biodegradable phospholipid such as phosphatidylethanolamine. In some embodiments, the targeting compound described herein has as a hydrophobic group (e.g., lipid) a glycerophospholipid. In some embodiments, the phospholipid is selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof. In some embodiments, as a phospholipid, DSPE will be used for its qualities of stability in the particles described herein. Moreover, as hydrophobic group (e.g., lipid), a compound having at least one alkyl chain providing hydrophobic anchoring to a particle as described herein may be used.

In some embodiments, the targeting compound comprises a polymer. In some embodiments, the hydrophobic moiety (e.g., lipid) of the targeting compound and the binding moiety of the targeting compound are connected through the polymer.

In some embodiments, the polymer is a hydrophilic polymer and the targeting compound comprises an amphiphilic derivative of the polymer. In some embodiments, the amphiphilic derivative of a polymer comprises a hydrophobic component (e.g., lipid component) which allows it to be anchored in the particle and a hydrophilic component of the polymer facing the outside of said particle, conferring hydrophilic properties at the surface thereof. In some embodiments, the amphiphilic derivatives of a polymer is inserted into the particle via its hydrophobic end. Consequently, the polymer component faces the outside of said particle and forms a protective hydrophilic shell surrounding the particle. In some embodiments, the polymer portion of the amphiphilic derivative contributes to conferring stealth properties on the particles. In some embodiments, the plasmatic half-life of the particles described herein is greater than 2 hours, e.g., between 3 and 10 hours. This characteristic advantageously allows the particles to accumulate at the target cells and to liberate therein their contents (payload) within reasonable amounts of time. The effectiveness of the targeted delivery described herein therefore increases as a result.

The term “stealth” is used herein to describe the ability of the particles described herein not to be detected and then sequestered and/or degraded, or to be hardly detected and then sequestered and/or degraded, and/or to be detected and then sequestered and/or degraded late, by the immune system of the host to which they are administered.

Macrophages constitute one of the most important components of the immune system and play a predominant role in eliminating foreign particles, including liposomes and other colloidal particles, from the blood circulation. At the molecular level, the clearance of particles takes place in two steps: opsonization by the depositing of serum proteins (or “opsonins”) at the surface of the particles followed by recognition and capture of the opsonized particles by macrophages.

Modification of the surface of particles with chains of hydrophilic and flexible polymers, e.g., polymers of the poly(ethylene glycol) type, confers them a steric protection by preventing the opsonins reaching the surface of the particles.

In some embodiments, the amphiphilic derivative of a polymer used herein has as a hydrophobic group (e.g., lipid) as specified herein. In some embodiments, the amphiphilic derivative of a polymer used herein has as a hydrophobic group (e.g., lipid) a phospholipid, e.g., a biodegradable phospholipid such as phosphatidylethanolamine. In some embodiments, the phospholipid is selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof. In some embodiments, as a phospholipid, DSPE will be used for its qualities of stability in the particles described herein. Moreover, as hydrophobic group (e.g., lipid), a compound having at least one alkyl chain providing hydrophobic anchoring to a particle as described herein may be used.

In some embodiments, the polymer for use herein is selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof).

In some embodiments, a polymer is designed to sterically stabilize a particle by forming a protective hydrophilic layer. In some embodiments, a polymer can reduce association of a particle with serum proteins and/or the resulting uptake by the reticuloendothelial system when such particles are administered in vivo.

In some embodiments, the PEG is an optionally substituted linear or branched polymer of ethylene glycol or ethylene oxide. In some embodiments, the PEG is unsubstituted. In some embodiments, the PEG is substituted, e.g., by one or more alkyl, alkoxy, acyl, hydroxy or aryl groups. In some embodiments, the PEG has a molecular weight of from about 130 to about 50,000, in another embodiment about 150 to about 30,000, in another embodiment about 150 to about 20,000, in another embodiment about 150 to about 15,000, in another embodiment about 150 to about 10,000, in another embodiment about 150 to about 6000, in another embodiment about 150 to about 5000, in another embodiment about 150 to about 4000, in another embodiment about 150 to about 3000, in another embodiment about 300 to about 3000, in another embodiment about 1000 to about 3000, and in still another embodiment about 1500 to about 2500.

In some embodiments, the PEG moiety of the amphiphilic derivative of a polymer has a molecular weight of 1000 or more. In some embodiments, the PEG moiety of the amphiphilic derivative of a polymer comprises 10 units or more of formula (O—CH₂—CH₂)ₙ. In some embodiments, the PEG comprises from 20 to 200 ethylene oxide units, such as about 45 ethylene oxide units.

In some embodiments, the PEG comprises “PEG2k”, also termed “PEG 2000”, which has an average molecular weight of about 2000 Daltons.

In some embodiments, DSPE-PEG₂₀₀₀, DSPE-PEG₃₀₀₀ and DSPE-PEG₅₀₀₀ are used as the amphiphilic derivative of a polymer.

In some embodiments, a pSar comprises between 2 and 200 sarcosine units, such as between 5 and 100 sarcosine units, between 10 and 50 sarcosine units, between 15 and 40 sarcosine units, e.g., about 23 sarcosine units.

In some embodiments, a pSar comprises the structure of the following general formula:

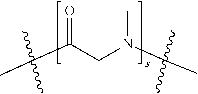

wherein s is the number of sarcosine units.

In some embodiments, the POX and/or POZ polymer comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 POX and/or POZ repeating units.

In some embodiments, the POX and/or POZ polymer comprises the following general formula:

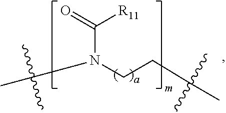

wherein a is an integer between 1 and 2; Ru is alkyl, in particular C₁₋₃ alkyl, such as methyl, ethyl, iso-propyl, or n-propyl, and is independently selected for each repeating unit; and m refers to the number of POX and/or POZ repeating units.

In some embodiments, the POX and/or POZ polymer is a polymer of POX and comprises repeating units of the following general formula:

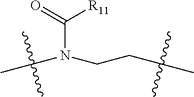

In some embodiments, the POX and/or POZ polymer is a polymer of POZ and comprises repeating units of the following general formula:

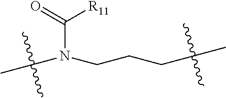

In any of the above embodiments of formulas, m (i.e., the number of repeating units in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments, m is 2 to 180, such as 4 to 160, 6 to 140, 8 to 120 or 10 to 100, e.g., 20 to 80, 30 to 70, or 40 to 50.

In some embodiments, the POX and/or POZ polymer is a copolymer comprising repeating units of the following general formulas:

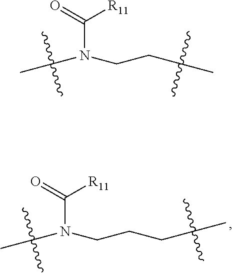

wherein the number of repeating units shown on the left in the copolymer is 1 to 199; the number of repeating units of formula on the right in the copolymer is 1 to 199; and the sum of the number of repeating units of formula on the left and the number of repeating units of formula on the right in the copolymer is 2 to 200.

In some embodiments of the oxazolinylated and/or oxazinylated hydrophobic moiety (e.g., lipid), the number of repeating units of formula on the left in the copolymer is 1 to 179, such as 1 to 159, 1 to 139, 1 to 119 or 1 to 99; the number of repeating units of formula on the right in the copolymer is 1 to 179, such as 1 to 159, 1 to 139, 1 to 119 or 1 to 99; and the sum of the number of repeating units of formula on the left and the number of repeating units of formula on the right in the copolymer is 2 to 180, such as 4 to 160, 6 to 140, 8 to 120 or 10 to 100, e.g., 20 to 80, 30 to 70, or 40 to 50.

In some of the above embodiments, Ru at each occurrence (i.e., in each repeating unit) may be the same alkyl group (e.g., Ru may be methyl in each repeating unit). In some alternative embodiments, Ru in at least one repeating unit differs from Ru in another repeating unit (e.g., for at least one repeating unit Ru is one specific alkyl (such as ethyl), and for at least one different repeating unit Ru is a different specific alkyl (such as methyl)). For example, each Ru may be selected from two different alkyl groups (such as methyl and ethyl) and not all Ru are the same alkyl.

In any of the above embodiments, Ru preferably is methyl or ethyl, more preferably methyl. Thus, in some embodiments, each Ru is methyl or each Ru is ethyl. In some alternative embodiments, Ru is independently selected from methyl and ethyl for each repeating unit, wherein in at least one repeating unit R₁₁ is methyl, and in at least one repeating unit R₁₁ is ethyl.

In some embodiments, the polymer comprises poly-2-(2-(2-aminoethoxy)ethoxy)acetic acid (pAEEA) or poly-2-(2-(2-methylaminoethoxy)ethoxy)acetic acid (pMAEEA), or a derivative thereof.

In some embodiments, the polymer comprises the following general formula:

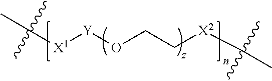

wherein
X₂ and X₁ taken together are optionally substituted amide, optionally substituted thioamide or ester;
Y is —CH₂—, —(CH₂)₂—, or —(CH₂)₃—;
z is 2 to 24; and
n is 1 to 100.

In some embodiments,
(i) when X¹ is —C(O)— then X² is —NR¹—;
(ii) when X¹ is —NR¹— then X² is —C(O)—;
(iii) when X¹ is —C(S)— then X² is —NR¹—;
(iv) when X¹ is —NR¹— then X² is —C(S)—;
(v) when X¹ is —C(O)— then X² is —O—; or
(vi) when X¹ is —O— then X² is —C(O)—;
wherein R¹ is hydrogen or C₁₋₈ alkyl.

In some embodiments, X¹ is —C(O)— and X² is —NR¹—, wherein R¹ is hydrogen or C₁₋₈ alkyl. In some embodiments, X¹ is —C(O)— and X² is —NR¹—, wherein R¹ is hydrogen or methyl. In some embodiments, X¹ is —C(O)— and X² is —NR¹—, wherein R¹ is hydrogen.

In some embodiments, Y is —CH₂— or —(CH₂)₂—. In some embodiments, Y is —CH₂—.

In some embodiments, the polymer comprises the following general formula:

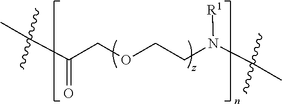

wherein
R¹ is hydrogen or C₁₋₈ alkyl;
z is 2 to 24; and
n is 1 to 100.

In some embodiments of the above formulas, z is 2 to 10. In some embodiments, z is 2 to 7. In some embodiments, z is 2 to 5. In some embodiments, z is 2 or 3. In some embodiments, z is 2.

In some embodiments, the polymer comprises the following general formula:

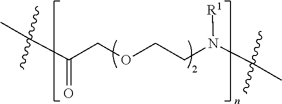

wherein
R¹ is hydrogen or C₁₋₈ alkyl; and
n is 1 to 100.

In some embodiments of the above formulas, R¹ is hydrogen or methyl. In some embodiments, R¹ is hydrogen.

In some embodiments, the polymer comprises the following general formula:

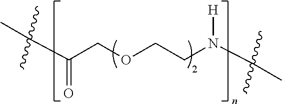

wherein
n is 1 to 100.

In some embodiments of the above formulas, n is 5 to 50. In some embodiments, n is 5 to 25. In some embodiments, n is 7 to 14. In some embodiments, n is 10 to 25. In some embodiments, n is 14 to 17. In some embodiments, n is 8 or 14.

In some embodiments, the molar proportion of the amphiphilic derivative of a polymer integrated into the particles is between 0.5 and 20 mol % of the lipid molecules making up the particle, preferably between 1 and 10 mol %.

In some embodiments, the targeting compound comprises the following general formula:

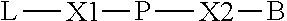

wherein
P comprises a polymer;
L comprises a hydrophobic moiety (e.g., lipid) attached to a first end of the polymer;
B comprises a binding moiety attached to a second end of the polymer;
X1 is absent or a first linking moiety; and
X2 is absent or a second linking moiety.

In some embodiments, X1 comprises a carbonyl group. In some embodiments, L comprises a phosphatidylethanolamine which may be linked to P by an amide group.

In some embodiments, X2 comprises the reaction product of a thiol or cysteine reactive group, e.g., a maleimide group, with a thiol or cysteine group of a compound comprising the binding moiety.

In some embodiments, L comprises a lipid as described above. In some embodiments, L comprises DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine) which may be linked to P by an amide group.

In some embodiments, P comprises a polymer as described above. In some embodiments, P comprises a polymer which provides stealth property, extends circulation half-life and/or reduces non-specific protein binding or cell adhesion. In some embodiments, P comprises a polymer selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof). In some embodiments, P comprises polyethyleneglycol (PEG); e.g., PEG as described above.

In some embodiments, L-X1-P comprises an amphiphilic derivative of a polymer as described above. In some embodiments, the amphiphilic derivative of a polymer comprises a conjugate of disteroyl-glycero-phosphoethanolamine (DSPE) and a polymer, e.g., a polymer as described above. In some embodiments, the amphiphilic derivative of a polymer comprises a disteroyl-glycero-phosphoethanolamine-polyethyleneglycol-conjugate (DSPE-PEG).

In some embodiments, the targeting compound is obtainable by reacting the thiol or cysteine reactive group of a reagent comprising an amphiphilic derivative of a polymer, e.g., a PEG reagent comprising a hydrophobic moiety (e.g., lipid), with a thiol or cysteine group of a compound comprising the binding moiety.

In some embodiments, the thiol or cysteine reactive group comprises a maleimide group.

In some embodiments, the PEG reagent comprises DSPE-PEG-maleimide. In some embodiments, the compound comprising the binding moiety comprises the formula SH(CH₂)nC(O)—B, wherein n ranges from 1 to 5 and B comprises the binding moiety. In some embodiments, n is 2.

In some embodiments, the targeting compound comprises the reaction product of 1,2-distearoyl-sn-glycero-3-phosphoethanolamine-N-[maleimide(polyethylene glycol)] with a compound comprising the formula SH(CH₂)ₙC(O)—B, wherein n ranges from 1 to 5 and B comprises the binding moiety. In some embodiments, n is 2.

In some embodiments, the targeting compound (hydrophobic moiety having a binding moiety covalently attached thereto) comprises the following general formula: L-X1-P-X2-B

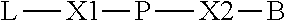

wherein L, X1, P and B are as described above and X2 comprises a thiosuccinimide moiety.

In some embodiments, the targeting compound (hydrophobic moiety having a binding moiety covalently attached thereto) comprises the following general formula:

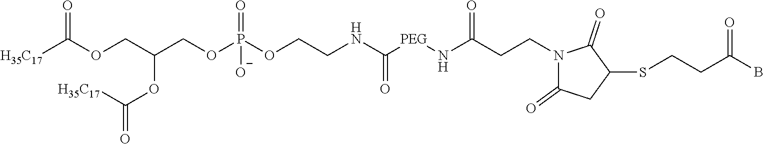

wherein B comprises the binding moiety.

In some embodiments of the above formulas, B comprises a moiety comprising the structure —N-peptide-C(O)—NH2.

In some embodiments, the targeting compound (hydrophobic moiety having a binding moiety covalently attached thereto) comprises the following general formula:

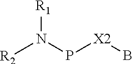

wherein P, X2 and B are as described above and R₁ and R₂ independently comprise an alkyl moiety. In some embodiments, at least one, e.g., each alkyl moiety is straight or branched, preferably straight. In some embodiments, at least one, e.g., each alkyl moiety has at least 8 carbon atoms, e.g., 8 to 24 such as 10 to 18 carbon atoms. Preferably, at least one, e.g., each alkyl moiety is the alkyl moiety of a fatty acid alcohol, more preferably at least one, e.g., each alkyl moiety is the alkyl moiety of a fatty acid alcohol having at least 8 carbon atoms, e.g., 8 to 24 such as 10 to 18 carbon atoms. Examples of alkyl moieties include —(CH₂)₁₇CH₃ (stearyl), —(CH₂)₁₅CH₃ (palmityl), and —(CH₂)₁₃CH₃ (myristyl). In some embodiments, R₁R₂N— in the above formula is 1,2-dimyristylamine, wherein both alkyl groups are —(CH₂)₁₃CH₃ (myristyl). In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

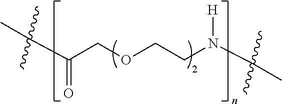

wherein n is 5 to 50, e.g., 5 to 25, e.g., 7 to 14, e.g., 10 to 25, e.g., 14 to 17. In some embodiments, n is 8 or 14. In some embodiments, n is 14. In some embodiments, R₁ and R₂ in the above formula are —(CH₂)₁₃CH₃ (myristyl) and the polymer P comprises the following general formula:

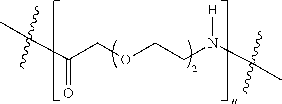

wherein n is 14.

In some embodiments, the targeting compound (hydrophobic moiety having a binding moiety covalently attached thereto) comprises the following general formula:

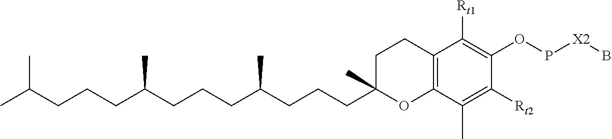

wherein P, X2 and B are as described above and each of Rₜ₁ and Rₜ₂ is independently H or methyl. In some embodiments, Rₜ₁ and Rₜ₂ are both methyl. In some embodiments, Rₜ₁ is methyl, and Rₜ₂ is H. In some embodiments, Rₜ₁ is H, and Rₜ₂ is methyl. In some embodiments, Rₜ₁ and Rₜ₂ are both H.

In some embodiments, the targeting compound (hydrophobic moiety having a binding moiety covalently attached thereto) comprises the following general formula:

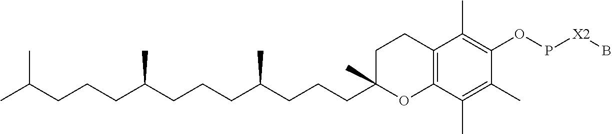

wherein P, X2 and B are as described above.

In some embodiments, the polymer P in the above formulas comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

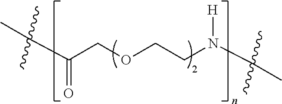

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, the targeting compound (hydrophobic moiety having a binding moiety covalently attached thereto) comprises the following general formula:

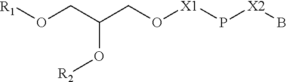

wherein X1, P, X2 and B are as described above and R₁ and R₂ independently comprise an acyl moiety. In some embodiments, at least one, e.g., each acyl moiety is straight or branched, preferably straight. In some embodiments, at least one, e.g., each acyl moiety has at least 8 carbon atoms, e.g., 8 to 24 such as 10 to 18 carbon atoms. Preferably, at least one, e.g., each acyl moiety is the acyl moiety of a fatty acid, more preferably at least one, e.g., each acyl moiety is the acyl moiety of a fatty acid having at least 8 carbon atoms, e.g., 8 to 24 such as 10 to 18 carbon atoms. Examples of acyl moieties include CH₃(CH₂)₁₆C(O)— (stearoyl), CH₃(CH₂)₁₄C(O)— (palmitoyl), and CH₃(CH₂)₁₂C(O)— (myristoyl). In some embodiments, both acyl groups are CH₃(CH₂)₁₆C(O)— (stearoyl). In some embodiments, both acyl groups are CH₃(CH₂)₁₂C(O)— (myristoyl). In some embodiments, X1 is absent or comprises —HPO₃—(CH₂)n-NH—, wherein n is 1 to 5, e.g., 2.

In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

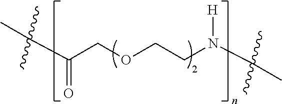

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, the polymer P comprises a pSar. In some embodiments, the polymer P comprises the following general formula:

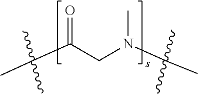

wherein s is 2 to 200, e.g., 5 to 100, e.g., 10 to 50, e.g., 15 to 40. In some embodiments, s is 20 or 23.

In some embodiments, the targeting compound (hydrophobic moiety having a binding moiety covalently attached thereto) comprises the following general formula:

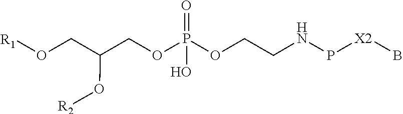

wherein P, X2 and B are as described above and R₁ and R₂ independently comprise an acyl moiety. In some embodiments, at least one, e.g., each acyl moiety is straight or branched, preferably straight. In some embodiments, at least one, e.g., each acyl moiety has at least 8 carbon atoms, e.g., 8 to 24 such as 10 to 18 carbon atoms. Preferably, at least one, e.g., each acyl moiety is the acyl moiety of a fatty acid, more preferably at least one, e.g., each acyl moiety is the acyl moiety of a fatty acid having at least 8 carbon atoms, e.g., 8 to 24 such as 10 to 18 carbon atoms. Examples of acyl moieties include CH₃(CH₂)₁₆C(O)— (stearoyl), CH₃(CH₂)₁₄C(O)— (palmitoyl), and CH₃(CH₂)₁₂C(O)— (myristoyl). In some embodiments, both acyl groups are CH₃(CH₂)₁₆C(O)— (stearoyl). In some embodiments, both acyl groups are CH₃(CH₂)₁₂C(O)— (myristoyl).

In some embodiments, the polymer P comprises poly-2-(2-(2-aminoethoxy)ethoxy)acetic acid (pAEEA) or poly-2-(2-(2-methylaminoethoxy)ethoxy)acetic acid (pMAEEA), or a derivative thereof. In some embodiments, the polymer P comprises the following general formula:

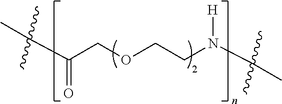

wherein n is 5 to 50, e.g., 5 to 25, e.g., 7 to 14, e.g., 10 to 25, e.g., 14 to 17. In some embodiments, n is 8 or 14. In some embodiments, n is 8. In some embodiments, n is 14.

In some embodiments, n is 8 and R₁ and R₂ are CH₃(CH₂)₁₆C(O)— (stearoyl). In some embodiments, n is 14 and R₁ and R₂ are CH₃(CH₂)₁₆C(O)— (stearoyl).

In some embodiments, n is 8 and R₁ and R₂ are CH₃(CH₂)₁₂C(O)— (myristoyl). In some embodiments, n is 14 and R₁ and R₂ are CH₃(CH₂)₁₂C(O)— (myristoyl).

In some embodiments, the polymer P comprises a pSar. In some embodiments, the polymer P comprises the following general formula:

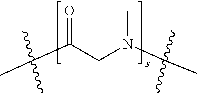

wherein s is 2 to 200, e.g., 5 to 100, e.g., 10 to 50, e.g., 15 to 40. In some embodiments, s is 20 or 23.

In some embodiments, s is 20 and R₁ and R₂ are CH₃(CH₂)₁₆C(O)— (stearoyl).

In some embodiments, s is 20 and R₁ and R₂ are CH₃(CH₂)₁₂C(O)— (myristoyl).

In some embodiments, X2 in the above formulas comprises the reaction product of a thiol or cysteine reactive group, e.g., a maleimide group, with a compound comprising a thiol or cysteine group. In some embodiments, the compound comprising a thiol or cysteine group comprises the formula SH(CH₂)ₙC(O)—, wherein n ranges from 1 to 5. In some embodiments, n is 2. In some embodiments, X2 comprises a thiosuccinimide moiety.

In some embodiments, X2 comprises the following general formula:

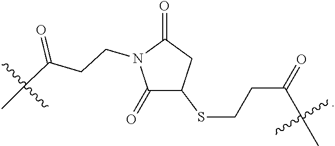

In some embodiments, X2 comprises the following general formula:

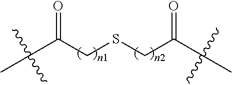

wherein n1 and n2 are independently 1 to 5. In some embodiments, n1 is 1 and n2 is 2. In some embodiments, n1 is 2 and n2 is 1.

The present disclosure provides in one aspect, a targeting compound as described herein. In some embodiments of the targeting compound, the binding moiety comprises a moiety binding to a cell surface antigen, e.g., a primary targeting moiety described herein. In some embodiments of the targeting compound, the binding moiety comprises a moiety binding to a docking compound. In some embodiments of the targeting compound, the binding moiety comprises an epitope tag, e.g., an ALFA-tag such as an ALFA-tag described herein.

Accordingly, the present disclosure provides in one aspect, a compound the following general formula: L-X1-P-X2-B

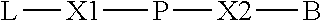

wherein
P comprises a polymer;
L comprises a hydrophobic moiety (e.g., lipid) attached to a first end of the polymer;
B comprises a primary targeting moiety described herein, attached to a second end of the polymer;
X1 is absent or a first linking moiety; and
X2 is absent or a second linking moiety.

The present disclosure further provides in one aspect, a compound the following general formula:

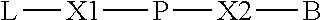

wherein
P comprises a polymer;
L comprises a hydrophobic moiety (e.g., lipid) attached to a first end of the polymer;
B comprises an epitope tag, e.g., an ALFA-tag such as an ALFA-tag described herein, attached to a second end of the polymer;
X1 is absent or a first linking moiety; and
X2 is absent or a second linking moiety.

In some embodiments, X1 comprises a carbonyl group. In some embodiments, L comprises a phosphatidylethanolamine which may be linked to P by an amide group.

In some embodiments, X2 comprises the reaction product of a thiol or cysteine reactive group, e.g., a maleimide group, with a thiol or cysteine group of a compound comprising the epitope tag. In some embodiments, X2 comprises a thiosuccinimide moiety.

In some embodiments, L comprises a lipid as described above. In some embodiments, L comprises DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine) which may be linked to P by an amide group.

In some embodiments, P comprises a polymer as described above. In some embodiments, P comprises a polymer which provides stealth property, extends circulation half-life and/or reduces non-specific protein binding or cell adhesion. In some embodiments, P comprises a polymer selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof). In some embodiments, P comprises polyethyleneglycol (PEG); e.g., PEG as described above.

In some embodiments, L-X1-P comprises an amphiphilic derivative of a polymer as described above. In some embodiments, the amphiphilic derivative of a polymer comprises a conjugate of disteroyl-glycero-phosphoethanolamine (DSPE) and a polymer, e.g., a polymer as described above. In some embodiments, the amphiphilic derivative of a polymer comprises a disteroyl-glycero-phosphoethanolamine-polyethyleneglycol-conjugate (DSPE-PEG).

In some embodiments, the targeting compound is obtainable by reacting the thiol or cysteine reactive group of a reagent comprising an amphiphilic derivative of a polymer, e.g., a PEG reagent comprising a hydrophobic moiety (e.g., lipid), with a thiol or cysteine group of a compound comprising the primary targeting moiety or epitope tag.

In some embodiments, the thiol or cysteine reactive group comprises a maleimide group.

In some embodiments, the PEG reagent comprises DSPE-PEG-maleimide. In some embodiments, the compound comprising the primary targeting moiety or epitope tag comprises the formula SH(CH₂)ₙC(O)—B, wherein n ranges from 1 to 5 and B comprises the primary targeting moiety or epitope tag. In some embodiments, n is 2.

In some embodiments, the targeting compound comprises the reaction product of 1,2-distearoyl-sn-glycero-3-phosphoethanolamine-N-[maleimide(polyethylene glycol)] with a compound comprising the formula SH(CH₂)ₙC(O)—B, wherein n ranges from 1 to 5 and B comprises the primary targeting moiety or epitope tag. In some embodiments, n is 2.

In some embodiments, the targeting compound (hydrophobic moiety having a binding moiety covalently attached thereto) comprises the following general formula:

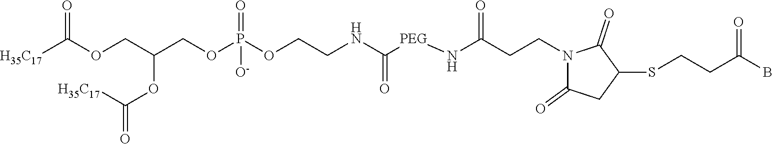

wherein B comprises an epitope tag, e.g., an ALFA-tag such as an ALFA-tag described herein.

In some embodiments, the targeting compound (hydrophobic moiety having a binding moiety covalently attached thereto) comprises the following general formula:

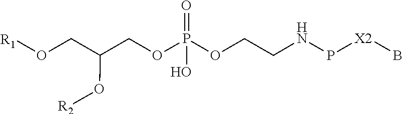

wherein X2 is as described above, R₁ and R₂ are CH₃(CH₂)₁₆C(O)— (stearoyl) or CH₃(CH₂)₁₂C(O)— (myristoyl), polymer P comprises the following general formula:

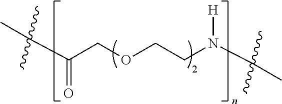

wherein n is 5 to 50, e.g., 5 to 25, e.g., 7 to 14, e.g., 10 to 25, e.g., 14 to 17, e.g., 8 or 14, and B comprises an epitope tag, e.g., an ALFA-tag such as an ALFA-tag described herein.

In some embodiments, n is 8 and R₁ and R₂ are CH₃(CH₂)₁₆C(O)— (stearoyl). In some embodiments, n is 14 and R₁ and R₂ are CH₃(CH₂)₁₆C(O)— (stearoyl).

In some embodiments, n is 8 and R₁ and R₂ are CH₃(CH₂)₁₂C(O)— (myristoyl). In some embodiments, n is 14 and R₁ and R₂ are CH₃(CH₂)₁₂C(O)— (myristoyl).

In some embodiments, X2 comprises the following general formula:

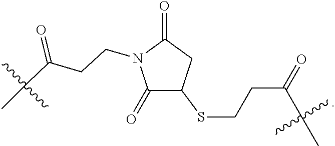

In some embodiments, the targeting compound (hydrophobic moiety having a binding moiety covalently attached thereto) comprises the following general formula:

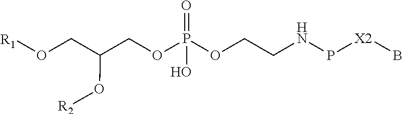

wherein X2 is as described above, R₁ and R₂ are CH₃(CH₂)₁₆C(O)— (stearoyl) or CH₃(CH₂)₁₂C(O)— (myristoyl), polymer P comprises the following general formula:

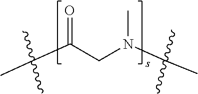

wherein s is 2 to 200, e.g., 5 to 100, e.g., 10 to 50, e.g., 15 to 40, e.g., 20 or 23, and B comprises an epitope tag, e.g., an ALFA-tag such as an ALFA-tag described herein.

In some embodiments, s is 20 and R₁ and R₂ are CH₃(CH₂)₁₆C(O)— (stearoyl).

In some embodiments, s is 20 and R₁ and R₂ are CH₃(CH₂)₁₂C(O)— (myristoyl).

In some embodiments, X2 comprises the following general formula:

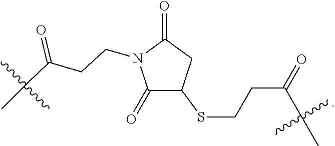

In some embodiments, B comprises a moiety comprising the structure —N-peptide-C(O)—NH2, wherein peptide comprises an epitope tag, e.g., an ALFA-tag such as an ALFA-tag described herein.

The present disclosure provides in one aspect, a targeting compound as described above which is integrated in a particle (e.g., a particle as described herein) via a hydrophobic component (e.g., lipid component) of the targeting compound.

### Primary Targeting Moiety

According to the disclosure, a nucleic acid payload is delivered specifically to a target cell by providing a moiety that binds to a target on target cells, e.g., an antigen on target cells, thus targeting particles comprising the nucleic acid payload to the target cells.

In some embodiments, the moiety that binds to a target on target cells is comprised by a compound (targeting compound) which is an integral part of a particle carrying the payload. In these embodiments, the targeting compound comprises a binding moiety that binds to target cells.

In some embodiments, the moiety that binds to a target on target cells is comprised by a compound (docking compound) further comprising a moiety that binds to a compound (targeting compound) which is an integral part of a particle carrying the payload and comprising a moiety for binding to the docking compound. In these embodiments, the targeting compound itself preferably does not comprise a moiety that binds to a target on target cells. Rather, the targeting compound comprises a binding moiety that forms the binding partner for a docking compound which binds to target cells.

The target on target cells is also referred to herein as “primary target”. In some embodiments, a primary target is a cell surface antigen on target cells.

A “primary targeting moiety” as used herein relates to the part of the targeting compound or docking compound which binds to a primary target, e.g., a cell surface antigen on target cells. Such targeting moieties are typically moieties that have affinity for cell surface targets. These moieties can be any peptide or protein (e.g. antibodies or antibody fragments) binding to the primary target. Particular embodiments of suitable primary targeting moieties for use herein include cell surface antigen binding moieties, such as antibodies, antibody fragments and DARPins. Other examples of primary targeting moieties are peptides or proteins which bind to a receptor.

A primary targeting moiety preferably binds with high specificity and/or high affinity and the bond with the primary target is preferably stable within the body.

In order to allow specific targeting of primary targets, the primary targeting moiety of the targeting compound or docking compound can comprise compounds including but not limited to antibodies, antibody fragments, e.g. Fab2, Fab, scFV, VHH domains, and other proteins or peptides.

According to some embodiments, the primary target is a cell surface antigen such as a T cell antigen, e.g., CD3, such as CD3e, CD8 or CD4, and suitable primary targeting moieties include but are not limited to, peptides and polypeptides targeting the cell surface antigen, e.g., antibodies, antibody fragments and DARPins.

According to some embodiments, the primary target is a receptor and suitable primary targeting moieties include but are not limited to, the ligand of such a receptor or a part thereof which still binds to the receptor, e.g., a receptor binding peptide in the case of receptor binding protein ligands.

Other examples of primary targeting moieties of protein nature include interferons, e.g. alpha, beta, and gamma interferon, interleukins, and protein growth factors, such as transforming growth factor (TGF), or platelet-derived growth factor (PDGF).

According to some embodiments, the primary target and primary targeting moiety are selected so as to result in the specific or increased targeting of certain cells. This can be achieved by selecting primary targets with cell-specific expression. For example, T cell antigens, e.g., those described herein, may be expressed in T cells while they are not expressed or expressed in a lower amount in other cells.

### Docking Compound

In some embodiments, a “docking compound” is used to form a connection between a primary target, e.g., a target cell or an antigen on target cells, and a targeting compound which is integrated into a particle comprising a nucleic acid payload to be delivered to a target cell. In some embodiments, a connection between a primary target, e.g., a target cell or an antigen on target cells, and a docking compound is a non-covalent connection. In some embodiments, a connection between a docking compound and a targeting compound is a non-covalent or covalent connection. In some embodiments, the targeting compound comprises a binding moiety for binding to the docking compound which is covalently attached to a hydrophobic moiety (e.g., lipid). The hydrophobic moiety (e.g., lipid) forms part of said particle.

In some embodiments, a docking compound comprises a “primary targeting moiety”, e.g., a moiety targeting a cell surface antigen on target cells, that is capable of binding to the primary target of interest, e.g., a cell surface antigen on target cells. In some embodiments, a “primary targeting moiety” as used herein relates to the part of the docking compound which binds to a primary target.

The docking compound further comprises a group which serves as a binding partner for a respective binding moiety of a targeting compound. The portion of the targeting compound comprising the hydrophobic moiety (e.g., lipid) (having a binding moiety for the docking compound covalently attached) integrates into a particle carrying a payload and thus forms a connection between the particle and the docking compound. The moiety of the docking compound binding to the targeting compound and the primary targeting moiety are linked to each other, preferably by a covalent linkage.

According to some embodiments, the docking compound comprises a bispecific molecule, such as a bispecific polypeptide, e.g., a bispecific antibody. In some embodiments, the docking compound comprises a binding domain binding to a primary target and a binding domain binding to a targeting compound. In some embodiments, the docking compound comprises an antibody or antibody fragment binding to a primary target and an antibody or antibody fragment binding to a targeting compound. In some embodiments, at least one binding domain comprises a heavy chain variable region (VH) and a light chain variable region (VL) of an antibody. In some embodiments, each binding domain comprises a heavy chain variable region (VH) and a light chain variable region (VL) of an antibody. In some embodiments, at least one binding domain comprises a single-domain antibody such as a VHH. In some embodiments, each binding domain comprises a single-domain antibody such as a VHH. In some embodiments, one binding domain comprises a heavy chain variable region (VH) and a light chain variable region (VL) of an antibody and the other binding domain comprises a single-domain antibody such as a VHH. In some embodiments, the binding domain binding to a primary target comprises a heavy chain variable region (VH) and a light chain variable region (VL) of an antibody. In some embodiments, the binding domain binding to a primary target comprises a single-domain antibody such as a VHH. In some embodiments, the binding domain binding to a targeting compound comprises a heavy chain variable region (VH) and a light chain variable region (VL) of an antibody. In some embodiments, the binding domain binding to a targeting compound comprises a single-domain antibody such as a VHH.

In some embodiments, the docking compound comprises a fusion protein which comprises a binding domain binding to a primary target and a binding domain binding to a targeting compound.

In some embodiments, the docking compound comprises a single peptide chain. In some embodiments, the single peptide chain comprises a portion, e.g., antibody, antibody fragment or DARPin, binding to a primary target and a portion, e.g., antibody or antibody fragment, binding to a targeting compound. In some embodiments, the antibody fragments are VHH, scFv, or a mixture thereof. In different embodiments, the docking compound comprises one of the following structures (from N-to C-terminus):
VHH (a targeting compound)-optional linker-VHH (a primary target)
VHH (a primary target)-optional linker-VHH (a targeting compound)
VHH (a targeting compound)-optional linker-scFv (a primary target)
scFv (a primary target)-optional linker-VHH (a targeting compound)
VHH (a primary target)-optional linker-scFv (a targeting compound)
scFv (a targeting compound)-optional linker-VHH (a primary target)
scFv (a targeting compound)-optional linker-scFv (a primary target)
scFv (a primary target)-optional linker-scFv (a targeting compound)

The present disclosure provides in one aspect, a docking compound as described herein. In some embodiments, the docking compound comprises a bispecific molecule, such as a bispecific polypeptide, e.g., a bispecific antibody, wherein one specificity binds to an epitope tag, e.g., an ALFA-tag and the other specificity binds to a primary target, e.g., a cell surface antigen on target cells. In some embodiments, the specificity which binds to an epitope tag is an antibody or antibody fragment such as an NbALFA-nanobody (NbALFA). In some embodiments, the specificity which binds to a primary target is an antibody, antibody fragment or DARPin. In some embodiments, the moiety targeting a primary target of the docking compound is selected from the group consisting of an anti-primary target DARPin, an anti-primary target VHH and an anti-primary target scFv and/or the moiety binding to a targeting compound of the docking compound is an NbALFA-nanobody (NbALFA). In some embodiments, the docking compound has a structure selected from the group consisting of NbALFA×anti-primary target DARPin, NbALFA×anti-primary target VHH and NbALFA×anti-primary target scFv. In some embodiments, the primary target is a T cell antigen, e.g., CD3, such CD3e, CD4 or CD8. In some embodiments, the docking compound comprises a bispecific antibody comprising a nanobody which binds to an epitope tag, e.g., an ALFA-tag, and an anti-CD3 VHH. In some embodiments, the docking compound comprises a bispecific antibody comprising a nanobody which binds to an epitope tag, e.g., an ALFA-tag, and an anti-CD3 scFv.

In some embodiments, the docking compound comprises a bispecific molecule comprising a nanobody which binds to an epitope tag, e.g., an ALFA-tag, and an anti-CD3 DARPin. In some embodiments, the docking compound comprises a bispecific antibody comprising a nanobody which binds to an epitope tag, e.g., an ALFA-tag, and an anti-CD4 VHH. In some embodiments, the docking compound comprises a bispecific antibody comprising a nanobody which binds to an epitope tag, e.g., an ALFA-tag, and an anti-CD4 scFv. In some embodiments, the docking compound comprises a bispecific molecule comprising a nanobody which binds to an epitope tag, e.g., an ALFA-tag, and an anti-CD4 DARPin. In some embodiments, the docking compound comprises a bispecific antibody comprising a nanobody which binds to an epitope tag, e.g., an ALFA-tag, and an anti-CD8 VHH. In some embodiments, the docking compound comprises a bispecific antibody comprising a nanobody which binds to an epitope tag, e.g., an ALFA-tag, and an anti-CD8 scFv. In some embodiments, the docking compound comprises a bispecific molecule comprising a nanobody which binds to an epitope tag, e.g., an ALFA-tag, and an anti-CD8 DARPin.

### Interacting Moieties on the Targeting Compound and on the Docking Compound

In some embodiments, the moiety on the targeting compound (binding moiety covalently attached to a hydrophobic moiety) and the moiety on the docking compound (moiety binding to the binding moiety covalently attached to a hydrophobic moiety) interacting which each other non-covalently bind to each other.

In some embodiments, the moieties on the targeting compound and on the docking compound interacting which each other bind to each other under physiological conditions.

In some embodiments, the moieties on the targeting compound and on the docking compound interacting which each other are antibody/antigen systems.

In some embodiments, the moiety of the targeting compound binding to the docking compound comprises a peptide or protein, e.g., a peptide tag, and the moiety of the docking compound binding to the targeting compound comprises a binder, e.g., an antibody or antibody fragment, binding to the peptide or protein.

In some embodiments, the moiety of the docking compound binding to the targeting compound comprises a peptide or protein, e.g., a peptide tag, and the moiety of the targeting compound binding to the docking compound comprises a binder, e.g., an antibody or antibody fragment, binding to the peptide or protein. In some embodiments, the moieties on the targeting compound and on the docking compound interacting which each other comprise an epitope tag/binder system.

As used herein, an “epitope tag” refers to a stretch of amino acids to which an antibody or proteinaceous molecule with antibody-like function can bind.

In some embodiments, the epitope tag comprises an ALFA-tag. In some embodiments, the epitope tag/binder system comprises an ALFA-tag and an ALFA-specific single-domain antibody (sdAb), NbALFA-nanobody.

In some embodiments, an ALFA-tag comprises the amino acid sequence

-AA0-AA1-AA2-AA3-AA4-AA5-AA6-AA7-AA8-AA9-AA10-
AA11-AA12-AA13-AA14-,

wherein the amino acids of AA0, AA1, AA2, AA3, AA4, AA5, AA6, AA7, AA8, AA9, AA10, AA11, AA12, AA13 and AA14 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, an ALFA-tag comprises a sequence selected from the group consisting of SRLEEELRRRLTE, PSRLEEELRRRLTE, SRLEEELRRRLTEP, and PSRLEEELRRRLTEP.

In some embodiments, an ALFA-tag comprises the cyclized amino acid sequence

-AA0-AA1-AA2-AA3-AA4-AA5-AA6-AA7-AA8-AA9-AA10-
AA11-AA12-AA13-AA14-,

wherein the side-chains of any two of the amino acids of AA0, AA1, AA2, AA3, AA4, AA5, AA6, AA7, AA8, AA9, AA10, AA11, AA12, AA13 and AA14 (X1, X2) are connected covalently; and
wherein the amino acids of AA0, AA1, AA2, AA3, AA4, AA5, AA6, AA7, AA8, AA9, AA10, AA11, AA12, AA13 and AA14 which are not X1 and X2 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X1 and X2 are separated by 2 or 3 amino acids.

In some embodiments, AA5 is X1 and AA9 is X2, AA5 is X1 and AA8 is X2, AA9 is X1 and AA13 is X2, AA6 is X1 and AA9 is X2, AA9 is X1 and AA12 is X2, AA10 is X1 and AA13 is X2, AA6 is X1 and AA10 is X2 or AA4 is X1 and AA8 is X2.

In some embodiments, an ALFA-tag comprises a cyclized amino acid sequence selected from the group consisting of

a. -AA0-AA1-AA2-AA3-AA4-cyclo(X1-AA6-AA7-AA8-X2)-
Arg-Leu-AA12-AA13-AA14-,
b. -AA0-AA1-AA2-AA3-AA4-cyclo(X1-AA6-AA7-X2)-
AA9-Arg-Leu-AA12-AA13-AA14-,
c. -AA0-AA1-AA2-AA3-AA4-AA5-AA6-AA7-AA8-cyclo
(X1-Arg-Leu-AA12-X2)-AA14-,
d. -AA0-AA1-AA2-AA3-AA4-AA5-cyclo(X1-AA7-AA8-X2)-
Arg-Leu-AA12-AA13-AA14-,
e. -AA0-AA1-AA2-AA3-AA4-AA5-AA6-AA7-AA8-cyclo
(X1-Arg-Leu-X2)-AA13-AA14-,
f. -AA0-AA1-AA2-AA3-AA4-AA5-AA6-AA7-AA8-AA9-
cyclo(X1-Leu-AA12-X2)-AA14-,
g. -AA0-AA1-AA2-AA3-AA4-AA5-cyclo(X1-AA7-AA8-
AA9-X2)-Leu-AA12-AA13-AA14-,
and
h. -AA0-AA1-AA2-AA3-cyclo(X1-AA5-AA6-AA7-X2)-
AA9-Arg-Leu-AA12-AA13-AA14-,

wherein the side-chains of X₁ and X₂ amino acid residues are connected covalently;
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X₁ and X₂ in the peptides disclosed herein are connected covalently via an amide, disulfide, thioether, ether, ester, thioester, thioamide, alkylene, alkenylene, alkynylene, and/or 1,2,3-triazole.

In some embodiments, a cyclized amino acid sequence described herein is generated by linking an amino group of a side-chain of one of X1 and X₂ to the carboxyl group of a side-chain of the other of X1 and X₂ via an amide bond. The amino group of the side chain of an amino acid that possesses a pendant amine group, e.g., lysine or a lysine derivative, and the carboxyl group of the side chain of an acidic amino acid, e.g., aspartic acid, glutamic acid or a derivative thereof, can be used to generate a cyclized amino acid sequence via an amide bond.

In some embodiments, a cyclized amino acid sequence described herein is generated by linking a sulfhydryl group of a side-chain of one of X₁ and X₂ to the sulfhydryl group of a side-chain of the other of X₁ and X₂ via a disulfide bond. Sulfhydryl group-containing amino acids include cysteine and other sulfhydryl-containing amino acids as Pen.

In some embodiments, X1 and X₂ are, independently, selected from the group consisting of Glu, DGlu, Asp, DAsp, Lys, DLys, hLys, DhLys, Orn, DOrn, Dab, DDab, Dap, DDap, Cys, DCys, hCys, DhCys, Pen, and DPen, with the proviso that when X₁ is Glu, DGlu, Asp, or DAsp, X₂ is Lys, DLys, hLys, DhLys, Orn, DOrn, Dab, DDab, Dap, or DDap; when X₁ is Lys, DLys, hLys, DhLys, Orn, DOrn, Dab, DDab, Dap, or DDap, X₂ is Glu, DGlu, Asp, or DAsp; and when X₁ is Cys, DCys, hCys, DhCys, Pen, or DPen, X₂ is Cys, DCys, hCys, DhCys, Pen, or DPen.

In some embodiments, X₁ is Glu and X₂ is Lys. In some embodiments, -cyclo(Glu--------Lys)-, -c(Glu-------Lys)-, -cyclo(E-------K)-, -c(E---------K)-, -E------K- cyclo, or -cycloE----cycloK- comprises the following structure:

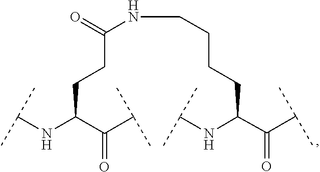

In some embodiments, X₁ is Lys and X₂ is Glu. In some embodiments, -cyclo(Lys------Glu)-, -c(Lys------Glu)--cyclo(K------E)-, -c(K-------E)-, -K-----E- cyclo, or cycloK------cycloE- comprises the following structure:

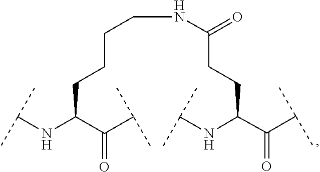

In some embodiments, X₁ is Cys and X₂ is Cys. In some embodiments, -cyclo(Cys------Cys)-, c(Cys------Cys)--cyclo(C------C)-, -c(C--------C)-, -C----C- cyclo, or -cycloC-----cycloC- comprises the following structure:

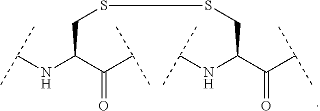

Particular cyclized amino acid sequences of the above-identified generic formulas include, for example,

-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
-Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-,
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-,
-Pro-Ser-Arg-Leu-Glu-cyclo(Glu-Glu-Leu-Arg-DLys)-Arg-Leu-Thr-Glu-,
-Pro-Ser-Arg-Leu-Glu-cyclo(Lys-Glu-Leu-Arg-Glu)-Arg-Leu-Thr-Glu-,
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-,
-Ser-Arg-Leu-Glu-cyclo(DLys-Glu-Leu-Arg-Glu)-Arg-Leu-Thr-Glu-,
-Ser-Arg-Leu-Glu-cyclo(Lys-Glu-Leu-Arg-DGlu)-Arg-Leu-Thr-Glu-,
-Ser-Arg-Leu-Glu-cyclo(DLys-Glu-Leu-Arg-DGlu)-Arg-Leu-Thr-Glu-,
-Pro-Ser-Arg-Leu-Glu-cyclo(DGlu-Glu-Leu-Arg-DLys)-Arg-Leu-Thr-Glu-,
-Pro-Ser-Arg-Leu-Glu-cyclo(DLys-Glu-Leu-Arg-Glu)-Arg-Leu-Thr-Glu-,
-Pro-Ser-Arg-Leu-Glu-cyclo(Lys-Glu-Leu-Arg-DGlu)-Arg-Leu-Thr-Glu-,
-Pro-Ser-Arg-Leu-Glu-cyclo(DLys-Glu-Leu-Arg-DGlu)-Arg-Leu-Thr-Glu-,
-Ser-Arg-Leu-Glu-cyclo(DLys-Glu-Leu-Arg-Asp)-Arg-Leu-Thr-Glu-,
-Ser-Arg-Leu-Glu-cyclo(DAsp-Glu-Leu-Arg-DLys)-Arg-Leu-Thr-Glu-,
-Ser-Arg-Leu-Glu-cyclo(DAsp-Glu-Leu-Arg-Lys)-Arg-Leu-Thr-Glu-,
-Ser-Arg-Leu-Glu-cyclo(Asp-Glu-Leu-Arg-DLys)-Arg-Leu-Thr-Glu-,
-Ser-Arg-Leu-Glu-cyclo(Lys-Glu-Leu-Arg-DAsp)-Arg-Leu-Thr-Glu-,
-Ser-Arg-Leu-Glu-cyclo(DLys-Glu-Leu-Arg-DAsp)-Arg-Leu-Thr-Glu-,
-Pro-Ser-Arg-Leu-Glu-cyclo(DLys-Glu-Leu-Arg-Asp)-Arg-Leu-Thr-Glu-,
-Pro-Ser-Arg-Leu-Glu-cyclo(DAsp-Glu-Leu-Arg-DLys)-Arg-Leu-Thr-Glu-,
-Pro-Ser-Arg-Leu-Glu-cyclo(DAsp-Glu-Leu-Arg-Lys)-Arg-Leu-Thr-Glu-,
-Pro-Ser-Arg-Leu-Glu-cyclo(Asp-Glu-Leu-Arg-DLys)-Arg-Leu-Thr-Glu-,
-Pro-Ser-Arg-Leu-Glu-cyclo(Lys-Glu-Leu-Arg-DAsp)-Arg-Leu-Thr-Glu-,
-Pro-Ser-Arg-Leu-Glu-cyclo(DLys-Glu-Leu-Arg-DAsp)-Arg-Leu-Thr-Glu-,
-Ser-Arg-Leu-Glu-cyclo(DGlu-Glu-Leu-Lys)-Arg-Arg-Leu-Thr-Glu-,
-Ser-Arg-Leu-Glu-cyclo(Glu-Glu-Leu-DLys)-Arg-Arg-Leu-Thr-Glu-,
-Ser-Arg-Leu-Glu-cyclo(DGlu-Glu-Leu-DLys)-Arg-Arg-Leu-Thr-Glu-,
-Ser-Arg-Leu-Glu-cyclo(Lys-Glu-Leu-Glu)-Arg-Arg-Leu-Thr-Glu-,
-Ser-Arg-Leu-Glu-cyclo(Lys-Glu-Leu-DGlu)-Arg-Arg-Leu-Thr-Glu-,
-Ser-Arg-Leu-Glu-cyclo(DLys-Glu-Leu-DGlu)-Arg-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-cyclo(Asp-Glu-Leu-DLys)-Arg-Arg-Leu-Thr-Glu-,
-Ser-Arg-Leu-Glu-cyclo(Lys-Glu-Leu-Asp)-Arg-Arg-Leu-Thr-Glu-,
-Ser-Arg-Leu-Glu-cyclo(Lys-Glu-Leu-DAsp)-Arg-Arg-Leu-Thr-Glu-,
-Ser-Arg-Leu-Glu-cyclo(DAsp-Glu-Leu-Lys)-Arg-Arg-Leu-Thr-Glu-,
-Ser-Arg-Leu-Glu-cyclo(DAsp-Glu-Leu-DLys)-Arg-Arg-Leu-Thr-Glu-,
-Ser-Arg-Leu-Glu-cyclo(DLys-Glu-Leu-DAsp)-Arg-Arg-Leu-Thr-Glu-,
-Ser-Arg-Leu-Glu-cyclo(DLys-Glu-Leu-Asp)-Arg-Arg-Leu-Thr-Glu-,
-Pro-Ser-Arg-Leu-Glu-cyclo(Glu-Glu-Leu-DLys)-Arg-Arg-Leu-Thr-Glu-,
-Pro-Ser-Arg-Leu-Glu-cyclo(DGlu-Glu-Leu-DLys)-Arg-Arg-Leu-Thr-Glu-
-Pro-Ser-Arg-Leu-Glu-cyclo(Lys-Glu-Leu-Glu)-Arg-Arg-Leu-Thr-Glu-,
-Pro-Ser-Arg-Leu-Glu-cyclo(Lys-Glu-Leu-DGlu)-Arg-Arg-Leu-Thr-Glu-,
-Pro-Ser-Arg-Leu-Glu-cyclo(DLys-Glu-Leu-DGlu)-Arg-Arg-Leu-Thr-Glu-,
-Pro-Ser-Arg-Leu-Glu-cyclo(Asp-Glu-Leu-Lys)-Arg-Arg-Leu-Thr-Glu-,
-Pro-Ser-Arg-Leu-Glu-cyclo(Asp-Glu-Leu-DLys)-Arg-Arg-Leu-Thr-Glu-,
-Pro-Ser-Arg-Leu-Glu-cyclo(Lys-Glu-Leu-Asp)-Arg-Arg-Leu-Thr-Glu-,
-Pro-Ser-Arg-Leu-Glu-cyclo(Lys-Glu-Leu-DAsp)-Arg-Arg-Leu-Thr-Glu-,
-Pro-Ser-Arg-Leu-Glu-cyclo(DAsp-Glu-Leu-Lys)-Arg-Arg-Leu-Thr-Glu-,
-Pro-Ser-Arg-Leu-Glu-cyclo(DAsp-Glu-Leu-DLys)-Arg-Arg-Leu-Thr-Glu-,
-Pro-Ser-Arg-Leu-Glu-cyclo(DLys-Glu-Leu-DAsp)-Arg-Arg-Leu-Thr-Glu-,
-Pro-Ser-Arg-Leu-Glu-cyclo(DLys-Glu-Leu-Asp)-Arg-Arg-Leu-Thr-Glu-,
-Ser-Arg-Leu-Glu-Glu-cyclo(Asp-Leu-Arg-Lys)-Arg-Leu-Thr-Glu-,
-Ser-Arg-Leu-Glu-Glu-cyclo(Lys-Leu-Arg-Asp)-Arg-Leu-Thr-Glu-,
-Ser-Arg-Leu-Glu-Glu-cyclo(DAsp-Leu-Arg-Lys)-Arg-Leu-Thr-Glu-,
-Ser-Arg-Leu-Glu-Glu-cyclo(Lys-Leu-Arg-DAsp)-Arg-Leu-Thr-Glu-,
-Ser-Arg-Leu-Glu-Glu-cyclo(Asp-Leu-Arg-DLys)-Arg-Leu-Thr-Glu-,
-Ser-Arg-Leu-Glu-Glu-cyclo(DLys-Leu-Arg-Asp)-Arg-Leu-Thr-Glu-,
-Ser-Arg-Leu-Glu-Glu-cyclo(DLys-Leu-Arg-DAsp)-Arg-Leu-Thr-Glu-,
-Ser-Arg-Leu-Glu-Glu-cyclo(DAsp-Leu-Arg-DLys)-Arg-Leu-Thr-Glu-,
-Pro-Ser-Arg-Leu-Glu-Glu-cyclo(Asp-Leu-Arg-Lys)-Arg-Leu-Thr-Glu-,
-Pro-Ser-Arg-Leu-Glu-Glu-cyclo(Lys-Leu-Arg-Asp)-Arg-Leu-Thr-Glu-,
-Pro-Ser-Arg-Leu-Glu-Glu-cyclo(DAsp-Leu-Arg-Lys)-Arg-Leu-Thr-Glu-,
-Pro-Ser-Arg-Leu-Glu-Glu-cyclo(Lys-Leu-Arg-DAsp)-Arg-Leu-Thr-Glu-,
-Pro-Ser-Arg-Leu-Glu-Glu-cyclo(Asp-Leu-Arg-DLys)-Arg-Leu-Thr-Glu-,
-Pro-Ser-Arg-Leu-Glu-Glu-cyclo(DLys-Leu-Arg-Asp)-Arg-Leu-Thr-Glu-,
-Pro-Ser-Arg-Leu-Glu-Glu-cyclo(DLys-Leu-Arg-DAsp)-Arg-Leu-Thr-Glu-,
-Pro-Ser-Arg-Leu-Glu-Glu-cyclo(DAsp-Leu-Arg-DLys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-Glu-Leu-Arg-cyclo(Cys-Arg-Leu-Cys)-Glu-,
-Ser-Arg-Leu-Glu-Glu-cyclo(Cys-Leu-Arg-Cys)-Arg-Leu-Thr-Glu-,
-Pro-Ser-Arg-Leu-Glu-cyclo(Cys-Glu-Leu-Cys)-Arg-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-Glu-Leu-Arg-cyclo(Cys-Arg-Leu-Cys)-Glu-,
-Pro-Ser-Arg-Leu-Glu-Glu-cyclo(Cys-Leu-Arg-Cys)-Arg-Leu-Thr-Glu-,
-Ser-Arg-Leu-Glu-cyclo(DCys-Glu-Leu-DCys)-Arg-Arg-Leu-Thr-Glu-,
-Ser-Arg-Leu-Glu-cyclo(DCys-Glu-Leu-Cys)-Arg-Arg-Leu-Thr-Glu-,
-Ser-Arg-Leu-Glu-cyclo(hCys-Glu-Leu-Cys)-Arg-Arg-Leu-Thr-Glu-,
-Ser-Arg-Leu-Glu-cyclo(hCys-Glu-Leu-hCys)-Arg-Arg-Leu-Thr-Glu-,
-Ser-Arg-Leu-Glu-cyclo(Cys-Glu-Leu-hCys)-Arg-Arg-Leu-Thr-Glu-,
-Ser-Arg-Leu-Glu-cyclo(hCys-Glu-Leu-DCys)-Arg-Arg-Leu-Thr-Glu-,
-Ser-Arg-Leu-Glu-cyclo(DCys-Glu-Leu-hCys)-Arg-Arg-Leu-Thr-Glu-,
-Ser-Arg-Leu-Glu-cyclo(hCys-Glu-Leu-DhCys)-Arg-Arg-Leu-Thr-Glu-,
-Ser-Arg-Leu-Glu-cyclo(DhCys-Glu-Leu-DhCys)-Arg-Arg-Leu-Thr-Glu-,
-Ser-Arg-Leu-Glu-cyclo(Cys-Glu-Leu-DhCys)-Arg-Arg-Leu-Thr-Glu-,
-Ser-Arg-Leu-Glu-cyclo(DCys-Glu-Leu-DhCys)-Arg-Arg-Leu-Thr-Glu-,
-Ser-Arg-Leu-Glu-cyclo(DhCys-Glu-Leu-Cys)-Arg-Arg-Leu-Thr-Glu-,
-Ser-Arg-Leu-Glu-cyclo(DhCys-Glu-Leu-DCys)-Arg-Arg-Leu-Thr-Glu-,
-Pro-Ser-Arg-Leu-Glu-cyclo(DCys-Glu-Leu-DCys)-Arg-Arg-Leu-Thr-Glu-,
-Pro-Ser-Arg-Leu-Glu-cyclo(DCys-Glu-Leu-Cys)-Arg-Arg-Leu-Thr-Glu-,
-Pro-Ser-Arg-Leu-Glu-cyclo(hCys-Glu-Leu-Cys)-Arg-Arg-Leu-Thr-Glu-,
-Pro-Ser-Arg-Leu-Glu-cyclo(hCys-Glu-Leu-hCys)-Arg-Arg-Leu-Thr-Glu-,
-Pro-Ser-Arg-Leu-Glu-cyclo(Cys-Glu-Leu-hCys)-Arg-Arg-Leu-Thr-Glu-,
-Pro-Ser-Arg-Leu-Glu-cyclo(hCys-Glu-Leu-DCys)-Arg-Arg-Leu-Thr-Glu-,
-Pro-Ser-Arg-Leu-Glu-cyclo(DCys-Glu-Leu-hCys)-Arg-Arg-Leu-Thr-Glu-,
-Pro-Ser-Arg-Leu-Glu-cyclo(hCys-Glu-Leu-DhCys)-Arg-Arg-Leu-Thr-Glu-,
-Pro-Ser-Arg-Leu-Glu-cyclo(DhCys-Glu-Leu-DhCys)-Arg-Arg-Leu-Thr-Glu-,
-Pro-Ser-Arg-Leu-Glu-cyclo(Cys-Glu-Leu-DhCys)-Arg-Arg-Leu-Thr-Glu-,
-Pro-Ser-Arg-Leu-Glu-cyclo(DCys-Glu-Leu-DhCys)-Arg-Arg-Leu-Thr-Glu-,
-Pro-Ser-Arg-Leu-Glu-cyclo(DhCys-Glu-Leu-Cys)-Arg-Arg-Leu-Thr-Glu-,
-Pro-Ser-Arg-Leu-Glu-cyclo(DhCys-Glu-Leu-DCys)-Arg-Arg-Leu-Thr-Glu-,
-Ser-Arg-Leu-Glu-Glu-Glu-Leu-Arg-Arg-cyclo(Cys-Leu-Thr-DCys)-,
-Ser-Arg-Leu-Glu-Glu-Glu-Leu-Arg-Arg-cyclo(DCys-Leu-Thr-Cys)-,
-Ser-Arg-Leu-Glu-Glu-Glu-Leu-Arg-Arg-cyclo(DCys-Leu-Thr-DCys)-,
-Ser-Arg-Leu-Glu-Glu-Glu-Leu-Arg-Arg-cyclo(hCys-Leu-Thr-Cys)-,
-Ser-Arg-Leu-Glu-Glu-Glu-Leu-Arg-Arg-cyclo(hCys-Leu-Thr-DCys)-,
-Ser-Arg-Leu-Glu-Glu-Glu-Leu-Arg-Arg-cyclo(hCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DhCys-Leu-Thr-Cys)-,
-Ser-Arg-Leu-Glu-Glu-Glu-Leu-Arg-Arg-cyclo(DhCys-Leu-Thr-DCys)-,
-Ser-Arg-Leu-Glu-Glu-Glu-Leu-Arg-Arg-cyclo(DhCys-Leu-Thr-hCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(DCys-Leu-Thr-DhCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(Pen-Leu-Thr-Pen)-,
-Ser-Arg-Leu-Glu-Glu-Glu-Leu-Arg-Arg-cyclo(Pen-Leu-Thr-DPen)-,
-Ser-Arg-Leu-Glu-Glu-Glu-Leu-Arg-Arg-cyclo(DPen-Leu-Thr-Pen)-,
-Ser-Arg-Leu-Glu-Glu-Glu-Leu-Arg-Arg-cyclo(DPen-Leu-Thr-DPen)-,
-Ser-Arg-Leu-Glu-Glu-Glu-Leu-Arg-cyclo(DCys-Arg-Leu-Cys)-Glu-,
-Ser-Arg-Leu-Glu-Glu-Glu-Leu-Arg-cyclo(DCys-Arg-Leu-DCys)-Glu-,
-Ser-Arg-Leu-Glu-Glu-Glu-Leu-Arg-cyclo(Cys-Arg-Leu-DCys)-Glu-,
-Ser-Arg-Leu-Glu-Glu-Glu-Leu-Arg-cyclo(hCys-Arg-Leu-Cys)-Glu-,
-Ser-Arg-Leu-Glu-Glu-Glu-Leu-Arg-cyclo(hCys-Arg-Leu-DCys)-Glu-,
-Ser-Arg-Leu-Glu-Glu-Glu-Leu-Arg-cyclo(Cys-Arg-Leu-hCys)-Glu-,
-Ser-Arg-Leu-Glu-Glu-Glu-Leu-Arg-cyclo(hCys-Arg-Leu-hCys)-Glu-,
-Ser-Arg-Leu-Glu-Glu-Glu-Leu-Arg-cyclo(hCys-Arg-Leu-DhCys)-Glu-,
-Ser-Arg-Leu-Glu-Glu-Glu-Leu-Arg-cyclo(DhCys-Arg-Leu-hCys)-Glu-,
-Pro-Ser-Arg-Leu-Glu-Glu-Glu-Leu-Arg-Arg-cyclo(Cys-Leu-Thr-DCys)-,
-Pro-Ser-Arg-Leu-Glu-Glu-Glu-Leu-Arg-Arg-cyclo(DCys-Leu-Thr-Cys)-,
-Pro-Ser-Arg-Leu-Glu-Glu-Glu-Leu-Arg-Arg-cyclo(DCys-Leu-Thr-DCys)-,
-Pro-Ser-Arg-Leu-Glu-Glu-Glu-Leu-Arg-Arg-cyclo(hCys-Leu-Thr-Cys)-,
-Pro-Ser-Arg-Leu-Glu-Glu-Glu-Leu-Arg-Arg-cyclo(hCys-Leu-Thr-DCys)-,
-Pro-Ser-Arg-Leu-Glu-Glu-Glu-Leu-Arg-Arg-cyclo(hCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DhCys-Leu-Thr-Cys)-,
-Pro-Ser-Arg-Leu-Glu-Glu-Glu-Leu-Arg-Arg-cyclo(DhCys-Leu-Thr-DCys)-,
-Pro-Ser-Arg-Leu-Glu-Glu-Glu-Leu-Arg-Arg-cyclo(DhCys-Leu-Thr-hCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(DCys-Leu-Thr-DhCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(Pen-Leu-Thr-Pen)-,
-Pro-Ser-Arg-Leu-Glu-Glu-Glu-Leu-Arg-Arg-cyclo(Pen-Leu-Thr-DPen)-,
-Pro-Ser-Arg-Leu-Glu-Glu-Glu-Leu-Arg-Arg-cyclo(DPen-Leu-Thr-Pen)-,
-Pro-Ser-Arg-Leu-Glu-Glu-Glu-Leu-Arg-Arg-cyclo(DPen-Leu-Thr-DPen)-,
-Pro-Ser-Arg-Leu-Glu-Glu-Glu-Leu-Arg-cyclo(DCys-Arg-Leu-Cys)-Glu-,
-Pro-Ser-Arg-Leu-Glu-Glu-Glu-Leu-Arg-cyclo(DCys-Arg-Leu-DCys)-Glu-,
-Pro-Ser-Arg-Leu-Glu-Glu-Glu-Leu-Arg-cyclo(Cys-Arg-Leu-DCys)-Glu-,
-Pro-Ser-Arg-Leu-Glu-Glu-Glu-Leu-Arg-cyclo(hCys-Arg-Leu-Cys)-Glu-,
-Pro-Ser-Arg-Leu-Glu-Glu-Glu-Leu-Arg-cyclo(hCys-Arg-Leu-DCys)-Glu-,
-Pro-Ser-Arg-Leu-Glu-Glu-Glu-Leu-Arg-cyclo(Cys-Arg-Leu-hCys)-Glu-,
-Pro-Ser-Arg-Leu-Glu-Glu-Glu-Leu-Arg-cyclo(hCys-Arg-Leu-hCys)-Glu-,
-Pro-Ser-Arg-Leu-Glu-Glu-Glu-Leu-Arg-cyclo(hCys-Arg-Leu-DhCys)-Glu-,
-Pro-Ser-Arg-Leu-Glu-Glu-Glu-Leu-Arg-cyclo(DhCys-Arg-Leu-hCys)-Glu-,
-Ser-Arg-Leu-Glu-Glu-Glu-Leu-Arg-cyclo(Lys-Arg-Leu-Asp)-Glu-,
-Ser-Arg-Leu-Glu-Glu-Glu-Leu-Arg-cyclo(Asp-Arg-Leu-Lys)-Glu-,
-Ser-Arg-Leu-Glu-Glu-Glu-Leu-Arg-cyclo(Lys-Arg-Leu-Glu)-Glu-,
-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Lys-Arg-Leu-Asp)-Glu-,
-Pro-Ser-Arg-Leu-Glu-Glu-Glu-Leu-Arg-cyclo(Asp-Arg-Leu-Lys)-Glu-,
-Pro-Ser-Arg-Leu-Glu-Glu-Glu-Leu-Arg-cyclo(Lys-Arg-Leu-Glu)-Glu-,
-Pro-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-Pro-,
-Ser-Arg-Leu-Glu-cyclo(DLys-Glu-Leu-Arg-Glu)-Arg-Leu-Thr-Glu-Pro-,
-Ser-Arg-Leu-Glu-cyclo(Lys-Glu-Leu-Arg-DGlu)-Arg-Leu-Thr-Glu-Pro-,
-Ser-Arg-Leu-Glu-cyclo(DLys-Glu-Leu-Arg-DGlu)-Arg-Leu-Thr-Glu-Pro-,
-Pro-Ser-Arg-Leu-Glu-cyclo(DGlu-Glu-Leu-Arg-DLys)-Arg-Leu-Thr-Glu-Pro-,
-Pro-Ser-Arg-Leu-Glu-cyclo(DLys-Glu-Leu-Arg-Glu)-Arg-Leu-Thr-Glu-Pro-,
-Pro-Ser-Arg-Leu-Glu-cyclo(Lys-Glu-Leu-Arg-DGlu)-Arg-Leu-Thr-Glu-Pro-,
-Pro-Ser-Arg-Leu-Glu-cyclo(DLys-Glu-Leu-Arg-DGlu)-Arg-Leu-Thr-Glu-Pro-,
-Ser-Arg-Leu-Glu-cyclo(DLys-Glu-Leu-Arg-Asp)-Arg-Leu-Thr-Glu-Pro-,
-Ser-Arg-Leu-Glu-cyclo(DAsp-Glu-Leu-Arg-DLys)-Arg-Leu-Thr-Glu-Pro-,
-Ser-Arg-Leu-Glu-cyclo(DAsp-Glu-Leu-Arg-Lys)-Arg-Leu-Thr-Glu-Pro-,
-Ser-Arg-Leu-Glu-cyclo(Asp-Glu-Leu-Arg-DLys)-Arg-Leu-Thr-Glu-Pro-,
-Ser-Arg-Leu-Glu-cyclo(Lys-Glu-Leu-Arg-DAsp)-Arg-Leu-Thr-Glu-Pro-,
-Ser-Arg-Leu-Glu-cyclo(DLys-Glu-Leu-Arg-DAsp)-Arg-Leu-Thr-Glu-Pro-,
-Pro-Ser-Arg-Leu-Glu-cyclo(DLys-Glu-Leu-Arg-Asp)-Arg-Leu-Thr-Glu-Pro-,
-Pro-Ser-Arg-Leu-Glu-cyclo(DAsp-Glu-Leu-Arg-DLys)-Arg-Leu-Thr-Glu-Pro-,
-Pro-Ser-Arg-Leu-Glu-cyclo(DAsp-Glu-Leu-Arg-Lys)-Arg-Leu-Thr-Glu-Pro-,
-Pro-Ser-Arg-Leu-Glu-cyclo(Asp-Glu-Leu-Arg-DLys)-Arg-Leu-Thr-Glu-Pro-,
-Pro-Ser-Arg-Leu-Glu-cyclo(Lys-Glu-Leu-Arg-DAsp)-Arg-Leu-Thr-Glu-Pro-,
-Pro-Ser-Arg-Leu-Glu-cyclo(DLys-Glu-Leu-Arg-DAsp)-Arg-Leu-Thr-Glu-Pro-,
-Ser-Arg-Leu-Glu-cyclo(DGlu-Glu-Leu-Lys)-Arg-Arg-Leu-Thr-Glu-Pro-,
-Ser-Arg-Leu-Glu-cyclo(Glu-Glu-Leu-DLys)-Arg-Arg-Leu-Thr-Glu-Pro-,
-Ser-Arg-Leu-Glu-cyclo(DGlu-Glu-Leu-DLys)-Arg-Arg-Leu-Thr-Glu-Pro-,
-Ser-Arg-Leu-Glu-cyclo(Lys-Glu-Leu-Glu)-Arg-Arg-Leu-Thr-Glu-Pro-,
-Ser-Arg-Leu-Glu-cyclo(Lys-Glu-Leu-DGlu)-Arg-Arg-Leu-Thr-Glu-Pro-,
-Ser-Arg-Leu-Glu-cyclo(DLys-Glu-Leu-DGlu)-Arg-Arg-Leu-Thr-Glu-Pro-,
-Ser-Arg-Leu-Glu-cyclo(Asp-Glu-Leu-Lys)-Arg-Arg-Leu-Thr-Glu-Pro-,
-Ser-Arg-Leu-Glu-cyclo(Asp-Glu-Leu-DLys)-Arg-Arg-Leu-Thr-Glu-Pro-,
-Ser-Arg-Leu-Glu-cyclo(Lys-Glu-Leu-Asp)-Arg-Arg-Leu-Thr-Glu-Pro-,
-Ser-Arg-Leu-Glu-cyclo(Lys-Glu-Leu-DAsp)-Arg-Arg-Leu-Thr-Glu-Pro-,
-Ser-Arg-Leu-Glu-cyclo(DAsp-Glu-Leu-Lys)-Arg-Arg-Leu-Thr-Glu-Pro-,
-Ser-Arg-Leu-Glu-cyclo(DAsp-Glu-Leu-DLys)-Arg-Arg-Leu-Thr-Glu-Pro-,
-Ser-Arg-Leu-Glu-cyclo(DLys-Glu-Leu-DAsp)-Arg-Arg-Leu-Thr-Glu-Pro-,
-Ser-Arg-Leu-Glu-cyclo(DLys-Glu-Leu-Asp)-Arg-Arg-Leu-Thr-Glu-Pro-,
-Pro-Ser-Arg-Leu-Glu-cyclo(Glu-Glu-Leu-DLys)-Arg-Arg-Leu-Thr-Glu-Pro-,
-Pro-Ser-Arg-Leu-Glu-cyclo(DGlu-Glu-Leu-DLys)-Arg-Arg-Leu-Thr-Glu-Pro-,
-Pro-Ser-Arg-Leu-Glu-cyclo(Lys-Glu-Leu-Glu)-Arg-Arg-Leu-Thr-Glu-Pro-,
-Pro-Ser-Arg-Leu-Glu-cyclo(Lys-Glu-Leu-DGlu)-Arg-Arg-Leu-Thr-Glu-Pro-,
-Pro-Ser-Arg-Leu-Glu-cyclo(DLys-Glu-Leu-DGlu)-Arg-Arg-Leu-Thr-Glu-Pro-,
-Pro-Ser-Arg-Leu-Glu-cyclo(Asp-Glu-Leu-Lys)-Arg-Arg-Leu-Thr-Glu-Pro-,
-Pro-Ser-Arg-Leu-Glu-cyclo(Asp-Glu-Leu-DLys)-Arg-Arg-Leu-Thr-Glu-Pro-,
-Pro-Ser-Arg-Leu-Glu-cyclo(Lys-Glu-Leu-Asp)-Arg-Arg-Leu-Thr-Glu-Pro-,
-Pro-Ser-Arg-Leu-Glu-cyclo(Lys-Glu-Leu-DAsp)-Arg-Arg-Leu-Thr-Glu-Pro-,
-Pro-Ser-Arg-Leu-Glu-cyclo(DAsp-Glu-Leu-Lys)-Arg-Arg-Leu-Thr-Glu-Pro-,
-Pro-Ser-Arg-Leu-Glu-cyclo(DAsp-Glu-Leu-DLys)-Arg-Arg-Leu-Thr-Glu-Pro-,
-Pro-Ser-Arg-Leu-Glu-cyclo(DLys-Glu-Leu-DAsp)-Arg-Arg-Leu-Thr-Glu-Pro-,
-Pro-Ser-Arg-Leu-Glu-cyclo(DLys-Glu-Leu-Asp)-Arg-Arg-Leu-Thr-Glu-Pro-,
-Ser-Arg-Leu-Glu-Glu-cyclo(Asp-Leu-Arg-Lys)-Arg-Leu-Thr-Glu-Pro-,
-Ser-Arg-Leu-Glu-Glu-cyclo(Lys-Leu-Arg-Asp)-Arg-Leu-Thr-Glu-Pro-,
-Ser-Arg-Leu-Glu-Glu-cyclo(DAsp-Leu-Arg-Lys)-Arg-Leu-Thr-Glu-Pro-,
-Ser-Arg-Leu-Glu-Glu-cyclo(Lys-Leu-Arg-DAsp)-Arg-Leu-Thr-Glu-Pro-,
-Ser-Arg-Leu-Glu-Glu-cyclo(Asp-Leu-Arg-DLys)-Arg-Leu-Thr-Glu-Pro-,
-Ser-Arg-Leu-Glu-Glu-cyclo(DLys-Leu-Arg-Asp)-Arg-Leu-Thr-Glu-Pro-,
-Ser-Arg-Leu-Glu-Glu-cyclo(DLys-Leu-Arg-DAsp)-Arg-Leu-Thr-Glu-Pro-,
-Ser-Arg-Leu-Glu-Glu-cyclo(DAsp-Leu-Arg-DLys)-Arg-Leu-Thr-Glu-Pro-,
-Pro-Ser-Arg-Leu-Glu-Glu-cyclo(Asp-Leu-Arg-Lys)-Arg-Leu-Thr-Glu-Pro-,
-Pro-Ser-Arg-Leu-Glu-Glu-cyclo(Lys-Leu-Arg-Asp)-Arg-Leu-Thr-Glu-Pro-,
-Pro-Ser-Arg-Leu-Glu-Glu-cyclo(DAsp-Leu-Arg-Lys)-Arg-Leu-Thr-Glu-Pro-,
-Pro-Ser-Arg-Leu-Glu-Glu-cyclo(Lys-Leu-Arg-DAsp)-Arg-Leu-Thr-Glu-Pro-,
-Pro-Ser-Arg-Leu-Glu-Glu-cyclo(Asp-Leu-Arg-DLys)-Arg-Leu-Thr-Glu-Pro-,
-Pro-Ser-Arg-Leu-Glu-Glu-cyclo(DLys-Leu-Arg-Asp)-Arg-Leu-Thr-Glu-Pro-,
-Pro-Ser-Arg-Leu-Glu-Glu-cyclo(DLys-Leu-Arg-DAsp)-Arg-Leu-Thr-Glu-Pro-,
-Pro-Ser-Arg-Leu-Glu-Glu-cyclo(DAsp-Leu-Arg-DLys)-Arg-Leu-Thr-Glu-Pro-,
-Ser-Arg-Leu-Glu-cyclo(Cys-Glu-Leu-Cys)-Arg-Arg-Leu-Thr-Glu-Pro-,
-Ser-Arg-Leu-Glu-Glu-Glu-Leu-Arg-Arg-cyclo(Cys-Leu-Thr-Cys)-Pro-,
-Ser-Arg-Leu-Glu-Glu-Glu-Leu-Arg-cyclo(Cys-Arg-Leu-Cys)-Glu-Pro-,
-Ser-Arg-Leu-Glu-Glu-cyclo(Cys-Leu-Arg-Cys)-Arg-Leu-Thr-Glu-Pro-,
-Pro-Ser-Arg-Leu-Glu-cyclo(Cys-Glu-Leu-Cys)-Arg-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-Glu-Leu-Arg-cyclo(Cys-Arg-Leu-Cys)-Glu-Pro-,
-Pro-Ser-Arg-Leu-Glu-Glu-cyclo(Cys-Leu-Arg-Cys)-Arg-Leu-Thr-Glu-Pro-,
-Ser-Arg-Leu-Glu-cyclo(DCys-Glu-Leu-DCys)-Arg-Arg-Leu-Thr-Glu-Pro-,
-Ser-Arg-Leu-Glu-cyclo(DCys-Glu-Leu-Cys)-Arg-Arg-Leu-Thr-Glu-Pro-,
-Ser-Arg-Leu-Glu-cyclo(hCys-Glu-Leu-Cys)-Arg-Arg-Leu-Thr-Glu-Pro-,
-Ser-Arg-Leu-Glu-cyclo(hCys-Glu-Leu-hCys)-Arg-Arg-Leu-Thr-Glu-Pro-,
-Ser-Arg-Leu-Glu-cyclo(Cys-Glu-Leu-hCys)-Arg-Arg-Leu-Thr-Glu-Pro-,
-Ser-Arg-Leu-Glu-cyclo(hCys-Glu-Leu-DCys)-Arg-Arg-Leu-Thr-Glu-Pro-,
-Ser-Arg-Leu-Glu-cyclo(DCys-Glu-Leu-hCys)-Arg-Arg-Leu-Thr-Glu-Pro-,
-Ser-Arg-Leu-Glu-cyclo(hCys-Glu-Leu-DhCys)-Arg-Arg-Leu-Thr-Glu-Pro-,
-Ser-Arg-Leu-Glu-cyclo(DhCys-Glu-Leu-DhCys)-Arg-Arg-Leu-Thr-Glu-Pro-,
-Ser-Arg-Leu-Glu-cyclo(Cys-Glu-Leu-DhCys)-Arg-Arg-Leu-Thr-Glu-Pro-,
-Ser-Arg-Leu-Glu-cyclo(DCys-Glu-Leu-DhCys)-Arg-Arg-Leu-Thr-Glu-Pro-,
-Ser-Arg-Leu-Glu-cyclo(DhCys-Glu-Leu-Cys)-Arg-Arg-Leu-Thr-Glu-Pro-,
-Ser-Arg-Leu-Glu-cyclo(DhCys-Glu-Leu-DCys)-Arg-Arg-Leu-Thr-Glu-Pro-,
-Pro-Ser-Arg-Leu-Glu-cyclo(DCys-Glu-Leu-DCys)-Arg-Arg-Leu-Thr-Glu-Pro-,
-Pro-Ser-Arg-Leu-Glu-cyclo(DCys-Glu-Leu-Cys)-Arg-Arg-Leu-Thr-Glu-Pro-,
-Pro-Ser-Arg-Leu-Glu-cyclo(hCys-Glu-Leu-Cys)-Arg-Arg-Leu-Thr-Glu-Pro-,
-Pro-Ser-Arg-Leu-Glu-cyclo(hCys-Glu-Leu-hCys)-Arg-Arg-Leu-Thr-Glu-Pro-,
-Pro-Ser-Arg-Leu-Glu-cyclo(Cys-Glu-Leu-hCys)-Arg-Arg-Leu-Thr-Glu-Pro-,
-Pro-Ser-Arg-Leu-Glu-cyclo(hCys-Glu-Leu-DCys)-Arg-Arg-Leu-Thr-Glu-Pro-,
-Pro-Ser-Arg-Leu-Glu-cyclo(DCys-Glu-Leu-hCys)-Arg-Arg-Leu-Thr-Glu-Pro-,
-Pro-Ser-Arg-Leu-Glu-cyclo(hCys-Glu-Leu-DhCys)-Arg-Arg-Leu-Thr-Glu-Pro-,
-Pro-Ser-Arg-Leu-Glu-cyclo(DhCys-Glu-Leu-DhCys)-Arg-Arg-Leu-Thr-Glu-Pro-,
-Pro-Ser-Arg-Leu-Glu-cyclo(Cys-Glu-Leu-DhCys)-Arg-Arg-Leu-Thr-Glu-Pro-,
-Pro-Ser-Arg-Leu-Glu-cyclo(DCys-Glu-Leu-DhCys)-Arg-Arg-Leu-Thr-Glu-Pro-,
-Pro-Ser-Arg-Leu-Glu-cyclo(DhCys-Glu-Leu-Cys)-Arg-Arg-Leu-Thr-Glu-Pro-,
-Pro-Ser-Arg-Leu-Glu-cyclo(DhCys-Glu-Leu-DCys)-Arg-Arg-Leu-Thr-Glu-Pro-,
-Ser-Arg-Leu-Glu-Glu-Glu-Leu-Arg-Arg-cyclo(Cys-Leu-Thr-DCys)-Pro-,
-Ser-Arg-Leu-Glu-Glu-Glu-Leu-Arg-Arg-cyclo(DCys-Leu-Thr-Cys)-Pro-,
-Ser-Arg-Leu-Glu-Glu-Glu-Leu-Arg-Arg-cyclo(DCys-Leu-Thr-DCys)-Pro-,
-Ser-Arg-Leu-Glu-Glu-Glu-Leu-Arg-Arg-cyclo(hCys-Leu-Thr-Cys)-Pro-,
-Ser-Arg-Leu-Glu-Glu-Glu-Leu-Arg-Arg-cyclo(hCys-Leu-Thr-DCys)-Pro-,
-Ser-Arg-Leu-Glu-Glu-Glu-Leu-Arg-Arg-cyclo(hCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DhCys-Leu-Thr-Cys)-Pro-,
-Ser-Arg-Leu-Glu-Glu-Glu-Leu-Arg-Arg-cyclo(DhCys-Leu-Thr-DCys)-Pro-,
-Ser-Arg-Leu-Glu-Glu-Glu-Leu-Arg-Arg-cyclo(DhCys-Leu-Thr-hCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(DCys-Leu-Thr-DhCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(Pen-Leu-Thr-Pen)-Pro-,
-Ser-Arg-Leu-Glu-Glu-Glu-Leu-Arg-Arg-cyclo(Pen-Leu-Thr-DPen)-Pro-,
-Ser-Arg-Leu-Glu-Glu-Glu-Leu-Arg-Arg-cyclo(DPen-Leu-Thr-Pen)-Pro-,
-Ser-Arg-Leu-Glu-Glu-Glu-Leu-Arg-Arg-cyclo(DPen-Leu-Thr-DPen)-Pro-,
-Ser-Arg-Leu-Glu-Glu-Glu-Leu-Arg-cyclo(DCys-Arg-Leu-Cys)-Glu-Pro-,
-Ser-Arg-Leu-Glu-Glu-Glu-Leu-Arg-cyclo(DCys-Arg-Leu-DCys)-Glu-Pro-,
-Ser-Arg-Leu-Glu-Glu-Glu-Leu-Arg-cyclo(Cys-Arg-Leu-DCys)-Glu-Pro-,
-Ser-Arg-Leu-Glu-Glu-Glu-Leu-Arg-cyclo(hCys-Arg-Leu-Cys)-Glu-Pro-,
-Ser-Arg-Leu-Glu-Glu-Glu-Leu-Arg-cyclo(hCys-Arg-Leu-DCys)-Glu-Pro-,
-Ser-Arg-Leu-Glu-Glu-Glu-Leu-Arg-cyclo(Cys-Arg-Leu-hCys)-Glu-Pro-,
-Ser-Arg-Leu-Glu-Glu-Glu-Leu-Arg-cyclo(hCys-Arg-Leu-hCys)-Glu-Pro-,
-Ser-Arg-Leu-Glu-Glu-Glu-Leu-Arg-cyclo(hCys-Arg-Leu-DhCys)-Glu-Pro-,
-Ser-Arg-Leu-Glu-Glu-Glu-Leu-Arg-cyclo(DhCys-Arg-Leu-hCys)-Glu-Pro-,
-Pro-Ser-Arg-Leu-Glu-Glu-Glu-Leu-Arg-Arg-cyclo(Cys-Leu-Thr-DCys)-Pro-,
-Pro-Ser-Arg-Leu-Glu-Glu-Glu-Leu-Arg-Arg-cyclo(DCys-Leu-Thr-Cys)-Pro-,
-Pro-Ser-Arg-Leu-Glu-Glu-Glu-Leu-Arg-Arg-cyclo(DCys-Leu-Thr-DCys)-Pro-,
-Pro-Ser-Arg-Leu-Glu-Glu-Glu-Leu-Arg-Arg-cyclo(hCys-Leu-Thr-Cys)-Pro-,
-Pro-Ser-Arg-Leu-Glu-Glu-Glu-Leu-Arg-Arg-cyclo(hCys-Leu-Thr-DCys)-Pro-,
-Pro-Ser-Arg-Leu-Glu-Glu-Glu-Leu-Arg-Arg-cyclo(hCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DhCys-Leu-Thr-Cys)-Pro-,
-Pro-Ser-Arg-Leu-Glu-Glu-Glu-Leu-Arg-Arg-cyclo(DhCys-Leu-Thr-DCys)-Pro-,
-Pro-Ser-Arg-Leu-Glu-Glu-Glu-Leu-Arg-Arg-cyclo(DhCys-Leu-Thr-hCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(DCys-Leu-Thr-DhCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(Pen-Leu-Thr-Pen)-Pro-,
-Pro-Ser-Arg-Leu-Glu-Glu-Glu-Leu-Arg-Arg-cyclo(Pen-Leu-Thr-DPen)-Pro-,
-Pro-Ser-Arg-Leu-Glu-Glu-Glu-Leu-Arg-Arg-cyclo(DPen-Leu-Thr-Pen)-Pro-,
-Pro-Ser-Arg-Leu-Glu-Glu-Glu-Leu-Arg-Arg-cyclo(DPen-Leu-Thr-DPen)-Pro-,
-Pro-Ser-Arg-Leu-Glu-Glu-Glu-Leu-Arg-cyclo(DCys-Arg-Leu-Cys)-Glu-Pro-,
-Pro-Ser-Arg-Leu-Glu-Glu-Glu-Leu-Arg-cyclo(DCys-Arg-Leu-DCys)-Glu-Pro-,
-Pro-Ser-Arg-Leu-Glu-Glu-Glu-Leu-Arg-cyclo(Cys-Arg-Leu-DCys)-Glu-Pro-,
-Pro-Ser-Arg-Leu-Glu-Glu-Glu-Leu-Arg-cyclo(hCys-Arg-Leu-Cys)-Glu-Pro-,
-Pro-Ser-Arg-Leu-Glu-Glu-Glu-Leu-Arg-cyclo(hCys-Arg-Leu-DCys)-Glu-Pro-,
-Pro-Ser-Arg-Leu-Glu-Glu-Glu-Leu-Arg-cyclo(Cys-Arg-Leu-hCys)-Glu-Pro-,
-Pro-Ser-Arg-Leu-Glu-Glu-Glu-Leu-Arg-cyclo(hCys-Arg-Leu-hCys)-Glu-Pro-,
-Pro-Ser-Arg-Leu-Glu-Glu-Glu-Leu-Arg-cyclo(hCys-Arg-Leu-DhCys)-Glu-Pro-,
-Pro-Ser-Arg-Leu-Glu-Glu-Glu-Leu-Arg-cyclo(DhCys-Arg-Leu-hCys)-Glu-Pro-,
-Ser-Arg-Leu-Glu-Glu-Glu-Leu-Arg-cyclo(Lys-Arg-Leu-Asp)-Glu-Pro-,
-Ser-Arg-Leu-Glu-Glu-Glu-Leu-Arg-cyclo(Asp-Arg-Leu-Lys)-Glu-Pro-,
-Ser-Arg-Leu-Glu-Glu-Glu-Leu-Arg-cyclo(Lys-Arg-Leu-Glu)-Glu-Pro-,
-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Lys-Arg-Leu-Asp)-Glu-Pro-,
-Pro-Ser-Arg-Leu-Glu-Glu-Glu-Leu-Arg-cyclo(Asp-Arg-Leu-Lys)-Glu-Pro-,
-Pro-Ser-Arg-Leu-Glu-Glu-Glu-Leu-Arg-cyclo(Lys-Arg-Leu-Glu)-Glu-Pro-,
-Pro-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Cys-Arg-Leu-Thr-Cys)-Pro-,
-Pro-Ser-Arg-Leu-Glu-cyclo(Cys-Glu-Leu-Arg-Cys)-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-cyclo(Cys-Leu-Arg-Arg-Cys)-Leu-Thr-Glu-Pro-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu -.

In some embodiments, the cyclic peptide is attached to a 3-mercaptopropionyl moiety through an a-amine moiety of the leftmost amino acid in the cyclic peptide. In some embodiments, the rightmost amino acid in the cyclic peptide comprises an amide.

In some embodiments, the cyclized amino acid sequence is one selected from the group consisting of

-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
-Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-
-Pro-Ser-Arg-Leu-Glu-cyclo(Glu-Glu-Leu-Arg-DLys)-Arg-Leu-Thr-Glu-
-Pro-Ser-Arg-Leu-Glu-cyclo(Lys-Glu-Leu-Arg-Glu)-Arg-Leu-Thr-Glu-
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu -.

In some embodiments, the cyclized amino acid sequence is -Ser-Arg-Leu-Glu-cyclo(Glu-Glu-Leu-Arg-Lys)-Arg-Leu-Thr-Glu-. In some other embodiments, the cyclized amino acid sequence is -Ser-Arg-Leu-Glu-cyclo(Asp-Glu-Leu-Arg-Lys)-Arg-Leu-Thr-Glu-. In yet some other embodiments, the cyclized amino acid sequence is -Ser-Arg-Leu-Glu-cyclo(Glu-Glu-Leu-Lys)-Arg-Arg-Leu-Thr-Glu-. In still some other embodiments, the cyclized amino acid sequence is -Ser-Arg-Leu-Glu-Glu-Glu-Leu-Arg-cyclo(Lys-Arg-Leu-Thr-Glu)-.

The cyclic peptides may have different cyclic bridging moieties forming the ring structure. Preferably, chemically stable bridging moieties are included in the ring structure such as, for example, an amide group, a lactone group, an ether group, a thioether group, a disulfide group, an alkylene group, an alkenyl group, or a 1,2,3-triazole. The following are examples illustrating the variability of bridging moieties in a peptide:

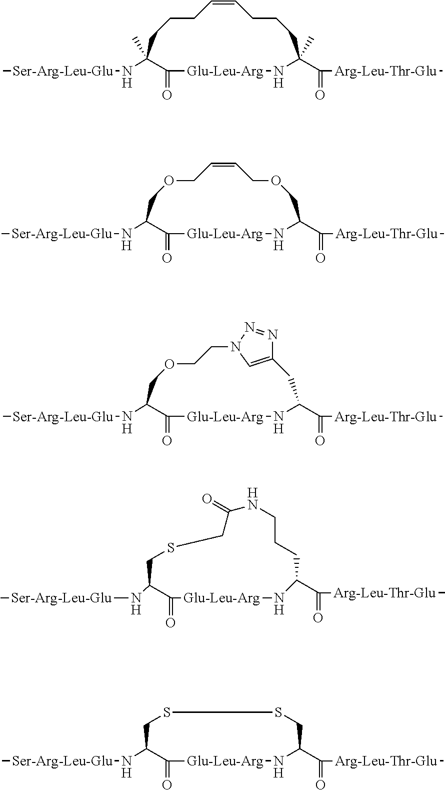

In some embodiments, an ALFA-tag binding moiety comprises an antibody or antibody fragment, e.g., a camelid VHH domain. In some embodiments, an ALFA-tag binding moiety comprises a single-domain antibody (sdAb), NbALFA-nanobody.

In some embodiments, an ALFA-tag binding moiety comprises a single domain antibody, e.g., a camelid VHH domain comprising the CDR1 sequence VTX₁SALNAMAMG, wherein X₁ is I or V, the CDR2 sequence AVSX₂RGNAM, wherein X₂ is E, H, N, D, or S, and the CDR3 sequence LEDRVDSFHDY.

In some embodiments, an ALFA-tag binding moiety comprises a single domain antibody, e.g., a camelid VHH domain comprising the CDR1 sequence GVTX₁SALNAMAMG, wherein X₁ is I or V, the CDR2 sequence AVSX₂RGNAM, wherein X₂ is E, H, N, D, or S, and the CDR3 sequence LEDRVDSFHDY.

In some embodiments, an ALFA-tag binding moiety comprises a single domain antibody, e.g., a camelid VHH domain comprising the CDR1 sequence VTISALNAMAMG, the CDR2 sequence AVSERGNAM, and the CDR3 sequence LEDRVDSFHDY.

In some embodiments, an ALFA-tag binding moiety comprises a single domain antibody, e.g., a camelid VHH domain comprising the CDR1 sequence GVTISALNAMAMG, the CDR2 sequence AVSERGNAM, and the CDR3 sequence LEDRVDSFHDY.

In some embodiments, an ALFA-tag binding moiety comprises a single domain antibody, e.g., a camelid VHH domain comprising the amino acid sequence EVQLQESGGGLVQPGGSLRLSCTASGVTISALNAMAMGWYRQAPGERRVMVAAVSERGNAMYRESVQGRFTVTR DFTNKMVSLQMDNLKPEDTAVYYCHVLEDRVDSFHDYWGQGTQVTVSS, an amino acid sequence having at least 99%, 98%, 97%, 96%, 95%, 90%, 85%, or 80% identity to said amino acid sequence, or a fragment of said amino acid sequence or the amino acid sequence having at least 99%, 98%, 97%, 96%, 95%, 90%, 85%, or 80% identity to said amino acid sequence. In some embodiments, the amino acid sequence comprises CDR1, CDR2 and CDR3 sequences as described above.

In some embodiments, the epitope tag/binder system comprises an epitope tag comprising the sequence PDRVRAVSHWSS (Spot-tag) and the binder comprises a single-domain antibody (sdAb, or nanobody) (Spot-nanobody (14.7 kD)) that specifically binds to the Spot-tag.

In some embodiments, following binding of the moieties on the targeting compound and on the docking compound interacting which each other, a covalent connection is formed. In these embodiments, the system used herein may comprise a Tag/Catcher system forming a covalent bond, e.g., SpyTag/SpyCatcher forming an isopeptide bond.

The SpyTag/SpyCatcher system is a technology for irreversible conjugation of recombinant proteins. The peptide SpyTag spontaneously reacts with the protein SpyCatcher to form an intermolecular isopeptide bond between the pair. Using the Tag/Catcher pair, bioconjugation can be achieved between two recombinant proteins.

The present disclosure provides in one aspect, a complex wherein a particle comprising a targeting compound (hydrophobic moiety having a binding moiety covalently attached thereto) is bound to a docking compound (compound comprising (i) a moiety binding to the binding moiety covalently attached to a hydrophobic moiety and (ii) a moiety targeting a cell surface antigen). Thus, the targeting compound and the docking compound comprise moieties interacting which each other.

Accordingly, the present disclosure provides in one aspect, a complex comprising:
(a) a particle comprising a hydrophobic moiety having a binding moiety covalently attached thereto, and
(b) a compound comprising (i) a moiety binding to the binding moiety covalently attached to the hydrophobic moiety and (ii) a moiety targeting a cell surface antigen.

Different embodiments of the targeting compound and the docking compound which are complexed are described herein.

In some embodiments, the targeting compound comprises an ALFA-tag. In these embodiments, the moiety binding to a targeting compound of the docking compound may be a NbALFA-nanobody (NbALFA). In some embodiments, the docking compound may have a structure selected from the group consisting of NbALFA×anti-primary target DARPin, NbALFA×anti-primary target VHH and NbALFA×anti-primary target scFv.

### Embodiments of Targeting Compound

In some embodiments, the targeting compound described herein is a lipid-PEG-peptide conjugate compound having the structure (I):

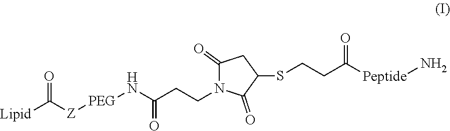

wherein the Lipid is a phospholipid attached to the carbonyl through the amino group of the ethanolamine moiety thereof, PEG has a molecular weight of from about 130 to about 50,000, α-amino group of the left most amino acid group of the Peptide is attached to the carbonyl group of the 3-mercaptopropionyl moiety and the Peptide comprises from about 11 to about 15 amino acids, and Z is a bond or —CH₂—. In some embodiments, the lipid-PEG-peptide conjugate has the following structure (II)

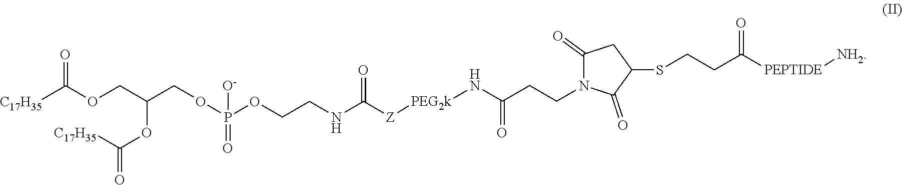

A particular embodiment of the lipid-PEG-peptide conjugate compound having the structure (I) or (II) is where Z is a bond. In one embodiment, the PEPTIDE has the sequence -SRLEEELRRRLTE-. In another embodiment, the PEPTIDE has the sequence -PSRLEEELRRRLTE-. In other embodiments, the PEPTIDE is a cyclic peptide selected from the group consisting of -Ser-Arg-Leu-Glu-cyclo(Glu-Glu-Leu-Arg-Lys)-Arg-Leu-Thr-Glu-, -Ser-Arg-Leu-Glu-cyclo(Asp-Glu-Leu-Arg-Lys)-Arg-Leu-Thr-Glu-, -Ser-Arg-Leu-Glu-cyclo(Glu-Glu-Leu-Lys)-Arg-Arg-Leu-Thr-Glu-, -Ser-Arg-Leu-Glu-Glu-Glu-Leu-Arg-cyclo(Lys-Arg-Leu-Thr-Glu)-, -Ser-Arg-Leu-Glu-cyclo(Cys-Glu-Leu-Arg-Cys)-Arg-Leu-Thr-Glu-, -Ser-Arg-Leu-Glu-cyclo(Asp-Glu-Leu-Lys)-Arg-Arg-Leu-Thr-Glu-, -Ser-Arg-Leu-Glu-Glu-Glu-Leu-Arg-cyclo(Lys-Arg-Leu-Thr-Asp)-, -Pro-Ser-Arg-Leu-Glu-cyclo(Glu-Glu-Leu-Arg-Lys)-Arg-Leu-Thr-Glu-, -Pro-Ser-Arg-Leu-Glu-cyclo(DGlu-Glu-Leu-Arg-Lys)-Arg-Leu-Thr-Glu-, -Pro-Ser-Arg-Leu-Glu-cyclo(Glu-Glu-Leu-Arg-DLys)-Arg-Leu-Thr-Glu-, -Pro-Ser-Arg-Leu-Glu-cyclo(Lys-Glu-Leu-Arg-Glu)-Arg-Leu-Thr-Glu-, -Pro-Ser-Arg-Leu-cyclo(Glu-Glu-Glu-Leu-Lys)-Arg-Arg-Leu-Thr-Glu-, -Pro-Ser-Arg-Leu-Glu-cyclo(Cys-Glu-Leu-Cys)-Arg-Arg-Leu-Thr-Glu-, -Pro-Ser-Arg-Leu-Glu-Glu-Glu-Leu-Arg-cyclo(Cys-Arg-Leu-Thr-Cys)-, -Pro-Ser-Arg-Leu-Glu-cyclo(Cys-Glu-Leu-Arg-Cys)-Arg-Leu-Thr-Glu-, -Pro-Ser-Arg-Leu-Glu-Glu-Glu-Leu-Arg-Arg-cyclo(Cys-Leu-Thr-Cys)-, and -Pro-Ser-Arg-Leu-Glu-Glu-cyclo(Cys-Leu-Arg-Arg-Cys)-Leu-Thr-Glu-.

The term “PEG” as used in the above formula I means any polyethylene glycol or other polyalkylene ether polymer. In one embodiment, PEG is an optionally substituted linear or branched polymer of ethylene glycol or ethylene oxide. In one embodiment PEG is unsubstituted. In one embodiment the PEG is substituted, e.g., by one or more alkyl, alkoxy, acyl, hydroxy or aryl groups. In one embodiment, the PEG has a molecular weight of from about 130 to about 50,000, in another embodiment about 150 to about 30,000, in another embodiment about 150 to about 20,000, in another embodiment about 150 to about 15,000, in another embodiment about 150 to about 10,000, in another embodiment about 150 to about 6000, in another embodiment about 150 to about 5000, in another embodiment about 150 to about 4000, in another embodiment about 150 to about 3000, in another embodiment about 300 to about 3000, in another embodiment about 1000 to about 3000, and in still another embodiment about 1500 to about 2500.

In certain embodiments the PEG (conjugated to the lipid) is a “PEG2k”, also termed “PEG 2000”, which has an average molecular weight of about 2000 Daltons. Another name for a lipid comprising polyethylene glycol PEG, such as, for example PEG2K, is a “pegylated lipid” and, if a phosphorous-containing linkage is present, the lipid is generally referred to herein as a “phospholipid” or a “pegylated phospholipid.”

In some embodiments, the lipid portion to which the PEG is bonded in the functionalized stealth lipid disclosed in the above formula I comprises a neutral phospholipid. Examples of neutral phospholipids include, but are not limited to: dipalmitoylphosphatidylcholine (DPPC), distearoylphosphatidylcholine (DSPC), dioleoyl-sn-glycero-3-phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), I-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, a preferred lipid is distearoyl-phosphatidylethanolamine (DSPE).

The term “PEPTIDE” or “Peptide” are used interchangeably and as used in the above formula I or 11 refers to a series of amino acids connected one to another by peptide bonds between the amino and carboxy groups of adjacent residues. “PEPTIDE-NH₂” represents that the C-terminus carboxyl group of the peptide is an amide. In some embodiments, the term “PEPTIDE” or “Peptide” refers to an epitope tag, e.g., an ALFA-tag such as an ALFA-tag described herein.

### Particles Comprising a Nucleic Acid Payload

The particles described herein comprise one or more particle forming agents, a nucleic acid payload to be delivered to a target cell and a hydrophobic moiety having a binding moiety covalently attached thereto for binding to the target cells or the docking compound.

### Nucleic Acid Payload

In some embodiments, a nucleic acid payload, e.g., to be delivered to target cells to genetically modify the target cells and enable the target cells to express a biomolecule, e.g., peptide or protein, encoded by the nucleic acid, comprises DNA, RNA, or a mixture thereof.

In some embodiments, the agents and methods described herein are used for targeted therapy. This may be achieved by making use of a payload comprising one or more nucleic acids which are pharmaceutically active agents.

The term “pharmaceutically active agent” relates to any agent such as compound or cell being therapeutically effective when administered to an individual. The term “pharmaceutically active agent” further relates to any agent that changes, preferably cures, alleviates or partially arrests the clinical manifestations of a given disease and its complications in a therapeutic intervention comprising the administration of said agent.

In some embodiments, a pharmaceutically active agent comprises pharmaceutically active nucleic acid such as pharmaceutically active RNA.

A “pharmaceutically active nucleic acid” is nucleic acid, e.g., RNA, that encodes a pharmaceutically active peptide or protein or is pharmaceutically active in its own, e.g., it has one or more pharmaceutical activities such as those described for pharmaceutically active proteins. For example, the RNA may be one or more strands of RNA interference (RNAi). Such agents include short interfering RNAs (siRNAs), or short hairpin RNAs (shRNAs), or precursor of a siRNA or microRNA-like RNA, targeted to a target transcript, e.g., a transcript of an endogenous disease-related transcript of a subject.

A “pharmaceutically active peptide or protein” has a positive or advantageous effect on the condition or disease state of a subject when provided to the subject in a therapeutically effective amount. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., administration of the peptide or protein to a subject elicits an immune response in a subject which may be therapeutic or partially or fully protective.

Examples of pharmaceutically active proteins include, but are not limited to, cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, immunoglobulins, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Wilebrand factor, antithrombin III, glucocerebrosidase, erythropoietin granulocyte colony stimulating factor (GCSF) or modified Factor VIII, anticoagulants and the like.

In some embodiments, the pharmaceutically active protein is a cytokine which is involved in regulating lymphoid homeostasis, preferably a cytokine which is involved in and preferably induces or enhances development, priming, expansion, differentiation and/or survival of T cells. In some embodiments, the cytokine is an interleukin. In some embodiments, the pharmaceutically active protein according to the disclosure is an interleukin selected from the group consisting of IL-2, IL-7, IL-12, IL-15, and IL-21.

In some embodiments, a nucleic acid to be delivered to a target cell is nucleic acid encoding an antigen receptor. In these embodiments, the target cells may be immune cells or immune effector cells.

### Particles

A nucleic acid payload may be administered with one or more delivery vehicles that protect the payload from degradation, maximize delivery to on-target cells and minimize exposure to off-target cells. Such delivery vehicles may complex or encapsulate the payload and include a range of materials, including polymers, lipids and mixtures thereof. In some embodiments, such delivery vehicles may form particles with the payload.

In the context of the present disclosure, the term “particle” relates to a structured entity formed by molecules or molecule complexes, in particular particle forming compounds. In some embodiments, a particle is a nucleic acid containing particle such as a particle comprising DNA, RNA or a mixture thereof. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes nanoparticles.

The term “nanoparticle” relates to a nano-sized particle comprising at least one particle forming agent, e.g., at least one cationic or cationically ionizable lipid, wherein all three external dimensions of the particle are in the nanoscale, i.e., at least about 1 nm and below about 1000 nm. Preferably, the size of a particle is its diameter.

In some embodiments, the particles described herein have a size (such as a diameter) in the range of about 10 to about 2000 nm, such as at least about 15 nm (e.g., at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most about 1900 nm (e.g., at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), such as in the range of about 20 to about 1500 nm, such as about 30 to about 1200 nm, about 40 to about 1100 nm, about 50 to about 1000 nm, about 60 to about 900 nm, about 70 to about 800 nm, about 80 to about 700 nm, about 90 to about 600 nm, or about 50 to about 500 nm or about 100 to about 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, 70 to 150 nm, or 80 to 150 nm. In some embodiments, the particles described herein have a size (such as a diameter) in the range of from about 40 nm to about 200 nm, such as from about 50 nm to about 180 nm, from about 60 nm to about 160 nm, from about 80 nm to about 150 nm or from about 80 nm to about 120 nm.

In some embodiments, the particles described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, from about 200 nm to about 250 nm, or from about 80 to about 150 nm. In some embodiments, the particles described herein have an average diameter that in some embodiments ranges from about 40 nm to about 200 nm, such as from about 50 nm to about 180 nm, from about 60 nm to about 160 nm, from about 80 nm to about 150 nm or from about 80 nm to about 120 nm.

Particles described herein may exhibit a polydispersity index (PDI) less than about 0.5, less than about 0.4, less than about 0.3, less than about 0.2, less than about 0.1, or less than about 0.05. By way of example, the particles can exhibit a polydispersity index in a range of about 0.01 to about 0.4 or about 0.1 to about 0.3.

A “nucleic acid particle” can be used to deliver nucleic acid to a target site of interest (e.g., cell, tissue, organ, and the like). A nucleic acid particle may be formed from at least one cationic or cationically ionizable compound such as a polymer or lipid complexing the nucleic acid. Without intending to be bound by any theory, it is believed that the cationic or cationically ionizable compound combines together with the nucleic acid to form aggregates, and this aggregation results in colloidally stable particles.

In some embodiments, nucleic acid may be noncovalently associated with a particle. In some embodiments, the nucleic acid may be adhered to the outer surface of the particle (surface nucleic acid) and/or may be contained in the particle (encapsulated nucleic acid).

The N/P ratio gives the ratio of the nitrogen groups in the lipid to the number of phosphate groups in the nucleic acid. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, nucleic acid is considered to be completely bound to nanoparticles.

Particles described herein can be prepared using a wide range of methods. For example, methods for preparing nucleic acid particles may involve obtaining a colloid from at least one cationic or cationically ionizable lipid and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, lipoplex particles are obtainable by adding nucleic acid to a colloidal liposome dispersion. Using the ethanol injection technique, such colloidal liposome dispersion is, in some embodiments, formed as follows: an ethanol solution comprising lipids, such as cationic or cationically ionizable lipids and additional lipids, is injected into an aqueous solution under stirring.

The term “particle forming components” or “particle forming agents” relates to any components which form particles, e.g., by associating with a payload. Delivery vehicles such as particle forming agents useful herein include polymers, polymer derivatives, lipids, e.g., as described herein, and mixtures thereof. Such components include any component which can be part of nucleic acid particles, e.g., cationic or cationically ionizable lipids.

### Polymers

Given their high degree of chemical flexibility, polymers are commonly used materials for nanoparticle-based delivery. Typically, cationic polymers are used to electrostatically condense negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In some embodiments, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI). Cationic polymers (including polycationic polymers) contemplated for use herein include any cationic polymers which are able to electrostatically bind nucleic acid. In some embodiments, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

Particles comprising nucleic acid are also referred to as “polyplexes (PLX)” herein. Such PLX may comprise a lipid component, e.g., the lipid component of a targeting compound. Such particles containing polymer and lipid, e.g., functionalized lipid, are also referred to as lipidated polyplexes (LPLX).

### Polyamine Derivatives (Viromers)

In some embodiments, the delivery vehicle comprises a polyamine derivative, e.g., a carboxylated polyamine derivative. Polyamines form polycations in solution, which facilitates the complex formation with polyanions such as nucleic acids.

In some embodiments, a polyamine derivative which is useful herein as delivery vehicle for polyanions comprises:
a polyamine moiety comprising a plurality of amino groups;
a plurality of carboxylated substituents comprising a carboxyl group bonded via a hydrophobic linker to amino groups of said polyamine moiety; and
a plurality of hydrophobic substituents bonded to amino groups of said polyamine moiety.

In some embodiments, a polyamine derivative which is useful herein as delivery vehicle for polyanions comprises:
a polyamine moiety comprising a plurality of amino groups;
a plurality of carboxylated substituents comprising a carboxyl group bonded via a hydrophobic linker to amino groups of said polyamine moiety, wherein each of said carboxylated substituents comprises from 6 to 40 carbon atoms, preferably from 6 to 20 carbon atoms, and more preferably from 8 to 16 carbon atoms, and each of said hydrophobic linker may comprise from 1 to 3 heteroatoms selected from O, N, and S; and
a plurality of hydrophobic substituents bonded to amino groups of said polyamine moiety, wherein each of said hydrophobic substituents comprises at least 2 carbon atoms, preferably from 6 to 40 carbon atoms, and may comprise from 1 to 3 heteroatoms selected from O, N, and S provided said hydrophobic substituent has at least 6 carbon atoms.

In some embodiments, each of said carboxylated substituents of said polyamine derivative comprises any one or more of the following moieties as said hydrophobic linker: alkylene, alkenylene, alkynylene, cycloalkylene, cycloalkenylene, arylene, and combinations thereof; and/or

each of said hydrophobic substituents of said polyamine derivative comprises any one or more of the following moieties: alkyl, alkenyl, alkynyl, cycloalkyl, cycloalkenyl, aryl, and combinations thereof.

In some embodiments, a polyamine derivative which is useful herein as delivery vehicle for polyanions is a polyalkylenimine derivative having one or more carboxyalkyl substituents comprising from 6 to 40 carbon atoms, and one or more hydrophobic substituents selected from hydrocarbon substituents having at least 2 carbon atoms, preferably from 6 to 40 carbon atoms, wherein each of said hydrophobic substituents may be or may comprise an alkyl group and/or each of said hydrophobic substituents may be or may comprise an aryl group.

In some embodiments, the polyalkylenimine is selected from the group consisting of polyethylenimines, polypropylenimines, and polybutylenimines.

In some embodiments, the polyamine moiety of said polyamine derivative may comprise from 4 to 20000 nitrogen atoms, more preferably from 6 to 10000 nitrogen atoms, e.g., from 6 to 1000 nitrogen atoms, or from 6 to 100 nitrogen atoms per polyamine molecule.

In some embodiments, the polyamine moiety of said polyamine derivative may be a branched polyamine, preferably a branched polyalkylenimine.

In some embodiments, a carboxylated substitutent comprises one or two carboxyl groups, preferably one carboxyl group. In some embodiments, each carboxylated substitutent comprises from 6 to 40 carbon atoms, preferably from 6 to 20 carbon atoms, and more preferably from 8 to 16 carbon atoms. The hydrophobic linkers of said carboxylated substituents may comprise from 1 to 3, preferably, 1 or 2, heteroatoms selected from O, N, and S. Preferably, the heteroatoms are selected from O and S. In one embodiment, 1 or 2 heteroatoms selected from O, N and S, preferably O and S, may be contained in the hydrophobic linker. Thus, the carboxylated substituents may be carboxyhydrocarbyl groups, or they may be carboxyheterohydrocarbyl groups comprising from 1 to 3 heteroatoms selected from O, N, and S, preferably selected from O and S.

Among the plurality of carboxylated substituents of a molecule of said polyamine derivative, there may be exclusively carboxyhydrocarbyl groups, exclusively carboxyheterohydrocarbyl groups, or there may be carboxyhydrocarbyl groups and carboxyheterohydrocarbyl groups. In some embodiments, the plurality of carboxylated substituents are all carboxyhydrocarbyl groups. In some embodiments, the plurality of carboxylated substituents are all carboxyheterohydrocarbyl groups.

Where the carboxylated substituents are carboxyhydrocarbyl groups, the hydrocarbyl moieties of said carboxyhydrocarbyl groups may be saturated aliphatic hydrocarbyl moieties, unsaturated aliphatic hydrocarbyl moieties, alicyclic hydrocarbyl moieties, aromatic hydrocarbyl moieties, or moieties comprising two or more moieties from the aforementioned list.

Examples of the carboxyhydrocarbyl groups are carboxyalkyl groups, carboxyalkenyl groups, carboxyalkynyl groups, carboxycycloalkyl groups, carboxycycloalkenyl groups, carboxyalkylcycloalkyl groups, carboxycycloalkylalkyl groups, carboxyalkylcycloalkylalkyl groups, carboxyaryl groups, carboxyalkylaryl groups, carboxyarylalkyl groups, and carboxyalkylarylalkyl groups. It is possible to replace 1, 2 or 3, preferably 1 or 2, of the carbon atoms of the hydrocarbyl moieties of the carboxylated substituents by oxygen, nitrogen or sulfur, thereby forming carboxyheterohydrocarbyl moieties. It is understood that any such formal replacement by a heteroatom will include adjustment of bound hydrogen atoms to adjust to the valency of the exchanged heteroatom. In preferred embodiments, such carboxyheterohydrocarbyl moieties comprise one or more functional group selected from —O—, —S—, —N(H)C(O)—, —C(O)O—, —OC(O)N(H)—, —C(O)—, —C(O)—N(H)—, —N(H)—C(O)—O—, —O—C(O)—, or —S—S— in the hydrophobic linker.

In some embodiments, the hydrophobic linkers are or comprise alkylene groups such as linear or branched alkylene groups, or the linkers are or comprise cycloalkylene groups. Alkylene groups may be n-alkylene or isoalkylene groups. Examples of alkylene groups are propylene, butylene, pentylene, hexylene, heptylene, octylene, nonylene, decylene, undecylene, dodecylene, tetradecylene or hexadecylene groups. Examples of cycloalkylene groups are cyclopentylene, cyclohexylene and cycloheptylene groups. Examples of alkylcycloalkyl groups are methylcyclopentylene, ethylcyclopentylene, propylcyclopentylene, butylcyclopentylene, pentylcyclopentylene, hexylcylopentylene, methylcyclohexylene, ethylcyclohexylene, propylcyclohexylene, butylcyclohexylene, pentylcyclohexylene and hexylcylohexylene. One or more of these may be combined in a hydrophobic linker.

In some embodiments, the carboxylated substituents are or comprise carboxyalkyl or carboxycycloalkyl groups comprising from 6 to 20 carbon atoms. Such carboxylated substituents may be selected from the group consisting of carboxy-n-alkyl groups, branched carboxyalkyl groups or cyclic carboxyalkyl groups and their constitution or conformation isomers. In a preferred embodiment, the carboxylalkyl groups are radicals of acids selected from hexanoic acid, heptanoic acid, octanoic acid, nonanoic acid, decanoic acid, undecanoic acid, dodecanoic acid, tetradecanoic acid, hexadecanoic acid, 2-cyclohexylacetic acid, 4-cyclohexylbutyric acid, 6-cyclohexylhexanoic acid, 2-(2′, 3′ or 4′ ethylcyclohexyl)-acetic acid or 4-(2′, 3′ or 4′ ethylcyclohexyl)-butyric acid or 6-(2′, 3′ or 4′ ethylcyclohexyl)-hexanoic acid.

In some embodiments, the hydrophobic linkers are or comprise arylene groups and have from 6 to 20 carbon atoms. Aryl groups forming said arylene groups include aromatic hydrocarbyl groups (carbon-only aryl groups) and aromatic heterohydrocarbyl groups (heteroaryl groups). Examples of the former are phenyl, naphthyl, anthracenyl and phenanthryl. In some embodiments, nitrogen-containing heteroaryl groups have a pK value of <5 for avoiding additional cationic charges at neutral pH. Examples of such nitrogen-containing heteroaryl groups are indolyl groups pyrazinyl groups, pyridazinyl groups, pyrimidinyl groups, cinnolinyl groups, phthalazinyl groups and purinyl groups. In some embodiments, oxygen-containing heterohydrocarbyl groups that form hydroxy groups have a pK>12 for avoiding negative charges at neutral pH.

Examples of alkylaryl groups are methylphenyl (tolyl), ethylphenyl, 4-isopropylphenyl, and xylyl groups. Examples of arylalkyl (aralkyl) groups are benzyl, phenylethyl and trityl groups. Examples of alkylarylalkyl groups are methylbenzyl and 4-isopropyl benzyl groups. Carboxyarylalkyl moieties may for example be radicals derived from from o, m or p-methyl benzoic acid, or o-, m- or p-ethyl benzoic acid. Carboxyalkylarylalkyl moieties may for example be o-, m- or p-methyl phenylacetic acid. Carboxyalkenylarylalkyl moieties may for example be or from o-, m- or p-methyl cinnamic acid.

Multiple carboxylated substituents such as those being or comprising carboxyalkyl groups present on the polyamine derivative may be the same or different. For simplicity, they may be the same. The carboxy group of the carboxylated substituent may be bound to any carbon atom of the hydrophobic linker. Preferably, the carboxy group is bound to a carbon atom as follows: if z is the number of carbon atoms in the longest carbon chain in the carboxylated substituent (such as the carboxyalkyl group) to the carbon atom that is bound to a polyamine nitrogen atom, the carboxy group is bound to a carbon atom at a position that is more than z/2 atom positions away from the polyamine nitrogen, if the carbon atom bound to the polyamine nitrogen is counted as position 1. If the value of z/2 is not an integer, the above definition leads to the position defined by the next integer>z/2. In one embodiment, the carboxy group is bound to the carbon atom of the hydrophobic linker that is most remote (in terms of the number of carbon atoms) from the polyamine nitrogen atom to which the hydrophobic linker (alkylene chain in the case of carboxyalkyl groups) is connected. The carboxy group may be bound to the carbon atom that is farthest away from the polyamine nitrogen within the carboxylated substituent (or carboxyalkyl group), such as to the terminal (omega position) carbon atom of the carboxylated substituents (or carboxyalkyl group) in case of a linear carboxylated substituent.

In some embodiments, the hydrophobic substituents comprise from 2 to 40 carbon atoms, in some embodiments, from 3 to 40 carbon atoms, in some embodiments from 6 to 40 carbon atoms and in some embodiments from 6 to 20 carbon atoms. The hydrophobic substituents may comprise from 1 to 3, preferably 1 or 2, heteroatoms selected from O, N, and S, provided said hydrophobic substituents comprise 6 or more carbon atoms. Preferably, the heteroatoms are selected from O and S. Thus, the hydrophobic substituents may be hydrocarbyl groups or heterohydrocarbyl groups, the latter comprising from 1 to 3 heteroatoms as mentioned before. Among the plurality of hydrophobic substituents of a molecule of said polyamine derivative, there may be exclusively hydrocarbyl groups, exclusively heterohydrocarbyl groups, or there may be hydrocarbyl groups and heterohydrocarbyl groups. In some embodiments, the plurality of hydrophobic substituents are all hydrocarbyl groups. In some embodiments, the plurality of hydrophobic substituents are all heterohydrocarbyl groups.

Where the hydrophobic substituents are hydrocarbyl groups, they may be selected from alkyl groups, alkenyl groups, alkynyl groups, cycloalkyl groups, cycloalkenyl groups, cycloalkylalkyl groups, alkylcycloalkyl groups, alkylcycloalkylalkyl groups, aryl groups, alkylaryl groups, arylalkyl groups, and alkylarylalkyl groups and groups comprising two or more groups from the aforementioned list. Provided the hydrophobic substituent comprises 6 or more carbon atoms, it is possible to replace 1, 2 or 3 of the carbon atoms of said hydrocarbyl groups by oxygen, nitrogen or sulfur, preferably oxygen or sulfur, thereby forming heterohydrocarbyl substituents. Such heterohydrocarbyl substituents may comprise functional groups selected from —O—, —S—, —N(H)C(O)—, —C(O)O—, —OC(O)N(H)—, —C(O)—, —C(O)—N(H)—, —N(H)—C(O)—O—, —O—C(O)—, or —S—S—.

In some embodiments, the hydrophobic substituents are or comprise alkyl groups such as linear or branched alkyl groups, or cycloalkyl groups. Alkyl groups may be n-alkyl or isoalkyl groups. Examples of alkyl groups are propyl, butyl, pentyl, hexyl, heptyl, octyl, nonyl, decyl, undecyl, dodecyl, tetradecyl or hexadecyl groups. Examples of cycloalkyl groups are cyclopentyl, cyclohexyl and cycloheptyl groups.

Examples of alkenyl groups are propenyl, butenyl, pentenyl, hexenyl, heptenyl, octenyl, nonenyl, decenyl, undecenyl, dodecenyl, tetradecenyl and hexadecenyl groups. Examples of alkynyl groups are propynyl, butynyl, pentynyl, hexynyl, heptynyl, octynyl, nonynyl, decynyl, undecynyl, dodecynyl, tetradecynyl and hexadecynyl groups.

Examples of cycloalkenyl groups are cyclopentenyl, cyclohexenyl and cycloheptenyl groups. Cycloalkylalkyl groups are groups wherein a cycloalkyl group is linked to an alkylene group corresponding to an alkyl group. Examples are cyclopentylmethyl, cyclopentylethyl, cyclohexylmethyl, cyclohexylethyl etc.

Alkylcycloalkyl groups are groups wherein an alkyl group is linked to a cycloalkylene group corresponding to a cycloalkyl group. Examples of alkylcycloalkyl groups are methylcyclopentyl, ethylcyclopentyl, propylcyclopentyl, butylcyclopentyl, pentylcyclopentyl, hexylcylopentyl, methylcyclohexyl, ethylcyclohexyl, propylcyclohexyl, butylcyclohexyl, pentylcyclohexyl and hexylcylohexyl.

Alkylcycloalkylalkyl groups are groups wherein an alkyl group is linked to a cycloalkylalkylene group.

In some embodiments, the hydrophobic substituent comprises an aryl group and has from 6 to 20, preferably from 7 to 15 carbon atoms. Aryl groups include aromatic hydrocarbyl groups (carbon-only aryl groups) and aromatic heterohydrocarbyl groups (heteroaryl groups). Examples of the former are phenyl, naphthyl and phenanthryl. In some embodiments, nitrogen-containing heteroaryl groups have a pK value of <5 for avoiding additional cationic charges at neutral pH. Examples of such nitrogen-containing heteroaryl groups are indolyl groups pyrazinyl groups, pyridazinyl groups, pyrimidinyl groups, cinnolinyl groups, phthalazinyl groups and purinyl groups. In some embodiments, oxygen-containing heterohydrocarbyl groups that form hydroxy groups have a pK>12 for avoiding negative charges at neutral pH.

Examples of alkylaryl groups are methylphenyl (tolyl), ethylphenyl, 4-isopropylphenyl, methylindolyl and xylyl groups. Examples of arylalkyl (aralkyl) groups are benzyl, phenylethyl, indolylmethyl and trityl groups. Examples of alkylarylalkyl groups are methylbenzyl and 4˜isopropylbenzyl groups.

Different hydrophobic substituents on a molecule of the polyamine derivative may be the same or may be different. For simplicity, they may be the same.

In some embodiments, the polyamine derivative has a linear polyethylenimine moiety of from 2 to 500 kDa (in terms of number average molecular weight), the carboxylated substituents have from 10 to 16 carbon atoms and are n-alkylcarboxylic acids and the hydrophobic substituents have from 1 to 12 carbon atoms and are alkyls, preferably n-alkyls, and/or alkylarylalkyls.

In some embodiments, the polyamine derivative has a branched polyethylenimine moiety of from 0.5 to 200 kDa (in terms of number average molecular weight), the carboxylated substituents have from 10 to 16 carbon atoms and are n-alkylcarboxylic acids and the hydrophobic substituents have from 1 to 12 carbon atoms and are alkyls, preferably n-alkyls, and/or alkylarylalkyls.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have one to thirty, typically one to twenty, often six to eighteen carbon atoms. Exemplary nonpolar alkyl groups include, but are not limited to, methyl, ethyl, propyl, isopropyl, hexyl, decyl, dodecyl, tetradecyl, hexadecyl, octadecyl, and the like.

The term “alkenyl” refers to a linear or branched monovalent hydrocarbon moiety having at least one carbon-carbon double bond in which the total carbon atoms may be two to thirty, typically six to twenty often six to eighteen. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds.

The term “alkynyl” refers to a linear or branched monovalent hydrocarbon moiety having at least one carbon-carbon triple bond in which the total carbon atoms may be two to thirty, typically six to twenty, often six to eighteen. Alkynyl groups can optionally have one or more carbon-carbon double bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds.

The term “alkylene” refers to a saturated linear or branched divalent hydrocarbon moiety which may have one to thirty, typically two to twenty, often four to twelve carbon atoms. Exemplary nonpolar alkylene groups include, but are not limited to, methylene, ethylene, trimethylene, hexamethylene, decamethylene, dodecamethylene, tetradecamethylene, hexadecamethylene, octadecmethylene, and the like.

The term “alkenylene” refers to a linear or branched divalent hydrocarbon moiety having at least one carbon-carbon double bond in which the total carbon atoms may be two to thirty, typically two to twenty, often four to twelve. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds.

The term “alkynylene” refers to a linear or branched divalent hydrocarbon moiety having at least one carbon-carbon triple bond in which the total carbon atoms may be two to thirty, typically two to twenty, often four to twelve. Alkynyl groups can optionally have one or more carbon carbon double bonds.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or more than two rings (polycyclic).

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic).

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more cis double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic/Cationically Ionizable Lipids

In some embodiments, the particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-l-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)—N-(3-aminopropyl)-N,N-dimethyl-2,3-bis(cis-9-tetradecenyloxy)-1-propanaminium bromide (GAP-DMORIE), (±)—N-(3-aminopropyl)-N,N-dimethyl-2,3-bis(dodecyloxy)-1-propanaminium bromide (GAP-DLRIE), (±)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic or cationically ionizable lipid is DOTMA. In some embodiments, the cationic or cationically ionizable lipid is DODMA.

DOTMA is a cationic lipid with a quaternary amine headgroup. The structure of DOTMA may be represented as follows:

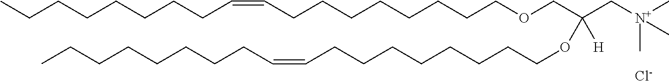 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

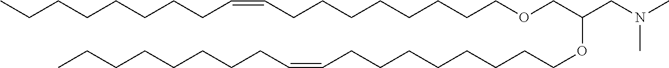 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle.

### Additional Lipids

The particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of nucleic acid particles by addition of other hydrophobic moieties, such as cholesterol and lipids, in addition to a cationic or cationically ionizable lipid may enhance particle stability and efficacy of nucleic acid delivery.

One or more additional lipids may or may not affect the overall charge of the nucleic acid particles. In some embodiments, the one or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the nucleic acid particles described herein comprise a cationic or cationically ionizable lipid and one or more additional lipids.

Without wishing to be bound by theory, the amount of the cationic or cationically ionizable lipid compared to the amount of the one or more additional lipids may affect important nucleic acid particle characteristics, such as charge, particle size, stability, tissue selectivity, and bioactivity of the nucleic acid. Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the nucleic acid particles described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoylphosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Thus, in some embodiments, the nucleic acid particles described herein comprise (1) a cationic or cationically ionizable lipid, and a phospholipid such as DSPC or DOPE or (2) a cationic or cationically ionizable lipid and a phospholipid such as DSPC or DOPE and cholesterol.

In some embodiments, the nucleic acid particles described herein comprise (1) DOTMA and DOPE, (2) DOTMA, DOPE and cholesterol, (3) DODMA and DOPE or (4) DODMA, DOPE and cholesterol.

DSPC is a neutral phospholipid. The structure of DSPC may be represented as follows:

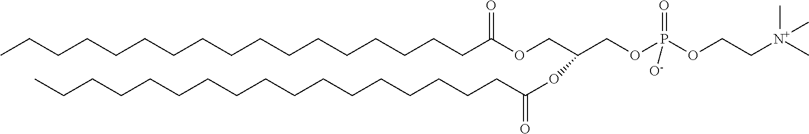 [SMILES: CCCCCCCCCCCCCCCCCC(=O)OC[C@H](COP(=O)([O-])OCC[N+](C)(C)C)OC(=O)CCCCCCCCCCCCCCCCC]

DOPE is a neutral phospholipid. The structure of DOPE may be represented as follows:

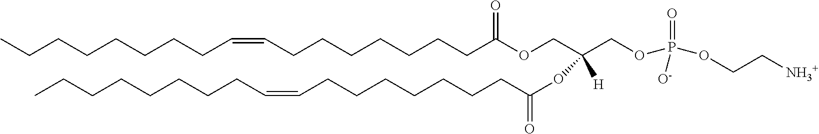 [SMILES: CCCCCCCC/C=C\CCCCCCCC(=O)OC[C@H](COP(=O)([O-])OCC[NH3+])OC(=O)CCCCCCC/C=C\CCCCCCCC]

The structure of cholesterol may be represented as follows:

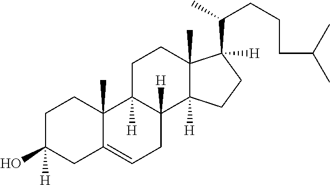

In some embodiments, nucleic acid particles described herein do not include a polymer conjugated lipid such as a pegylated lipid.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Polymer-Conjugated Lipids

In some embodiments, the particles described herein may comprise at least one polymer-conjugated lipid. In some embodiments, the polymer-conjugated lipid comprises an amphiphilic derivative of a polymer which is part of a targeting compound and/or a polymer-conjugated lipid which is not part of a targeting compound. A polymer-conjugated lipid is typically a molecule comprising a lipid portion and a polymer portion conjugated thereto. In some embodiments, the polymer of the polymer-conjugated lipid is a polymer as described herein for the targeting compound. In some embodiments, a polymer-conjugated lipid is a PEG-conjugated lipid, also referred to herein as pegylated lipid or PEG-lipid. The term “pegylated lipid” refers to a molecule comprising both a lipid portion and a polyethylene glycol portion. Pegylated lipids are known in the art. In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine portion.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

### Polyethyleneglycol (PEG)-Conjugated Lipids

In some embodiments, the particles described herein comprise a PEG-conjugated lipid.

In some embodiments, the PEG-conjugated lipid (pegylated lipid) is a lipid having the structure of the following general formula:

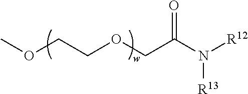

or a pharmaceutically acceptable salt, tautomer or stereoisomer thereof, wherein:
each of R¹² and R¹³ is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of this formula, each of R¹² and R¹³ is independently a straight alkyl chain containing from 10 to 18 carbon atoms, preferably from 12 to 16 carbon atoms.

In some embodiments of this formula, R¹² and R¹³ are identical. In some embodiments, each of R¹² and R¹³ is a straight alkyl chain containing 12 carbon atoms. In some embodiments, each of R¹² and R¹³ is a straight alkyl chain containing 14 carbon atoms. In some embodiments, each of R¹² and R¹³ is a straight alkyl chain containing 16 carbon atoms.

In some embodiments of this formula, R¹² and R¹³ are different. In some embodiments, one of R¹² and R¹³ is a straight alkyl chain containing 12 carbon atoms and the other of R¹² and R¹³ is a straight alkyl chain containing 14 carbon atoms.

In some embodiments of this formula, w has a mean value ranging from 40 to 50, such as a mean value of 45.

In some embodiments of this formula, w is within a range such that the PEG portion of the pegylated lipid has an average molecular weight of from about 400 to about 6000 g/mol, such as from about 1000 to about 5000 g/mol, from about 1500 to about 4000 g/mol, or from about 2000 to about 3000 g/mol.

In some embodiments, each of R¹² and R¹³ is a straight alkyl chain containing 14 carbon atoms and w has a mean value of 45.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O—(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

In some embodiments, the PEG-conjugated lipid (pegylated lipid) is or comprises 2-[(polyethylene glycol)-2000]—N,N-ditetradecylacetamide. In some embodiments, the pegylated lipid has the following structure:

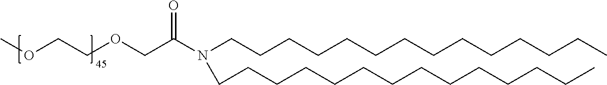

In some embodiments, the PEG-conjugated lipid (pegylated lipid) is DMG-PEG 2000, e.g., having the following structure:

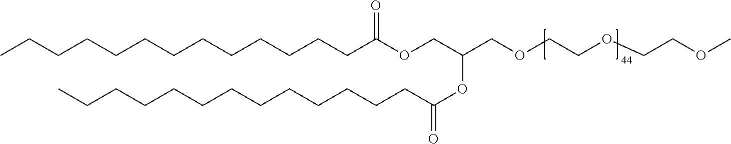

In some embodiments, the PEG-conjugated lipid (pegylated lipid) has the following structure: wherein n has a mean value ranging from 30 to 60, such as about 50. In some embodiments, the PEG-

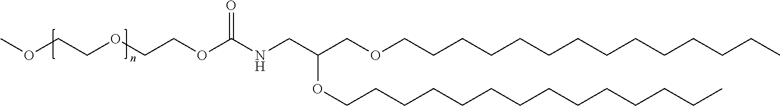

conjugate lipid (pegylated lipid) is PEG₂₀₀₀-C-DMA which preferably refers to 3-N—[(ω-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000).

In some embodiments, nucleic acid particles described herein may comprise one or more PEG-conjugated lipids or pegylated lipids as described in WO 2017/075531 and WO 2018/081480, the entire contents of each of which are incorporated herein by reference for the purposes described herein.

In some embodiments, the pegylated lipid comprises from about 1 mol % to about 10 mol %, preferably from about 1 mol % to about 5 mol %, more preferably from about 1 mol % to about 2.5 mol % of the total lipid present in the nucleic acid compositions/formulations and nucleic acid particles described herein.

### Embodiments of Lipoplex Particles

In some embodiments of the present disclosure, the nucleic acid such as RNA described herein may be present in lipoplex particles.

Lipoplexes (LPX) are electrostatic complexes which are generally formed by mixing preformed cationic lipid liposomes with anionic nucleic acid. Formed lipoplexes possess distinct internal arrangements of molecules that arise due to the transformation from liposomal structure into compact nucleic acid lipoplexes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In certain embodiments, the nucleic acid lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

Nucleic acid lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In some embodiments, the nucleic acid lipoplex particles have an average diameter that ranges from about 250 nm to about 700 nm. In some embodiments, the RNA lipoplex particles have an average diameter that ranges from about 300 nm to about 500 nm. In an exemplary embodiment, the RNA lipoplex particles have an average diameter of about 400 nm.

In some embodiments, the functionalized particles described herein are lipid particles in particular lipoplexes comprising RNA drug substance, 1,2-dioleyloxy-3-dimethylaminopropane (DODMA), cholesterol, 1,2-diastearoyl-sn-glycero-3-phosphocholine (DSPC), distearoyl-sn-glycero-3-phosphoethanolamine-N-[methoxy(polyethylene glycol)-2000-alfa peptide (DSPE-PEG2k-alfa), and tetradecyl-poly(sarcosine)23-acetate (C14-PSar(23)-Ac). The functionalized lipoplexes are manufactured using a two step protocol: (1) manufacturing of alfa tagged RNA particles followed by (2) functionalization of alfa tagged lipoplexes with ligand. The alfa tagged RNA lipid particles can be prepared using either aqueous/aqueous or aqueous/organic manufacturing protocols.

### Embodiments of Lipid Nanoparticles (LNPs)

In some embodiments, nucleic acid described herein is present in the form of lipid nanoparticles (LNPs). The LNP may comprise any lipid capable of forming a particle to which the one or more nucleic acid molecules are attached, or in which the one or more nucleic acid molecules are encapsulated.

In general, lipid nanoparticles are obtainable from direct mixing of nucleic acid, e.g., RNA, in an aqueous phase with lipids in a phase comprising an organic solvent, such as ethanol. In that case, lipids or lipid mixtures can be used for particle formation, which do not form lamellar (bilayer) phases in water.

LNPs typically comprise four components: cationically ionizable lipid, neutral lipids such as phospholipids, a steroid such as cholesterol, and a polymer-conjugated lipid such as PEG-lipid. LNPs may be prepared by mixing lipids dissolved in ethanol with nucleic acid in an aqueous buffer.

In some embodiments, the LNP comprises from 40 to 60 mol percent, 40 to 55 mol percent, from 45 to 55 mol percent, or from 45 to 50 mol percent of the cationically ionizable lipid.

In some embodiments, the neutral lipid is present in a concentration ranging from 5 to 15 mol percent, from 7 to 13 mol percent, or from 9 to 11 mol percent.

In some embodiments, the steroid is present in a concentration ranging from 30 to 50 mol percent, from 30 to 45 mol percent, from 35 to 45 mol percent or from 35 to 43 mol percent.

In some embodiments, the LNP comprises from 1 to 10 mol percent, from 1 to 5 mol percent, or from 1 to 2.5 mol percent of the polymer-conjugated lipid.

In some embodiments, the LNP comprises from 45 to 55 mol percent of a cationically ionizable lipid; from 5 to 15 mol percent of a neutral lipid; from 30 to 45 mol percent of a steroid; from 1 to 5 mol percent of a polymer-conjugated lipid; and the nucleic acid, encapsulated within or associated with the lipid nanoparticle.

In some embodiments, the mol percent is determined based on total mol of lipid present in the lipid nanoparticle. In some embodiments, the mol percent is determined based on total mol of cationically ionizable lipid, neutral lipid, steroid and polymer-conjugated lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid, e.g., a pegylated lipid as described above.

In some embodiments, the cationically ionizable lipid component of the LNPs has the structure of Formula (III):

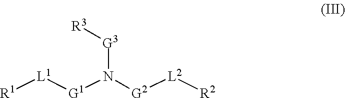

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(=O)—, —NR^{a}C(═O)—, —C(═O)NR^{a}—NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(=O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C1-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C1-C₆ alkyl; and
x is 0, 1 or 2.

In some of the foregoing embodiments of Formula (III), the lipid has one of the following structures (IIIA) or (IIIB):

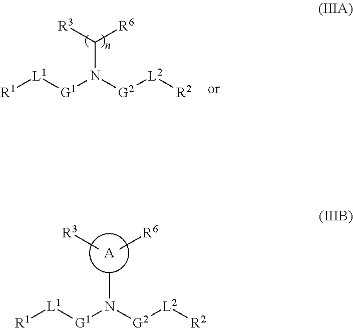

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

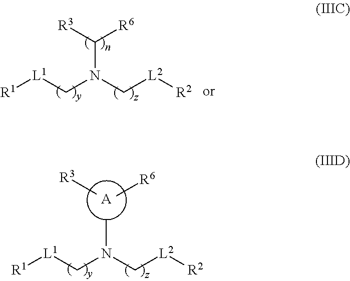

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

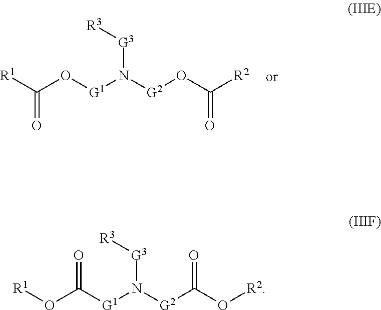

In some of the foregoing embodiments of Formula (III), the lipid has one of the following structures (IIIG), (IIIH), (IIII), or (IIIJ):

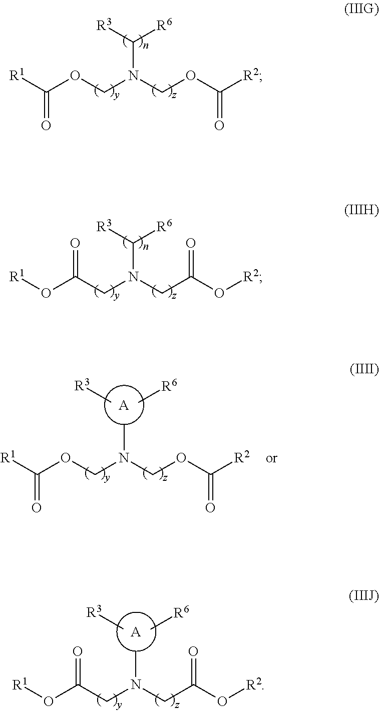

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

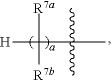

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

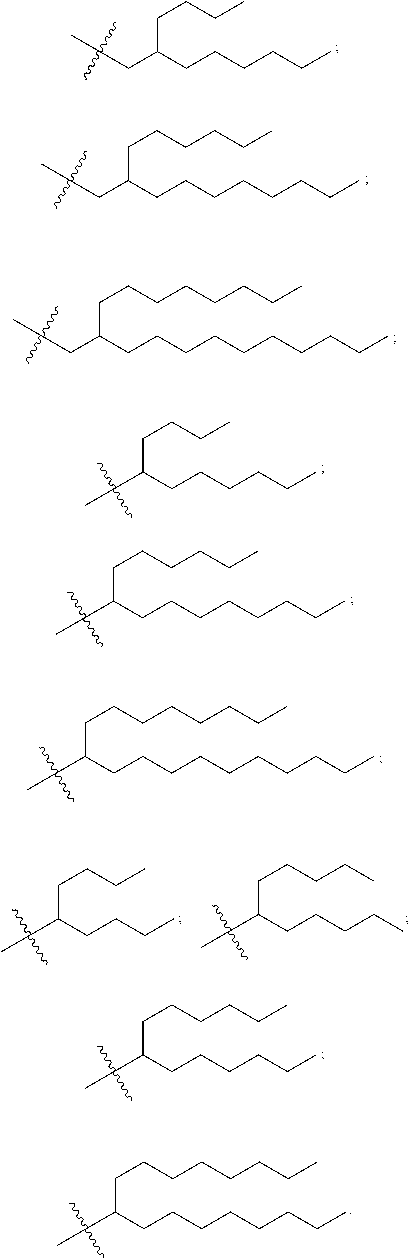

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

### Representative Compounds of Formula (III).

| No. | Structure |
|---|---|
| III-1 | 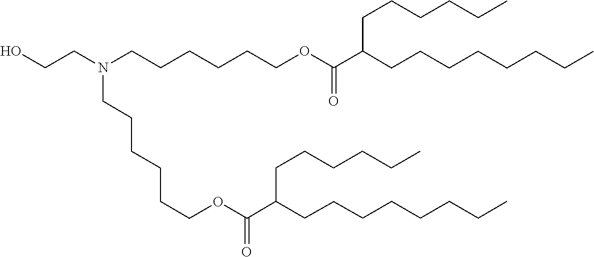 [SMILES: CCCCCCCCC(CCCCCC)C(=O)OCCCCCCN(CCO)CCCCCCOC(=O)C(CCCCCC)CCCCCCCC] |
| III-2 | 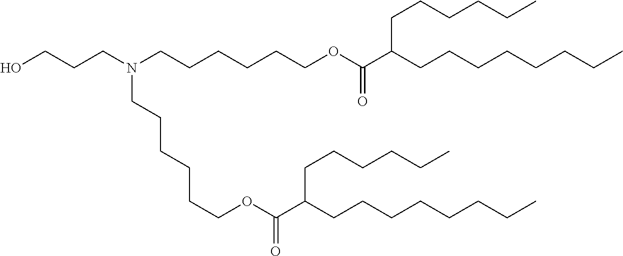 [SMILES: CCCCCCCCC(CCCCCC)C(=O)OCCCCCCN(CCCO)CCCCCCOC(=O)C(CCCCCC)CCCCCCCC] |
| III-3 | 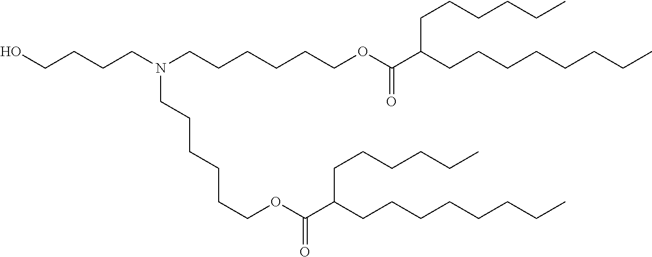 [SMILES: CCCCCCCCC(CCCCCC)C(=O)OCCCCCCN(CCCCO)CCCCCCOC(=O)C(CCCCCC)CCCCCCCC] |
| III-4 | 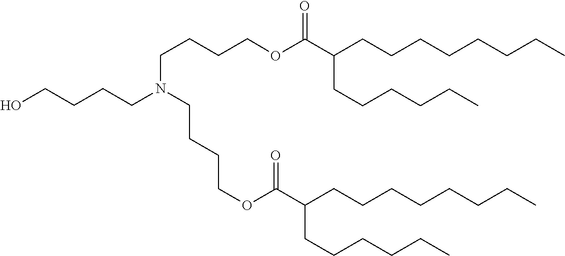 [SMILES: CCCCCCCCC(CCCCCC)C(=O)OCCCCN(CCCCO)CCCCOC(=O)C(CCCCCC)CCCCCCCC] |
| III-5 | 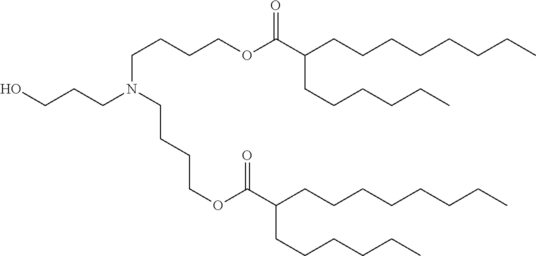 [SMILES: CCCCCCCCC(CCCCCC)C(=O)OCCCCN(CCCO)CCCCOC(=O)C(CCCCCC)CCCCCCCC] |
| III-6 | 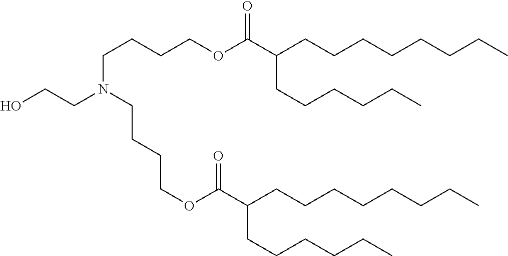 [SMILES: CCCCCCCCC(CCCCCC)C(=O)OCCCCN(CCO)CCCCOC(=O)C(CCCCCC)CCCCCCCC] |
| III-7 | 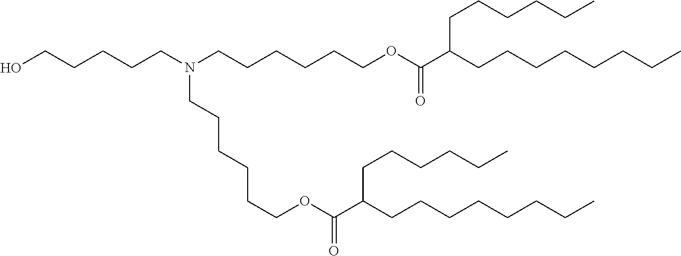 [SMILES: CCCCCCCCC(CCCCCC)C(=O)OCCCCCCN(CCCCCO)CCCCCCOC(=O)C(CCCCCC)CCCCCCCC] |
| III-8 | 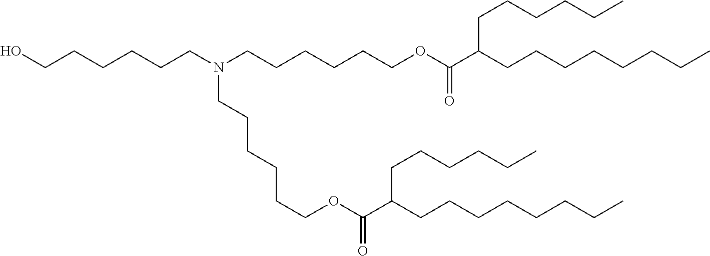 [SMILES: CCCCCCCCC(CCCCCC)C(=O)OCCCCCCN(CCCCCCO)CCCCCCOC(=O)C(CCCCCC)CCCCCCCC] |
| III-9 | 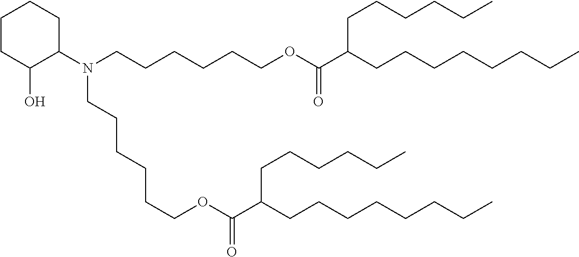 [SMILES: CCCCCCCCC(CCCCCC)C(=O)OCCCCCCN(CCCCCCOC(=O)C(CCCCCC)CCCCCCCC)C1CCCCC1O] |
| III-10 | 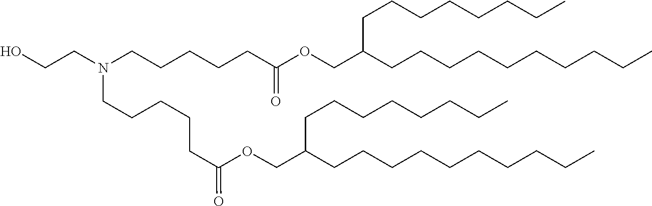 [SMILES: CCCCCCCCCCC(CCCCCCCC)COC(=O)CCCCCN(CCO)CCCCCC(=O)OCC(CCCCCCCC)CCCCCCCCCC] |
| III-11 | 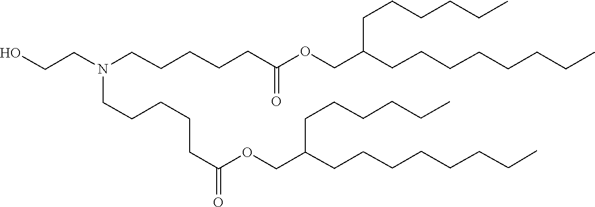 [SMILES: CCCCCCCCC(CCCCCC)COC(=O)CCCCCN(CCO)CCCCCC(=O)OCC(CCCCCC)CCCCCCCC] |
| III-12 | 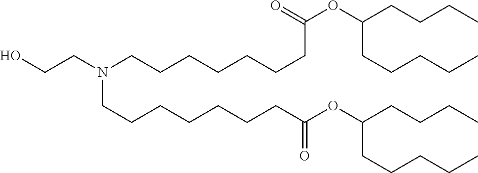 [SMILES: CCCCCC(CCCC)OC(=O)CCCCCCCN(CCO)CCCCCCCC(=O)OC(CCCC)CCCCC] |
| III-13 | 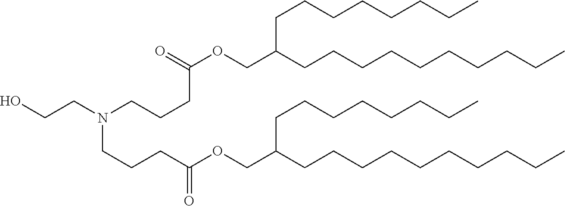 [SMILES: CCCCCCCCCCC(CCCCCCCC)COC(=O)CCCN(CCO)CCCC(=O)OCC(CCCCCCCC)CCCCCCCCCC] |
| III-14 | 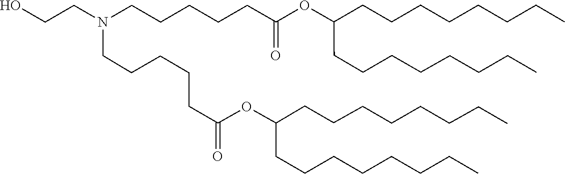 [SMILES: CCCCCCCCC(CCCCCCCC)OC(=O)CCCCCN(CCO)CCCCCC(=O)OC(CCCCCCCC)CCCCCCCC] |
| III-15 | 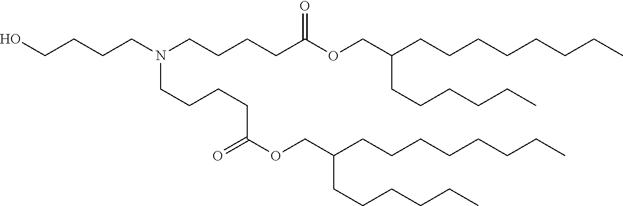 [SMILES: CCCCCCCCC(CCCCCC)COC(=O)CCCCN(CCCCO)CCCCC(=O)OCC(CCCCCC)CCCCCCCC] |
| III-16 | 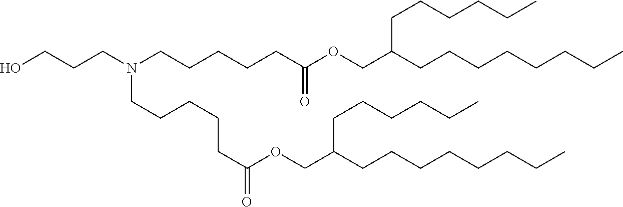 [SMILES: CCCCCCCCC(CCCCCC)COC(=O)CCCCCN(CCCO)CCCCCC(=O)OCC(CCCCCC)CCCCCCCC] |
| III-17 | 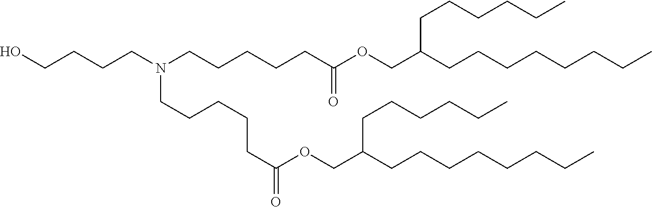 [SMILES: CCCCCCCCC(CCCCCC)COC(=O)CCCCCN(CCCCO)CCCCCC(=O)OCC(CCCCCC)CCCCCCCC] |
| III-18 | 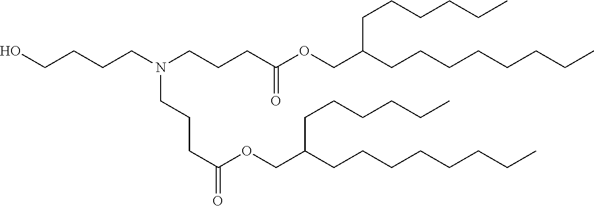 [SMILES: CCCCCCCCC(CCCCCC)COC(=O)CCCN(CCCCO)CCCC(=O)OCC(CCCCCC)CCCCCCCC] |
| III-19 | 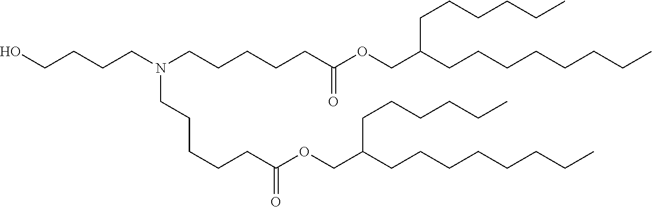 [SMILES: CCCCCCCCC(CCCCCC)COC(=O)CCCCCN(CCCCO)CCCCCC(=O)OCC(CCCCCC)CCCCCCCC] |
| III-20 | 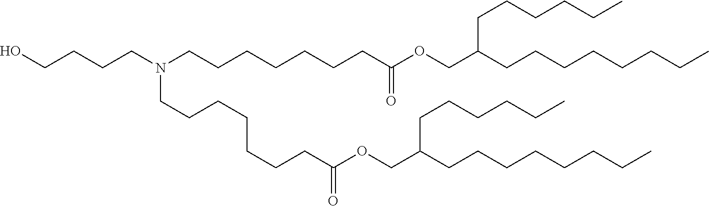 [SMILES: CCCCCCCCC(CCCCCC)COC(=O)CCCCCCCN(CCCCO)CCCCCCCC(=O)OCC(CCCCCC)CCCCCCCC] |
| III-21 | 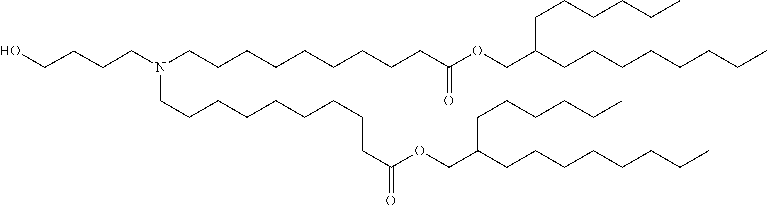 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 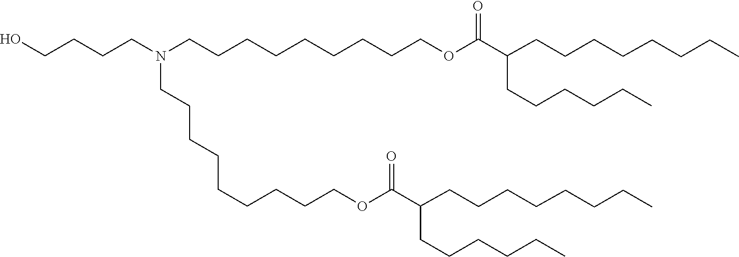 [SMILES: CCCCCCCCC(CCCCCC)C(=O)OCCCCCCCCCN(CCCCO)CCCCCCCCCOC(=O)C(CCCCCC)CCCCCCCC] |
| III-23 | 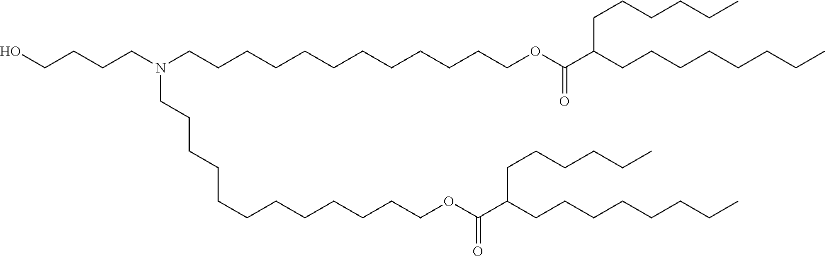 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 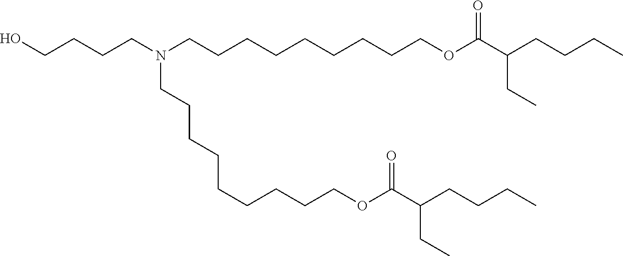 [SMILES: CCCCC(CC)C(=O)OCCCCCCCCCN(CCCCO)CCCCCCCCCOC(=O)C(CC)CCCC] |
| III-25 | 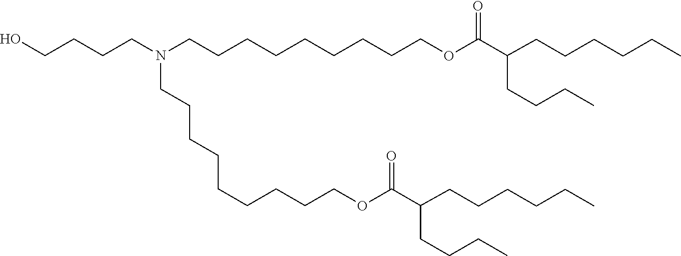 [SMILES: CCCCCCC(CCCC)C(=O)OCCCCCCCCCN(CCCCO)CCCCCCCCCOC(=O)C(CCCC)CCCCCC] |
| III-26 | 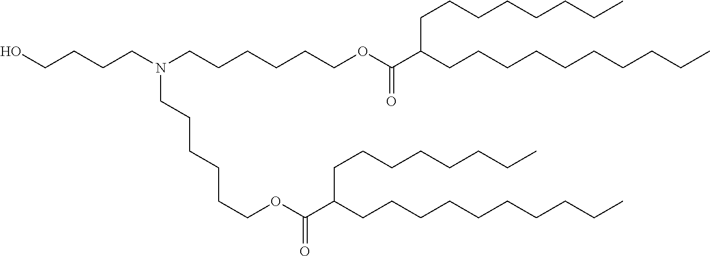 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 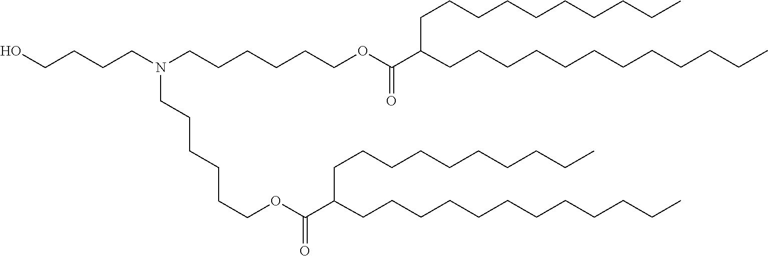 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 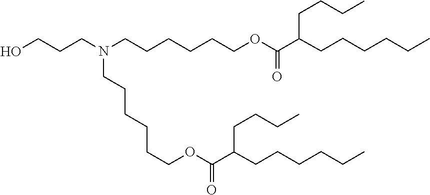 [SMILES: CCCCCCC(CCCC)C(=O)OCCCCCCN(CCCO)CCCCCCOC(=O)C(CCCC)CCCCCC] |
| III-29 | 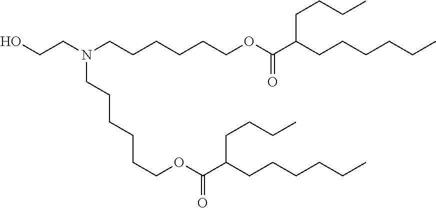 [SMILES: CCCCCCC(CCCC)C(=O)OCCCCCCN(CCO)CCCCCCOC(=O)C(CCCC)CCCCCC] |
| III-30 | 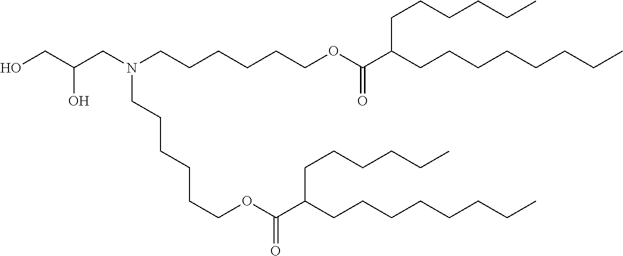 [SMILES: CCCCCCCCC(CCCCCC)C(=O)OCCCCCCN(CCCCCCOC(=O)C(CCCCCC)CCCCCCCC)CC(O)CO] |
| III-31 | 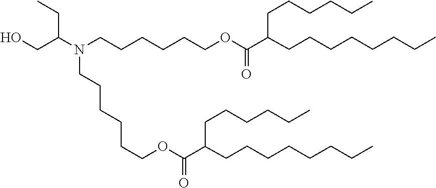 [SMILES: CCCCCCCCC(CCCCCC)C(=O)OCCCCCCN(CCCCCCOC(=O)C(CCCCCC)CCCCCCCC)C(CC)CO] |
| III-32 | 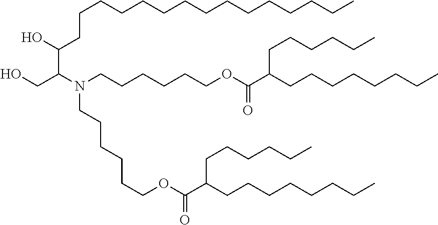 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 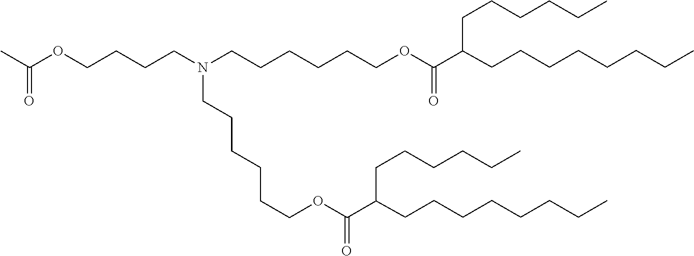 [SMILES: CCCCCCCCC(CCCCCC)C(=O)OCCCCCCN(CCCCCCOC(=O)C(CCCCCC)CCCCCCCC)CCCCOC(C)=O] |
| III-34 | 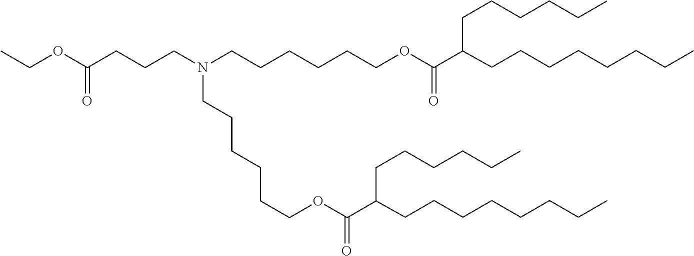 [SMILES: CCCCCCCCC(CCCCCC)C(=O)OCCCCCCN(CCCCCCOC(=O)C(CCCCCC)CCCCCCCC)CCCC(=O)OCC] |
| III-35 | 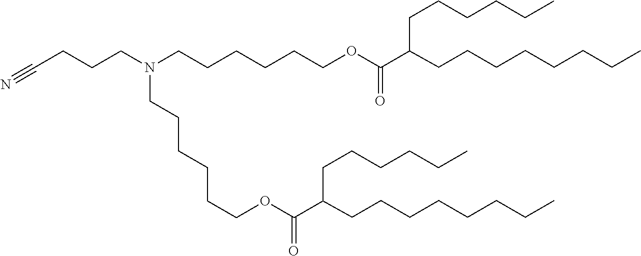 [SMILES: CCCCCCCCC(CCCCCC)C(=O)OCCCCCCN(CCCC#N)CCCCCCOC(=O)C(CCCCCC)CCCCCCCC] |
| III-36 | 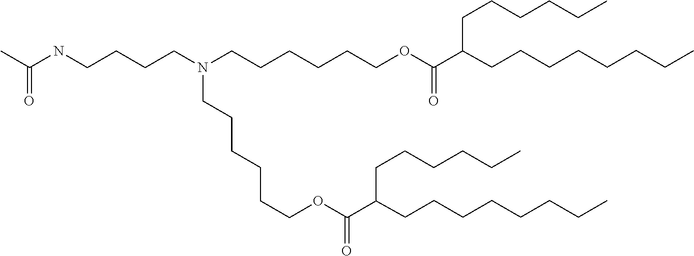 [SMILES: CCCCCCCCC(CCCCCC)C(=O)OCCCCCCN(CCCCCCOC(=O)C(CCCCCC)CCCCCCCC)CCCCNC(C)=O] |
| III-37 | 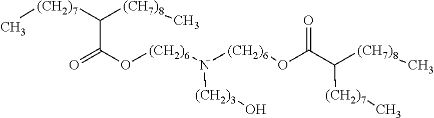 |
| III-38 | 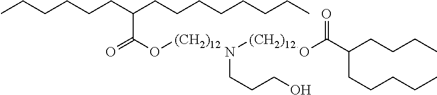 |
| III-39 | 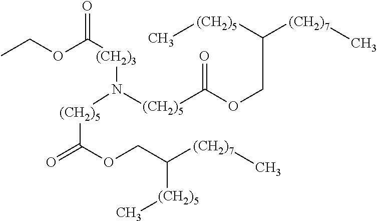 |
| III-40 | 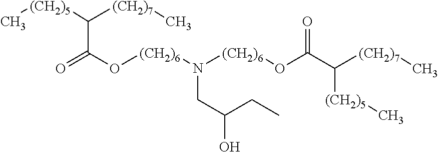 |
| III-41 | 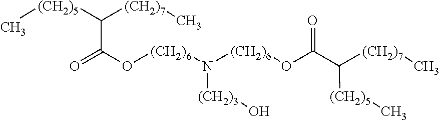 |
| III-42 | 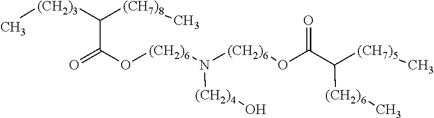 |
| III-43 | 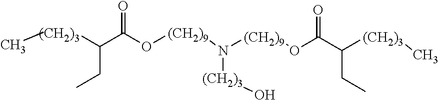 |
| III-44 | 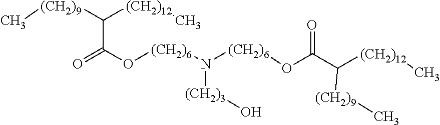 |
| III-45 | 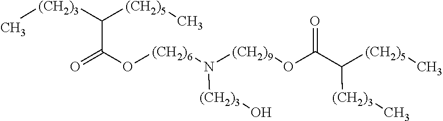 |
| III-46 | 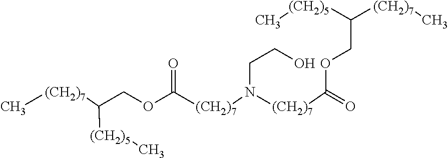 |
| III-47 | 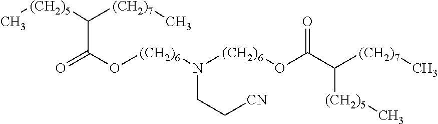 |
| III-48 | 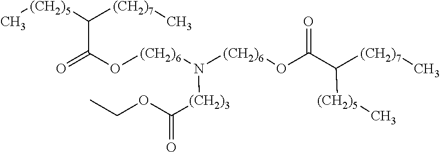 |
| III-49 | 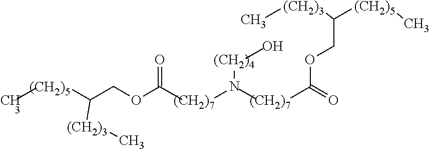 |

Further representative cationically ionizable lipids are as follows:

| No. | Structure |
|---|---|
| A | 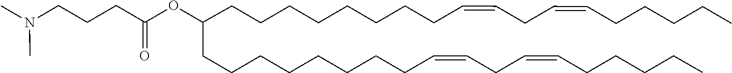 [SMILES: CCCCC/C=C\C/C=C\CCCCCCCCC(CCCCCCCC/C=C\C/C=C\CCCCC)OC(=O)CCCN(C)C] |
| B | 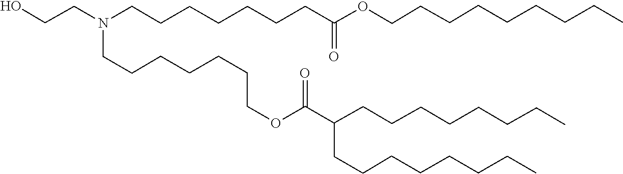 [SMILES: CCCCCCCCCOC(=O)CCCCCCCN(CCO)CCCCCCCOC(=O)C(CCCCCCCC)CCCCCCCC] |
| C | 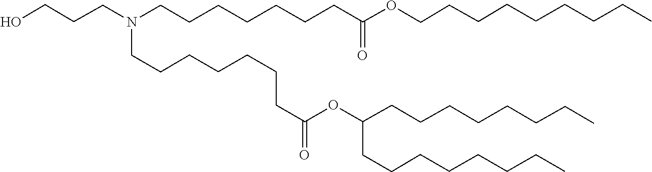 [SMILES: CCCCCCCCCOC(=O)CCCCCCCN(CCCO)CCCCCCCC(=O)OC(CCCCCCCC)CCCCCCCC] |
| D | 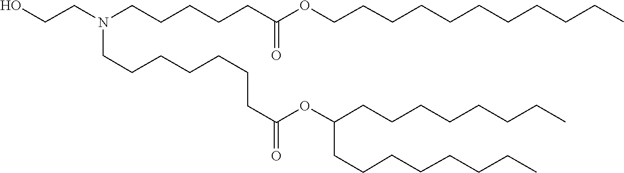 [SMILES: CCCCCCCCCCCOC(=O)CCCCCN(CCO)CCCCCCCC(=O)OC(CCCCCCCC)CCCCCCCC] |
| E | 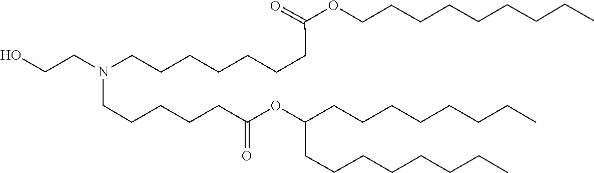 [SMILES: CCCCCCCCCOC(=O)CCCCCCCN(CCO)CCCCCC(=O)OC(CCCCCCCC)CCCCCCCC] |
| F | 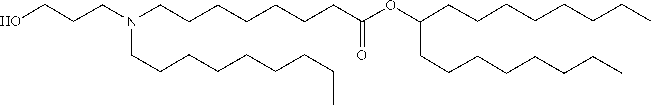 [SMILES: CCCCCCCCCN(CCCO)CCCCCCCC(=O)OC(CCCCCCCC)CCCCCCCC] |

In some embodiments, nucleic acid such as RNA described herein is formulated in an LNP composition comprising a cationically ionizable lipid, e.g., a cationically ionizable lipid as shown above, a neutral lipid, a steroid, and a polymer conjugated lipid.

In some embodiments, nucleic acid such as RNA described herein is formulated in an LNP composition comprising a cationically ionizable lipid of Formula III, a neutral lipid, a steroid, and a polymer conjugated lipid.

In some embodiments, nucleic acid such as RNA described herein is formulated in an LNP composition comprising a cationically ionizable lipid shown in the above tables, a neutral lipid, a steroid, and a polymer conjugated lipid.

In some embodiments, nucleic acid such as RNA described herein is formulated in an LNP composition comprising 3D-P-DMA, a neutral lipid, a steroid, and a polymer conjugated lipid.

In some embodiments, nucleic acid such as RNA described herein is formulated in an LNP composition comprising ALC-0366, a neutral lipid, a steroid, and a polymer conjugated lipid.

In some embodiments, nucleic acid such as RNA described herein is formulated in an LNP composition comprising ALC-0315, a neutral lipid, a steroid, and a polymer conjugated lipid.

In some embodiments, the neutral lipid is DSPC. In some embodiments, the steroid is cholesterol. In some embodiments, the polymer conjugated lipid is a pegylated lipid, e.g., DMG-PEG 2000, PEG₂₀₀₀-C-DMA, or ALC-0159.

In some embodiments, nucleic acid such as RNA described herein is formulated in an LNP composition comprising a cationically ionizable lipid, e.g., a cationically ionizable lipid as shown above, a neutral lipid, a steroid, and a pegylated lipid.

In some embodiments, nucleic acid such as RNA described herein is formulated in an LNP composition comprising a cationically ionizable lipid of Formula III, a neutral lipid, a steroid, and a pegylated lipid.

In some embodiments, nucleic acid such as RNA described herein is formulated in an LNP composition comprising a cationically ionizable lipid shown in the above tables, a neutral lipid, a steroid, and a pegylated lipid.

In some embodiments, nucleic acid such as RNA described herein is formulated in an LNP composition comprising 3D-P-DMA, a neutral lipid, a steroid, and a pegylated lipid.

In some embodiments, nucleic acid such as RNA described herein is formulated in an LNP composition comprising ALC-0366, a neutral lipid, a steroid, and a pegylated lipid.

In some embodiments, nucleic acid such as RNA described herein is formulated in an LNP composition comprising ALC-0315, a neutral lipid, a steroid, and a pegylated lipid.

In some embodiments, the neutral lipid is DSPC. In some embodiments, the steroid is cholesterol. In some embodiments, the pegylated lipid is DMG-PEG 2000, PEG₂₀₀₀-C-DMA, or ALC-0159.

In some embodiments, nucleic acid such as RNA described herein is formulated in an LNP composition comprising a cationically ionizable lipid, e.g., a cationically ionizable lipid as shown above, DSPC, cholesterol, and a pegylated lipid.

In some embodiments, nucleic acid such as RNA described herein is formulated in an LNP composition comprising a cationically ionizable lipid of Formula III, DSPC, cholesterol, and a pegylated lipid.

In some embodiments, nucleic acid such as RNA described herein is formulated in an LNP composition comprising a cationically ionizable lipid shown in the above tables, DSPC, cholesterol, and a pegylated lipid.

In some embodiments, nucleic acid such as RNA described herein is formulated in an LNP composition comprising 3D-P-DMA, DSPC, cholesterol, and a pegylated lipid.

In some embodiments, nucleic acid such as RNA described herein is formulated in an LNP composition comprising ALC-0366, DSPC, cholesterol, and a pegylated lipid.

In some embodiments, nucleic acid such as RNA described herein is formulated in an LNP composition comprising ALC-0315, DSPC, cholesterol, and a pegylated lipid.

In some embodiments, the pegylated lipid is DMG-PEG 2000, PEG₂₀₀₀-C-DMA, or ALC-0159.

In some embodiments, nucleic acid such as RNA described herein is formulated in an LNP composition comprising a cationically ionizable lipid, e.g., a cationically ionizable lipid as shown above, DSPC, cholesterol, and DMG-PEG 2000.

In some embodiments, nucleic acid such as RNA described herein is formulated in an LNP composition comprising a cationically ionizable lipid of Formula III, DSPC, cholesterol, and DMG-PEG 2000.

In some embodiments, nucleic acid such as RNA described herein is formulated in an LNP composition comprising a cationically ionizable lipid shown in the above tables, DSPC, cholesterol, and DMG-PEG 2000.

In some embodiments, nucleic acid such as RNA described herein is formulated in an LNP composition comprising 3D-P-DMA, DSPC, cholesterol, and DMG-PEG 2000.

In some embodiments, nucleic acid such as RNA described herein is formulated in an LNP composition comprising ALC-0366, DSPC, cholesterol, and DMG-PEG 2000.

In some embodiments, nucleic acid such as RNA described herein is formulated in an LNP composition comprising ALC-0315, DSPC, cholesterol, and DMG-PEG 2000.

In some embodiments, nucleic acid such as RNA described herein is formulated in an LNP composition comprising a cationically ionizable lipid, e.g., a cationically ionizable lipid as shown above, DSPC, cholesterol, and PEG₂₀₀₀-C-DMA.

In some embodiments, nucleic acid such as RNA described herein is formulated in an LNP composition comprising a cationically ionizable lipid of Formula III, DSPC, cholesterol, and PEG₂₀₀₀-C-DMA.

In some embodiments, nucleic acid such as RNA described herein is formulated in an LNP composition comprising a cationically ionizable lipid shown in the above tables, DSPC, cholesterol, and PEG₂₀₀₀-C-DMA.

In some embodiments, nucleic acid such as RNA described herein is formulated in an LNP composition comprising 3D-P-DMA, DSPC, cholesterol, and PEG₂₀₀₀-C-DMA.

In some embodiments, nucleic acid such as RNA described herein is formulated in an LNP composition comprising ALC-0366, DSPC, cholesterol, and PEG₂₀₀₀-C-DMA.

In some embodiments, nucleic acid such as RNA described herein is formulated in an LNP composition comprising ALC-0315, DSPC, cholesterol, and PEG₂₀₀₀-C-DMA.

In some embodiments, nucleic acid such as RNA described herein is formulated in an LNP composition comprising a cationically ionizable lipid, e.g., a cationically ionizable lipid as shown above, DSPC, cholesterol, and ALC-0159.

In some embodiments, nucleic acid such as RNA described herein is formulated in an LNP composition comprising a cationically ionizable lipid of Formula III, DSPC, cholesterol, and ALC-0159.

In some embodiments, nucleic acid such as RNA described herein is formulated in an LNP composition comprising a cationically ionizable lipid shown in the above tables, DSPC, cholesterol, and ALC-0159.

In some embodiments, nucleic acid such as RNA described herein is formulated in an LNP composition comprising 3D-P-DMA, DSPC, cholesterol, and ALC-0159.

In some embodiments, nucleic acid such as RNA described herein is formulated in an LNP composition comprising ALC-0366, DSPC, cholesterol, and ALC-0159.

In some embodiments, nucleic acid such as RNA described herein is formulated in an LNP composition comprising ALC-0315, DSPC, cholesterol, and ALC-0159.

### 3D-P-DMA: (6Z,16Z)-12-((Z)-dec-4-en-1-yl)docosa-6,16-dien-11-yl 5-(dimethylamino)pentanoate

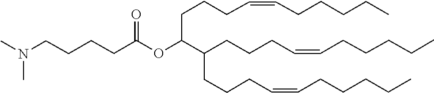 [SMILES: CCCCC/C=C\CCCC(CCC/C=C\CCCCC)C(CCC/C=C\CCCCC)OC(=O)CCCCN(C)C]

### ALC-0366: ((3-hydroxypropyl)azanediyl)bis(nonane-9,1-diyl)bis(2-butyloctanoate)

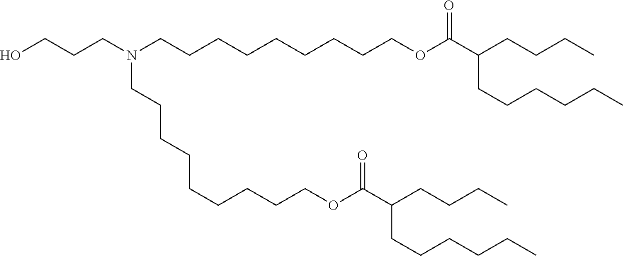 [SMILES: CCCCCCC(CCCC)C(=O)OCCCCCCCCCN(CCCO)CCCCCCCCCOC(=O)C(CCCC)CCCCCC]

### ALC-0315: ((4-hydroxybutyl)azanediyl)bis(hexane-6,1-diyl)bis(2-hexyldecanoate)/6-[N-6-(2-hexyldecanoyloxy)hexyl-N-(4-hydroxybutyl)amino]hexyl 2-hexyldecanoate

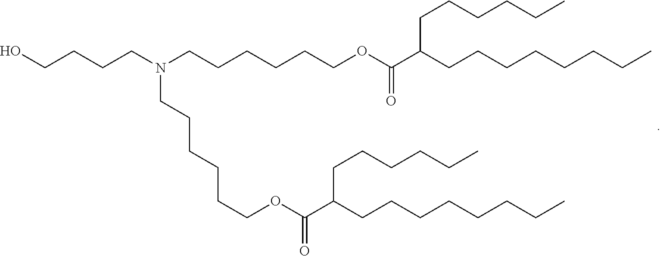 [SMILES: CCCCCCCCC(CCCCCC)C(=O)OCCCCCCN(CCCCO)CCCCCCOC(=O)C(CCCCCC)CCCCCCCC ; "."]

### DMG-PEG 2000

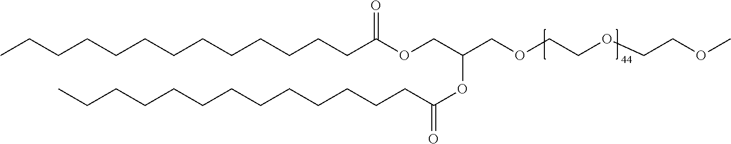

### PEG₂₀₀₀-C-DMA: 3-N—[(ω-Methoxy poly(ethylene glycol)2000) carbamoyl]-1,2-dimyristyloxy-propylamine (MPEG-(2 kDa)-C-DMA or Methoxy-polyethylene glycol-2,3-bis(tetradecyloxy)propylcarbamate (2000))

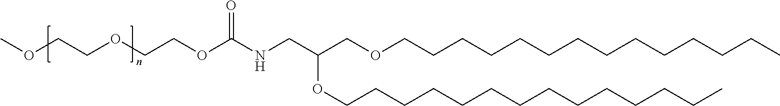

wherein n has a mean value ranging from 30 to 60, such as about 50.

### ALC-0159: 2-[(polyethylene glycol)-2000]—N,N-ditetradecylacetamide/2-[2-(ω-methoxy (polyethyleneglycol2000) ethoxy]-N,N-ditetradecylacetamide

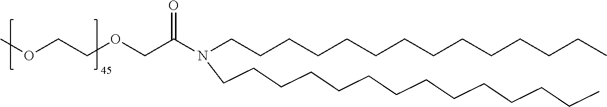

### DSPC: 1,2-Distearoyl-sn-glycero-3-phosphocholine

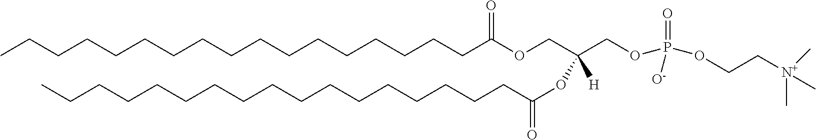 [SMILES: CCCCCCCCCCCCCCCCCC(=O)OC[C@H](COP(=O)([O-])OCC[N+](C)(C)C)OC(=O)CCCCCCCCCCCCCCCCC]

### Cholesterol:

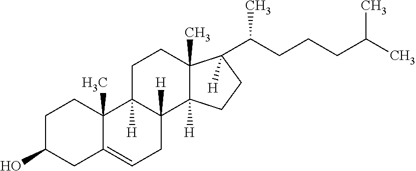

The N/P value is preferably at least about 4. In some embodiments, the N/P value ranges from 4 to 20, 4 to 12, 4 to 10, 4 to 8, or 5 to 7. In some embodiments, the N/P value is about 6.

### Cells for Targeted Delivery

According to the disclosure, a nucleic acid payload is delivered specifically to a target cell by targeting a target on target cells, e.g., an antigen on target cells, also referred to herein as “primary target”.

In some embodiments, the primary target is a structure such as a protein present on the surface of a target cell such as a cell surface antigen including a cell surface receptor.

Terms such as “expressed on the cell surface”, “associated with the cell surface” or “cell surface molecule” mean that a molecule such as a receptor or antigen is associated with and located at the plasma membrane of a cell, wherein at least a part of the molecule faces the extracellular space of said cell and is accessible from the outside of said cell, e.g., by a binding molecule such as an antibody located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells. In one embodiment, an antigen expressed on the surface of cells is an integral membrane protein having an extracellular portion recognized by a binding molecule such as an antibody.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell.

The primary target may be upregulated during a disease, e.g. infection or cancer. In diseased tissues, markers can differ from healthy tissue and offer unique possibilities for therapy, especially targeted therapy.

In some embodiments, the primary target is a disease-associated antigen, such as a tumor antigen, a viral antigen, or a bacterial antigen. This allows diseased cells to be targeted by the methods and agents described herein, e.g., for delivering a pharmaceutically active agent.

The term “disease-associated antigen” is used in its broadest sense to refer to any antigen associated with a disease. Disease-associated antigens may be associated with infection by microbes, typically microbial antigens, or associated with cancer, typically tumors.

In some embodiments, the primary target is a tumor antigen. In the context of the present disclosure, the term “tumor antigen” or “tumor-associated antigen” relates to proteins that are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2. The tumor antigens in the context of the present disclosure include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present disclosure, the tumor antigen is preferably associated with the cell surface of a cancer cell and is preferably not or only rarely expressed in normal tissues. Preferably, the tumor antigen or the aberrant expression of the tumor antigen identifies cancer cells. In the context of the present disclosure, the tumor antigen that is expressed by a cancer cell in a subject, e.g., a patient suffering from a cancer disease, is preferably a self-protein in said subject. In preferred embodiments, the tumor antigen in the context of the present disclosure is expressed under normal conditions specifically in a tissue or organ that is non-essential, i.e., tissues or organs which when damaged by the immune system do not lead to death of the subject, or in organs or structures of the body which are not or only hardly accessible by the immune system. Preferably, the amino acid sequence of the tumor antigen is identical between the tumor antigen which is expressed in normal tissues and the tumor antigen which is expressed in cancer tissues.

Examples for tumor antigens include p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY—BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT. Particularly preferred tumor antigens include CLAUDIN-18.2 (CLDN18.2) and CLAUDIN-6 (CLDN6).

In some embodiments, the primary target is a structure such as a protein present on the surface of a target cell such as a cell surface antigen or cell surface receptor the presence or amount of which is characteristic for certain cell types compared to others. This allows certain cell types characterized by the presence or increased amounts to be targeted by the methods and agents described herein. In some embodiments, the cells for targeted delivery are immune effector cells and the primary target is a cell surface antigen that is characteristic for immune effector cells. Targeting of immune effector cells by the methods and agents described herein allows the transfection of these cells with nucleic acid encoding an antigen receptor and the generation of immune effector cells genetically modified to express an antigen receptor.

### Immune Effector Cells

The immune effector cells used in connection with the methods and agents described herein and into which nucleic acids (DNA or RNA) encoding antigen receptors may be introduced include, in particular, immune effector cells such as cells with lytic potential, in particular lymphoid cells, and are preferably cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present disclosure will preferably be autologous cells, although heterologous cells or allogenic cells can be used.

The term “effector functions” in the context of the present disclosure includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present disclosure are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-a, and specific cytolytic killing of antigen expressing target cells.

The term “immune effector cell” or “immunoreactive cell” in the context of the present disclosure relates to a cell which exerts effector functions during an immune reaction. An “immune effector cell” in some embodiments is capable of binding an antigen such as an antigen presented by in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present disclosure, “immune effector cells” are T cells, preferably CD4⁺ and/or CD8⁺ T cells, most preferably CD8⁺ T cells. The term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

In some embodiments, the genetically modified immune effector cells are CAR-expressing immune effector cells. In some embodiments, the genetically modified immune effector cells are TCR-expressing immune effector cells.

The immune effector cells to be used herein may express an endogenous antigen receptor such as T cell receptor or B cell receptor or may lack expression of an endogenous antigen receptor.

A “lymphoid cell” is a cell which, optionally after suitable modification, e.g. after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In some embodiments, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45RO isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4+ or CD8′ and usually express CD45RO.

As used herein, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the at T cells.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

### Targeted Delivery of Nucleic Acid Payloads

The agents and methods described herein find use in a variety of applications in which it is desired to introduce a nucleic acid payload, e.g., an exogenous nucleic acid sequence, into a target cell, and are particularly of interest where it is desired to express peptide or polypeptide encoded by a nucleic acid in a target cell into which the nucleic acid has been introduced. The agents described herein may be administered by in vitro or in vivo protocols.

Delivery of nucleic acid payloads, e.g., nucleic acid transfection, using the methods and agents described herein can be used with a variety of target cells such that the nucleic acid payload is introduced into the target cells. The present disclosure may provide for in vitro or in vivo introduction of the nucleic acid payload into the target cell, depending on the location of the target cell. For example, where the target cell is an isolated cell, the nucleic acid payload may be introduced directly into the cell under cell culture conditions permissive of viability of the target cell. Alternatively, where the target cell or cells are part of a multicellular organism, the targeting particles described herein may be administered to the organism or host in a manner such that the targeting particles are able to enter the target cell(s). By “in vivo” it is meant in the targeting particles are administered to a living body of an animal. By “ex vivo” it is meant that cells are modified outside of the body. Such cells may be returned to a living body. The route of administration of the targeting particles to the multicellular organism depends on several parameters, including the nature of the targeting particles. Of particular interest as systemic routes are vascular routes, by which the targeting particles are introduced into the vascular system of the host, e.g., an artery or vein, where intravenous routes of administration are of particular interest in many embodiments. For administration, targeting particles typically are present in a pharmaceutical preparation, e.g., comprising a pharmaceutically acceptable carrier, diluent and/or adjuvant, and include an effective amount of the payload. In certain embodiments, the targeting particles are administered in an aqueous delivery vehicle, e.g., a saline solution. As such, in many embodiments, the targeting particles are administered intravascularly, e.g., intraarterially or intravenously, employing an aqueous based delivery vehicle, e.g., a saline solution.

In many embodiments, the targeting particles are administered to a multicellular organism in an in vivo manner such that the nucleic acid payload is introduced into a target cell of the multicellular organism.

In the case of nucleic acid payloads, administration is typically under conditions sufficient for expression of the nucleic acid to occur. In some embodiments, the agents and methods described herein result in persistent expression of the nucleic acid payload, as opposed to transient expression, as indicated above. By persistent expression is meant that the expression of nucleic acid at a detectable level persists for an extended period of time, if not indefinitely, following administration of the nucleic acid payload. By extended period of time is meant at least 1 week, usually at least 2 months and more usually at least 6 months. By detectable level is meant that the expression of the nucleic acid is at a level such that one can detect the encoded protein in the mammal, e.g., in the serum of the mammal, at a therapeutic concentration.

In some embodiments, the above-described persistent expression is achieved with or without integration of the nucleic acid payload into the target cell genome of the host. In some embodiments, the nucleic acid introduced into the target cells integrates into the target cell genome, i.e., one or more chromosomes of the target cell. In some embodiments, the nucleic acid is maintained episomally, e.g., it is an episomal vector that provides for persistent expression.

Accordingly, cells described herein, e.g., immune effector cells, may be genetically modified ex vivo/in vitro or in vivo in a subject being treated to express a peptide or polypeptide, e.g., an antigen receptor such as a chimeric antigen receptor (CAR) or a T cell receptor (TCR) binding antigen or a procession product thereof, in particular when present on or presented by a target cell, e.g., an antigen presenting cell or a diseased cell. In some embodiments, modification to express a peptide or polypeptide, e.g., an antigen receptor, takes place in vivo. The cells may be endogenous cells of the patient or may have been administered to a patient. In some embodiments, modification to express a peptide or polypeptide, e.g., an antigen receptor, takes place ex vivo/in vitro. Subsequently, modified cells may be administered to a patient.

In some embodiments, the methods and agents described herein are used to transfect immune effector cells with nucleic acid encoding an antigen receptor for generating immune effector cells genetically modified to express an antigen receptor.

### T Cell Receptor (TCR)

The term “T cell receptor” or “TCR” as used herein refers to a protein receptor on T cells that is composed of a heterodimer of an alpha (α) and beta (β) chain, although in some cells the TCR consists of gamma and delta (γδ) chains. In some embodiments, the TCR may be derived from any cell comprising a TCR, including a helper T cell, a cytotoxic T cell, a memory T cell, regulatory T cell, natural killer T cell, and gamma delta T cell, for example. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (MHC).

### Chimeric Antigen Receptors (CAR)

Adoptive cell transfer therapy with CAR-engineered T cells expressing chimeric antigen receptors is a promising anti-cancer therapeutic as CAR-modified T cells can be engineered to target virtually any tumor antigen, preferably in an MHC-independent manner. For example, patient's T cells may be genetically engineered (genetically modified) to express CARs specifically directed towards antigens on the patient's tumor cells.

As used herein, the term “CAR” (or “chimeric antigen receptor”) is synonymous with the terms “chimeric T cell receptor” and “artificial T cell receptor” and relates to an artificial receptor comprising a single molecule or a complex of molecules which recognizes, i.e., binds to, a target structure (e.g. an antigen) on a target cell such as a cancer cell (e.g. by binding of an antigen binding domain to an antigen expressed on the surface of the target cell) and may confer specificity onto an immune effector cell such as a T cell expressing said CAR on the cell surface. Such cells do not necessarily require processing and presentation of an antigen for recognition of the target cell but rather may recognize preferably with specificity any antigen present on a target cell. Preferably, recognition of the target structure by a CAR results in activation of an immune effector cell expressing said CAR. A CAR may comprise one or more protein units said protein units comprising one or more domains as described herein. The term “CAR” does not include T cell receptors.

A CAR comprises a target-specific binding element otherwise referred to as an antigen binding moiety or antigen binding domain that is generally part of the extracellular domain of the CAR. Specifically, the CAR may target an antigen on target cells, e.g., diseased cells such as tumor cells.

In some embodiments, an antigen binding domain comprises a variable region of a heavy chain of an immunoglobulin (VH) with a specificity for the antigen and a variable region of a light chain of an immunoglobulin (VL) with a specificity for the antigen. In some embodiments, an immunoglobulin is an antibody. In some embodiments, said heavy chain variable region (VH) and the corresponding light chain variable region (VL) are connected via a peptide linker. Preferably, the antigen binding moiety portion in the CAR is a scFv. In some embodiments, an antigen binding domain comprises a VHH domain.

The CAR is preferably designed to comprise a transmembrane domain that is fused to the extracellular domain of the CAR. In some embodiments, the transmembrane domain is not naturally associated with one of the domains in the CAR. In some embodiments, the transmembrane domain is naturally associated with one of the domains in the CAR. In some embodiments, the transmembrane domain is modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use herein may be derived from (i.e. comprise at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some instances, the CAR comprises a hinge domain which forms the linkage between the transmembrane domain and the extracellular domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

In some embodiments, the CAR comprises a primary cytoplasmic signaling sequence derived from CD3-zeta. Further, the cytoplasmic domain of the CAR may comprise the CD3-zeta signaling domain combined with a costimulatory signaling region.

The identity of the co-stimulation domain is limited only in that it has the ability to enhance cellular proliferation and survival upon binding of the targeted moiety by the CAR. Suitable co-stimulation domains include CD28, CD137 (4-1BB), a member of the tumor necrosis factor receptor (TNFR) superfamily, CD134 (OX40), a member of the TNFR-superfamily of receptors, and CD278 (ICOS), a CD28-superfamily co-stimulatory molecule expressed on activated T cells. The skilled person will understand that sequence variants of these noted co-stimulation domains can be used without adversely impacting the disclosure, where the variants have the same or similar activity as the domain on which they are modeled. Such variants will have at least about 80% sequence identity to the amino acid sequence of the domain from which they are derived. In some embodiments, the CAR constructs comprise two co-stimulation domains. While the particular combinations include all possible variations of the four noted domains, specific examples include CD28+CD137 (4-1BB) and CD28+CD134 (OX40).

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In some embodiments, the CAR comprises a signal peptide which directs the nascent protein into the endoplasmic reticulum. In some embodiments, the signal peptide precedes the antigen binding domain. In some embodiments, the signal peptide is derived from an immunoglobulin such as IgG.

A CAR may comprise the above domains, together in the form of a fusion protein. Such fusion proteins will generally comprise an antigen binding domain, one or more co-stimulation domains, and a signaling sequence, linked in a N-terminal to C-terminal direction. However, the CARs are not limited to this arrangement and other arrangements are acceptable and include a binding domain, a signaling domain, and one or more co-stimulation domains. It will be understood that because the binding domain must be free to bind antigen, the placement of the binding domain in the fusion protein will generally be such that display of the region on the exterior of the cell is achieved. In the same manner, because the co-stimulation and signaling domains serve to induce activity and proliferation of the cytotoxic lymphocytes, the fusion protein will generally display these two domains in the interior of the cell.

In some embodiments, a CAR molecule comprises:
i) a target antigen binding domain;
ii) a transmembrane domain; and
iii) an intracellular domain that comprises a signaling domain, e.g., a CD3-zeta signaling domain, optionally in combination with one or more costimulatory domains, e.g., an intracellular domain that comprises a 4-1BB costimulatory domain.

In some embodiments, the antigen binding domain comprises an scFv. In some embodiments, the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD154, KIRD52, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGBI, CD29, ITGB2, CD18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGLI, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the transmembrane domain comprises a CD8a transmembrane domain. In some embodiments, the antigen binding domain is connected to the transmembrane domain by a hinge domain. In some embodiments, the hinge domain is a CD8a hinge domain.

In some embodiments, the CAR molecule comprises:
i) a target antigen binding domain;
ii) a CD8a hinge domain;
iii) a CD8a transmembrane domain; and
iv) an intracellular domain that comprises a 4-1BB costimulatory domain, and a CD3-zeta signaling domain.

### Genetic Modification of Immune Effector Cells

Particles which are functionalized as described herein (i.e., functionalized with a targeting compound comprising a moiety targeting a cell surface antigen on immune effector cells, or functionalized with a targeting compound comprising a moiety targeting a docking compound, said docking compound comprising a moiety targeting a cell surface antigen on immune effector cells) may be used ex vivo/in vitro or in vivo for delivering a nucleic acid encoding an antigen receptor to immune effector cells such as B cells or T cells, in particular CD8+ T cells, thus producing immune effector cells genetically modified to express an antigen receptor.

If the immune effector cell to be targeted is a T cell, the primary target is in some embodiments a cell surface molecule on T cells, e.g., a T cell marker.

As used herein, the term “T cell marker” refers to surface molecules on T cells which are specific for particular T cells. T cell markers suitable for use herein include, but are not limited to surface CD3, CD4, CD8, CD45RO or any other CD antigen specific for T cells.

If the immune effector cell to be targeted is a B cell, the primary target is in some embodiments a cell surface molecule on B cells, e.g., a B cell marker.

As used herein, the term “B cell marker” refers to surface molecules on B cells which are specific for antigen-specific IgG-producing B cells. B cell markers suitable for use herein include, but are not limited to surface IgG, kappa and lambda chains, Ig-alpha (CD79alpha), Ig-beta (CD79beta), CD19, la, Fc receptors, B220 (CD45R), CD20, CD21, CD22, CD23, CD81 (TAPA-1) or any other CD antigen specific for B cells.

In some embodiments, the immune effector cell to be targeted is a T cell.

In some embodiments, the moiety targeting a primary target of the targeting compound or docking compound is directed against CD8. In some embodiments, the moiety targeting a primary target of the targeting compound or docking compound directed against CD8 is selected from the group consisting of an anti-CD8 DARPin, an anti-CD8 VHH and an anti-CD8 scFv. In some embodiments, the moiety of a docking compound binding to a targeting compound is a NbALFA-nanobody (NbALFA). Accordingly, in some embodiments, the docking compound may have a structure selected from the group consisting of NbALFA×anti-CD8 DARPin, NbALFA×anti-CD8 VHH and NbALFA×anti-CD8 scFv. In these embodiments, the targeting compound may comprise the structure L-X1-P-X2-B described above, wherein B comprises an ALFA-tag.

In some embodiments, the moiety targeting a primary target of the targeting compound or docking compound is directed against CD4. In some embodiments, the moiety targeting a primary target of the targeting compound or docking compound directed against CD4 is selected from the group consisting of an anti-CD4 DARPin, an anti-CD4 VHH and an anti-CD4 scFv. In some embodiments, the moiety of a docking compound binding to a targeting compound is a NbALFA-nanobody (NbALFA). Accordingly, in some embodiments, the docking compound may have a structure selected from the group consisting of NbALFA×anti-CD4 DARPin, NbALFA×anti-CD4 VHH and NbALFA×anti-CD4 scFv. In these embodiments, the targeting compound may comprise the structure L-X1-P-X2-B described above, wherein B comprises an ALFA-tag.

In some embodiments, the moiety targeting a primary target of the targeting compound or docking compound is directed against CD3. In some embodiments, the moiety targeting a primary target of the targeting compound or docking compound directed against CD3 is selected from the group consisting of an anti-CD3 DARPin, an anti-CD3 VHH and an anti-CD3 scFv. In some embodiments, the moiety of a docking compound binding to a targeting compound is a NbALFA-nanobody (NbALFA). Accordingly, in some embodiments, the docking compound may have a structure selected from the group consisting of NbALFA×anti-CD3 DARPin, NbALFA×anti-CD3 VHH and NbALFA×anti-CD3 scFv. In these embodiments, the targeting compound may comprise the structure L-X1-P-X2-B described above, wherein B comprises an ALFA-tag.

Genetic modification described herein includes non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of γ-retroviruses and lentiviral vectors. γ-Retroviruses are relatively easy to produce, efficiently and permanently transduce cells such as T cells, and have preliminarily proven safe from an integration standpoint in primary human T cells. Lentiviral vectors also efficiently and permanently transduce cells such as T cells but are more expensive to manufacture. They are also potentially safer than retrovirus based systems.

In some embodiments, T cells or T cell progenitors are transfected either ex vivo or in vivo with nucleic acid encoding an antigen receptor. In some embodiments, a combination of ex vivo and in vivo transfection may be used. In some embodiments, the T cells or T cell progenitors are from the subject to be treated. In some embodiments, the T cells or T cell progenitors are from a subject which is different to the subject to be treated.

In some embodiments, CAR T cells may be produced in vivo, and therefore nearly instantaneously, using particles such as nanoparticles described herein targeted to T cells. For example, particles may be coupled to a targeting compound comprising a moiety for binding to CD3, e.g., CD3e, on T cells, e.g., anti-CD3 VHH or anti-CD3 F(ab) fragment or a targeting compound bound to a docking compound, the docking compound comprising a moiety for binding to CD3, e.g., CD3e, on T cells, e.g., anti-CD3 VHH or anti-CD3 F(ab) fragment. Upon binding to T cells, these particles may be endocytosed. Their contents, for example nucleic acid encoding antigen receptor, e.g., plasmid DNA encoding an anti-tumor antigen CAR, may be directed to the T cell nucleus due to, for example, the inclusion of peptides containing microtubule-associated sequences (MTAS) and nuclear localization signals (NLSs). The inclusion of transposons flanking the nucleic acid encoding antigen receptor, e.g., the CAR gene expression cassette, and a separate nucleic acid, e.g., plasmid, encoding a hyperactive transposase, may allow for the efficient integration of the nucleic acid encoding antigen receptor, e.g., the CAR vector, into chromosomes.

Another possibility is to use the CRISPR/Cas9 method to deliberately place a peptide/polypeptide coding sequence, e.g., an antigen receptor coding sequence such as a CAR coding sequence, at a specific locus. For example, existing T cell receptors (TCR) may be knocked out, while knocking in the CAR and placing it under the dynamic regulatory control of the endogenous promoter that would otherwise moderate TCR expression.

Accordingly, besides nucleic acid encoding an antigen receptor the particles described herein may also deliver as cargo gene editing tools like CRISPR/Cas9 (or related) or transposon systems like sleeping beauty or piggy bag. Such tools (e.g. transposase, gene editing tools like CRISPR/Cas9) for genomic integration/editing may be delivered as protein or coding nucleic acid (DNA or RNA). Nevertheless, also delivery of mRNA is an option to induce transient expression of antigen receptors like CAR or TCR.

In some embodiments, the cells genetically modified to express an antigen receptor are stably or transiently transfected with nucleic acid encoding the antigen receptor. Thus, the nucleic acid encoding the antigen receptor is integrated or not integrated into the genome of the cells.

In some embodiments, the cells genetically modified to express an antigen receptor are inactivated for expression of an endogenous T cell receptor and/or endogenous HLA.

In some embodiments, the cells described herein may be autologous, allogeneic or syngeneic to the subject to be treated. In some embodiments, the present disclosure envisions the removal of cells from a patient and the subsequent re-delivery of the cells to the patient. In some embodiments, the present disclosure does not envision the removal of cells from a patient. In the latter case all steps of genetic modification of cells are performed in vivo.

### Binding Moieties and Agents

The present disclosure describes binding moieties or agents such as antibodies or antibody derivatives. Moreover, the disclosure describes bispecific or multispecific binding agents such as bispecific antibodies comprising a first and a second binding domain, wherein the first binding domain is capable of binding to a primary target and the second binding domain is capable of binding to a targeting compound.

The term “binding agent” as used herein includes any agent capable of binding to desired antigens. In certain embodiments, the binding agent is or comprises an antibody, antibody fragment, or any other binding protein, or any combination thereof.

The term “binding moiety” as used herein includes any moiety, group or domain capable of binding to desired antigens. In certain embodiments, the binding moiety is or comprises an antibody, antibody fragment, or any other binding protein, or any combination thereof.

As used herein, the term “antigen” is a molecule capable of being bound by a binding moiety or agent, such as an antibody. An antigen may additionally be capable of inducing a humoral immune response and/or cellular immune response leading to the production of B- and/or T-lymphocytes. An antigen may have one or more epitopes (B-cell and T-cell epitopes).

The term “epitope” refers to a part or fragment of a molecule or antigen that is recognized by a binding agent. For example, the epitope may be recognized by an antibody or any other binding protein. An epitope may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes structural epitopes.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)).

The term “antibody” (Ab) as used herein refers to an immunoglobulin molecule, a fragment of an immunoglobulin molecule, or a derivative of either thereof, which has the ability to bind, preferably specifically bind to an antigen. In some embodiments, binding takes place under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen). The variable regions of the heavy and light chains of the immunoglobulin molecule contain a binding domain that interacts with an antigen. The term “antigen-binding region”, “binding region” or “binding domain”, as used herein, refers to the region or domain which interacts with the antigen and typically comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

The phrase “single chain Fv” or “scFv” refers to an antibody in which the variable domains of the heavy chain and of the light chain (VH and VL) of a traditional two chain antibody have been joined to form one chain. Optionally, a linker (usually a peptide) is inserted between the two chains to allow for proper folding and creation of an active binding site.

A single-domain antibody, also known as a nanobody, is an antibody fragment consisting of a single monomeric variable antibody domain. In some embodiments, a single-domain antibody is a variable domain (V_{H}) of a heavy-chain antibody. These are called VHH fragments. Like a whole antibody, a single-domain antibody is able to bind selectively to a specific antigen. The first single-domain antibodies were engineered from heavy-chain antibodies found in camelids. Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

An antibody can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody may be a sequence variant of a naturally-occurring IgG1 antibody, including variations in the constant regions.

In various embodiments, an antibody is an IgG1 antibody, more particularly an IgG1, kappa or IgG1, lambda isotype (i.e. IgG1, K, A), an IgG2a antibody (e.g. IgG2a, K, A), an IgG2b antibody (e.g. IgG2b, K, A), an IgG3 antibody (e.g. IgG3, K, A) or an IgG4 antibody (e.g. IgG4, K, A).

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in some embodiments, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15(2):146-56.

When used herein, unless contradicted by context, the term “Fab-arm”, “binding arm” or “arm” includes one heavy chain-light chain pair and is used interchangeably with “half-molecule” herein.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

The present disclosure also envisions antibodies comprising functional variants of the VL regions, VH regions, or one or more CDRs of the antibodies described herein. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody.

Exemplary variants include those which differ from VH and/or VL and/or CDR regions of the parent antibody sequences mainly by conservative substitutions; for instance, up to 10, such as 9, 8, 7, 6, 5, 4, 3, 2 or 1 of the substitutions in the variant are conservative amino acid residue replacements.

Functional variants of antibody sequences described herein such as VL regions, or VH regions, or antibody sequences having a certain degree of homology or identity to antibody sequences described herein such as VL regions, or VH regions preferably comprise modifications or variations in the non-CDR sequences, while the CDR sequences preferably remain unchanged.

The term “specificity” as used herein is intended to have the following meaning unless contradicted by context. Two antibodies have the “same specificity” if they bind to the same antigen and the same epitope.

An antibody or fragment useful herein may compete with a specific antibody or fragment described herein.

The term “competes” and “competition” may refer to the competition between a first antibody and a second antibody to the same antigen. It is well known to a person skilled in the art how to test for competition of antibodies for binding to a target antigen. An example of such a method is a so-called cross-competition assay, which may e.g. be performed as an ELISA or by flow-cytometry. Alternatively, competition may be determined using biolayer interferometry.

Antibodies which compete for binding to a target antigen may bind different epitopes on the antigen, wherein the epitopes are so close to each other that a first antibody binding to one epitope prevents binding of a second antibody to the other epitope. In other situations, however, two different antibodies may bind the same epitope on the antigen and would compete for binding in a competition binding assay. Such antibodies binding to the same epitope are considered to have the same specificity herein. Thus, in some embodiments, antibodies binding to the same epitope are considered to bind to the same amino acids on the target molecule. That antibodies bind to the same epitope on a target antigen may be determined by standard alanine scanning experiments or antibody-antigen crystallization experiments known to a person skilled in the art. Preferably, antibodies or binding domains binding to different epitopes are not competing with each other for binding to their respective epitopes.

Naturally occurring antibodies are generally monospecific, i.e. they bind to a single antigen. Described herein are binding agents, e.g., docking compounds, binding to different epitopes on e.g. a primary target and a targeting compound. Such binding agents are at least bispecific or multispecific such as trispecific, tetraspecific and so on. Thus, the binding agent may comprise two or more antibodies as described herein or fragments thereof. In particular, a binding agent described herein may be an artificial protein that is composed of two different antibodies, an antibody and a fragment of a different antibody, and fragments of two different antibodies (said fragments of two different antibodies forming two binding domains).

According to the disclosure, a bispecific binding agent, in particular a bispecific protein, such as a bispecific antibody is a molecule that has two different binding specificities and thus may bind to two epitopes. Particularly, the term “bispecific antibody” as used herein refers to an antibody comprising two antigen-binding sites, a first binding site having affinity for a first epitope and a second binding site having binding affinity for a second epitope distinct from the first.

The term “bispecific” as used herein refers to an agent having two different antigen-binding regions binding to different epitopes.

“Multispecific binding agents” are molecules which have more than two different binding specificities.

Many different formats and uses of bispecific antibodies are known in the art, and were reviewed by Kontermann; Drug Discov Today, 2015 July; 20(7):838-47 and; MAbs, 2012 March-April; 4(2):182-97.

A bispecific binding agent according to the present disclosure is not limited to any particular bispecific format or method of producing it.

Examples of bispecific antibody molecules which may be used herein comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

The term “bispecific antibody” includes diabodies. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90: 6444-6448; Poljak, R. J., et al. (1994) Structure 2: 1121-1123). Bispecific antibodies also include bispecific single chain antibodies. The term “bispecific single chain antibody” denotes a single polypeptide chain comprising two binding domains. In particular, the term “bispecific single chain antibody” or “single chain bispecific antibody” or related terms as used herein preferably mean antibody constructs resulting from joining at least two antibody variable regions in a single polypeptide chain devoid of the constant and/or Fc portion(s) present in full immunoglobulins. For example, a bispecific single chain antibody may be a construct with a total of two antibody variable regions, for example two VH regions, each capable of specifically binding to a separate epitope, and connected with one another through a short polypeptide spacer such that the two antibody variable regions with their interposed spacer exist as a single contiguous polypeptide chain. Another example of a bispecific single chain antibody may be a single polypeptide chain with three antibody variable regions. Here, two antibody variable regions, for example one VH and one VL, may make up an scFv, wherein the two antibody variable regions are connected to one another via a synthetic polypeptide linker, the latter often being genetically engineered so as to be minimally immunogenic while remaining maximally resistant to proteolysis. This scFv is capable of specifically binding to a particular epitope, and is connected to a further antibody variable region, for example a VH region, capable of binding to a different epitope than that bound by the scFv. Yet another example of a bispecific single chain antibody may be a single polypeptide chain with four antibody variable regions. Here, the first two antibody variable regions, for example a VH region and a VL region, may form one scFv capable of binding to one epitope, whereas the second VH region and VL region may form a second scFv capable of binding to another epitope. Within a single contiguous polypeptide chain, individual antibody variable regions of one specificity may advantageously be separated by a synthetic polypeptide linker, whereas the respective scFvs may advantageously be separated by a short polypeptide spacer as described above. According to some embodiments, the first binding domain of the bispecific antibody comprises one antibody variable domain, preferably a VHH domain. According to some embodiments, the first binding domain of the bispecific antibody comprises two antibody variable domains, preferably a scFv, i.e. VH-VL or VL-VH. According to some embodiments, the second binding domain of the bispecific antibody comprises one antibody variable domain, preferably a VHH domain. According to some embodiments, the second binding domain of the bispecific antibody comprises two antibody variable domains, preferably a scFv, i.e. VH-VL or VL-VH. In its minimal form, the total number of antibody variable regions in the bispecific antibody is thus only two. For example, such an antibody could comprise two VH or two VHH domains. According to some embodiments, the first binding domain and the second binding domain of the bispecific antibody each comprise one antibody variable domain, preferably a VHH domain. According to some embodiments, the first binding domain and the second binding domain of the bispecific antibody each comprise two antibody variable domains, preferably a scFv, i.e. VH-VL or VL-VH. In this embodiment, the binding agent preferably comprises (i) a heavy chain variable domain (VH) of a first antibody, (ii) a light chain variable domain (VL) of a first antibody, (iii) a heavy chain variable domain (VH) of a second antibody and (iv) a light chain variable domain (VL) of a second antibody.

In some embodiments, the bispecific molecules comprise two Fab regions, each being directed against different epitopes. In some embodiments, the molecule of the disclosure is an antigen binding fragment (Fab)2 complex. The Fab2 complex is composed of two Fab fragments, one Fab fragment comprising a Fv domain, i.e. VH and VL domains, specific for one epitope, and the other Fab fragment comprising a Fv domain specific for another epitope. Each of the Fab fragments may be composed of two single chains, a VL-CL module and a VH—CH module. Alternatively, each of the individual Fab fragments may be arranged in a single chain, preferably, VL-CL-CH—VH, and the individual variable and constant domains may be connected with a peptide linker.

In some embodiments, the binding agent according to the disclosure includes various types of bivalent and trivalent single-chain variable fragments (scFvs), fusion proteins mimicking the variable domains of two antibodies. Divalent (or bivalent) single-chain variable fragments (di-scFvs, bi-scFvs) can be engineered by linking two scFvs. This can be done by producing a single peptide chain with two VH and two VL regions, yielding tandem scFvs. The disclosure also includes multispecific molecules comprising more than two scFvs binding domains.

Another possibility is the creation of scFvs with linker peptides that are too short for the two variable regions to fold together (about five amino acids), forcing scFvs to dimerize. This type is known as diabodies. Still shorter linkers (one or two amino acids) lead to the formation of trimers, so-called triabodies or tribodies. Tetrabodies have also been produced. They exhibit an even higher affinity to their targets than diabodies.

A particularly preferred example of a bispecific antibody fragment is a diabody (Kipriyanov, Int. J. Cancer 77 (1998), 763-772), which is a small bivalent and bispecific antibody fragment. Diabodies comprise a heavy chain variable domain (VH) and a light chain variable domain (VL) on the same polypeptide chain (VH-VL) connected by a peptide linker that is too short to allow pairing between the two domains on the same chain. This forces pairing with the complementary domains of another chain and promotes the assembly of a dimeric molecule with two functional antigen binding sites.

In some embodiments, the bispecific or multispecific molecule according to the disclosure comprises variable (VH, VL) and constant domains (C) of immunoglobulins. In some embodiments the bispecific molecule is a minibody, preferably, a minibody comprising two single VH-VL-C chains that are connected with each other via the constant domains (C) of each chain. According to this aspect, the corresponding variable heavy chain regions (VH), corresponding variable light chain regions (VL) and constant domains (C) are arranged, from N-terminus to C-terminus, in the order VH(Epitope 1)-VL(Epitope 1)-(C) and VH(Epitope 2)-VL(Epitope 2)-C, wherein C is preferably a CH3 domain, Epitope 1 refers to a first epitope and Epitope 2 refers to a second epitope. Pairing of the constant domains results in formation of the minibody.

According to another aspect, the bispecific binding agent of the disclosure is in the format of a bispecific single chain antibody construct, whereby said construct comprises or consists of at least two binding domains. In some embodiments, each binding domain comprises one variable region from an antibody heavy chain (“VH region”), wherein the VH region of the first binding domain specifically binds to Epitope 1, and the VH region of the second binding domain specifically binds to Epitope 2. The two binding domains are optionally linked to one another by a short polypeptide spacer. Each binding domain may additionally comprise one variable region from an antibody light chain (“VL region”), the VH region and VL region within each of the first and second binding domains being linked to one another via a polypeptide linker long enough to allow the VH region and VL region of the first binding domain and the VH region and VL region of the second binding domain to pair with one another.

In some embodiments, the binding agent described herein comprises an antibody, e.g., a full-length antibody, comprising the first binding domain. In some embodiments, the binding agent described herein comprises an antibody fragment such as scFv or VHH comprising the second binding domain which is covalently linked to the antibody comprising the first binding domain. In some embodiments, the binding agent comprises the antibody fragment such as scFv or VHH covalently linked to the N-terminus or C-terminus of the light chain or heavy chain of the antibody.

In some embodiments, a binding moiety described herein, e.g., a binding moiety comprised in a docking compound binding to a primary target, comprises a DARPin. In some embodiments, the binding moiety directs a particle to immune effector cells, in particular T cells such as CD8⁺ T cells.

The term “DARPin” refers to designed ankyrin repeat proteins. DARPins are based on naturally occurring ankyrin repeat proteins, yet contain one or more amino acid mutations that can affect, for example, their binding affinity to a target molecule, their cell surface expression, and the like. DARPins preferably include 2 to 3 ankyrin repeat modules flanked by N- and C-capping repeats. Each ankyrin repeat module includes about 33 amino acid residues.

Ankyrin repeat proteins have been identified in 1987 through sequence comparisons between four such proteins in Saccharomyces cerevisiae, Drosophila melanogaster and Caenorhabditis elegans. Breeden and Nasmyth reported multiple copies of a repeat unit of approximately 33 residues in the sequences of swi6p, cddOp, notch and lin-12 (Breeden et al., Nature 329, 651-654 (1987)). The subsequent discovery of 24 copies of this repeat unit in the ankyrin protein led to the naming of this repeat unit as the ankyrin repeat (Lux et al., Nature 344, 36-42 (1990)). Later, this repeat unit has been identified in several hundreds of proteins of different organisms and viruses (Bork, Proteins 17(4), 363-74 (1993)). These proteins are located in the nucleus, the cytoplasm or the extracellular space. This is consistent with the fact that the ankyrin repeat domain of these proteins is independent of disulfide bridges and thus independent of the oxidation state of the environment. The number of repeat units per protein varies from two to more than twenty. Tertiary structures of ankyrin repeat units share a characteristic fold (Sedgwick and Smerdon, Trends Biochem Sci. 24(8), 311-6 (1999)) composed of a β-hairpin followed by two antiparallel α-helices and ending with a loop connecting the repeat unit with the next one. Domains built of ankyrin repeat units are formed by stacking the repeat units to an extended and curved structure. Proteins containing ankyrin repeat domains often contain additional domains. While the latter domains have variable functions, the function of the ankyrin repeat domain is most often the binding of other proteins. When analysing the repeat units of these proteins, the target interaction residues are mainly found in the β-hairpin and the exposed part of the first α-helix. These target interaction residues are hence forming a large contact surface on the ankyrin repeat domain. This contact surface is exposed on a framework built of stacked units of α-helix 1, α-helix 2 and the loop.

DARPins that bind to specific targets can be identified by screening combinatorial libraries of DARPins and selecting those with desired binding properties for the target. Such screening methods are described in, e.g., Muench et al., Molecular Therapy, 16(4), 686-693, 2011. For example, ribosomal display or phage display methods can be used to select target-specific DARPins from diverse libraries.

The term “repeat protein” refers to a (poly)peptide/protein comprising one or more repeat domains. In one embodiment, a repeat protein comprises up to four repeat domains. In one embodiment, a repeat protein comprises up to three repeat domains. In one embodiment, a repeat protein comprises up to two repeat domains. In the most preferred embodiment, a repeat protein comprises one repeat domain. The individual domains of a repeat protein may be connected to each other directly or via (poly)peptide linkers. The term “(poly)peptide linker” refers to an amino acid sequence which is able to link two protein domains. Such linkers include, for example, glycine-serine-linkers of variable lengths and are known to the person skilled in the relevant art.

The term “repeat domain” refers to a protein domain comprising two or more consecutive repeat units (modules). In one embodiment, said repeat units are structural units having the same or a similar folding structure, and preferably stack tightly to preferably create a superhelical structure having a joint hydrophobic core.

The term “structural unit” refers to a locally ordered part of a (poly)peptide, formed by three-dimensional interactions between two or more segments of secondary structure that are near one another along the (poly)peptide chain. Such a structural unit comprises a structural motif.

The term “structural motif” refers to a three-dimensional arrangement of secondary structure elements present in at least one structural unit. Structural motifs are well known to the person skilled in the relevant art. Said structural units may alone not be able to acquire a defined three-dimensional arrangement; however, their consecutive arrangement as repeat modules in a repeat domain leads to a mutual stabilization of neighbouring units which may result in a superhelical structure.

The term “repeat modules” refers to the repeated amino acid sequences of the repeat proteins, which are derived from the repeat units of naturally occurring proteins. Each repeat module comprised in a repeat domain is derived from one or more repeat units of a family of naturally occurring repeat proteins, e.g., ankyrin repeat proteins.

The term “set of repeat modules” refers to the total number of repeat modules present in a repeat domain. Such “set of repeat modules” present in a repeat domain comprises two or more consecutive repeat modules, and may comprise just one type of repeat module in two or more copies, or two or more different types of modules, each present in one or more copies. Such set of repeat modules comprising, for example, 3 repeat modules may comprise consecutively, form N- to C-terminus, repeat module 1, repeat module 2, and repeat module 3.

Different repeat domains may have an identical number of repeat modules per repeat domain or may differ in the number of repeat modules per repeat domain.

Preferably, the repeat modules comprised in a set are homologous repeat modules. In the context of the present disclosure, the term “homologous repeat modules” refers to repeat modules, wherein more than 70% of the framework residues of said repeat modules are homologous. Preferably, more than 80% of the framework residues of said repeat modules are homologous. Most preferably, more than 90% of the framework residues of said repeat modules are homologous. Computer programs to determine the percentage of homology between polypeptides, such as Fasta, Blast or Gap, are known to the person skilled in the relevant art.

The term “repeat unit” refers to amino acid sequences comprising sequence motifs of one or more naturally occurring proteins, wherein said “repeat units” are found in multiple copies, and which exhibit a defined folding topology common to all said motifs determining the fold of the protein. Such repeat units comprise framework residues and interaction residues.

One example of such repeat units is an ankyrin repeat unit. Naturally occurring proteins containing two or more such repeat units are referred to as “naturally occurring repeat proteins”. The amino acid sequences of the individual repeat units of a repeat protein may have a significant number of mutations, substitutions, additions and/or deletions when compared to each other, while still substantially retaining the general pattern, or motif, of the repeat units.

The term “repeat sequence motif” or “repeat consensus sequence” refers to an amino acid sequence, which is deduced from one or more repeat units. Such repeat sequence motifs comprise framework residue positions and target interaction residue positions. Said framework residue positions correspond to the positions of framework residues of said repeat units. Said target interaction residue positions correspond to the positions of target interaction residues of said repeat units. Such repeat sequence motifs comprise fixed positions and randomized positions. The term “fixed position” refers to an amino acid position in a repeat sequence motif, wherein said position is set to a particular amino acid. Frequently, such fixed positions correspond to the positions of framework residues.

The term “randomized position” refers to an amino acid position in a repeat sequence motif, wherein two or more amino acids are allowed at said amino acid position. Frequently, such randomized positions correspond to the positions of target target interaction residues. However, some positions of framework residues may also be randomized.

The term “folding topology” refers to the tertiary structure of said repeat units. The folding topology will be determined by stretches of amino acids forming at least parts of α-helices or β-sheets, or amino acid stretches forming linear polypeptides or loops, or any combination of α-helices, β-sheets and/or linear polypeptides/loops.

The term “consecutive” refers to an arrangement, wherein said modules are arranged in tandem.

In repeat proteins, there are at least 2, frequently 6 or more, 10 or more, or 20 or more repeat units, usually about 2 to 6 repeat units. For the most part, the repeat proteins are structural proteins and/or adhesive proteins, being present in prokaryotes and eukaryotes, including vertebrates and non-vertebrates.

In most cases, said repeat units will exhibit a high degree of sequence identity (same amino acid residues at corresponding positions) or sequence similarity (amino acid residues being different, but having similar physicochemical properties), and some of the amino acid residues might be key residues being strongly conserved in the different repeat units found in naturally occurring proteins.

However, a high degree of sequence variability by amino acid insertions and/or deletions, and/or substitutions between the different repeat units found in naturally occurring proteins will be possible as long as the common folding topology is maintained.

The term “framework residues” relates to amino acid residues of the repeat units, or the corresponding amino acid residues of the repeat modules, which contribute to the folding topology, i.e. which contribute to the fold of said repeat unit (or module) or which contribute to the interaction with a neighboring unit (or module). Such contribution might be the interaction with other residues in the repeat unit (module), or the influence on the polypeptide backbone conformation as found in α-helices or β-sheets, or amino acid stretches forming linear polypeptides or loops.

The term “target interaction residues” refers to amino acid residues of the repeat units, or the corresponding amino acid residues of the repeat modules, which contribute to the interaction with target substances. Such contribution might be the direct interaction with the target substances, or the influence on other directly interacting residues, e.g. by stabilising the conformation of the (poly)peptide of said repeat unit (module) to allow or enhance the interaction of said directly interacting residues with said target.

A “target” may be an individual molecule such as a nucleic acid molecule, a (poly)peptide protein, a carbohydrate, or any other naturally occurring molecule, including any part of such individual molecule, or complexes of two or more of such molecules. The target may be, in particular, a molecule on immune effector cells, in particular CD8.

In one embodiment, the repeat modules are directly connected. In the context of the present invention, the term “directly connected” refers to repeat modules, which are arranged as direct repeats in a repeat protein without an intervening amino acid sequence.

In another embodiment, the repeat modules are connected by a (poly)peptide linker. Thus, the repeat modules may be linked indirectly via a (poly)peptide linker as intervening sequence separating the individual modules. An “intervening sequence” may be any amino acid sequence, which allows to connect the individual modules without interfering with the folding topology or the stacking of the modules. Preferentially, said intervening sequences are short (poly)peptide linkers of less than 10, and even more preferably, of less than 5 amino acid residues.

In one embodiment, a repeat protein further comprises an N- and/or a C-terminal capping module having an amino acid sequence different from any one of said repeat modules. The term “capping module” refers to a polypeptide fused to the N- or C-terminal repeat module of a repeat domain, wherein said capping module forms tight tertiary interactions with said repeat module thereby providing a cap that shields the hydrophobic core of said repeat module at the side not in contact with the consecutive repeat module from the solvent.

Said N- and/or C-terminal capping module may be, or may be derived from, a capping unit or other domain found in a naturally occurring repeat protein adjacent to a repeat unit.

The term “capping unit” refers to a naturally occurring folded (poly)peptide, wherein said (poly)peptide defines a particular structural unit which is N- or C-terminally fused to a repeat unit, wherein said (poly)peptide forms tight tertiary interactions with said repeat unit thereby providing a cap that shields the hydrophobic core of said repeat unit at one side from the solvent. Such capping units may have sequence similarities to said repeat sequence motif.

### Nucleic Acids

The disclosure described herein provides for the delivery of a payload which comprises one or more nucleic acids. Such nucleic acids may be coding nucleic acids, e.g., nucleic acids encoding an antigen receptor, e.g., for genetic modification of immune effector cells, and/or non-coding nucleic acids.

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In some embodiments, a nucleic acid is DNA. In some embodiments, a nucleic acid is RNA. In some embodiments, a nucleic acid is a mixture of DNA and RNA. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is, in some embodiments, modified by one or more alkyl groups, e.g., one or more C₁₋₄ alkyl groups, e.g., one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N⁶—C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

### DNA

Herein, the term “DNA” relates to a nucleic acid molecule which includes deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

### RNA

The term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

According to the present disclosure, the term “RNA” includes “mRNA”. According to the present disclosure, the term “mRNA” means “messenger-RNA” and includes a “transcript” which may be generated by using a DNA template. Generally, mRNA encodes a peptide or polypeptide. mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In some embodiments, the mRNA which preferably encodes a peptide or polypeptide has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, mRNA generally contains a 5′ untranslated region (5′-UTR), a peptide/polypeptide coding region and a 3′ untranslated region (3′-UTR). In some embodiments, the mRNA is produced by in vitro transcription or chemical synthesis. In some embodiments, the mRNA is produced by in vitro transcription using a DNA template. The in vitro transcription methodology is known to the skilled person; cf., e.g., Molecular Cloning: A Laboratory Manual, 4^{th} Edition, M. R. Green and J. Sambrook eds., Cold Spring Harbor Laboratory Press, Cold Spring Harbor 2012. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified mRNA, correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA is “replicon RNA” or simply a “replicon”, in particular “self-replicating RNA” or “self-amplifying RNA”. In certain embodiments, the replicon or self-replicating RNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et a., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et a., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication (trans-amplification) systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA (in particular, mRNA) described herein contains one or more modifications, e.g., in order to increase its stability and/or increase translation efficiency and/or decrease immunogenicity and/or decrease cytotoxicity. For example, in order to increase expression of the RNA (in particular, mRNA), it may be modified within the coding region, i.e., the sequence encoding the expressed peptide or polypeptide, preferably without altering the sequence of the expressed peptide or polypeptide. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA).

In some embodiments, the RNA (in particular, mRNA) described herein comprises a 5′-cap structure. In some embodiments, the RNA does not have uncapped 5′-triphosphates. In some embodiments, the RNA (in particular, mRNA) may comprise a conventional 5′-cap and/or a 5′-cap analog. The term “conventional 5′-cap” refers to a cap structure found on the 5′-end of an RNA molecule and generally comprises a guanosine 5′-triphosphate (Gppp) which is connected via its triphosphate moiety to the 5′-end of the next nucleotide of the RNA (i.e., the guanosine is connected via a 5′ to 5′ triphosphate linkage to the rest of the RNA). The guanosine may be methylated at position N⁷ (resulting in the cap structure m⁷Gppp). The term “5′-cap analog” includes a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂₇′2° G(5′)ppSp(5′)G (referred to as beta-S-ARCA or 3-S-ARCA)), as described in PCT/EP2019/056502. Providing an RNA (in particular, mRNA) with a 5′-cap structure as described herein may be achieved by in vitro transcription of a DNA template in presence of a corresponding 5′-cap compound, wherein said 5′-cap structure is co-transcriptionally incorporated into the generated RNA (in particular, mRNA) strand, or the RNA (in particular, mRNA) may be generated, for example, by in vitro transcription, and the 5′-cap structure may be attached to the RNA post-transcriptionally using capping enzymes, for example, capping enzymes of vaccinia virus.

In some embodiments, the RNA (in particular, mRNA) comprises a 5′-cap structure selected from the group consisting of m₂₇₂G(5′)ppSp(5′)G (in particular its D1 diastereomer), m₂^{7,3′O}G(5′)ppp(5′)G, and m₂^{7,3′-O}Gppp(m₁^{2′-O})ApG.

In some embodiments, the RNA (in particular, mRNA) comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The 5′-cap analog beta-S-ARCA (P—S-ARCA) has the following structure:

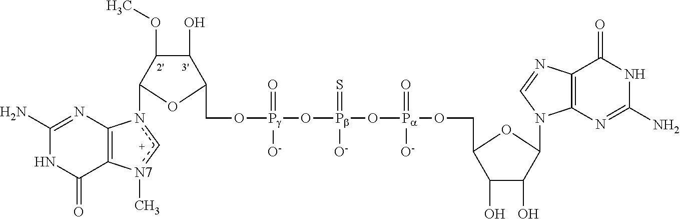

The “D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In some embodiments, a Supelcosil LC-18-T RP column, preferably of the format: 5 μm, 4.6×250 mm is used for separation, whereby a flow rate of 1.3 ml/min can be applied. In some embodiments, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp(m₁₂-)ApG (also referred to as m₂^{7,3′O}G(5′)ppp(5′)m^{2′O}-ApG) which is a building block of a cap1 has the following structure:

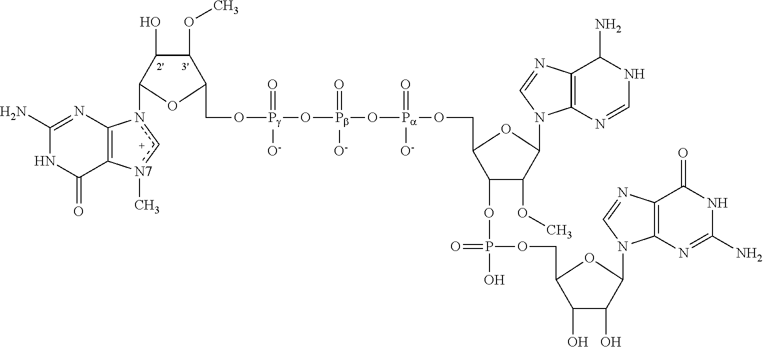

An exemplary cap0 mRNA comprising β-S-ARCA and mRNA has the following structure:

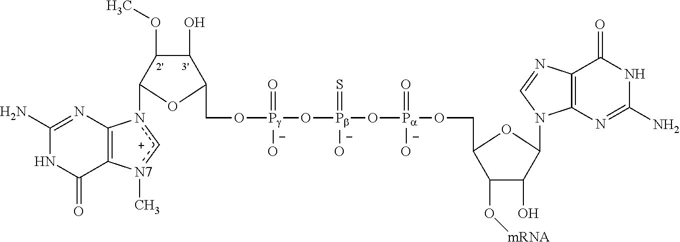

An exemplary cap0 mRNA comprising m₂^{7,3′O}G(5′)ppp(5′)G and mRNA has the following structure:

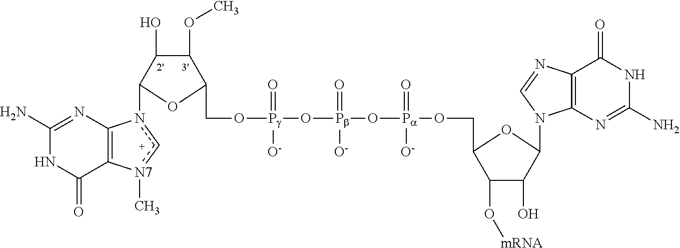

An exemplary cap1 mRNA comprising m₂^{7,3′-O}Gppp(m₁^{2′-O})ApG and mRNA has the following structure:

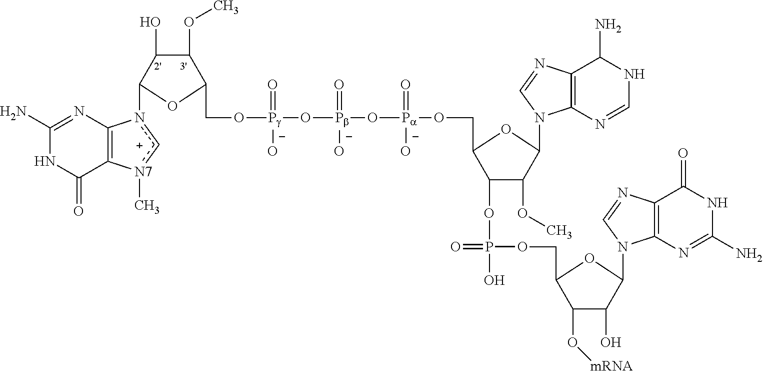

As used herein, the term “poly-A tail” or “poly-A sequence” refers to an uninterrupted or interrupted sequence of adenylate residues which is typically located at the 3′-end of an RNA (in particular, mRNA) molecule. Poly-A tails or poly-A sequences are known to those of skill in the art and may follow the 3′-UTR in the RNAs (in particular, mRNAs) described herein. An uninterrupted poly-A tail is characterized by consecutive adenylate residues. In nature, an uninterrupted poly-A tail is typical. RNAs (in particular, mRNAs) disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA (in particular, mRNA) molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, the poly(A) tail comprises 30 adenine nucleotides followed by 70 adenine nucleotides, wherein the 30 adenine nucleotides and 70 adenine nucleotides are separated by a linker sequence of 10 nucleotides.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides.

In some embodiments, RNA (in particular, mRNA) described in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does generally not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (e.g., mRNA) into which they are introduced. In certain embodiments, the 3′-UTR is derived from a globin gene or mRNA, such as a gene or mRNA of alpha2-globin, alpha1-globin, or beta-globin, e.g., beta-globin, e.g., human beta-globin. For example, the RNA (e.g., mRNA) may be modified by the replacement of the existing 3′-UTR with or the insertion of one or more, e.g., two copies of a 3′-UTR derived from a globin gene, such as alpha2-globin, alpha1-globin, beta-globin, e.g., beta-globin, e.g., human beta-globin.

In some embodiments, a 5′-UTR is or comprises a modified human alpha-globin 5′-UTR. In some embodiments, a 3′-UTR comprises a first sequence from the amino terminal enhancer of split (AES) messenger RNA and a second sequence from the mitochondrial encoded 12S ribosomal RNA.

The RNA (in particular, mRNA) described herein may have modified ribonucleotides in order to increase its stability and/or decrease immunogenicity and/or decrease cytotoxicity. For example, in some embodiments, uridine in the RNA (in particular, mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N1-methyl-pseudouridine (m1ψ), 5-methyl-uridine (m5U), and combinations thereof.

In some embodiments, the modified nucleoside replacing (partially or completely, preferably completely) uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (m1s4 ψ), 4-thio-1-methyl-pseudouridine, 3-methyl-pseudouridine (m3 ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

An RNA (preferably mRNA) which is modified by pseudouridine (replacing partially or completely, preferably completely, uridine) is referred to herein as “ψ-modified”, whereas the term “m1ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such ψ- or m1ψ- or m5U-modified RNAs usually exhibit decreased immunogenicity compared to their unmodified forms and, thus, are preferred in applications where the induction of an immune response is to be avoided or minimized. In some embodiments, the RNA (preferably mRNA) contains N(1)-methylpseudouridine replacing completely uridine.

The codons of the RNA (in particular, mRNA) described in the present disclosure may further be optimized, e.g., to increase the GC content of the RNA and/or to replace codons which are rare in the cell (or subject) in which the peptide or polypeptide of interest is to be expressed by codons which are synonymous frequent codons in said cell (or subject). In some embodiments, the amino acid sequence encoded by the RNA (in particular, mRNA) described in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions may be codon-optimized for optimal expression in a subject to be treated using the RNA (in particular, mRNA) described herein. Codon-optimization is based on the finding that the translation efficiency is also determined by a different frequency in the occurrence of tRNAs in cells. Thus, the sequence of RNA (in particular, mRNA) may be modified such that codons for which frequently occurring tRNAs are available are inserted in place of “rare codons”.

In some embodiments, the guanosine/cytosine (G/C) content of the coding region of the RNA (in particular, mRNA) described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that RNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA (in particular, mRNA) described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (ψ) or N(1)-methylpseudouridine (m1ψ) or 5-methyluridine (m5U) for uridine), and codon optimization, has a synergistic influence on the stability of RNA (preferably mRNA) and increase in translation efficiency. Thus, in some embodiments, the RNA (in particular, mRNA) described in the present disclosure contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (ψ) or N(1)-methylpseudouridine (m1ψ) or 5-methyluridine (m5U) for uridine), and (v) codon optimization.

### Pharmaceutical Compositions

The agents described herein may be administered in pharmaceutical compositions and may be administered in the form of any suitable pharmaceutical composition.

In some embodiments, the agents described herein may be administered in a pharmaceutical composition which may comprise a pharmaceutically acceptable carrier and may optionally comprise one or more stabilizers etc. In some embodiments, the pharmaceutical composition is for therapeutic or prophylactic treatments, e.g., for use in treating or preventing a disease.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses and/or agents. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline. Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In some embodiments, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical composition is formulated for systemic administration, e.g., for intravenous administration.

### Treatments

The agents, compositions and methods described herein can be used to treat a subject with a disease, e.g., a disease characterized by the presence of diseased cells expressing an antigen. The agents, compositions and methods described herein may be used in the therapeutic or prophylactic treatment of various diseases. Particularly preferred diseases are cancer diseases. In some embodiments, the agents, compositions and methods described herein are useful in a prophylactic and/or therapeutic treatment of a disease involving an antigen.

Such antigen may serve as primary target or as target for immune effector cells genetically modified to express an antigen receptor. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to deliver a pharmaceutically active nucleic acid to diseased cells expressing an antigen such as cancer cells expressing a tumor antigen, and to treat a disease such as a cancer disease involving cells expressing an antigen such as a tumor antigen.

In some embodiments of the disclosure, the aim is to deliver a nucleic acid encoding an antigen receptor to immune effector cells to generate immune effector cells genetically modified to express the antigen receptor. In some embodiments, immune effector cells genetically modified to express an antigen receptor are for targeting diseased cells expressing an antigen such as cancer cells expressing a tumor antigen, and treating a disease such as a cancer disease involving cells expressing an antigen such as a tumor antigen. In some embodiments, immune effector cells expressing an antigen receptor exert one or more immune effector functions on diseased cells, e.g., kill diseased cells by means of a cellular immune response.

The term “disease involving an antigen”, “disease involving cells expressing an antigen” or similar terms refer to any disease which implicates an antigen, e.g. a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In some embodiments, a disease involving an antigen is a disease involving cells expressing an antigen, preferably on the cell surface.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

The term “solid tumor” or “solid cancer” as used herein refers to the manifestation of a cancerous mass, as is well known in the art for example in Harrison's Principles of Internal Medicine, 14th edition. Preferably, the term refers to a cancer or carcinoma of body tissues other than blood, preferably other than blood, bone marrow, and lymphoid system. For example, but not by way of limitation, solid tumors include cancers of the prostate, lung cancer, colorectal tissue, bladder, oropharyngeal/laryngeal tissue, kidney, breast, endometrium, ovary, cervix, stomach, pancrease, brain, and central nervous system.

The methods and agents described herein are, in particular, useful for the treatment of cancers, e.g., solid cancers, characterized by diseased cells expressing an antigen a targeting compound, docking compound or antigen receptor is directed to.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naïve T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages. The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Formulation and Characterization of Functionalized Lipid Nanoparticles (LNPs) Containing RNA

LNPs were formulated with varying mol % of Alfa tag lipid (Lipid mix: DODMA/Chol/DOPE/C16-PEG-Ceramide/DSPE-PEG2k-Alfa 40/48/10/2-x/x=0, 0.2, 0.5, 1; N/P ratio: 4; Cargo: Thy1.1/Luc RNA 1:1 w/w; RNA concentration: 0.1 μg/μl). aCD8-DARPin X NbAlfa was added via post-functionalization [w/w*=ligand to cargo ratio 0.55;). Successful RNA incorporation was verified via Agarose gel electrophoresis. Size and PDI were measured via dynamic light scattering at 0.005 g/L RNA concentration for LNPs with or without ligand. ** No PDI measurable.

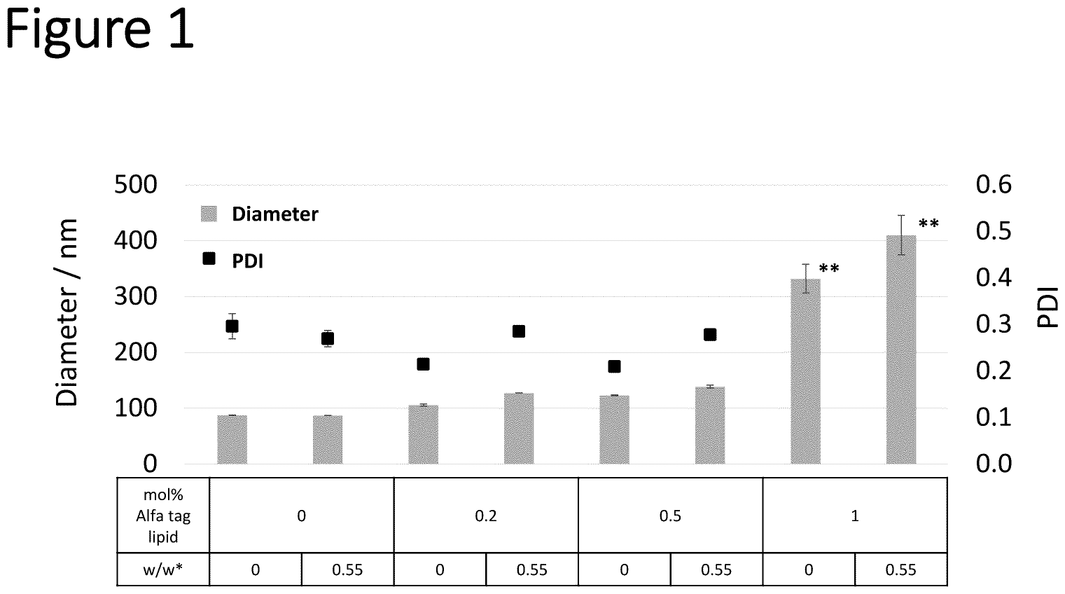

As shown in FIG. 1, the formation of colloidal stable particles is observed for inclusion of up to 0.5 mol % of Alfa tag lipid in the LNPs. Addition of ligand leads to a slight increase in size and PDI. Inclusion of 1 mol % of Alfa tag lipid in the LNP leads to large and multimodal particles.

### Example 2: T-Cell Targeting Using LNPs Decorated with aCD3 Ligand

LNPs were formulated with or without ligand (Cargo: Thy1.1/Luc RNA 1:1 w/w; N/P ratio: 4; Lipid mix: DODMA/Cholesterol/DOPE/C16 PEG2k Ceramide/DSPE-PEG2k-Alfa [40/48/10/1.8/0.2]; aCD3-VHH×NbAlfa post-functionalization [w/w*=ligand to cargo ratio 1.15]; RNA concentration: 0.1 μg/μl). Successful RNA incorporation was verified via Agarose gel electrophoresis. For transfection studies, 15 μl of respective formulations were pre-diluted in 50 μl X-Vivo 15 in an ultra-low adhesion 96 well plate. 1e6 thawed human PBMC were diluted in 50 μl X-Vivo 15 (with 10% PHS) and added to nanoparticle dilution (1500 ng RNA). After 30 min of incubation (37° C., 5% CO2) 200 μl of X-Vivo15+5% PHS was added per well and cells were cultivated for additional 18 h (37° C., 5% CO2). In the following cell-type specific transfection (Thy1.1) and T cell activation (CD69) were analyzed via flowcytometry. (A) LNP characteristics. Diameter and PDI were determined via DLS measurement. (B) Depicted are the cell type specific Thy1.1 (Transfection, y-axes) signals within viable CD14+ Monocytes, CD56+NK cells, CD19+ B cells, CD3+ T cells, CD4+ T cells or CD8+ T cells. (C) Histograms show CD69 activation marker expression on the T cell population within the treated PBMCs.

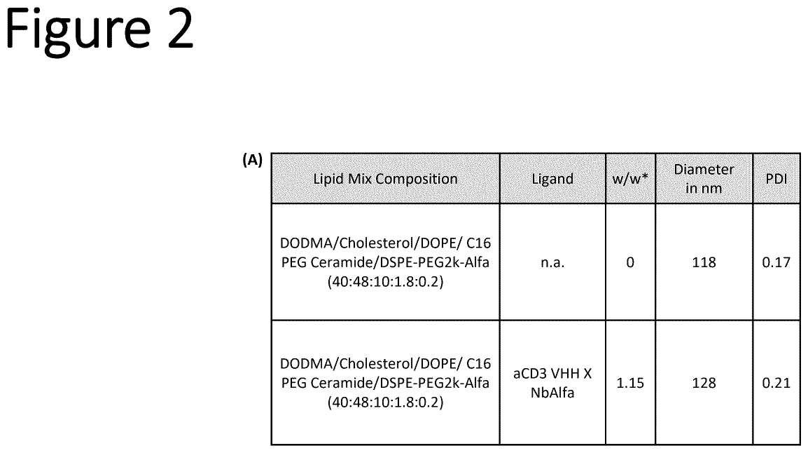

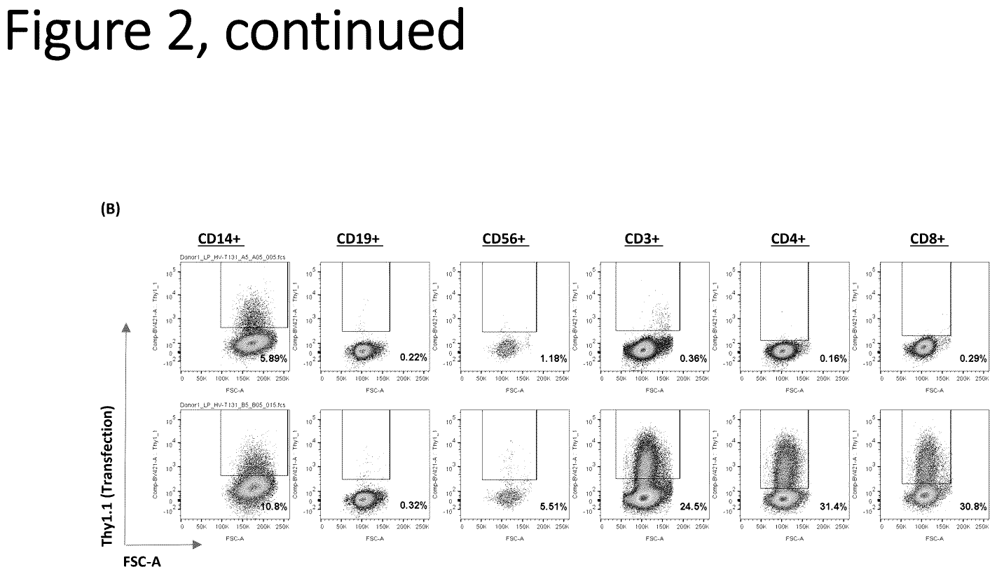

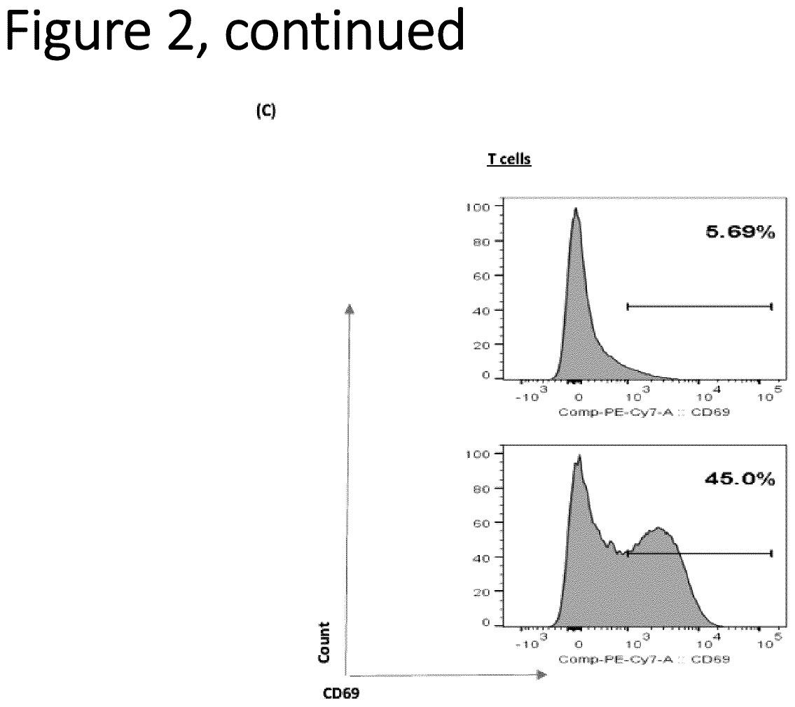

As shown in FIG. 2, LNPs without ligand decoration showed a diameter of 118 nm at a PDI of 0.17 (see in A). Treatment of PBMCs with this formulation resulted in exclusive and low transfection of monocytes (5%) (see in B). In line with these findings, T cell did not show any CD69 expression (see in C) indicating no T cell activation in response to unfunctionalized LNP treatment. Upon post-functionalization with the aCD3-VHH X NbAlfa ligand, the size of the corresponding LNPs slightly increased to 128 nm in diameter at a PDI of 0.21 (see in A). Also the functionalized LNPs showed a low transfection rate of monocytes, however the transfection efficiency in T cells was strongly increased by the ligand-decoration (see in B). 30% of all CD4+ and CD8+ T cells could be transfected via the ahCD3-LNP treatment. In addition, the treatment of PBMCs with ligand-decorated LNPs resulted in strong T cell activation, indicated by CD69 upregulation (see in C).

### Example 3: T-Cell Targeting Using LPLX Decorated with aCD8 DARPin

LPLX were functionalized with two different ligands: aCD8 DARPin X Spycatcher or aCD8 DARPin X NbAlfa. LPLX contain Viromer L3 for RNA encapsulation and a functionalized lipid (Cargo: Thy1.1/Luc RNA 1:1 w/w; RNA concentration: 0.1 μg/μL; N/P ratio: 7.5; ligand to cargo w/w ratio 0.25 for aCD8 DAPRin X Spycatcher and 0.1 for aCD8 DARPin X NbAlfa; molar ratio ligand to functionalized lipid 1.5:1 for aCD8 DAPRin X Spycatcher/Spy tag PEG2k-DSPE and 3.5:1 for aCD8 DARPin X NbAlfa/Alfa tag PEG2k-DSPE). Diameter and PDI were determined via DLS measurement. Successful RNA incorporation was verified via Agarose gel electrophoresis. For transfection studies, 0.5 or 0.1 μl of respective formulations were pre-diluted in 50 μl X-Vivo 15 in a 96 well plate. 1e6 thawed human T-cells were diluted in 50 μl X-Vivo 15 and added to nanoparticle dilution (50 or 100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Depicted are Thy1.1 (Transfection, y-axes) signals within viable CD4+ T cells or CD8+ T cells.

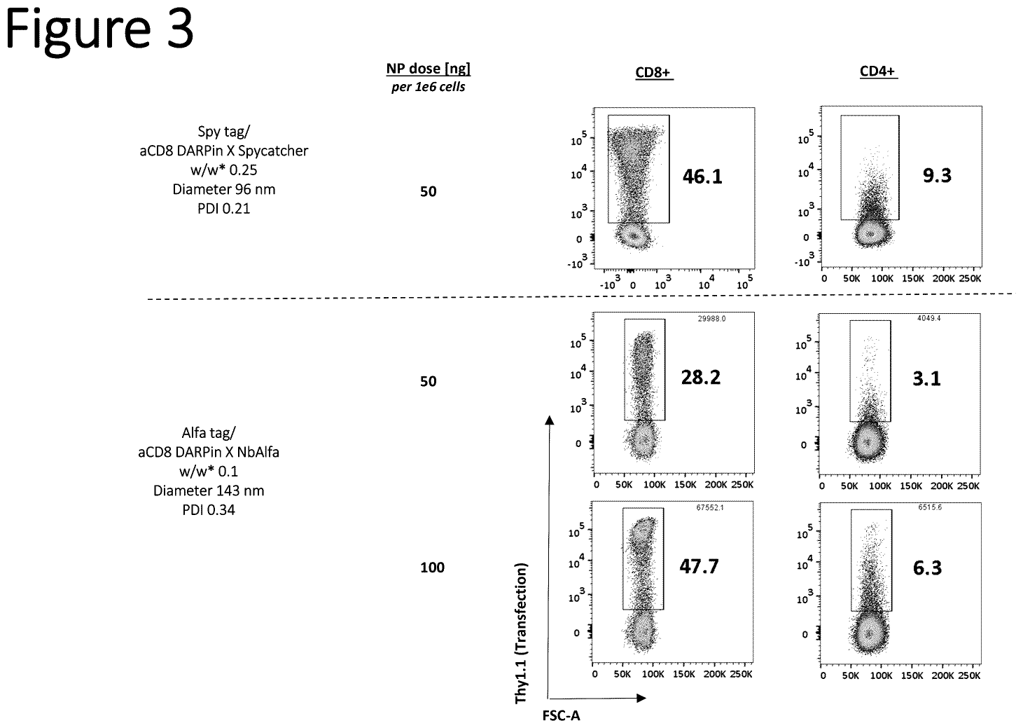

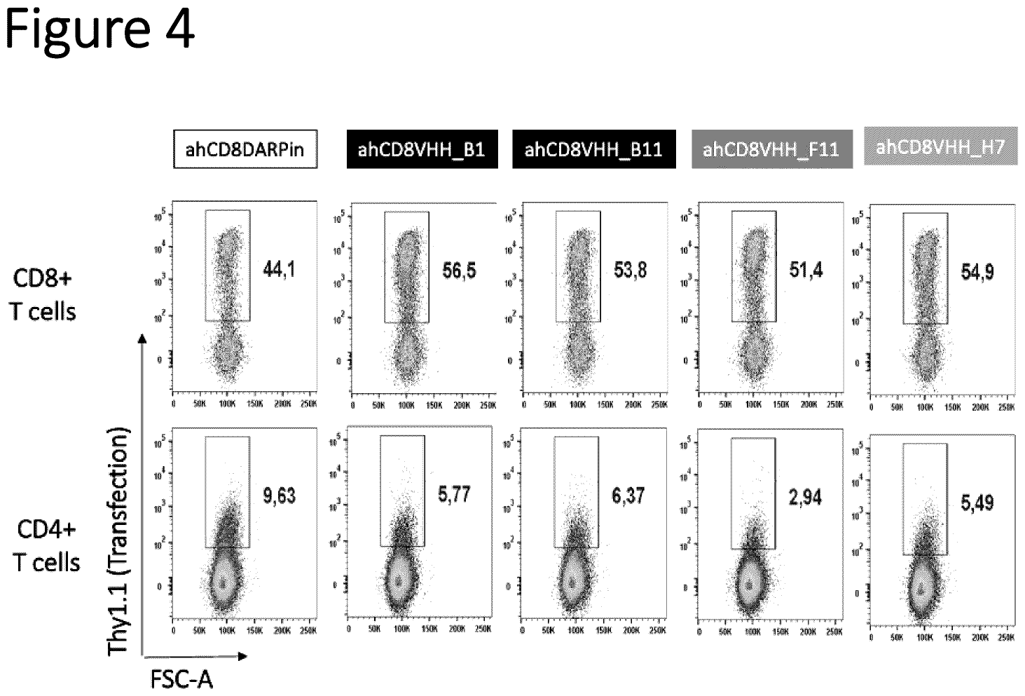

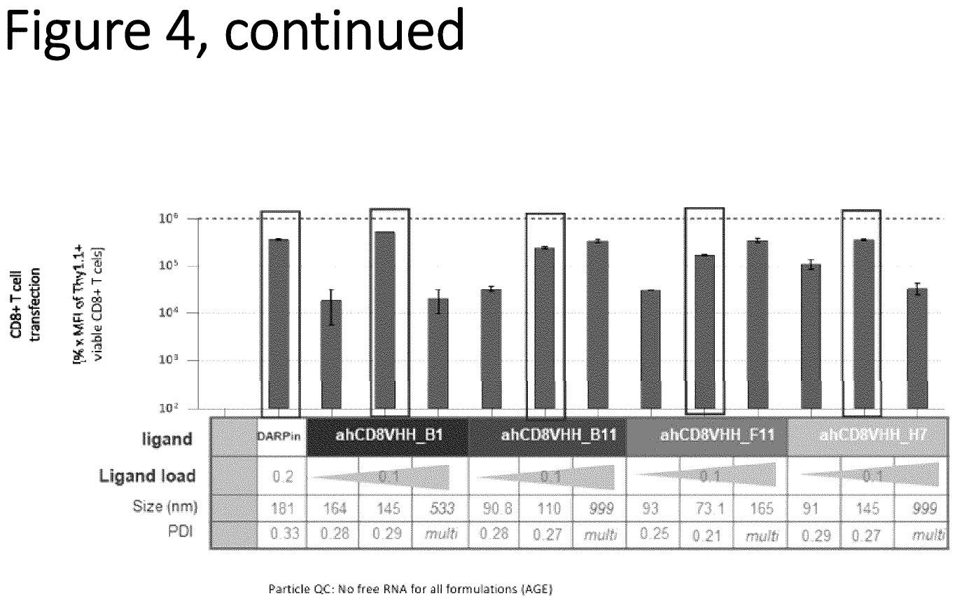

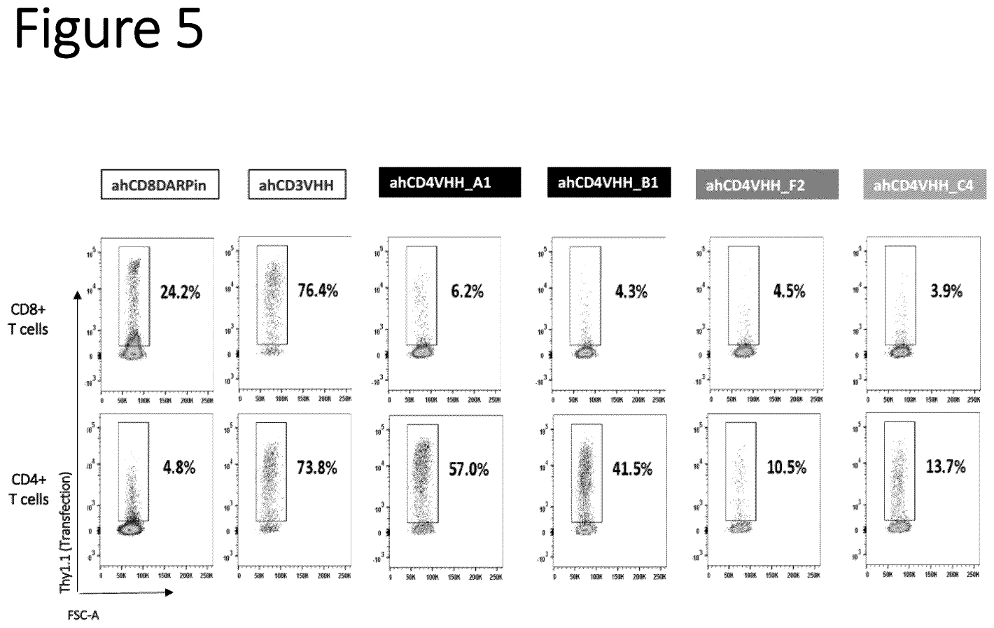

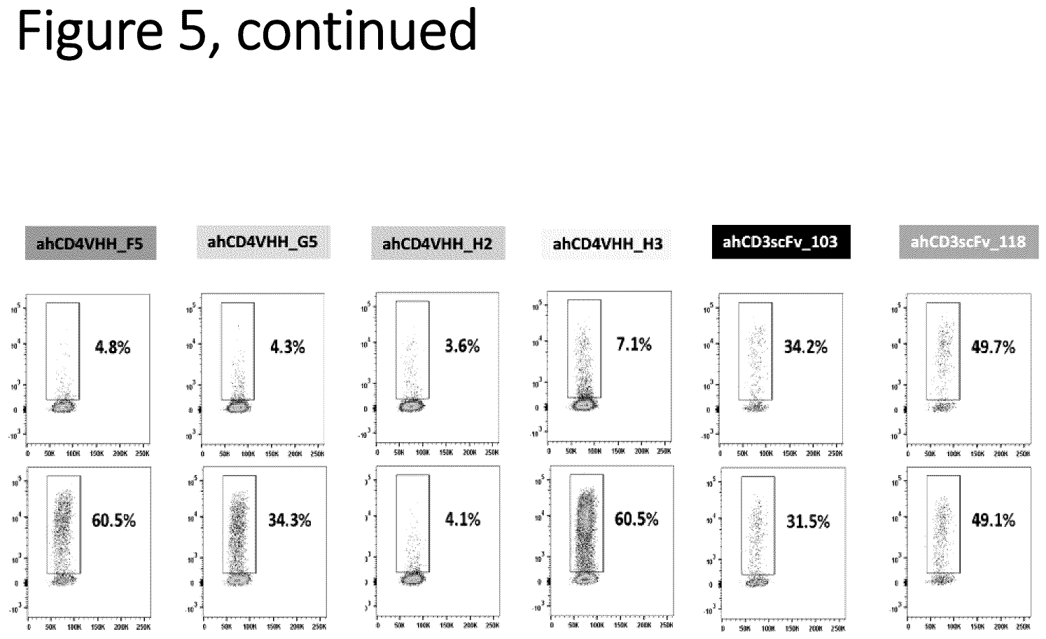

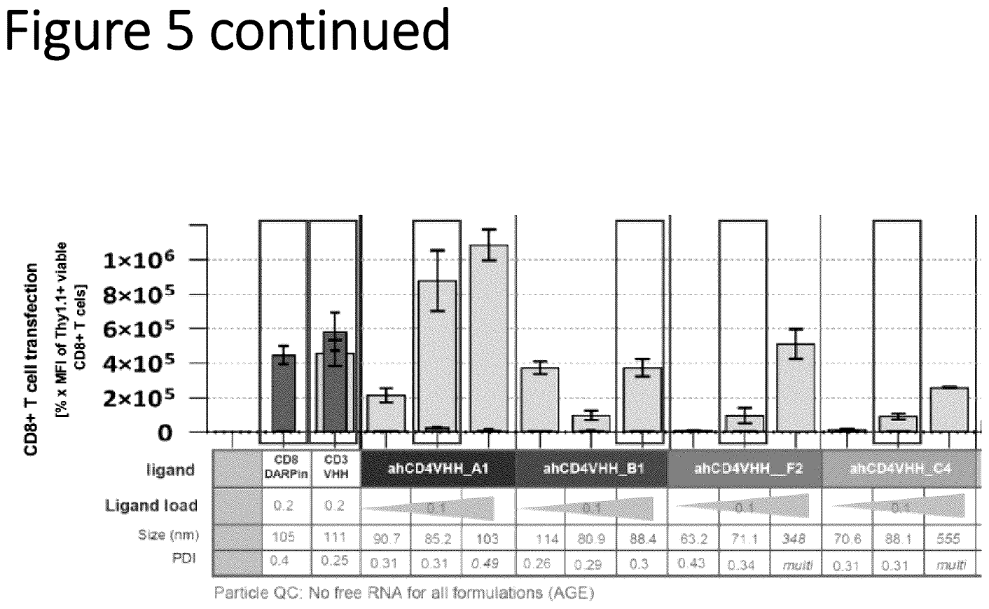

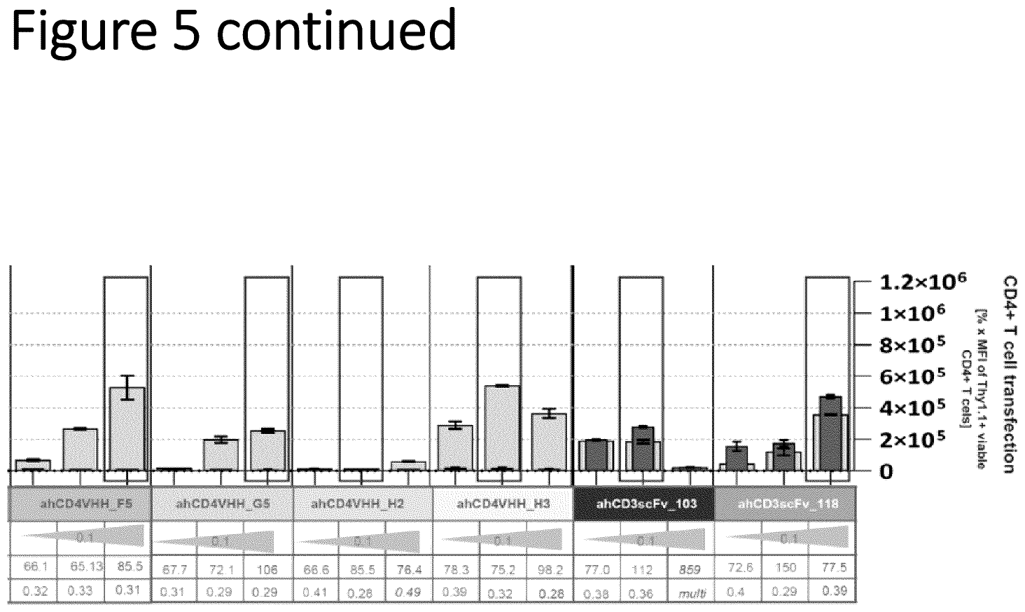

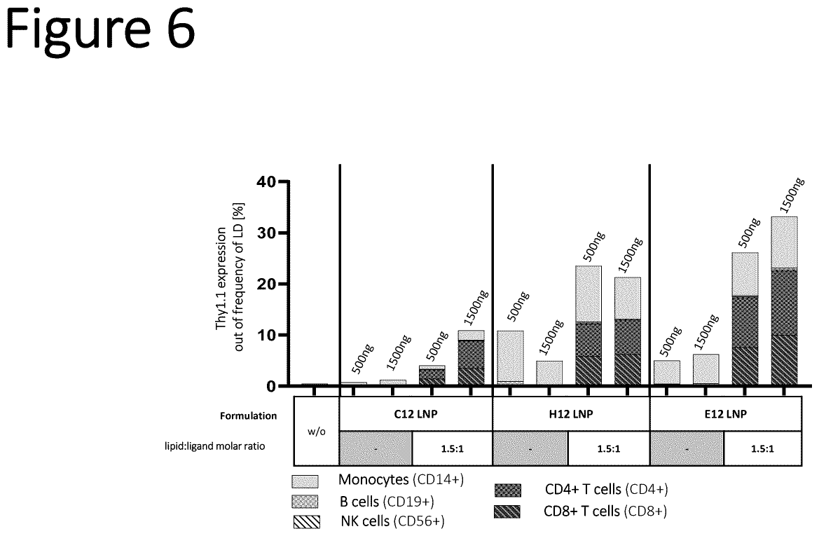

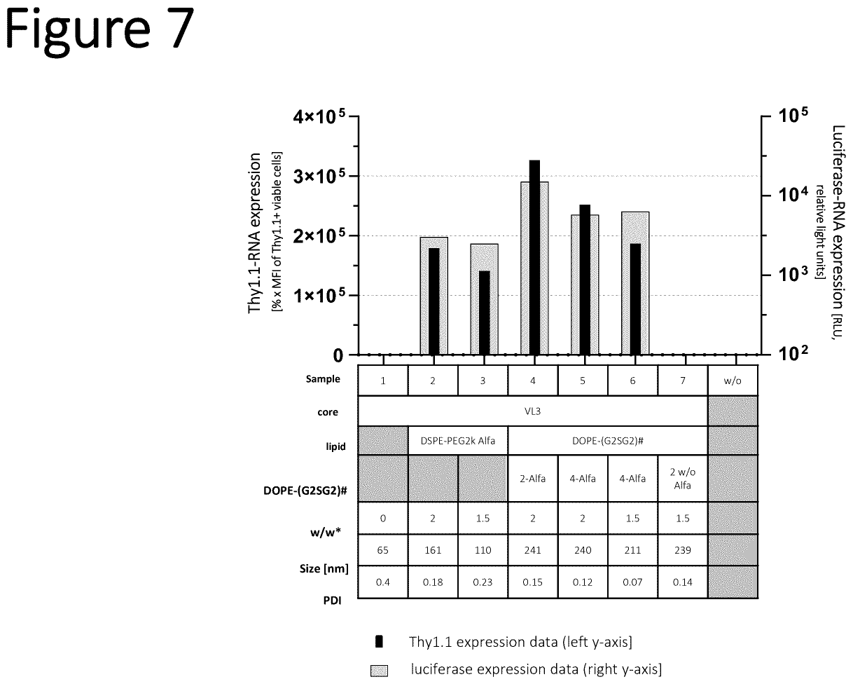

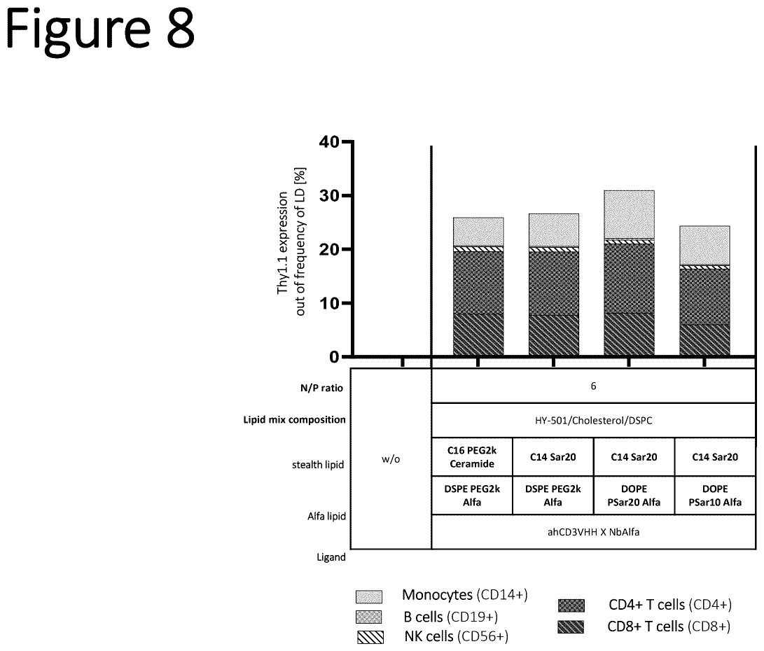

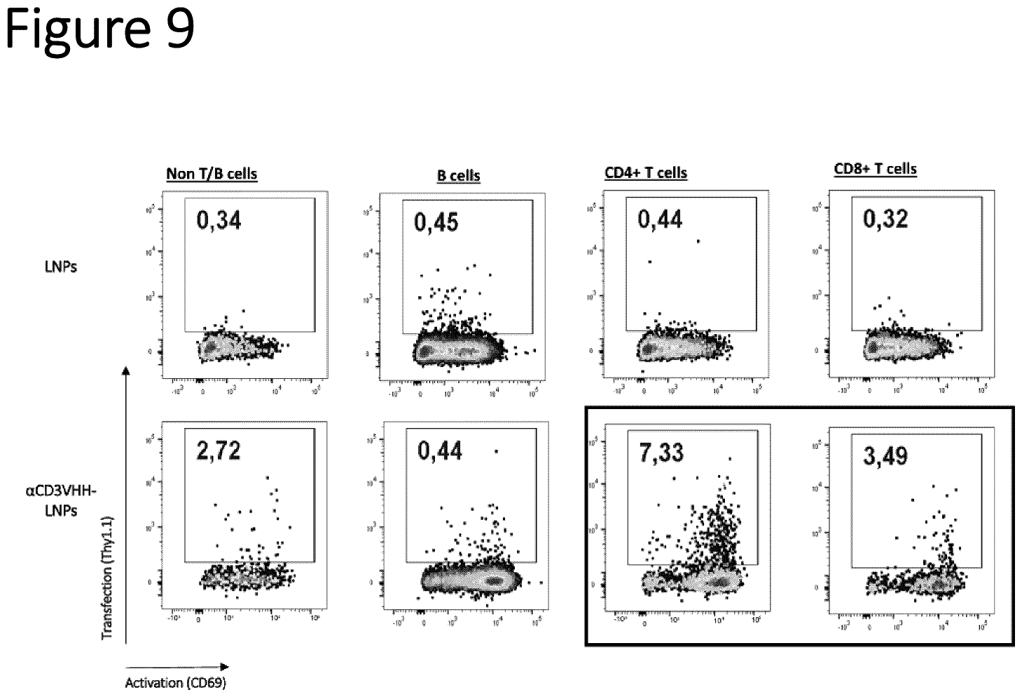

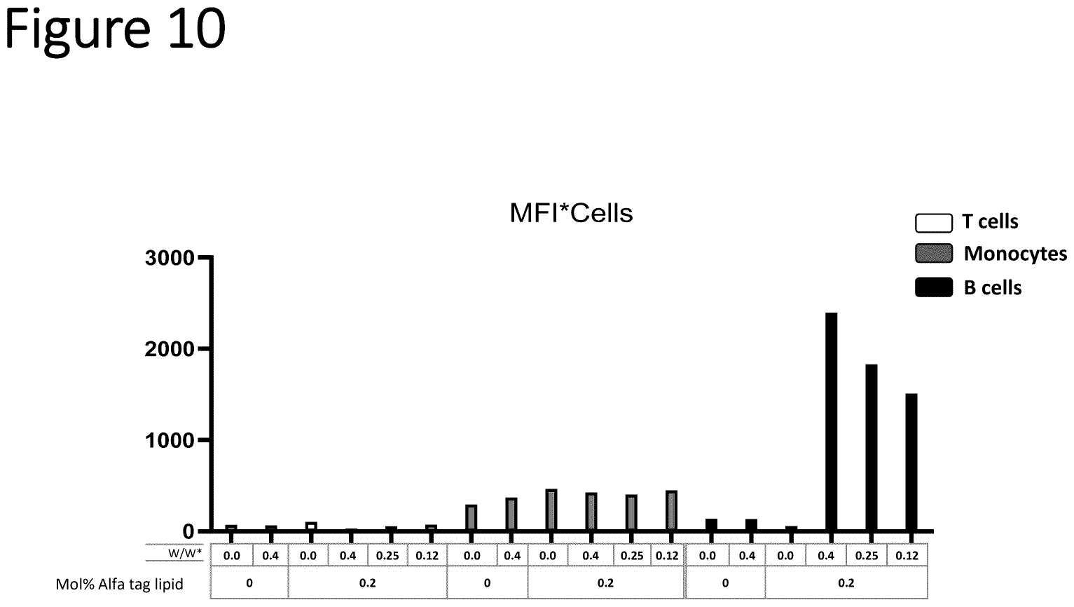

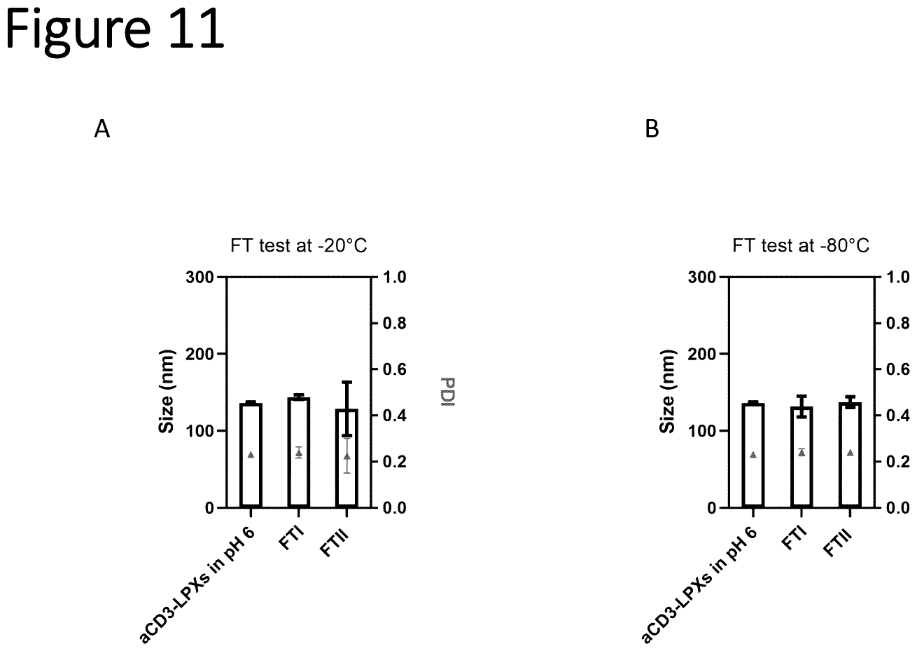

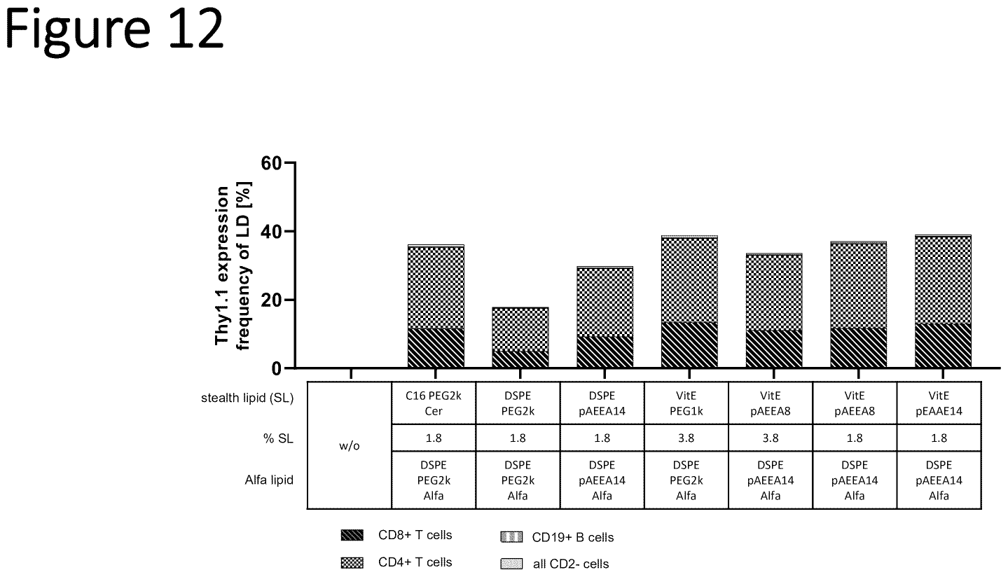

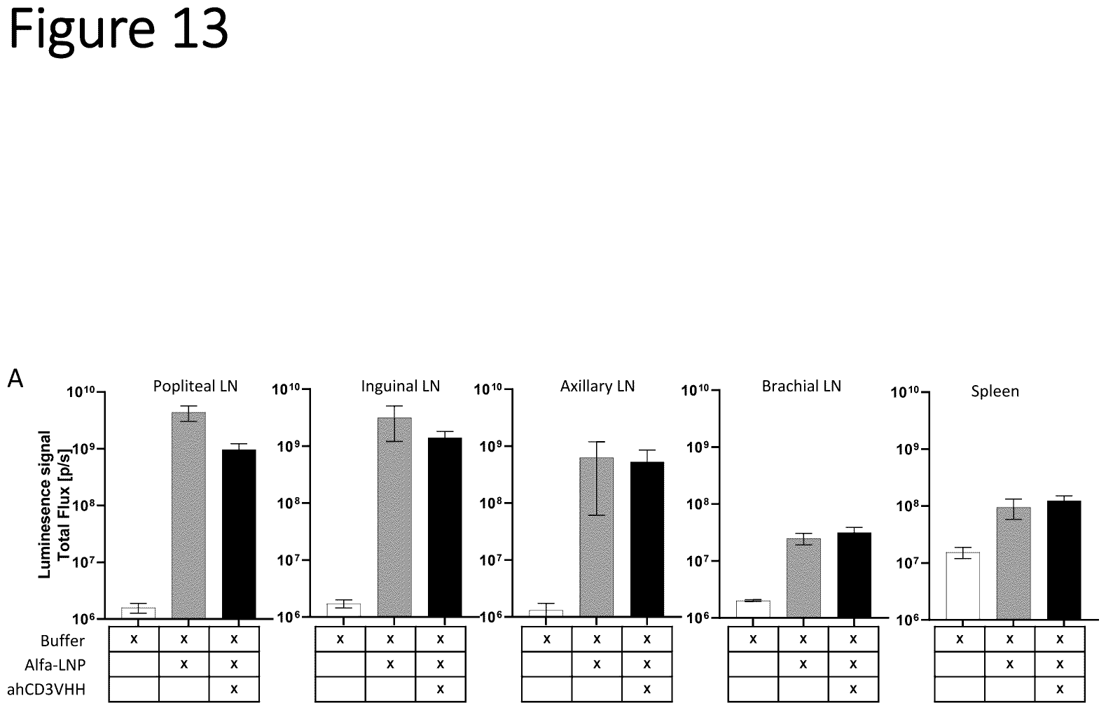

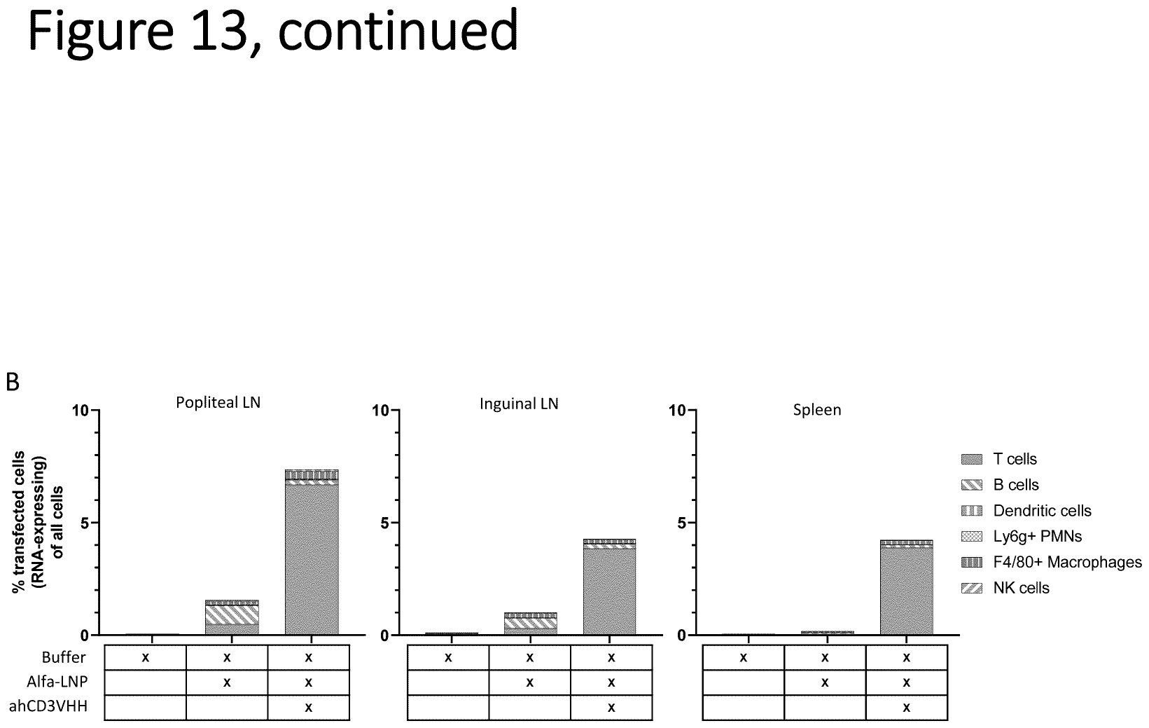

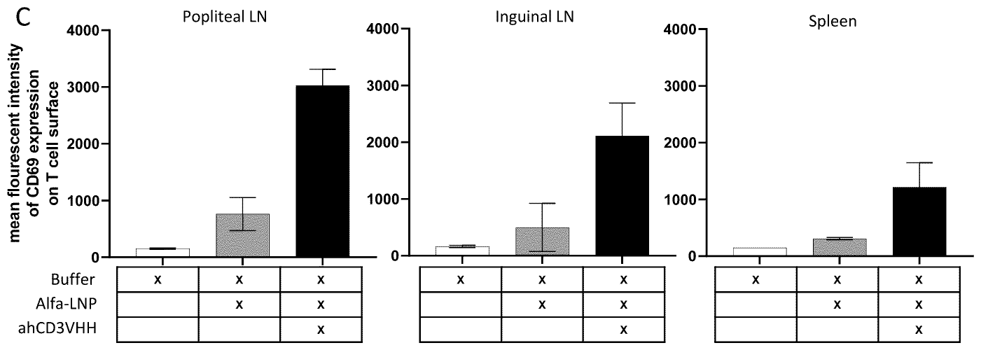

As shown in FIG. 3, LPLX can be functionalized with ligands via two different functionalization strategies resulting in sub 200 nm particles with PDI below 0.5. Both strategies are based on a peptide tag attached to a lipid. The incorporation of functionalized lipids into the formulation is based on the interaction with hydrophobic parts of Viromer. Strong targeted transfection of CD8+ T-cells can be observed for both particles with only minor transfection in CD4+ T-cells (off target). Off note, LPLX functionalized with ahCD8DARPin X Nb Alfa require an increased nanoparticle dose (2 fold) to reach similar transfection rates as LPLX ahCD8DARpin X Spycatcher nanoparticles.

### Example 4: T-Cell Targeting Using LPLX Decorated with Different Ligands

LPLX were functionalized with fusion proteins comprised of a CD8 targeting ligand fused to X NbAlfa. The CD8 targeting domain is either a DARPin or a VHH nanobody. One DARPin and four different nanobodies were tested. LPLX contain Viromer L3 for RNA encapsulation and DSPE-PEG2k Alfa tag for ligand conjugation (Cargo: Thy1.1/Luc RNA 1:1 w/w; RNA concentration: 0.1 μg/μL; N/P ratio: 7.5; ligand to cargo w/w ratio 0.05, 0.1 and 0.2 for aCD8 VHH X NbAlfa; ligand to cargo w/w ratio 0.2 for aCD8 DARPin×NbAlfa; molar ratio ligand to functionalized lipid 3.5:1). Diameter and PDI were determined via DLS measurement. Successful RNA incorporation was verified via Agarose gel electrophoresis. For transfection studies, 0.1 μl of respective formulations were pre-diluted in 50 μl X-Vivo 15 in a 96 well plate. 1e6 thawed human T-cells were diluted in 50 μl X-Vivo 15 and added to nanoparticle dilution (100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Middle graph shows particle condition (see lower table) specific CD8+ T cell transfection efficiency calculated via multiplying the percentage of transfected (Thy1.1+) of CD8+ T cells with the MFI of the Thy1.1+CD8+ cell population. Dot blots corresponding to conditions in the graph marker with a box are depicted in the upper part of the figure. These blots show Thy1.1 signals within viable CD8+ T cells (upper blots) or viable CD4+ T cells (lower blots).

As shown in FIG. 4, LPLX can be functionalized with various ligands via the Alfa tag/NbAlfa functionalization. Formulation conditions resulting in sub 200 nm particles with PDI below 0.5 can be identified for all ligands. Independent from the ligand format, strong and selective transfection of CD8+ T-cells can be observed for all particles demonstrating the modular nature of the selected conjugation strategy.

### Example 5: T-Cell Targeting Using LPLX Decorated with Different Ligands

LPLX were functionalized with fusion proteins comprised of a CD8, CD4 or CD3 targeting ligand fused to X NbAlfa. The targeting domain is either a DARPin, a VHH nanobody or a scFv. One DARPin, nine different nanobodies and two different scFvs were tested. LPLX contain Viromer L3 for RNA encapsulation and DSPE-PEG2k Alfa tag for ligand conjugation (Cargo: Thy1.1/Luc RNA 1:1 w/w; RNA concentration: 0.1 μg/μL; N/P ratio: 7.5; ligand to cargo w/w ratio 0.05, 0.1 and 0.2 for aCD4 VHH X NbAlfa and aCD3 scFv×NbAlfa; ligand to cargo w/w ratio 0.2 for aCD8 DARPin X NbAlfa and aCD3 VHH X NbAlfa; molar ratio ligand to functionalized lipid 3.5:1). Diameter and PDI were determined via DLS measurement. Successful RNA incorporation was verified via Agarose gel electrophoresis. For transfection studies, 0.1 μl of respective formulations were pre-diluted in 50 μl X-Vivo 15 in a 96 well plate. 1e6 thawed human T-cells were diluted in 50 μl X-Vivo 15 and added to nanoparticle dilution (100 ng RNA). After 30 min of incubation (37° C., 5% CO2) 200 μl of X-Vivo15+5% PHS was added per well and cells were cultivated for additional 18 h (37° C., 5% CO2). In the following CD4 and CD8 specific transfection (Thy1.1) was analyzed via flowcytometry. Middle Graph shows particle condition (see lower table) specific CD8+(left y-axis; dark grey bars) and CD4+(right y-axis; light grey bars) T cell transfection efficiency. Values are calculated via multiplying the percentage of transfected (Thy1.1+) of CD8 or CD4+ T cells with the MFI of the respective Thy1.1+ cell population. Dot blots corresponding to conditions in the graph marker with a box are depicted in the upper part of the figure. These blots show Thy1.1 signals within viable CD8+ T cells (upper blots) or viable CD4+ T cells (lower blots).

As shown in FIG. 5, LPLX can be functionalized with various ligands via the Alfa tag/NbAlfa functionalization. Formulation conditions resulting in sub 200 nm particles with PDI below 0.5 can be identified for all ligands. Except for two ligand candidates (αhCD4VHH—F2 and αhCD4VHH—H2) all other ligands successfully directed the transfection potential of the LPLX nanoparticles towards the targeted T cell population. Thus, ahCD3VHH-LPLX showed strong transfection of both CD4+ and CD8+ human T cells while LPLX functionalization with ahCD4VHH-ligand candidates let to selective CD4+ T cell transfection. Also, αhCD3scFv ligand scaffolds could be successfully used to drive CD4 and CD8+ T cell transfection of LPLX nanoparticles, however the transfection efficiency was higher for αhCD3VHH-LPLXs. Taken together these findings demonstrate the modular nature (in terms of ligand scaffold and ligand specificity) of the selected conjugation strategy.

### Example 6: T-Cell Targeting Using LNPs with Different Lipid Compositions

LNPs were formulated with different lipid compositions and equipped with aCD3 VHH X NbAlfa ligand (Cargo: Thy1.1/Luc RNA 1:1 w/w; N/P ratio: 4; Lipid mix: Ionizable lipid/Cholesterol/DOPE/C16 PEG2k Ceramide/DSPE-PEG2k-Alfa [40/48/10/1.8/0.2] with DODMA (C12), HY-501 (H12) or EA-405 (E12) as ionizable lipid; aCD3-VHH X NbAlfa post-functionalization [w/w*=ligand to cargo ratio 1.15; molar ratio ligand to functionalized lipid 1.5:1]; RNA concentration: 0.1 μg/μl). Diameter of all LNPs is below 120 nm with a PDI below 0.2 as determined via DLS measurement. Successful RNA incorporation was verified via Agarose gel electrophoresis. For transfection studies, 5 or 15 μl (500 ng or 1500 ng dose) of respective formulations were pre-diluted in 50 μl X-Vivo 15 in an ultra-low adhesion 96 well plate. 1e6 thawed human PBMC were diluted in 50 μl X-Vivo 15 (with 10% PHS) and added to nanoparticle dilution. After 30 min of incubation (37° C., 5% CO2) 200 μl of X-Vivo15+5% PHS was added per well and cells were cultivated for additional 18 h (37° C., 5% C02). In the following cell-type specific transfection (Thy1.1) was analyzed via flowcytometry. Depicted are the percentages of transfected cell (CD14+ Monocytes, CD56+NK cells, CD19+ B cells, CD3+ T cells, CD4+ T cells or CD8+ T cells) within all transfected PBMCs (Transfection, y-axes) per tested formulation condition (see lower table).

As shown in FIG. 6, treatment of PBMCs with LNPs without ligand resulted in almost no transfection of T cells. Upon post-functionalization with the aCD3-VHH X NbAlfa ligand, the transfection efficiency in T cells was strongly increased, independent of the lipid mix used for the LNP formulation. Via the selection of the ionizable lipid, the T cell transfection efficiency can be highly improved.

### Example 7: T-Cell Targeting Using LPLX with Different Lipids and Linkers

LPLX were functionalized with aCD8 DARPin X NbAlfa using three different Alfa tag lipids as well as one control lipid without Alfa tag: DSPE-PEG2k Alfa, DOPE-(G2SG2)2 Alfa, DOPE-(G2SG2)4 Alfa and DOPE-(G2SG2)2. All LPLX contain Viromer L3 for RNA encapsulation (Cargo: Thy1.1/Luc RNA 1:1 w/w; RNA concentration: 0.1 μg/μL; N/P ratio: 15; ligand to cargo w/w ratio 0, 1.5 or 2; molar ratio ligand to functionalized lipid 1.5:1). Diameter and PDI were determined via DLS measurement. Successful RNA incorporation was verified via Agarose gel electrophoresis. For transfection studies, 1e6 CD8+ Jurkat cells were seeded per well (96 deep well plate) in 100 μl RMPI+10% FBS. 3 μl of respective formulations (300 ng RNA) were applied per well followed by resuspension of NP-cell solution and incubation for 10 min at 37° C. Afterwards 900 μl of RPMI+10% FBS were added per well to reach a cell concentration of 1e6 cells/ml. 0.1e6 cells in 100 μl were seeded for luciferase-expression readout on day 1. 0.3e6 cells in 300 μl were seeded for FACs-based detection of Thy1.1 expression on day 1.

As shown in FIG. 7, ligands can be attached to nanoparticles using tag lipids with varying lipid backbone as demonstrated for LPLX using DSPE or DOPE as lipid backbone. Additionally, the spacer between the lipid and the tag can be varied as shown for PEG2k as well as for the peptide spacer G2SG2 (two or four repeating units). Formulation conditions resulted in colloidal stable particles with PDI below 0.5. All formulations containing Alfa tag lipid and ligand result in increased transfection efficiency of CD8+ Jurkat cells. The control particle without lipid (sample 1) as well as with lipid but without tag (sample 7) does not lead to targeted transfection efficiency.

### Example 8: T-Cell Targeting Using LNP with Different Stealth Lipids and Alfa-Lipids

LNPs were formulated with different lipid compositions (Cargo: Thy1.1/Luc RNA 1:1 w/w; N/P ratio: 4; Lipid mix: HY501/Cholesterol/DSPC/stealth lipid/Alfa lipid with C16 PEG2k Ceramide [47.5/40.5/10/1.8/0.2] or C14 Sar20 [47.5/38/10/4.3/0.2] as stealth lipid and DSPE PEG2k Alfa, DOPE PSar20 or Alfa DOPE PSar10 Alfa. The LNPs were equipped with aCD3 VHH X NbAlfa ligand via post functionalization [w/w*=ligand to cargo ratio 0.48; molar ratio ligand to functionalized lipid 3.5:1]; RNA concentration: 0.1 μg/μl). Diameter of all LNPs is between 90 to 120 nm with a PDI below 0.3 as determined via DLS measurement. Successful RNA incorporation was verified via Agarose gel electrophoresis. For transfection studies, 10 μl (1000 ng dose) of respective formulations were pre-diluted in 50 μl X-Vivo 15 in an ultra-low adhesion 96 well plate. 1e6 thawed human PBMC were diluted in 100% PHS and added to nanoparticle dilution. Plates were incubated over night (37° C., 5% CO2). In the following cell-type specific transfection (Thy1.1) was analyzed via flowcytometry. Depicted are the percentages of transfected cell (CD14+ Monocytes, CD56+NK cells, CD19+ B cells, CD3+ T cells, CD4+ T cells or CD8+ T cells) within all transfected PBMCs (Transfection, y-axes) per tested formulation condition (see lower table).

As shown in FIG. 8, the experiment demonstrates the modular nature of the conjugation approach. As shown for LNPs, ligands can be attached to nanoparticles using Alfa lipids with varying lipid backbone and spacer type as well as spacer length. Both stealth lipid and Alfa-lipid can be altered in the LNP formulation without altering the physicochemical properties of the nanoparticles (size, PDI and RNA encapsulation) and without reduction of T cell targeting efficiency.

### Example 9: T-Cell Targeting Using LNP in Transgenic Mouse Model

LNPs were formulated with different lipid compositions and equipped with aCD3 VHH X NbAlfa ligand (Cargo: Thy1.1/Luc RNA 1:1 w/w; N/P ratio: 4; Lipid mix: DODMA/Cholesterol/DOPE/C16 PEG2k Ceramide/DSPE-PEG2k-Alfa [40/48/10/1.8/0.2]. aCD3-VHH X NbAlfa was added via post functionalization [w/w*=ligand to cargo ratio 1.15; molar ratio ligand to functionalized lipid 1.5:1]; RNA concentration: 0.1 μg/μl). Diameter of all LNPs is between 90 to 120 nm with a PDI below 0.3 as determined via DLS measurement. Successful RNA incorporation was verified via Agarose gel electrophoresis. 20 μg of the respective formulation was injected i.v. in the tail vain of B6-hCD3EDG transgenic mice. Prior to the injection the transgenic human CD3 expression on mouse T cells and the specific binding of the anti-hCD3VHH X NbAlfa ligand to the transgenic receptor was verified in vitro on B6-hCD3EDG splenocytes. 18 h after LNP injection animals were sacrificed and splenocytes were for cell-type specific Thy1.1 expression via flowcytometry. Depicted are Thy1.1 (Transfection, y-axes) signals against CD69 activation marker expression on non T/B cells, mCD19+ B cells and mCD4+ or mCD8+ T cells.

As shown in FIG. 9, injection of aCD3VHH-LNPs lead to specific hCD3EDG transgenic mouse T cell transfection (both CD4+ and CD8+ mouse T cells). In line with transfection T cells were activated, indicated by CD69 upregulation. Both effects were aCD3 VHH X NbAlfa ligand driven since it could not be observed following injection of LNPs without ligand modification. Taken together the data indicate that αhCD3VHH-LNPs are stable and transfect in a ligand-specific manner T cells in vivo.

### Example 10: B-Cell Targeting Using LNP with algM

LNP were formulated with or without ligand (Cargo: Thy1.1/Luc RNA 1:1 w/w; N/P ratio: 4; Lipid mix: DODMA/Chol/DOPE/C16-PEG-Ceramide/DSPE-PEG2k-Alfa [40/48/10/2-x/x=0, 0.2]; algM-VHH×NbAlfa post-functionalization [w/w*=ligand to cargo ratio 0, 0.12, 0.25, 0.4; Final RNA concentration: 0.1 μg/μl). All formulation conditions lead to colloidal stable particles with a diameter between 100 to 150 nm and a PDI below 0.5. Diameter and PDI were determined via DLS measurement. Successful RNA incorporation was verified via Agarose gel electrophoresis. For transfection studies, 10 μl of respective formulations were pre-diluted in 50 μl X-Vivo 15 in a 96 deep-well plate. 1e6 thawed human PBMC were diluted in IMDM media without FCS and added to nanoparticle dilution (1000 ng RNA). After 30 min of incubation (37° C., 5% CO2) 900 μl IMDM+ HI FCS +IL4 and IL21 was added per well and cells were cultivated for additional 18 h (37° C., 5% CO2). In the following cell-type specific transfection (Thy1.1) was analyzed via flowcytometry. Depicted are the cell type specific Thy1.1 (Transfection in % multiplied with MFI, y-axes divided by 100) signals within viable CD14+ Monocytes, CD19+ B cells, CD3+ T cells.

As shown in FIG. 10, LNPs containing Alfa tag lipid and algM×NbAlfa show increased specific transfection of B-cells. Transfection of B cells is increased with increasing ligand amount while transfection of T cells and monocytes is not affected. Incubation of LNPs which do not contain the Alfa tag lipid with the algM×NbAlfa ligand does not increase transfection in any of the cell types proofing the successful conjugation strategy.

### Example 11: Preparation and Characterization of Functionalized RNA Lipoplexes

### 1. Description of the Methods

### 1.1 Preparation of Functionalized RNA Lipoplexes

Alfa tagged lipid particles/lipoplex can be prepared using a known aqueous/organic manufacturing protocol such as described in WO 2013/143683 or WO 2020/201383.

For preparing alfa-tagged liposomes a lipid mix consisting of DODMA, DSPC, cholesterol, C14-PSar(23)-Ac and DSPE-PEG2k-alfa peptide in a molar ratio of 47.5:10:38.3:4:0.2 was dissolved in organic solvent at 50 mM total lipid concentration and mixed with an aqueous phase using a standard syringe pump based set-up and a T mixing element.

RNA lipoplexes were prepared by an aqueous-aqueous protocol.

The RNA-lipoplex stock was then functionalized with the aCD3-VHH ligand and further diluted with respective buffer of choice supplemented with sucrose so that final conc. of RNA in the lipoplexes was 0.1 mg/ml and sucrose content in the final matrix was 10% w/v. In the benchmark settings, the RNA-lipoplexes were prepared at N/P ratio of 6:1.

### 1.2 Characterization of the RNA Formulations

Particle size was determined by dynamic light scattering using a DynaPro Plate Reader II (Wyatt, Dernbach, Germany). From the measurements, size (Z-average) and polydispersity indices (Pdl) were calculated from the cumulant analysis using Dynamics 7.8.1.3 software. For the measurement, samples were diluted 1:10 in water and analysis was performed in triplicates. pH was measured on 150 μL sample after calibration of the device with pH 4, 7, and 7 standards.

### 1.3 Freeze Thaw Studies

Freeze thaw studies were conducted by cycling the formulations from −20° C. (overnight) to +25° C. (2 h) for at least two times. The particles size and polydispersity index of the formulations was measured for freeze-thaw samples.

### 2. Description of the Experimental Results.

Size and PDI of alfa-tagged liposomes was determined for alfa-liposomes before LPXs manufacturing, alfa-tagged lipoplexes immediately after manufacturing (raw alfa-LPXs) and functionalized LPXs stored in 20 mM HEPES pH 6.0, 10% sucrose (aCD3-LPXs in pH 6) at final RNA concentration of 0.1 mg/mL. Functionalization was performed in a 13:1 v/v raw alfa-LPXs:aCD3-VHH ratio, at ligand/cargo ratio of 0.48 w/w. It was observed that alfa-tagged lipoplexes can be manufactured with microfluidics starting from alfa-tagged liposomes stabilized in the presence of sucrose, with controlled particles sizes. Moreover, alfa-tagged lipoplexes can be successfully functionalized with aCD3-VHH protein, resulting in particles of around ^{˜}137 nm in size, when stored in 20 mM HEPES pH 6, 10% sucrose.

FIGS. 11A and 11B show the results from freezing of aCD3-LPXs in 20 mM HEPES pH 6.0 at −20° C. and −80° C., respectively, and storage for at least 2 freeze/thaw cycles. As can be seen, no significant changes in size and PDI could be observed at both freezing temperatures tested.

### Example 12: T-Cell Targeting Using LNPs with Different Stealth Lipids and Alfa Lipids

LNPs were formulated with different lipid compositions (Cargo: Thy1.1 RNA/Luc RNA/Np proxy Venus 1:1:2 w/w; N/P ratio: 6; Lipid mix: HY501/Cholesterol/DSPC/stealth lipid/Alfa lipid. The lipid ratio has been selected as [47.5/40.5/10/1.8/0.2] for the following combinations of stealth lipid and Alfa lipid: C16 PEG2k Ceramide/DSPE PEG2k Alfa, DSPE PEG2k/DSPE PEG2k Alfa, DSPE pAEEA14/DSPE pAEEA14 Alfa or VitE pAEEA8/DSPE pAEEA14 Alfa. The lipid ratio has been selected as [47.5/38.5/10/3.8/0.2] for the following combinations of stealth lipid and Alfa lipid: VitE PEG1k/DSPE PEG2k Alfa or VitE pAEEA8/DSPE pAEEA14 Alfa. The LNPs were equipped with aCD3 VHH X NbAlfa ligand via post functionalization [w/w*=ligand to cargo ratio 0.48]; RNA concentration: 0.1 μg/μl). Diameter of all LNPs is between 100 to 170 nm with a PDI below 0.4 as determined via DLS measurement.

For transfection studies, 10 μl (1000 ng dose) of respective formulations were pre-diluted in 50 μl X-Vivo 15 in an ultra-low adhesion 96 well plate. 1e6 thawed human PBMC were diluted in 50 μl of 100% clotted PHS and added to nanoparticle dilution. After 30 min of incubation (37° C., 5% CO2) 30 μl of each transfection reaction was transferred to second ultra-low adhesion 96 well plate and 170 μl of X-Vivo 15 medium+100 U/ml IL2 was added per well. Cell dilutions were cultivated for additional 18 h (37° C., 5% CO2). In the following cell-type specific transfection (Thy1.1) was analyzed via flowcytometry. Depicted are the percentages of transfected cell (CD2 negative cells, CD19+ B cells, CD4+ T cells and CD8+ T cells) within all transfected PBMCs (Transfection, y-axes) per tested formulation condition (see lower table in FIG. 12).

As shown in FIG. 12, the experiment demonstrates the modular nature of the conjugation approach. As shown for LNPs, ligands can be attached to nanoparticles using Alfa lipids with varying lipid backbone and spacer type as well as spacer length. Good physicochemical properties of the nanoparticles (size, PDI and RNA encapsulation) can be achieved with all tested stealth/Alfa lipid combinations. The selection of the stealth lipid anchor has an impact on the T cell transfection efficiency. DSPE has a tendency towards lower transfection efficiency.

### Example 13: Ligand-Mediated Transfection of T Cells In Vivo

LNP were formulated with or without ligand (Cargo: Thy1.1/Luc RNA 1:1 w/w; N/P ratio: 12; Lipid mix: HY501/Chol/DSPC/C16-PEG-Ceramide/DSPE-PEG2k-Alfa [47.5/40.5/10/1.8/0.2].

The LNPs were equipped with aCD3 VHH X NbAlfa ligand via post functionalization [w/w*=ligand to cargo ratio 0.48]; RNA concentration: 0.1 μg/μl).

Diameter of all LNPs is between 90 to 110 nm with a PDI 0.3 as determined via DLS measurement. Successful RNA incorporation was verified via Agarose gel electrophoresis.

LNPs injected intramuscularly drain to popliteal, inguinal, axillary and brachial lymph nodes as well as to the spleen independently of ligand functionalization (see FIG. 13A). Within drainage organs, only ligand-functionalized LNPs showed highly specific T-cell transfection (see FIG. 13B) and T-cell activation (see FIG. 13C).

1. A compound of the formula: 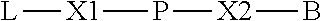
wherein
P comprises a polymer;
L comprises a hydrophobic moiety attached to the polymer;
B comprises a moiety attached to the polymer selected from the group consisting of an antibody or antibody-like molecule binding to a cell surface antigen, optionally wherein the antibody-like molecule comprises an antibody binding fragment or DARPin, an ALFA tag, and a moiety binding to an ALFA tag;
X1 is absent or a first linking moiety; and
X2 is absent or a second linking moiety.

2. The compound of claim 1, wherein
(A) the hydrophobic moiety comprises:
a moiety selected from vitamin E, dialkylamine, diacylglyceride and ceramide;
two C8-C24 hydrocarbon chains;
a lipid; and/or
a moiety selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof;
(B) the polymer is a hydrophilic polymer, optionally wherein the polymer is selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA), and combinations thereof; and/or
(C) X2 comprises the reaction product of a thiol or cysteine reactive group with a thiol or cysteine group of a compound comprising the moiety B, optionally wherein the thiol or cysteine reactive group comprises a maleimide group.

3-10. (canceled)

11. The compound of claim 1, which compound comprises the reaction product of 1,2-distearoyl-sn-glycero-3-phosphoethanolamine-N-[maleimide(polyethylene glycol)] with a compound comprising the formula SH(CH2)nC(O)—B, wherein n ranges from 1 to 5 and preferably n is 2, or which compound is a compound of the formula 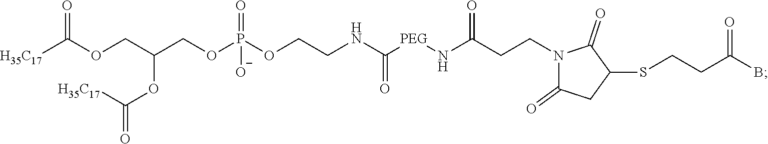
and/or
wherein the cell surface antigen is selected from the group consisting of CD4, CD8 and CD3.

12-18. (canceled)

19. A functionalized particle comprising one or more particle forming components, a nucleic acid payload, and a compound of claim 1, wherein the compound is incorporated into the particle through the hydrophobic moiety.

20. (canceled)

21. The functionalized particle of claim 19, wherein B comprises an ALFA tag or a moiety binding to an ALFA tag and the functionalized particle further comprises a compound of the formula: 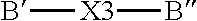
wherein
B′ comprises a moiety binding to B;
X3 is absent or a linking moiety; and
B″ comprises a moiety binding to a cell surface antigen, optionally wherein the compound of the formula B′-X3-B″ comprises a peptide or polypeptide.

22-26. (canceled)

27. The functionalized particle of claim 21, wherein B comprises an ALFA tag and B′ comprises a moiety binding to the ALFA tag, optionally wherein B′ comprises an ALFA tag and B comprises a moiety binding to the ALFA tag.

28. (canceled)

29. The functionalized particle of claim 27, wherein the moiety binding to an ALFA tag comprises an antibody or antibody-like molecule, optionally wherein the antibody-like molecule comprises an antibody binding fragment or DARPin.

30. (canceled)

31. (canceled)

32. The functionalized particle of claim 27, wherein moiety binding to the ALFA tag comprises a VHH domain comprising the CDR1 sequence VTISALNAMAMG, the CDR2 sequence AVSERGNAM, and the CDR3 sequence LEDRVDSFHDY.

33. The functionalized particle of claim 19, wherein the particle is a lipid particle, a polymer particle, or a mixture thereof, or wherein the particle is a non-viral particle or wherein the particle is a nanoparticle.

34. (canceled)

35. (canceled)

36. The functionalized particle of claim 19, wherein the nucleic acid comprises DNA and/or RNA, optionally wherein the nucleic acid comprises a nucleic acid encoding an antigen receptor.

37. (canceled)

38. The functionalized particle of claim 36, wherein the antigen receptor comprises a chimeric antigen receptor (CAR) or T cell receptor (TCR).

39. A method for delivering a nucleic acid to a cell expressing a cell surface antigen, comprising adding to the cell a composition comprising the functionalized particle of claim 19.

40. (canceled)

41. The method of claim 39, wherein the cell is present ex vivo.

42. The method of claim 39, wherein the cell is present in a subject and the method comprises administering the composition to the subject.

43. The method of claim 39, wherein the cell comprises an immune effector cell, optionally wherein the immune effector cell comprises a T cell or wherein the immune effector cell comprises a CD8+ and/or CD4+ T cell.

44. (canceled)

45. (canceled)

46. The method of claim 39, wherein the cell surface antigen is characteristic for an immune effector cell.

47. The method of claim 39, wherein the cell surface antigen is selected from the group consisting of CD4, CD8 and CD3 and/or wherein the nucleic acid comprises a nucleic acid encoding an antigen receptor, optionally wherein the antigen receptor comprises a chimeric antigen receptor (CAR) or T cell receptor (TCR).

48. (canceled)

49. (canceled)

50. The method of claim 39 which method is a method for preparing a genetically modified cell and/or a method for preparing an immune effector cell genetically modified to express an antigen receptor, optionally wherein the genetic modification is transient or stable and/or wherein the genetic modification takes place by a virus-based method, transposon-based method, or a gene editing-based method.

51. (canceled)

52. (canceled)

53. (canceled)

54. A method for preparing an immune effector cell genetically modified to express an antigen receptor, comprising adding to an immune effector cell a composition comprising the functionalized particle of claim 19, wherein the nucleic acid comprises a nucleic acid encoding an antigen receptor, optionally wherein the immune effector cell is present ex vivo or wherein the immune effector cell is present in a subject and the method comprises administering the composition to the subject.

55. The method of claim 54, wherein B or B″ comprises a moiety binding to a cell surface antigen on the immune effector cell.

56. (canceled)

57. (canceled)

58. The method of claim 54, wherein the immune effector cell comprises a T cell or wherein the immune effector cell comprises a CD8+ and/or CD4+ T cell.

59. (canceled)

60. The method of claim 54, wherein the cell surface antigen is characteristic for the immune effector cell, or wherein the cell surface antigen is selected from the group consisting of CD4, CD8 and CD3, or wherein the antigen receptor comprises a chimeric antigen receptor (CAR) or T cell receptor (TCR).

61. (canceled)

62. (canceled)

63. The method of claim 54, wherein the genetic modification is transient or stable or wherein the genetic modification takes place by a virus-based method, transposon-based method, or a gene editing-based method.

64. (canceled)

65. A method for treating a subject comprising:
(A)
(I) preparing ex vivo an immune effector cell genetically modified to express an antigen receptor using the method of claim 54, and
(II) administering the immune effector cell genetically modified to express an antigen receptor to the subject, or
(B)
preparing in vivo an immune effector cell genetically modified to express an antigen receptor using the method of claim 54.

66. (canceled)
